(19) United States
(12) Patent Application Publication
(10) Pub. No.: US 2024/0404625 A1
(43) Pub. Date: Dec. 5, 2024

(54) METHOD FOR ASSESSING HOMOLOGOUS RECOMBINATION DEFICIENCY IN OVARIAN CANCER CELLS

(71) Applicants: ZENTRUM FAMILIÄRER BRUST- UND EIERSTOCKKREBS UNIVERSITÄTSKLINIK KÖLN, Köln (DE); STICHTING HET NETHERLANDS KANKER INSITUUT - ANTONI VAN LEEUWENHOEK ZIEKENHUIS, Amsterdam (NL)

(72) Inventors: Rita SCHMUTZLER, Köln (DE); Philip SCHOUTEN, Cambridge (GB); Eric HAHNEN, Bonn (DE); Sabine LINN, Amsterdam (NL)

(21) Appl. No.: 18/688,194

(22) PCT Filed: Aug. 29, 2022
(86) PCT No.: PCT/EP2022/073958

§ 371 Date: Feb. 29, 2024

(30) Foreign Application Priority Data
Aug. 30, 2021 (EP) 21193861.8

(51) Int. Cl. G16B 20/10; G16H 20/10; G16H 40/67

(52) U.S. Cl. CPC G16B 20/10; G16H 20/10; G16H 40/67

(57) ABSTRACT

Provided are methods involved in assessing homologous recombination deficiency (HRD) status in a patient having ovarian cancer based on the determination of copy number variations (CNVs) in a genomic locus. Furthermore methods for predicting a patient's response to an anti-cancer therapy and thus to the identification of ovarian cancer patients likely to respond to a particular cancer treatment regime based on their HRD status are provided.

### CROSS-REFERENCE TO RELATED APPLICATIONS

This application is a 35 U.S.C. § 371 National Stage patent application of PCT/EP2022/073958, filed on 29 Aug. 2022, which claims the benefit of European patent application 21193861.8, filed on 30 Aug. 2021, the disclosures of which are incorporated herein by reference in their entirety.

### TECHNICAL FIELD

The present disclosure relates to methods involved in assessing homologous recombination deficiency (HRD) status in a patient having ovarian cancer based on the determination of copy number variants (CNVs) in a genomic locus. The disclosure also provides methods for predicting a patient's response to an anti-cancer therapy and thus to the identification of ovarian cancer patients likely to respond to a particular cancer treatment regime based on their HRD status.

### BACKGROUND

Epithelial ovarian cancer (OC) is the second leading cause of death among gynecologic cancers worldwide as there were 286,000 incident cases and mortality of 176,000 reported in 2017 (1). Since screening tools are ineffective and early clinical warning signs are rare, the majority of OC cases present in late clinical stages. Even though therapeutic strategies improved within the past years, the prognosis is still poor, with an average five-year survival-rate of 48.6% (2). Cytoreductive surgery aiming for complete resection, followed by platinum-based chemotherapy, has been the backbone of OC treatment for decades (3,4). Carboplatin, combined with paclitaxel+/−bevacizumab, as an initial systemic regimen leads to a response rate of approximately 80%, especially in high-grade serous ovarian cancer (HGSOC). Nevertheless, the disease of most patients recurs over time (5).

Recently, Poly (ADP-ribose) polymerase inhibitors (PARPi) were added to the therapeutic arsenal. Both the mainstay carboplatin/paclitaxel and these new strategies yield high responses in the overall population, which could be explained by homologous recombination deficiency (HRD) in a substantial proportion of OC (6).

Platinum compounds and PARPi exploit the HRD, by inducing DNA double-strand breaks or impeding its repair via synthetic lethality, leading to cell cycle arrest or death. The breast and ovarian cancer germline predisposition genes BRCA1 and BRCA2 play crucial roles in homologous recombination (7, 8), an essential, highly accurate DNA-repair process fixing double-strand breaks. Deleterious germline mutations with subsequent loss of heterozygosity (LOH) in BRCA1/2 can explain a subset of HR-deficient ovarian cancers, resulting in the registration of BRCA mutation analyses as companion diagnostics in specific OC (9) and metastasized breast cancer (10) settings. However, it was found that a larger proportion of OC displays a phenotype similar to germline (g) BRCA1/2-mutated cancers, so-called BRCAness (11). This is supported by preclinical analyses (12) and clinical trials of the three registered PARPi in OC (13-16) demonstrating survival benefits for a larger subgroup, and has led to their approval, independent of mutation status in most indications. Since these additional patients might benefit from a specific therapy, various potential biomarkers are under investigation, including single gene methylation, gene expression (profiles), copy number/LOH based assays, mutational signatures, and combinations (17-21).

Of these, the LOH-score (15) (Foundation Medicine, Cambridge, U.S.) and MyChoice® (22) (Myriad, Salt Lake City, U.S.) were applied within the trials mentioned above and demonstrated the ability to narrow the respective subgroup. This led to the first approval of an HRD assay (MyChoice®) as a companion diagnostic for applying niraparib in heavily pretreated OC patients (23). Two recent clinical trials (24, 25) evaluated this biomarker in the first-line setting, showing a benefit of a PARPi predominantly in the HRD-positive, but also in the HRD-negative subgroup. In contrast, within the PAOLA-1 trial (26), the test defined a subpopulation beyond BRCA mutation-carriers benefitting most from the addition of olaparib to bevacizumab maintenance therapy after carboplatin and paclitaxel. Only recently, the EMA recommended the approval of this combination therapy for HR-deficient OC defined by the presence of BRCA-mutations or genomic instability for first-line maintenance treatment accordingly (27).

Nonetheless, the exploration of further transparent tests that can easily be implemented and further elaborated on decentral platforms is still ongoing to improve the quest for predictive markers.

### SUMMARY

In general, the present disclosure relates to materials and methods involved in assessing cancer cells, in particular ovarian cancer cells, for homologous recombination deficiency (HRD). In particular, the present disclosure relates to a method of determining or predicting HRD status, i.e. the presence or absence of a HRD signature, in a subject having ovarian cancer and to a method of diagnosing the presence or absence of HRD in a patient sample, respectively.

The method comprises the determination of copy number variation (CNV) of a genomic locus in a test sample from a subject comprising cancer cells, e.g. a DNA test sample of cancer cells, wherein the locus is selected from a pool of genomic loci comprising or consisting of the loci set forth in Table 1 and/or in Table 2; and wherein similarity between the CNVs of the pool of genomic loci in the test sample from the subject and the CNVs of a corresponding pool of genomic loci in the DNA of a reference sample of BRCA-like mutated cancer cells and a control sample of BRCA non-mutated cancer cells, is indicative for the HR deficiency status in the subject, wherein (i) similarity between the CNVs of the test sample and the CNVs of the reference sample identifies the subject as HR deficient; and/or (ii) similarity between the CNVs of the test sample and the CNVs of the control sample identifies the subject as not being HR deficient. CNVs include losses and gains of genomic segments. The similarity is defined as distance measure between the centroids of the two classes, i.e. the control and reference sample, and the centroid of the test sample.

A centroid of a class, which is in the present case the control and reference sample, respectively, is constructed by shrinking the class centroid by an optimization routine towards the overall centroid of both classes after standardizing by the within-class standard deviation for each CNVs.

Preferably, a similarity score, i.e. a posterior probability score, is computed by providing a shrunken centroid value which is derived from the CNV of the genomic locus and wherein the comparison of the shrunken centroids of the test sample from the subject with the shrunken centroids of the reference sample and the shrunken centroids of the control sample is converted into a posterior probability score according to Tibshirani PNAS 2002 (29). This score ranges from 0-1, with 0-0.49 being non-BRCA-like and 0.5-1 being BRCA-like, wherein this definition is set during the training, wherein it has been defined that the BRCA-like class is the one of interest.

Thus, in one embodiment the method of the present disclosure comprises (a) determining copy number variation (CNV) of a genomic locus in a test sample from a subject comprising cancer cells, e.g. a DNA test sample of cancer cells, wherein the locus is selected from a pool of genomic loci comprising or consisting of the loci set forth in Table 1 and/or Table 2; and (b) providing a shrunken centroid value which is derived from the CNV of the genomic locus, and wherein a posterior probability score of ≥0.5 identifies the subject as being HRD; and/or wherein a posterior probability score of <0.5 identifies the subject as not being HRD, wherein the posterior probability score is obtainable by comparison of the shrunken centroid values derived from the CNVs of the pool of genomic loci in the DNA of the cancer cells in the test sample from the subject with the shrunken centroid values derived from the CNVs of a reference set of samples of BRCA-like mutated cancer cells and of control samples of BRCA non-mutated cancer cells.

The method of the present disclosure optionally comprises the transmission of the result to the subject or to a third party like a physician or genetic counselor.

Furthermore, a posterior probability score of ≥0.5 representing a higher similarity between the CNVs of the test sample and the CNVs of the reference sample than the control sample identifies the subject as being a likely responder to an anti-cancer therapy. A posterior probability score of <0.5 representing a higher similarity between the CNVs of the test sample and the CNVs of the control sample than the reference sample, respectively identifies the subject as not being a likely responder to an anti-cancer therapy.

The present disclosure is also directed to a method of predicting the response of a subject having cancer, in particular ovarian cancer, to an anti-cancer therapy, i.e. to a cancer treatment regimen, wherein the method comprises the steps as defined above. In particular, a posterior probability score of ≥0.5 and respectively a similarity between the CNVs of the test sample and the CNVs of the reference sample indicates an increased likelihood that the subject will respond to the anti-cancer therapy, and/or a posterior probability score of <0.5 and respectively a similarity between the CNVs of the test sample and the CNVs of the control sample indicates an increased likelihood that the subject will not respond to the anti-cancer therapy. The method of the present optionally comprises the transmission of the result to the subject or to a third party like a physician or genetic counselor.

In a preferred embodiment, the anti-cancer therapy/the cancer treatment regime is selected from homologous recombination deficiency-targeting drugs, drugs that directly cause double strand DNA breaks, and drugs that indirectly cause double strand DNA breaks or where the drug or dose of the drug has been identified in mechanistic studies to target homologous recombination deficiency, like for example (but not limited to) interstrand crosslinking agents, platinum compounds, PARP inhibitors, anthracyclines, as well as topoisomerase I and II inhibitors, preferably the anti-cancer therapy/the cancer treatment regime comprises PARP inhibitors or DNA double strand break inducing agents, e.g. platinum compounds and interstrand crosslinking agents.

In one embodiment of the method of the present disclosure, the determination of the CNV is performed by DNA sequencing, preferably by low coverage whole genome sequencing (LG-WGS). In one embodiment of the method of the present disclosure, determination of the CNVs is performed prior to administration of the anti-cancer therapy.

Optionally, the method of the present disclosure further comprises recommending a subject which has been identified as being HRD and/or having an increased likelihood of responding to the anti-cancer therapy, a treatment regimen comprising homologous recombination deficiency-targeting drugs, drugs that directly cause double strand DNA breaks, drugs that indirectly cause double strand DNA breaks; and/or drugs which have been identified in mechanistic studies to target homologous recombination deficiency, for example interstrand crosslinking agents, platinum compounds, PARP inhibitors, anthracyclines, and topoisomerase I and II inhibitors, preferably PARP inhibitors or DNA double strand break inducing agents, e.g. platinum compounds and interstrand crosslinking agents. Alternatively, the method of the present disclosure may optionally comprise recommending a subject which has been identified as not being HRD and/or not having an increased likelihood of responding to the anti-cancer therapy, a standard treatment in the specific clinical setting of the patient, which might not include homologous recombination deficiency-targeting drugs, drugs that directly cause double strand DNA breaks, drugs that indirectly cause double strand DNA breaks, and/or drugs which have been identified in mechanistic studies to target homologous recombination deficiency.

The disclosure further relates to a method of treating cancer, in particular ovarian cancer. Such method is in general analogous to the methods described above and differs in that a particular treatment regimen is administered (recommended, prescribed, initiated, continued, etc.) based at least in part of the calculated posterior probability scores and the similarity determinations as explained above. The treatment regime is preferably the treatment regime as defined above. Furthermore, the present disclosure relates to a treatment regime comprising homologous recombination deficiency-targeting drugs, drugs that directly cause double strand DNA breaks, drugs that indirectly cause double strand DNA breaks, or that otherwise have been identified as specifically targeting homologous recombination deficient tumors for use in treating ovarian cancer in a subject, wherein said subject has been identified as HRD and/or identified to have an increased likelihood of responding to the anti-cancer treatment regimen according to the method as described above. Thus, the present disclosure also relates to the use of one or more drugs selected from the group consisting of homologous recombination deficiency-targeting drugs, drugs that directly cause double strand DNA breaks, drugs that indirectly cause double strand DNA breaks; and/or drugs which have been identified in mechanistic studies to target homologous recombination deficiency, like for example DNA double strand break inducing agents, interstrand crosslinking agents, platinum compounds, and PARP inhibitors in the manufacture of a medicament useful for treating a cancer, in particular ovarian cancer, in a patient identified as having (or as having had) a cancer cell determined to have HRD (e.g. an HRD signature) as described herein.

Unless otherwise defined, all technical and scientific terms used herein have the same meaning as commonly understood by one of ordinary skill in the art to which this disclosure pertains. The details of one or more embodiments of the disclosure are set forth in the description and accompanying drawings as well as in the Examples below.

TABLE 1
BRCA-1 like classifier. The indicated locations are on genome version ‘hg18’. Of
course, in case a genome version changes, the person skilled in the art is able to
assign the below mentioned positions to any new and/or different genome version. Thus, variations
in the Start and End positions are also encompassed by the present disclosure. Furthermore, the
sc_control values, the sc_BRCA1 values and/or the sd values can vary dependent
on the number of probes used and/or if the classifiers are retrained.

| Order | Clone | chrom | Start | End | sc_control | sc_BRCA1 | sd |
|---|---|---|---|---|---|---|---|
| 3535 | GS-232-B23 | 1 | 125000 | 275000 | −0.1949 | −0.1766 | 0.5283 |
| 3537 | GS-62-L8 | 1 | 125050 | 275050 | −0.1949 | −0.1766 | 0.5283 |
| 2305 | RP4-785P20 | 1 | 3214521 | 3355092 | −0.1972 | −0.1322 | 0.4771 |
| 145 | RP1-37J18 | 1 | 4476787 | 4608114 | −0.1222 | −0.0946 | 0.4422 |
| 3 | RP11-484P7 | 1 | 9083937 | 9084661 | −0.0873 | −0.0568 | 0.4502 |
| 2313 | RP4-575L21 | 1 | 10075582 | 10170003 | −0.0480 | −0.0475 | 0.4569 |
| 153 | RP4-635E18 | 1 | 10986297 | 11099065 | −0.0480 | −0.0475 | 0.4569 |
| 2317 | RP4-539L13 | 1 | 11483207 | 11618991 | −0.0480 | −0.0475 | 0.4569 |
| 3358 | RP11-196P5 | 1 | 11765728 | 11932249 | −0.0480 | −0.0475 | 0.4569 |
| 57 | RP11-174G17 | 1 | 18255125 | 18370415 | −0.0880 | −0.0947 | 0.4135 |
| 2325 | RP4-540O3 | 1 | 19184476 | 19258712 | −0.0880 | −0.0947 | 0.4135 |
| 165 | RP4-745E8 | 1 | 20480049 | 20558818 | −0.0880 | −0.0947 | 0.4135 |
| 2221 | RP11-132G19 | 1 | 21921946 | 22097107 | −0.0586 | −0.0777 | 0.4213 |
| 2845 | RP1-184J9 | 1 | 23210636 | 23311487 | −0.0582 | −0.0646 | 0.4130 |
| 3491 | RP11-304H10 | 1 | 24599009 | 24781759 | −0.0580 | −0.0892 | 0.4081 |
| 685 | RP3-39819 | 1 | 25064029 | 25159269 | −0.0580 | −0.0892 | 0.4081 |
| 689 | RP4-633N17 | 1 | 27233517 | 27352531 | −0.0580 | −0.0821 | 0.4097 |
| 2849 | RP1-144C9 | 1 | 27575323 | 27652644 | −0.0580 | −0.0821 | 0.4097 |
| 900 | RP1-159A19 | 1 | 27730312 | 27853599 | −0.0580 | −0.0821 | 0.4097 |
| 693 | RP4-669K10 | 1 | 28699604 | 28835423 | −0.0577 | −0.0643 | 0.4013 |
| 2853 | RP3-437I16 | 1 | 29481122 | 29601506 | −0.0577 | −0.0643 | 0.4013 |
| 2857 | RP5-893G23 | 1 | 30314279 | 30401538 | −0.0577 | −0.0643 | 0.4013 |
| 2306 | RP4-739H11 | 1 | 40830672 | 40960522 | 0.0556 | 0.0493 | 0.3948 |
| 2761 | RP11-182I23 | 1 | 44522378 | 44608805 | 0.0554 | 0.0533 | 0.3974 |
| 3891 | RP11-420M12 | 1 | 45348865 | 45523040 | 0.0554 | 0.0533 | 0.3974 |
| 146 | RP4-534D1 | 1 | 45524153 | 45623939 | 0.0554 | 0.0533 | 0.3974 |
| 605 | RP11-330M19 | 1 | 48002300 | 48202667 | −0.0583 | 0.0255 | 0.3611 |
| 2769 | RP11-428D12 | 1 | 48917309 | 49103008 | −0.0583 | 0.0255 | 0.3611 |
| 2310 | RP5-1013G21 | 1 | 50343064 | 50459755 | −0.0583 | 0.0255 | 0.3611 |
| 150 | RP1-86A18 | 1 | 50594537 | 50802796 | −0.0583 | 0.0255 | 0.3611 |
| 609 | RP11-253A20 | 1 | 51655989 | 51797658 | −0.0583 | 0.0255 | 0.3611 |
| 2210 | RP11-117D22 | 1 | 53582566 | 53632120 | −0.0583 | 0.0442 | 0.3617 |
| 2500 | RP11-243A18 | 1 | 53802593 | 53986331 | −0.0583 | 0.0442 | 0.3617 |
| 154 | RP5-1043G4 | 1 | 54894393 | 55023548 | −0.0583 | 0.0442 | 0.3617 |
| 2318 | RP5-1070D5 | 1 | 55675959 | 55690981 | −0.0583 | 0.0442 | 0.3617 |
| 158 | RP4-710M16 | 1 | 56595677 | 56724344 | −0.0432 | 0.0284 | 0.3684 |
| 1286 | RP11-20F20 | 1 | 57456434 | 57628847 | −0.0434 | 0.0355 | 0.3646 |
| 2139 | RP11-20F20 | 1 | 57456434 | 57628847 | −0.0434 | 0.0355 | 0.3646 |
| 50 | RP11-342F23 | 1 | 58740479 | 58790441 | −0.0421 | 0.0389 | 0.3917 |
| 2214 | RP11-37M11 | 1 | 59939363 | 60081655 | −0.0421 | 0.0389 | 0.3917 |
| 2322 | RP4-668G5 | 1 | 61044546 | 61191260 | −0.0424 | 0.0312 | 0.3857 |
| 162 | RP4-662P1 | 1 | 62131197 | 62274422 | −0.0424 | 0.0312 | 0.3857 |
| 2218 | RP11-5P4 | 1 | 63222826 | 63340665 | −0.0434 | 0.0050 | 0.3647 |
| 2326 | RP4-537F10 | 1 | 64206685 | 64303918 | −0.0434 | 0.0050 | 0.3647 |
| 166 | RP4-700A9 | 1 | 65371357 | 65460412 | −0.0432 | −0.0003 | 0.3674 |
| 2846 | RP4-759M20 | 1 | 66677274 | 66805768 | −0.0432 | −0.0003 | 0.3674 |
| 686 | RP4-547N15 | 1 | 67239504 | 67370748 | −0.0432 | −0.0003 | 0.3674 |
| 2850 | RP5-1033K19 | 1 | 68609845 | 68712983 | −0.0432 | −0.0003 | 0.3674 |
| 58 | RP11-412F21 | 1 | 69215584 | 69364440 | −0.0432 | −0.0003 | 0.3674 |
| 690 | RP5-944F13 | 1 | 69875730 | 69989244 | −0.0432 | −0.0003 | 0.3674 |
| 2680 | RP11-419I14 | 1 | 70178367 | 70348261 | −0.0432 | −0.0003 | 0.3674 |
| 2854 | RP5-1006B11 | 1 | 70717489 | 70814713 | −0.0432 | −0.0003 | 0.3674 |
| 2222 | RP11-175G14 | 1 | 71927563 | 72045513 | −0.0432 | −0.0003 | 0.3674 |
| 62 | RP11-261J10 | 1 | 72813123 | 72983939 | −0.0432 | −0.0003 | 0.3674 |
| 2858 | RP4-716B13 | 1 | 73625850 | 73764334 | −0.0432 | −0.0003 | 0.3674 |
| 2226 | RP11-250D8 | 1 | 74474909 | 74503104 | −0.0431 | −0.0065 | 0.3701 |
| 66 | RP11-25F16 | 1 | 75234702 | 75388843 | −0.0431 | −0.0065 | 0.3701 |
| 698 | RP5-963M5 | 1 | 76640776 | 76740455 | −0.0431 | −0.0065 | 0.3701 |
| 2230 | RP11-306I4 | 1 | 77815414 | 77880693 | −0.0431 | −0.0065 | 0.3701 |
| 70 | RP11-143H12 | 1 | 78953798 | 79135725 | −0.0425 | −0.0019 | 0.3830 |
| 523 | RP11-111N8 | 1 | 78981093 | 79179795 | −0.0425 | −0.0019 | 0.3830 |
| 706 | RP4-653E17 | 1 | 81843695 | 81898453 | −0.0421 | 0.0044 | 0.3923 |
| 2750 | RP11-398N17 | 1 | 82504367 | 82595121 | −0.0423 | 0.0145 | 0.3883 |
| 590 | RP11-338F23 | 1 | 83494488 | 83678494 | −0.0423 | 0.0145 | 0.3883 |
| 1826 | RP11-28I21 | 1 | 83633989 | 83797164 | −0.0423 | 0.0145 | 0.3883 |
| 4296 | RP11-28I21 | 1 | 83633989 | 83797164 | −0.0423 | 0.0145 | 0.3883 |
| 2754 | RP11-118B23 | 1 | 84728027 | 84853591 | −0.0422 | 0.0199 | 0.3906 |
| 2307 | RP4-604P14 | 1 | 84964828 | 85107408 | −0.0422 | 0.0199 | 0.3906 |
| 147 | RP4-722L13 | 1 | 85912330 | 85939332 | −0.0422 | 0.0274 | 0.3892 |
| 2311 | RP4-612B15 | 1 | 86920706 | 87046530 | −0.0422 | 0.0274 | 0.3892 |
| 151 | RP5-1043L3 | 1 | 87450562 | 87550402 | −0.0422 | 0.0274 | 0.3892 |
| 594 | RP11-427B20 | 1 | 88626861 | 88735131 | −0.0424 | 0.0390 | 0.3858 |
| 2758 | RP11-413E1 | 1 | 89836299 | 90021480 | −0.0424 | 0.0390 | 0.3858 |
| 598 | RP11-298P9 | 1 | 90346809 | 90447017 | −0.0424 | 0.0390 | 0.3858 |
| 2315 | RP4-665J23 | 1 | 91067447 | 91151453 | −0.0424 | 0.0390 | 0.3858 |
| 155 | RP4-621B10 | 1 | 92289742 | 92432467 | −0.0426 | 0.0506 | 0.3824 |
| 2319 | RP5-1033H22 | 1 | 93768309 | 93910326 | −0.0425 | 0.0468 | 0.3844 |
| 2762 | RP11-366L18 | 1 | 94637145 | 94733145 | −0.0421 | 0.0420 | 0.3926 |
| 602 | RP11-14O19 | 1 | 95577488 | 95621690 | −0.0425 | 0.0311 | 0.3835 |
| 2766 | RP11-146P11 | 1 | 95756215 | 95929262 | −0.0425 | 0.0311 | 0.3835 |
| 606 | RP11-122C9 | 1 | 96868095 | 97055472 | −0.0425 | 0.0311 | 0.3835 |
| 2770 | RP11-128G13 | 1 | 97892513 | 98003512 | −0.0431 | 0.0390 | 0.3711 |
| 610 | RP11-17C2 | 1 | 98735430 | 98839846 | −0.0431 | 0.0390 | 0.3711 |
| 4184 | RP11-260K3 | 1 | 99670780 | 99835817 | −0.0431 | 0.0390 | 0.3711 |
| 2211 | RP11-413P11 | 1 | 99710406 | 99835827 | −0.0431 | 0.0390 | 0.3711 |
| 159 | RP4-549L20 | 1 | 101075342 | 101207111 | −0.0431 | 0.0390 | 0.3711 |
| 51 | RP11-421L21 | 1 | 101220661 | 101408985 | −0.0431 | 0.0390 | 0.3711 |
| 2215 | RP11-202K23 | 1 | 102366306 | 102506926 | −0.0431 | 0.0390 | 0.3711 |
| 55 | RP11-118G19 | 1 | 105468331 | 105581702 | −0.0431 | 0.0390 | 0.3711 |
| 2219 | RP11-90H3 | 1 | 106102370 | 106261386 | −0.0431 | 0.0390 | 0.3711 |
| 163 | RP5-916A15 | 1 | 106759849 | 106884451 | −0.0431 | 0.0390 | 0.3711 |
| 2327 | RP5-1077K16 | 1 | 107388307 | 107478600 | −0.0436 | 0.0503 | 0.3599 |
| 59 | RP11-28P8 | 1 | 108330015 | 108512967 | −0.0436 | 0.0503 | 0.3599 |
| 2847 | RP5-831G13 | 1 | 109816089 | 109885092 | −0.0436 | 0.0503 | 0.3599 |
| 2223 | RP11-284N8 | 1 | 110897276 | 111088458 | −0.0436 | 0.0503 | 0.3599 |
| 63 | RP11-88H9 | 1 | 112311566 | 112481164 | −0.0439 | 0.0442 | 0.3536 |
| 687 | RP4-770C6 | 1 | 112861328 | 112960667 | −0.0439 | 0.0442 | 0.3536 |
| 2227 | RP11-31F15 | 1 | 113304869 | 113373751 | −0.0216 | 0.0442 | 0.3542 |
| 2525 | RP5-1000E10 | 1 | 115009146 | 115149253 | −0.0215 | 0.0501 | 0.3566 |
| 691 | RP4-663N10 | 1 | 115660445 | 115837510 | −0.0216 | 0.0625 | 0.3541 |
| 2859 | RP4-610L12 | 1 | 119405867 | 119480650 | 0.3336 | 0.1175 | 0.6047 |
| 71 | RP11-418J17 | 1 | 119501341 | 119658498 | 0.3336 | 0.1175 | 0.6047 |
| 591 | RP11-326G21 | 1 | 143613589 | 143768403 | 0.3503 | 0.1544 | 0.5030 |
| 2755 | RP11-315I20 | 1 | 144149933 | 144341526 | 0.1930 | 0.1765 | 0.3689 |
| 595 | RP11-533N14 | 1 | 145509311 | 145622017 | 0.1930 | 0.1765 | 0.3689 |
| 2759 | RP11-301M17 | 1 | 146153735 | 146204124 | 0.1930 | 0.1765 | 0.3689 |
| 2763 | RP11-98D18 | 1 | 149916726 | 150080114 | 0.1938 | 0.1755 | 0.3533 |
| 603 | RP11-216N14 | 1 | 151954673 | 152104838 | 0.1933 | 0.1840 | 0.3644 |
| 607 | RP11-422P24 | 1 | 152158475 | 152334301 | 0.1933 | 0.1840 | 0.3644 |
| 2767 | RP11-307C12 | 1 | 153110663 | 153292828 | 0.1933 | 0.1840 | 0.3644 |
| 2771 | RP11-98F1 | 1 | 153539661 | 153543654 | 0.1933 | 0.1840 | 0.3644 |
| 611 | RP11-172I6 | 1 | 154346199 | 154350727 | 0.1933 | 0.1840 | 0.3644 |
| 60 | RP11-430G6 | 1 | 161223665 | 161388993 | 0.1627 | 0.1619 | 0.3808 |
| 3336 | RP11-180L13 | 1 | 163383027 | 163567283 | 0.1627 | 0.1619 | 0.3808 |
| 64 | RP11-306I1 | 1 | 163809021 | 163884934 | 0.1627 | 0.1619 | 0.3808 |
| 148 | RP1-97P20 | 1 | 168048730 | 168151811 | 0.1631 | 0.1473 | 0.3721 |
| 68 | RP11-277C14 | 1 | 170129727 | 170285667 | 0.1630 | 0.1462 | 0.3745 |
| 2232 | RP11-415M14 | 1 | 174374254 | 174386794 | 0.1629 | 0.1471 | 0.3772 |
| 2752 | RP11-12M5 | 1 | 177942453 | 178115483 | 0.1629 | 0.1419 | 0.3761 |
| 592 | RP11-46A10 | 1 | 179093654 | 179232659 | 0.1629 | 0.1419 | 0.3761 |
| 2756 | RP11-538D16 | 1 | 180286063 | 180360577 | 0.1629 | 0.1419 | 0.3761 |
| 596 | RP11-71D4 | 1 | 180974670 | 181046133 | 0.1628 | 0.1331 | 0.3776 |
| 3350 | RP11-181K3 | 1 | 181329737 | 181493820 | 0.1629 | 0.1412 | 0.3770 |
| 2760 | RP11-293B7 | 1 | 181919047 | 181949386 | 0.1624 | 0.1294 | 0.3864 |
| 164 | RP5-936P19 | 1 | 184949810 | 185050756 | 0.1926 | 0.1312 | 0.3804 |
| 2764 | RP11-108M21 | 1 | 185443394 | 185614032 | 0.1926 | 0.1312 | 0.3804 |
| 604 | RP11-336D15 | 1 | 187406225 | 187410218 | 0.1926 | 0.1312 | 0.3804 |
| 2768 | RP11-445K1 | 1 | 188252300 | 188442462 | 0.1929 | 0.1414 | 0.3727 |
| 612 | RP11-142L4 | 1 | 190417312 | 190511486 | 0.1929 | 0.1435 | 0.3726 |
| 2623 | RP11-239J11 | 1 | 191355372 | 191478235 | 0.1929 | 0.1390 | 0.3737 |
| 463 | RP11-435N12 | 1 | 193197924 | 193377705 | 0.1919 | 0.1487 | 0.3941 |
| 467 | RP11-75C23 | 1 | 195654060 | 195710013 | 0.1926 | 0.1344 | 0.3788 |
| 2631 | RP11-404H1 | 1 | 196603836 | 196750596 | 0.1926 | 0.1344 | 0.3788 |
| 471 | RP11-152M20 | 1 | 197828613 | 197913713 | 0.1926 | 0.1344 | 0.3788 |
| 2635 | RP11-469A15 | 1 | 198548937 | 198714432 | 0.1926 | 0.1344 | 0.3788 |
| 2639 | RP11-572A16 | 1 | 200418733 | 200596076 | 0.1919 | 0.0979 | 0.3946 |
| 479 | RP11-480I12 | 1 | 200974582 | 201126597 | 0.1919 | 0.0979 | 0.3946 |
| 1988 | RP11-739N20 | 1 | 202583794 | 202750872 | 0.1908 | 0.0824 | 0.4181 |
| 2643 | RP11-563I16 | 1 | 202668576 | 202832483 | 0.1908 | 0.0824 | 0.4181 |
| 3895 | RP11-430C7 | 1 | 202750873 | 202882857 | 0.1908 | 0.0718 | 0.4189 |
| 3163 | RP11-534L20 | 1 | 204706319 | 204814007 | 0.1562 | 0.0497 | 0.4209 |
| 1003 | RP11-564A8 | 1 | 205087940 | 205280504 | 0.1562 | 0.0497 | 0.4209 |
| 1007 | RP11-385M4 | 1 | 207338650 | 207512566 | 0.1556 | 0.0223 | 0.4334 |
| 3171 | RP11-354K1 | 1 | 209859608 | 209904751 | 0.1556 | 0.0223 | 0.4334 |
| 1011 | RP11-286E7 | 1 | 211304553 | 211481242 | 0.1556 | 0.0223 | 0.4334 |
| 3175 | RP11-434B7 | 1 | 211479243 | 211599451 | 0.1556 | 0.0182 | 0.4345 |
| 1015 | RP11-323K10 | 1 | 213140173 | 213231454 | 0.1560 | 0.0195 | 0.4249 |
| 3179 | RP11-438G15 | 1 | 213686067 | 213758612 | 0.1560 | 0.0195 | 0.4249 |
| 1019 | RP11-66M7 | 1 | 215209941 | 215373866 | 0.1560 | 0.0195 | 0.4249 |
| 3183 | RP11-241C9 | 1 | 215371867 | 215473408 | 0.1560 | 0.0195 | 0.4249 |
| 1718 | RP11-224O19 | 1 | 216532600 | 216705043 | 0.1561 | 0.0270 | 0.4233 |
| 1023 | RP11-392O17 | 1 | 217480858 | 217657906 | 0.1564 | 0.0419 | 0.4168 |
| 2624 | RP11-492K2 | 1 | 217542815 | 217743251 | 0.1564 | 0.0419 | 0.4168 |
| 464 | RP11-332J14 | 1 | 218698681 | 218866026 | 0.1564 | 0.0590 | 0.4167 |
| 2628 | RP11-308L13 | 1 | 219894313 | 219919554 | 0.2203 | 0.0689 | 0.4122 |
| 2632 | RP11-239E10 | 1 | 221352933 | 221534398 | 0.2203 | 0.0689 | 0.4122 |
| 472 | RP11-105I12 | 1 | 221890430 | 222001842 | 0.2203 | 0.0689 | 0.4122 |
| 2636 | RP11-100E13 | 1 | 222762747 | 222926492 | 0.2203 | 0.0689 | 0.4122 |
| 2640 | RP11-375H24 | 1 | 225133240 | 225194140 | 0.2604 | 0.0896 | 0.4223 |
| 480 | RP11-276J4 | 1 | 225649911 | 225807728 | 0.2604 | 0.0896 | 0.4223 |
| 2848 | RP4-799G3 | 1 | 226955298 | 227082936 | 0.2606 | 0.0982 | 0.4183 |
| 688 | RP4-679K16 | 1 | 227665287 | 227690355 | 0.2606 | 0.0982 | 0.4183 |
| 2644 | RP11-99J16 | 1 | 228957724 | 229009252 | 0.2606 | 0.0982 | 0.4183 |
| 484 | RP11-87P4 | 1 | 230784451 | 230922071 | 0.2603 | 0.0993 | 0.4253 |
| 3164 | RP11-528D17 | 1 | 231700764 | 231814183 | 0.2603 | 0.0993 | 0.4253 |
| 3168 | RP11-214M7 | 1 | 235542200 | 235725748 | 0.2606 | 0.0920 | 0.4181 |
| 1008 | RP11-433N10 | 1 | 236639314 | 236664614 | 0.2606 | 0.0920 | 0.4181 |
| 3172 | RP11-359A17 | 1 | 237591402 | 237780910 | 0.2606 | 0.0920 | 0.4181 |
| 1012 | RP11-80B9 | 1 | 238957512 | 239131145 | 0.2606 | 0.0920 | 0.4181 |
| 3176 | RP11-553N16 | 1 | 240002343 | 240146309 | 0.2606 | 0.0920 | 0.4181 |
| 1020 | RP11-399B15 | 1 | 243222295 | 243431391 | 0.2606 | 0.0920 | 0.4181 |
| 2625 | RP11-438H8 | 1 | 246013817 | 246181047 | 0.2606 | 0.0920 | 0.4181 |
| 1375 | GS-167-K11 | 1 | 246904719 | 247054719 | 0.2606 | 0.0920 | 0.4181 |
| 1377 | GS-160-H23 | 1 | 247124719 | 247274719 | 0.2606 | 0.0920 | 0.4181 |
| 3539 | GS-8-L3 | 2 | 255000 | 405000 | 0.1037 | 0.0763 | 0.4608 |
| 3541 | GS-892-G20 | 2 | 255050 | 405050 | 0.1037 | 0.0763 | 0.4608 |
| 3931 | RP11-352J11 | 2 | 2250133 | 2390017 | 0.1060 | 0.0999 | 0.4108 |
| 2484 | RP11-214N9 | 2 | 9588276 | 9685110 | 0.1068 | 0.1067 | 0.3946 |
| 648 | RP11-83M8 | 2 | 10189946 | 10392995 | 0.1061 | 0.1029 | 0.4094 |
| 3387 | RP11-345J13 | 2 | 11150863 | 11208496 | 0.1073 | 0.0818 | 0.3825 |
| 1226 | RP11-333O1 | 2 | 12700490 | 12904007 | 0.1078 | 0.0701 | 0.3708 |
| 293 | RP11-132H1 | 2 | 13501024 | 13636845 | 0.0674 | 0.0625 | 0.3712 |
| 3326 | RP11-163G14 | 2 | 14523373 | 14693180 | 0.0674 | 0.0625 | 0.3712 |
| 825 | RP11-136K18 | 2 | 14848829 | 14852339 | 0.0674 | 0.0625 | 0.3712 |
| 2019 | RP11-247H16 | 2 | 15119640 | 15208710 | 0.1015 | 0.0667 | 0.3590 |
| 4306 | RP11-247H16 | 2 | 15119640 | 15208710 | 0.1015 | 0.0667 | 0.3590 |
| 1199 | RP11-355H10 | 2 | 15932229 | 16129188 | 0.1015 | 0.0667 | 0.3590 |
| 4070 | RP11-549D18 | 2 | 16245950 | 16435643 | 0.1015 | 0.0667 | 0.3590 |
| 4192 | RP11-262D22 | 2 | 17121302 | 17130519 | 0.1015 | 0.0667 | 0.3590 |
| 2058 | RP11-111J6 | 2 | 18490085 | 18666774 | 0.1015 | 0.0667 | 0.3590 |
| 2135 | RP11-17L8 | 2 | 20037859 | 20215537 | 0.0992 | 0.0900 | 0.4091 |
| 3422 | RP11-17L8 | 2 | 20037859 | 20215537 | 0.0992 | 0.0900 | 0.4091 |
| 3412 | RP11-23F10 | 2 | 21327465 | 21369660 | 0.1016 | 0.0689 | 0.3589 |
| 311 | RP11-368O18 | 2 | 22411645 | 22617616 | 0.1016 | 0.0747 | 0.3586 |
| 2929 | RP11-560C7 | 2 | 22998568 | 23181018 | 0.1018 | 0.0876 | 0.3524 |
| 761 | RP11-557N21 | 2 | 23723602 | 23881796 | 0.1020 | 0.0946 | 0.3497 |
| 3627 | RP11-557N21 | 2 | 23723602 | 23881796 | 0.1020 | 0.0946 | 0.3497 |
| 3335 | RP11-169L20 | 2 | 24704977 | 24873681 | 0.1021 | 0.0880 | 0.3465 |
| 306 | RP11-404P12 | 2 | 25696967 | 25870860 | 0.1021 | 0.0880 | 0.3465 |
| 3666 | RP11-106G13 | 2 | 26821201 | 26988191 | 0.1021 | 0.0880 | 0.3465 |
| 843 | RP11-373D23 | 2 | 28414457 | 28572121 | 0.1021 | 0.0880 | 0.3465 |
| 3925 | RP11-328L16 | 2 | 29261112 | 29451760 | 0.1021 | 0.0880 | 0.3465 |
| 2477 | RP11-395B14 | 2 | 39539270 | 39691425 | 0.1339 | 0.1356 | 0.3440 |
| 3016 | RP11-389K20 | 2 | 52045693 | 52231824 | 0.1221 | 0.1286 | 0.3454 |
| 1234 | RP11-335O22 | 2 | 52193265 | 52373212 | 0.1224 | 0.1346 | 0.3525 |
| 2411 | RP11-7H13 | 2 | 52903958 | 53101085 | 0.1224 | 0.1317 | 0.3515 |
| 3527 | RP11-508L23 | 2 | 53793757 | 53906745 | 0.1242 | 0.1500 | 0.3906 |
| 971 | RP11-30C22 | 2 | 54809162 | 54917397 | 0.1234 | 0.1439 | 0.3740 |
| 1173 | RP11-152O18 | 2 | 55792089 | 55796228 | 0.1234 | 0.1439 | 0.3740 |
| 546 | RP11-481J13 | 2 | 56202050 | 56343886 | 0.1234 | 0.1439 | 0.3740 |
| 1249 | RP11-422B1 | 2 | 58184911 | 58334911 | 0.1238 | 0.1244 | 0.3810 |
| 2024 | RP11-260K8 | 2 | 58959329 | 59141348 | 0.1238 | 0.1244 | 0.3810 |
| 14 | RP11-479F13 | 2 | 61286584 | 61378667 | 0.1226 | 0.1146 | 0.4215 |
| 4188 | RP11-261A24 | 2 | 63239468 | 63339572 | 0.1246 | 0.1211 | 0.3795 |
| 2922 | RP11-568N6 | 2 | 64478270 | 64676357 | 0.1246 | 0.1211 | 0.3795 |
| 1481 | RP11-263L17 | 2 | 65586797 | 65745895 | 0.1249 | 0.1152 | 0.3731 |
| 233 | RP11-554F14 | 2 | 66394108 | 66576570 | 0.1249 | 0.1152 | 0.3731 |
| 2814 | RP11-67K11 | 2 | 68007238 | 68161125 | 0.1260 | 0.0867 | 0.3483 |
| 1327 | RP11-304A15 | 2 | 68860250 | 69024244 | 0.1063 | 0.0805 | 0.3358 |
| 2095 | RP11-304A15 | 2 | 68860250 | 69024244 | 0.1063 | 0.0805 | 0.3358 |
| 864 | RP11-245N4 | 2 | 70374522 | 70512358 | 0.1063 | 0.0772 | 0.3353 |
| 3613 | CTB-1011K1 | 2 | 70877487 | 70877988 | 0.1063 | 0.0772 | 0.3353 |
| 545 | RP11-467P9 | 2 | 71113938 | 71283193 | 0.0825 | 0.0771 | 0.3285 |
| 1782 | RP11-343N14 | 2 | 72247343 | 72428558 | 0.0827 | 0.0811 | 0.3233 |
| 1569 | RP11-434P11 | 2 | 73670503 | 73883575 | 0.0827 | 0.0811 | 0.3233 |
| 257 | RP11-1P9 | 2 | 74335426 | 74424778 | 0.0827 | 0.0811 | 0.3233 |
| 1230 | RP11-335E8 | 2 | 76173054 | 76356807 | 0.0832 | 0.0780 | 0.3136 |
| 4065 | RP11-535E19 | 2 | 77131545 | 77193800 | 0.0832 | 0.0780 | 0.3136 |
| 2955 | RP11-9O10 | 2 | 77953060 | 77979896 | 0.0808 | 0.0597 | 0.3641 |
| 4267 | RP11-419E14 | 2 | 78739646 | 78927111 | 0.0825 | 0.0688 | 0.3277 |
| 1 | RP11-458O14 | 2 | 79819775 | 79920395 | 0.0825 | 0.0688 | 0.3277 |
| 1323 | RP11-303I4 | 2 | 80657637 | 80808678 | 0.0825 | 0.0688 | 0.3277 |
| 2091 | RP11-303I4 | 2 | 80657637 | 80808678 | 0.0825 | 0.0688 | 0.3277 |
| 2035 | RP11-89C12 | 2 | 81618971 | 81763529 | 0.0825 | 0.0688 | 0.3277 |
| 559 | RP11-495B16 | 2 | 82611679 | 82714465 | 0.0825 | 0.0688 | 0.3277 |
| 4067 | RP11-513O19 | 2 | 85513676 | 85688465 | 0.0832 | 0.0782 | 0.3136 |
| 4038 | RP11-301O19 | 2 | 86132487 | 86301208 | 0.0832 | 0.0782 | 0.3136 |
| 3500 | RP11-316G9 | 2 | 89561552 | 89772753 | 0.0828 | 0.0655 | 0.3213 |
| 432 | RP11-34G16 | 2 | 95675761 | 95861540 | 0.1844 | 0.0860 | 0.3789 |
| 274 | RP11-139J6 | 2 | 96130286 | 96151127 | 0.1844 | 0.0882 | 0.3791 |
| 1780 | RP11-363D14 | 2 | 96531332 | 96597791 | 0.1840 | 0.0626 | 0.3885 |
| 3721 | RP11-363D14 | 2 | 96531332 | 96597791 | 0.1840 | 0.0626 | 0.3885 |
| 1366 | RP11-542D13 | 2 | 97472514 | 97700939 | 0.1840 | 0.0626 | 0.3885 |
| 4058 | RP11-547F10 | 2 | 98011952 | 98199250 | 0.1840 | 0.0626 | 0.3885 |
| 417 | RP11-38C17 | 2 | 99112042 | 99179521 | 0.1840 | 0.0600 | 0.3881 |
| 4074 | RP11-549H5 | 2 | 99955224 | 100125797 | 0.1842 | 0.0685 | 0.3839 |
| 2928 | RP11-30G7 | 2 | 100125798 | 100273807 | 0.1842 | 0.0685 | 0.3839 |
| 1862 | RP11-299H21 | 2 | 101071911 | 101277486 | 0.1842 | 0.0685 | 0.3839 |
| 4270 | RP11-451C2 | 2 | 101984766 | 102030378 | 0.0854 | 0.0679 | 0.3360 |
| 3007 | RP11-373G23 | 2 | 102712967 | 102891930 | 0.0850 | 0.0607 | 0.3455 |
| 2114 | RP11-451G1 | 2 | 103816333 | 103981851 | 0.0610 | 0.0488 | 0.3339 |
| 1239 | RP11-350B7 | 2 | 104912262 | 105047433 | 0.0610 | 0.0445 | 0.3344 |
| 4064 | RP11-526D2 | 2 | 105850219 | 106012104 | 0.0610 | 0.0445 | 0.3344 |
| 1360 | RP11-519H15 | 2 | 106847336 | 107035284 | 0.0612 | 0.0359 | 0.3302 |
| 234 | RP11-566O4 | 2 | 109113179 | 109319769 | 0.0612 | 0.0359 | 0.3302 |
| 1589 | RP11-438K19 | 2 | 111491772 | 111673269 | 0.0613 | 0.0397 | 0.3284 |
| 2467 | RP11-368A17 | 2 | 113091657 | 113230334 | 0.0613 | 0.0397 | 0.3284 |
| 560 | RP11-504G11 | 2 | 115568405 | 115730304 | 0.0523 | 0.0251 | 0.3316 |
| 2542 | RP11-412A2 | 2 | 115793564 | 115936236 | 0.0528 | 0.0322 | 0.3198 |
| 1246 | RP11-339P1 | 2 | 116380901 | 116514763 | 0.0531 | 0.0376 | 0.3142 |
| 2813 | RP11-59P4 | 2 | 117162762 | 117348560 | 0.0531 | 0.0376 | 0.3142 |
| 1546 | RP11-425F6 | 2 | 118087040 | 118291673 | 0.0531 | 0.0376 | 0.3142 |
| 1588 | RP11-425F6 | 2 | 118087040 | 118291673 | 0.0531 | 0.0376 | 0.3142 |
| 1790 | RP11-19E11 | 2 | 119282245 | 119406283 | 0.0531 | 0.0376 | 0.3142 |
| 659 | RP11-77A13 | 2 | 119406284 | 119549525 | 0.0531 | 0.0376 | 0.3142 |
| 99 | RP11-69O6 | 2 | 121704118 | 121873929 | 0.0531 | 0.0376 | 0.3142 |
| 2703 | RP11-490M17 | 2 | 122905850 | 122950222 | 0.0528 | 0.0451 | 0.3196 |
| 4227 | RP11-202H24 | 2 | 122950223 | 122990154 | 0.0528 | 0.0451 | 0.3196 |
| 1338 | RP11-298E18 | 2 | 124928149 | 125078149 | 0.0520 | 0.0413 | 0.3365 |
| 958 | RP11-48K7 | 2 | 127180319 | 127253883 | 0.0518 | 0.0340 | 0.3410 |
| 3696 | RP11-207G14 | 2 | 129652992 | 129799323 | 0.0518 | 0.0340 | 0.3410 |
| 2588 | RP11-32C20 | 2 | 130418035 | 130583445 | 0.0518 | 0.0340 | 0.3410 |
| 3531 | RP11-510C1 | 2 | 131164571 | 131306971 | 0.0518 | 0.0340 | 0.3410 |
| 4224 | RP11-209H16 | 2 | 131497953 | 131669023 | 0.0518 | 0.0340 | 0.3410 |
| 1796 | RP11-24O13 | 2 | 131993057 | 131995050 | 0.0518 | 0.0340 | 0.3410 |
| 2417 | RP11-1L22 | 2 | 132997590 | 132999583 | 0.0513 | 0.0459 | 0.3531 |
| 1498 | RP11-105K20 | 2 | 133513096 | 133688729 | 0.0513 | 0.0459 | 0.3531 |
| 4191 | RP11-250H22 | 2 | 133791576 | 133959201 | 0.0511 | 0.0507 | 0.3562 |
| 3737 | RP11-436E24 | 2 | 134654310 | 134835354 | 0.0509 | 0.0495 | 0.3603 |
| 2096 | RP11-355I13 | 2 | 136875551 | 137087299 | 0.0515 | 0.0385 | 0.3486 |
| 3943 | RP11-355I13 | 2 | 136875551 | 137087299 | 0.0515 | 0.0385 | 0.3486 |
| 858 | RP11-414I8 | 2 | 137366984 | 137538813 | 0.0613 | 0.0482 | 0.3541 |
| 113 | RP11-58C7 | 2 | 138125168 | 138212379 | 0.0613 | 0.0482 | 0.3541 |
| 2964 | RP11-12M21 | 2 | 139529620 | 139695761 | 0.0613 | 0.0482 | 0.3541 |
| 1252 | RP11-23H14 | 2 | 160688277 | 160856293 | 0.1116 | 0.1150 | 0.3743 |
| 4285 | RP11-440P12 | 2 | 161596464 | 161768144 | 0.1113 | 0.1408 | 0.3670 |
| 2383 | RP11-576I16 | 2 | 162682902 | 162846382 | 0.1113 | 0.1408 | 0.3670 |
| 798 | RP11-5J4 | 2 | 164165855 | 164240910 | 0.1112 | 0.1479 | 0.3650 |
| 4221 | RP11-218M2 | 2 | 165772205 | 165838175 | 0.1112 | 0.1479 | 0.3650 |
| 2953 | RP11-2I8 | 2 | 166510094 | 166698159 | 0.1110 | 0.1384 | 0.3591 |
| 2992 | RP11-125M15 | 2 | 167445843 | 167642208 | 0.1115 | 0.1469 | 0.3719 |
| 784 | RP11-11N16 | 2 | 168899618 | 169089882 | 0.1115 | 0.1469 | 0.3719 |
| 3481 | RP11-285F23 | 2 | 169206756 | 169395118 | 0.1115 | 0.1469 | 0.3719 |
| 1832 | RP11-141H20 | 2 | 169789426 | 169953565 | 0.1118 | 0.1542 | 0.3785 |
| 221 | RP11-551O2 | 2 | 169823899 | 169935265 | 0.1118 | 0.1542 | 0.3785 |
| 3623 | RP11-551O2 | 2 | 169823899 | 169935265 | 0.1118 | 0.1542 | 0.3785 |
| 3012 | RP11-389F18 | 2 | 170471086 | 170690261 | 0.1120 | 0.1559 | 0.3828 |
| 2011 | RP11-244E6 | 2 | 171080925 | 171261146 | 0.1120 | 0.1612 | 0.3824 |
| 3316 | RP11-176L20 | 2 | 172134584 | 172269397 | 0.1120 | 0.1612 | 0.3824 |
| 3942 | RP11-343H10 | 2 | 173168753 | 173325586 | 0.1126 | 0.1503 | 0.3949 |
| 1154 | RP11-158L8 | 2 | 173997059 | 174130810 | 0.1126 | 0.1503 | 0.3949 |
| 2718 | RP11-483E17 | 2 | 175049767 | 175231429 | 0.1140 | 0.1658 | 0.4258 |
| 3684 | RP11-205B19 | 2 | 175978396 | 176145371 | 0.1376 | 0.1658 | 0.4310 |
| 1713 | RP11-157E8 | 2 | 177052080 | 177205498 | 0.1376 | 0.1658 | 0.4310 |
| 2031 | RP11-250N10 | 2 | 178079879 | 178252093 | 0.1370 | 0.1576 | 0.4168 |
| 2802 | RP11-65L3 | 2 | 178966384 | 179139011 | 0.1368 | 0.1619 | 0.4126 |
| 1901 | RP11-535B12 | 2 | 179945353 | 180115969 | 0.1363 | 0.1588 | 0.4021 |
| 301 | RP11-391P1 | 2 | 180331396 | 180481646 | 0.1363 | 0.1588 | 0.4021 |
| 2720 | RP11-504G5 | 2 | 181817089 | 181987870 | 0.1363 | 0.1532 | 0.4028 |
| 421 | RP11-38H6 | 2 | 183145945 | 183309874 | 0.1363 | 0.1532 | 0.4028 |
| 2938 | RP11-571N13 | 2 | 184429689 | 184467238 | 0.1363 | 0.1532 | 0.4028 |
| 1784 | RP11-363K21 | 2 | 185140061 | 185332568 | 0.1357 | 0.1673 | 0.3878 |
| 1830 | RP11-29E4 | 2 | 185796056 | 186001245 | 0.1360 | 0.1490 | 0.3962 |
| 857 | RP11-400O18 | 2 | 186322693 | 186426356 | 0.1496 | 0.1521 | 0.4013 |
| 839 | RP11-372A6 | 2 | 187410393 | 187617316 | 0.1290 | 0.1631 | 0.3818 |
| 4249 | RP11-372A6 | 2 | 187410393 | 187617316 | 0.1290 | 0.1631 | 0.3818 |
| 2980 | RP11-123G24 | 2 | 188197232 | 188357186 | 0.1290 | 0.1631 | 0.3818 |
| 1680 | RP11-172N20 | 2 | 188855759 | 189025686 | 0.1290 | 0.1631 | 0.3818 |
| 2118 | RP11-455J20 | 2 | 190280560 | 190364670 | 0.1427 | 0.1735 | 0.3822 |
| 3609 | RP11-1021H16 | 2 | 190364671 | 190468806 | 0.1420 | 0.1669 | 0.3673 |
| 2074 | RP11-235L22 | 2 | 190468807 | 190574136 | 0.1420 | 0.1669 | 0.3673 |
| 2030 | RP11-284E5 | 2 | 191161960 | 191354774 | 0.1420 | 0.1669 | 0.3673 |
| 649 | RP11-59L22 | 2 | 192914919 | 193076759 | 0.1421 | 0.1617 | 0.3703 |
| 3944 | RP11-363G3 | 2 | 193770313 | 193936253 | 0.1421 | 0.1617 | 0.3703 |
| 2401 | RP11-1A1 | 2 | 195231805 | 195411755 | 0.1415 | 0.1519 | 0.3572 |
| 3751 | RP11-1A1 | 2 | 195231805 | 195411755 | 0.1415 | 0.1519 | 0.3572 |
| 1157 | RP11-149P12 | 2 | 195826913 | 195994198 | 0.1409 | 0.1424 | 0.3438 |
| 1789 | RP11-15J24 | 2 | 205174820 | 205347296 | 0.1091 | 0.1047 | 0.3471 |
| 1233 | RP11-325M10 | 2 | 206054358 | 206242420 | 0.1091 | 0.1047 | 0.3471 |
| 553 | RP11-470J24 | 2 | 206846958 | 206994419 | 0.1089 | 0.1067 | 0.3513 |
| 841 | RP11-396J8 | 2 | 210627910 | 210820013 | 0.1315 | 0.1185 | 0.3884 |
| 4030 | RP11-300D24 | 2 | 211890302 | 212037638 | 0.1313 | 0.1148 | 0.3914 |
| 769 | RP11-560C24 | 2 | 213160973 | 213345198 | 0.1318 | 0.1168 | 0.3820 |
| 3662 | RP11-105N14 | 2 | 213713153 | 213872184 | 0.1318 | 0.1168 | 0.3820 |
| 3710 | RP11-427F22 | 2 | 214750215 | 214905472 | 0.1162 | 0.0810 | 0.3587 |
| 4272 | RP11-427F22 | 2 | 214750215 | 214905472 | 0.1162 | 0.0810 | 0.3587 |
| 1179 | RP11-307A11 | 2 | 217064016 | 217224225 | 0.1163 | 0.0620 | 0.3568 |
| 1351 | RP11-506C8 | 2 | 217224025 | 217403884 | 0.1163 | 0.0620 | 0.3568 |
| 3413 | RP11-423F9 | 2 | 217797627 | 218010176 | 0.1157 | 0.0707 | 0.3689 |
| 2456 | RP11-378A13 | 2 | 218827093 | 219018647 | 0.1153 | 0.0561 | 0.3784 |
| 4063 | RP11-512O7 | 2 | 220236911 | 220431668 | 0.1159 | 0.0512 | 0.3655 |
| 1570 | RP11-444B5 | 2 | 221335168 | 221420064 | 0.1159 | 0.0512 | 0.3655 |
| 119 | RP11-71J24 | 2 | 222493290 | 222674059 | 0.1159 | 0.0512 | 0.3655 |
| 3289 | RP11-397D2 | 2 | 222855303 | 223012639 | 0.1159 | 0.0512 | 0.3655 |
| 250 | RP11-3I22 | 2 | 223189276 | 223293801 | 0.1159 | 0.0512 | 0.3655 |
| 2381 | RP11-551D18 | 2 | 224018057 | 224147409 | 0.1159 | 0.0512 | 0.3655 |
| 3512 | RP11-516C4 | 2 | 225556155 | 225739388 | 0.1159 | 0.0512 | 0.3655 |
| 1597 | RP11-86O17 | 2 | 226629534 | 226739370 | 0.1151 | 0.0628 | 0.3815 |
| 3655 | RP11-86O17 | 2 | 226629534 | 226739370 | 0.1151 | 0.0628 | 0.3815 |
| 4236 | RP11-211K17 | 2 | 227039788 | 227203287 | 0.1155 | 0.0435 | 0.3728 |
| 3317 | RP11-149O3 | 2 | 228366695 | 228540039 | 0.1165 | 0.0251 | 0.3505 |
| 107 | RP11-70L16 | 2 | 229316582 | 229434960 | 0.0954 | 0.0251 | 0.3519 |
| 4290 | RP11-457P23 | 2 | 230386956 | 230587338 | 0.0547 | 0.0252 | 0.3581 |
| 4271 | RP11-419H23 | 2 | 231787899 | 231964428 | 0.0543 | 0.0119 | 0.3671 |
| 978 | RP11-52C8 | 2 | 232425628 | 232539910 | 0.0392 | 0.0119 | 0.3693 |
| 2148 | RP11-52C8 | 2 | 232425628 | 232539910 | 0.0392 | 0.0119 | 0.3693 |
| 1512 | RP11-91N19 | 2 | 232987280 | 233143792 | 0.0390 | 0.0006 | 0.3751 |
| 3611 | RP11-534J17 | 2 | 233993547 | 234101158 | 0.0387 | 0.0082 | 0.3818 |
| 4057 | RP11-534J17 | 2 | 233993547 | 234101158 | 0.0387 | 0.0082 | 0.3818 |
| 1477 | RP11-263G22 | 2 | 234593055 | 234701766 | 0.0387 | 0.0082 | 0.3818 |
| 4314 | RP11-263G22 | 2 | 234593055 | 234701766 | 0.0387 | 0.0082 | 0.3818 |
| 2816 | RP11-84G18 | 2 | 235987110 | 236175494 | 0.0378 | −0.0116 | 0.4011 |
| 2168 | RP11-497D24 | 2 | 238251662 | 238463736 | 0.0376 | −0.0350 | 0.4053 |
| 3953 | RP11-15L18 | 2 | 238922713 | 239091229 | −0.0201 | −0.0578 | 0.4145 |
| 1894 | RP11-546M8 | 2 | 239997825 | 240140444 | −0.0201 | −0.0578 | 0.4145 |
| 9 | RP11-463B12 | 2 | 240559949 | 240731368 | −0.0201 | −0.0578 | 0.4145 |
| 1381 | GS-1011-O17 | 2 | 242636149 | 242786149 | −0.2528 | −0.2307 | 0.4603 |
| 3536 | RG-172-I13 | 2 | 242636199 | 242786199 | −0.2528 | −0.2307 | 0.4603 |
| 3545 | GS-1186-B18 | 3 | 225000 | 375000 | −0.0486 | −0.0119 | 0.5295 |
| 660 | RP11-86C13 | 3 | 768338 | 958276 | −0.0546 | −0.0427 | 0.3985 |
| 3661 | RP11-97C16 | 3 | 3117275 | 3274532 | −0.0546 | −0.0427 | 0.3985 |
| 4216 | RP11-95E11 | 3 | 3134172 | 3304965 | −0.0546 | −0.0427 | 0.3985 |
| 2404 | RP11-10H6 | 3 | 3156042 | 3326580 | −0.0546 | −0.0427 | 0.3985 |
| 67 | RP11-183N22 | 3 | 4492889 | 4493650 | −0.0546 | −0.0427 | 0.3985 |
| 4242 | RP11-238A9 | 3 | 4562897 | 4652965 | −0.0546 | −0.0427 | 0.3985 |
| 2445 | RP11-129J10 | 3 | 5125758 | 5180955 | −0.0546 | −0.0427 | 0.3985 |
| 2537 | RP11-277D17 | 3 | 6595704 | 6636734 | −0.0546 | −0.0322 | 0.3974 |
| 1714 | RP11-167K17 | 3 | 6749027 | 6942291 | −0.0546 | −0.0322 | 0.3974 |
| 3625 | CTB-1114A6 | 3 | 7498176 | 7537606 | −0.0546 | −0.0322 | 0.3974 |
| 3929 | RP11-329A2 | 3 | 7634301 | 7760384 | −0.0548 | −0.0238 | 0.3930 |
| 3658 | RP11-105K13 | 3 | 8869825 | 9037105 | −0.0547 | −0.0206 | 0.3955 |
| 2113 | RP11-439F4 | 3 | 9803852 | 9993473 | −0.0546 | −0.0161 | 0.3976 |
| 2093 | RP11-382A21 | 3 | 10066548 | 10067175 | −0.0530 | 0.0113 | 0.4325 |
| 3030 | RP11-382A21 | 3 | 10066548 | 10067175 | −0.0530 | 0.0113 | 0.4325 |
| 1739 | RP11-438J1 | 3 | 10137483 | 10347535 | −0.0544 | −0.0084 | 0.4035 |
| 2044 | RP11-94A14 | 3 | 10613350 | 10788431 | −0.0544 | −0.0084 | 0.4035 |
| 1804 | RP11-25C10 | 3 | 12420266 | 12590814 | −0.0534 | 0.0269 | 0.4247 |
| 3346 | RP11-163D23 | 3 | 12590779 | 12735990 | −0.0534 | 0.0269 | 0.4247 |
| 1698 | RP11-165B2 | 3 | 13797747 | 13964637 | −0.0534 | 0.0408 | 0.4246 |
| 1336 | RP11-316A10 | 3 | 14886274 | 15046975 | −0.0545 | 0.0213 | 0.4013 |
| 1335 | RP11-305L22 | 3 | 15628045 | 15764879 | −0.0545 | 0.0213 | 0.4013 |
| 1488 | RP11-255O19 | 3 | 15780358 | 15940642 | −0.0545 | 0.0213 | 0.4013 |
| 2280 | RP11-80D24 | 3 | 17181366 | 17345270 | −0.0546 | 0.0271 | 0.3974 |
| 259 | RP11-8D23 | 3 | 17814951 | 17960794 | −0.0546 | 0.0271 | 0.3974 |
| 961 | RP11-44M22 | 3 | 19336535 | 19494336 | −0.0546 | 0.0271 | 0.3974 |
| 955 | RP11-27J5 | 3 | 20636015 | 20828584 | −0.0546 | 0.0271 | 0.3974 |
| 1176 | RP11-180N14 | 3 | 21314358 | 21484066 | −0.0354 | 0.0272 | 0.3928 |
| 3434 | RP11-16E8 | 3 | 21574260 | 21742237 | −0.0354 | 0.0272 | 0.3928 |
| 1540 | RP11-208G16 | 3 | 22372629 | 22514821 | −0.0354 | 0.0272 | 0.3928 |
| 2043 | RP11-89F18 | 3 | 23404804 | 23575751 | −0.0354 | 0.0272 | 0.3928 |
| 1266 | RP11-18L17 | 3 | 24167259 | 24311442 | −0.0357 | 0.0359 | 0.3867 |
| 4287 | RP11-421F9 | 3 | 25447987 | 25518546 | −0.0356 | 0.0480 | 0.3885 |
| 4241 | RP11-226E22 | 3 | 26718003 | 26882790 | −0.0346 | 0.0301 | 0.4103 |
| 789 | RP11-2G22 | 3 | 27349564 | 27531283 | −0.0337 | 0.0427 | 0.4295 |
| 2420 | RP11-11L6 | 3 | 28645912 | 28824358 | −0.0346 | 0.0310 | 0.4107 |
| 1185 | RP11-30G4 | 3 | 28813263 | 28966852 | −0.0345 | 0.0355 | 0.4113 |
| 2049 | RP11-103N21 | 3 | 29910388 | 30018478 | −0.0346 | 0.0329 | 0.4099 |
| 658 | RP11-69K20 | 3 | 30275511 | 30447593 | −0.0346 | 0.0329 | 0.4099 |
| 1177 | RP11-7I16 | 3 | 30704315 | 30705070 | −0.0347 | 0.0142 | 0.4066 |
| 438 | RP11-48E16 | 3 | 31634821 | 31812073 | −0.0259 | 0.0272 | 0.4072 |
| 4056 | RP11-524O15 | 3 | 32472740 | 32650842 | −0.0255 | 0.0483 | 0.4155 |
| 109 | RP11-56P22 | 3 | 34981197 | 35131137 | −0.0255 | 0.0483 | 0.4155 |
| 308 | RP11-380G10 | 3 | 35590144 | 35766135 | −0.0255 | 0.0483 | 0.4155 |
| 2977 | RP11-134C18 | 3 | 36562366 | 36744618 | −0.0265 | 0.0615 | 0.3951 |
| 4293 | RP11-134C18 | 3 | 36562366 | 36744618 | −0.0265 | 0.0615 | 0.3951 |
| 3897 | RP11-129K12 | 3 | 36866838 | 37036150 | −0.0265 | 0.0615 | 0.3951 |
| 2707 | RP11-491D6 | 3 | 37036151 | 37136521 | −0.0265 | 0.0615 | 0.3951 |
| 1777 | RP11-331G2 | 3 | 39137073 | 39311657 | −0.0266 | 0.0767 | 0.3915 |
| 2806 | RP11-66G8 | 3 | 39724379 | 39836467 | −0.0266 | 0.0767 | 0.3915 |
| 1580 | RP11-424L2 | 3 | 40986834 | 41155572 | −0.0266 | 0.0767 | 0.3915 |
| 2105 | RP11-424L2 | 3 | 40986834 | 41155572 | −0.0266 | 0.0767 | 0.3915 |
| 1353 | RP11-527M19 | 3 | 41172081 | 41354719 | −0.0266 | 0.0767 | 0.3915 |
| 3569 | RP4-613B23 | 3 | 42629196 | 42784970 | −0.0271 | 0.0508 | 0.3815 |
| 3758 | RP11-189H19 | 3 | 44567485 | 44737886 | −0.0274 | 0.0427 | 0.3748 |
| 3876 | RP11-189H19 | 3 | 44567485 | 44737886 | −0.0274 | 0.0427 | 0.3748 |
| 2431 | RP11-111P21 | 3 | 45438197 | 45497978 | −0.0263 | 0.0583 | 0.3999 |
| 1504 | RP11-91E8 | 3 | 46036466 | 46093400 | −0.0731 | −0.0103 | 0.4072 |
| 1948 | RP4-544D10 | 3 | 47295529 | 47384745 | −0.0068 | 0.0104 | 0.3893 |
| 2741 | RP11-88B8 | 3 | 47860083 | 47993747 | −0.0068 | 0.0104 | 0.3893 |
| 2504 | RP1-289H18 | 3 | 48080483 | 48208886 | −0.0068 | 0.0104 | 0.3893 |
| 3424 | RP11-24C3 | 3 | 48357034 | 48512503 | −0.0068 | 0.0104 | 0.3893 |
| 242 | RP11-3B7 | 3 | 49256500 | 49393338 | −0.0068 | 0.0104 | 0.3893 |
| 108 | RP11-78O10 | 3 | 49843918 | 50006856 | −0.0069 | −0.0042 | 0.3880 |
| 2727 | RP11-314A5 | 3 | 51791953 | 51927169 | −0.0250 | −0.0233 | 0.3907 |
| 2728 | RP11-447A21 | 3 | 52556292 | 52743025 | −0.0250 | −0.0233 | 0.3907 |
| 1030 | RP5-966M1 | 3 | 52743026 | 52839812 | −0.0248 | −0.0123 | 0.3946 |
| 630 | RP11-122d19 | 3 | 54007389 | 54188449 | −0.0247 | −0.0169 | 0.3959 |
| 2972 | RP11-122D19 | 3 | 54007389 | 54188449 | −0.0247 | −0.0169 | 0.3959 |
| 2397 | RP11-554O1 | 3 | 54623583 | 54778937 | −0.0247 | −0.0169 | 0.3959 |
| 2801 | RP11-58O15 | 3 | 55610036 | 55797581 | −0.0250 | −0.0071 | 0.3900 |
| 276 | RP11-120C2 | 3 | 57340288 | 57375009 | −0.0250 | −0.0032 | 0.3892 |
| 3694 | RP11-229A12 | 3 | 57370434 | 57534933 | −0.0250 | −0.0032 | 0.3892 |
| 120 | RP11-80H18 | 3 | 58149203 | 58327212 | −0.0253 | 0.0068 | 0.3834 |
| 1695 | RP11-170K19 | 3 | 59701342 | 59875950 | −0.0256 | −0.0009 | 0.3778 |
| 2786 | RP11-94d19 | 3 | 60756896 | 60927079 | −0.0256 | −0.0009 | 0.3778 |
| 4208 | RP11-94D19 | 3 | 60756896 | 60927079 | −0.0256 | −0.0009 | 0.3778 |
| 2487 | RP11-154D3 | 3 | 62016747 | 62182595 | −0.0258 | 0.0040 | 0.3736 |
| 1606 | RP11-204J18 | 3 | 62190612 | 62347194 | −0.0259 | −0.0078 | 0.3715 |
| 2079 | RP11-204J18 | 3 | 62190612 | 62347194 | −0.0259 | −0.0078 | 0.3715 |
| 1518 | RP11-108A8 | 3 | 63254921 | 63396321 | −0.0260 | 0.0025 | 0.3675 |
| 783 | RP11-9A1 | 3 | 64198694 | 64357759 | −0.0260 | 0.0103 | 0.3678 |
| 3960 | RP11-24O17 | 3 | 66890138 | 67018894 | −0.0563 | 0.0216 | 0.3867 |
| 2272 | RP11-79C12 | 3 | 68008697 | 68146379 | −0.0563 | 0.0216 | 0.3867 |
| 3640 | RP11-253K11 | 3 | 68472738 | 68646025 | −0.0563 | 0.0216 | 0.3867 |
| 3333 | RP11-152N21 | 3 | 69417957 | 69541169 | −0.0560 | 0.0420 | 0.3939 |
| 2432 | RP11-118O11 | 3 | 70741634 | 70792905 | −0.0560 | 0.0420 | 0.3939 |
| 3861 | RP11-154H23 | 3 | 71622956 | 71816508 | −0.0560 | 0.0339 | 0.3941 |
| 3532 | RP11-522N9 | 3 | 72457282 | 72633500 | −0.0564 | 0.0525 | 0.3848 |
| 1476 | RP11-252O10 | 3 | 73463946 | 73612108 | −0.0564 | 0.0543 | 0.3853 |
| 1802 | RP11-20B7 | 3 | 73854898 | 74026003 | −0.0564 | 0.0543 | 0.3853 |
| 260 | RP11-11L10 | 3 | 74234096 | 74391234 | −0.0564 | 0.0543 | 0.3853 |
| 4071 | RP11-514J23 | 3 | 76310308 | 76477952 | −0.0562 | 0.0730 | 0.3885 |
| 4229 | RP11-220O14 | 3 | 77652537 | 77654544 | −0.0320 | 0.0860 | 0.3908 |
| 1809 | RP11-16M12 | 3 | 78395760 | 78564986 | 0.0076 | 0.0944 | 0.4012 |
| 1785 | RP11-333I20 | 3 | 78932852 | 79097134 | 0.0078 | 0.1077 | 0.4042 |
| 3720 | RP11-333I20 | 3 | 78932852 | 79097134 | 0.0078 | 0.1077 | 0.4042 |
| 423 | RP11-26H10 | 3 | 80133749 | 80297117 | 0.0071 | 0.0928 | 0.3906 |
| 1367 | RP11-510B7 | 3 | 80611850 | 80795918 | 0.0071 | 0.0928 | 0.3906 |
| 4289 | RP11-442C9 | 3 | 81649526 | 81740108 | 0.0069 | 0.0886 | 0.3857 |
| 3706 | RP11-425D6 | 3 | 81649532 | 81826325 | 0.0069 | 0.0886 | 0.3857 |
| 3748 | RP11-425D6 | 3 | 81649532 | 81826325 | 0.0069 | 0.0886 | 0.3857 |
| 1528 | RP11-206J21 | 3 | 82657795 | 82799473 | 0.0314 | 0.0936 | 0.3806 |
| 2476 | RP11-382L10 | 3 | 83811935 | 83922050 | 0.0311 | 0.1029 | 0.3752 |
| 2721 | RP11-474M18 | 3 | 84623842 | 84801874 | 0.0311 | 0.1029 | 0.3752 |
| 3750 | RP11-447J13 | 3 | 86090396 | 86268611 | 0.0308 | 0.0984 | 0.3692 |
| 640 | RP11-81P15 | 3 | 87044531 | 87223753 | 0.0308 | 0.0984 | 0.3692 |
| 2773 | RP11-81p15 | 3 | 87044531 | 87223753 | 0.0308 | 0.0984 | 0.3692 |
| 4039 | RP11-312H1 | 3 | 87569839 | 87730259 | 0.0308 | 0.0984 | 0.3692 |
| 1511 | RP11-88I7 | 3 | 95519622 | 95706420 | 0.1107 | 0.1335 | 0.3578 |
| 788 | RP11-12A13 | 3 | 96093190 | 96250469 | 0.1107 | 0.1335 | 0.3578 |
| 796 | RP11-12J13 | 3 | 97024240 | 97176860 | 0.0750 | 0.1229 | 0.3843 |
| 3521 | RP11-529P9 | 3 | 98524225 | 98714854 | 0.0744 | 0.1391 | 0.3704 |
| 3685 | RP11-214N20 | 3 | 99258539 | 99288219 | 0.0744 | 0.1391 | 0.3704 |
| 2448 | RP11-121C1 | 3 | 100490741 | 100545594 | 0.0744 | 0.1391 | 0.3704 |
| 2443 | RP11-114I8 | 3 | 101480701 | 101632377 | 0.1208 | 0.1276 | 0.3789 |
| 3122 | RP11-49I4 | 3 | 102993643 | 103144288 | 0.1214 | 0.1252 | 0.3933 |
| 562 | RP11-484I19 | 3 | 103202967 | 103378300 | 0.1214 | 0.1252 | 0.3933 |
| 2094 | RP11-484I19 | 3 | 103202967 | 103378300 | 0.1214 | 0.1252 | 0.3933 |
| 291 | RP11-115B22 | 3 | 105815245 | 105971443 | 0.1226 | 0.1375 | 0.4189 |
| 3664 | RP11-91B3 | 3 | 106787071 | 106966499 | 0.1226 | 0.1375 | 0.4189 |
| 2378 | RP11-561O4 | 3 | 108272967 | 108465288 | 0.1226 | 0.1375 | 0.4189 |
| 4204 | RP11-93N14 | 3 | 110118060 | 110272409 | 0.1226 | 0.1375 | 0.4189 |
| 1329 | RP11-286P15 | 3 | 111576106 | 111691384 | 0.1389 | 0.1466 | 0.4177 |
| 3855 | RP11-169N13 | 3 | 120928481 | 121091573 | 0.1343 | 0.1136 | 0.4165 |
| 3881 | RP11-73F12 | 3 | 121091573 | 121205101 | 0.1345 | 0.1207 | 0.4119 |
| 4156 | RP11-767L7 | 3 | 121148280 | 121326590 | 0.1345 | 0.1207 | 0.4119 |
| 1240 | RP11-359H3 | 3 | 121653170 | 121848109 | 0.1345 | 0.1207 | 0.4119 |
| 2182 | RP11-480B17 | 3 | 122380934 | 122541241 | 0.1345 | 0.1207 | 0.4119 |
| 4026 | RP11-299J3 | 3 | 123486719 | 123632323 | 0.1345 | 0.1246 | 0.4113 |
| 2122 | RP11-457E6 | 3 | 123901753 | 124000617 | 0.1345 | 0.1246 | 0.4113 |
| 791 | RP11-9N20 | 3 | 124855434 | 125034609 | 0.1346 | 0.1039 | 0.4084 |
| 3528 | RP11-521J5 | 3 | 125238538 | 125434264 | 0.1348 | 0.0978 | 0.4046 |
| 2279 | RP11-71H17 | 3 | 125910478 | 126076548 | 0.1348 | 0.0978 | 0.4046 |
| 1520 | RP11-205A6 | 3 | 127434149 | 127483270 | 0.1346 | 0.1175 | 0.4093 |
| 1868 | RP11-321A2 | 3 | 127987691 | 128108621 | 0.1347 | 0.1115 | 0.4072 |
| 645 | RP11-59J16 | 3 | 128599460 | 128752045 | 0.1347 | 0.1115 | 0.4072 |
| 2069 | RP11-221E20 | 3 | 129993751 | 130058432 | 0.1481 | 0.0984 | 0.4111 |
| 285 | RP11-129J11 | 3 | 131459233 | 131616358 | 0.1481 | 0.1157 | 0.4102 |
| 1352 | RP11-517B11 | 3 | 132615008 | 132784649 | 0.1973 | 0.1374 | 0.4063 |
| 3118 | RP11-48F15 | 3 | 133822599 | 133959231 | 0.2272 | 0.1454 | 0.4174 |
| 1508 | RP11-91K8 | 3 | 134620115 | 134771760 | 0.2272 | 0.1454 | 0.4174 |
| 3761 | RP11-91K8 | 3 | 134620115 | 134771760 | 0.2272 | 0.1454 | 0.4174 |
| 384 | RP11-22E12 | 3 | 135843620 | 136012651 | 0.2272 | 0.1454 | 0.4174 |
| 3676 | RP11-91O5 | 3 | 136539146 | 136692406 | 0.2274 | 0.1503 | 0.4121 |
| 1871 | RP11-311K2 | 3 | 137747908 | 137748612 | 0.2426 | 0.2230 | 0.4597 |
| 4263 | RP11-311K2 | 3 | 137747908 | 137748612 | 0.2426 | 0.2230 | 0.4597 |
| 4185 | RP11-269A14 | 3 | 138231890 | 138416930 | 0.2446 | 0.1863 | 0.4169 |
| 2421 | RP11-2A4 | 3 | 138893123 | 139048143 | 0.2448 | 0.2033 | 0.4119 |
| 2673 | RP11-80p20 | 3 | 139548619 | 139696434 | 0.2446 | 0.2096 | 0.4160 |
| 2084 | RP11-349D24 | 3 | 140626727 | 140627481 | 0.2446 | 0.2096 | 0.4160 |
| 3399 | RP11-349D24 | 3 | 140626727 | 140627481 | 0.2446 | 0.2096 | 0.4160 |
| 2039 | RP11-89E16 | 3 | 141265360 | 141410458 | 0.2446 | 0.2096 | 0.4160 |
| 2062 | RP11-231L11 | 3 | 142174414 | 142328307 | 0.2445 | 0.1988 | 0.4198 |
| 4234 | RP11-235I18 | 3 | 143078415 | 143237513 | 0.2445 | 0.2043 | 0.4195 |
| 80 | RP11-160a13 | 3 | 144154813 | 144312325 | 0.2572 | 0.2351 | 0.4346 |
| 3322 | RP11-160A13 | 3 | 144154813 | 144312325 | 0.2572 | 0.2351 | 0.4346 |
| 3866 | RP11-165M11 | 3 | 144868248 | 144912572 | 0.2572 | 0.2351 | 0.4346 |
| 2059 | RP11-200O17 | 3 | 145885183 | 146013682 | 0.2572 | 0.2351 | 0.4346 |
| 1507 | RP11-88H10 | 3 | 147013411 | 147184823 | 0.2572 | 0.2351 | 0.4346 |
| 368 | RP11-21M4 | 3 | 147739246 | 147851434 | 0.2568 | 0.2518 | 0.4427 |
| 16 | RP11-500K7 | 3 | 149208178 | 149208692 | 0.2568 | 0.2518 | 0.4427 |
| 2073 | RP11-223L18 | 3 | 154955751 | 155086892 | 0.2458 | 0.2562 | 0.4413 |
| 827 | RP11-117L15 | 3 | 156644066 | 156810056 | 0.2458 | 0.2562 | 0.4413 |
| 4218 | RP11-111F10 | 3 | 157115243 | 157286192 | 0.2458 | 0.2562 | 0.4413 |
| 243 | RP11-6F2 | 3 | 158177800 | 158323315 | 0.2103 | 0.2562 | 0.4387 |
| 94 | RP11-209h21 | 3 | 160726423 | 160903086 | 0.2101 | 0.2684 | 0.4348 |
| 2064 | RP11-209H21 | 3 | 160726423 | 160903086 | 0.2101 | 0.2684 | 0.4348 |
| 3770 | RP11-209H21 | 3 | 160726423 | 160903086 | 0.2101 | 0.2684 | 0.4348 |
| 646 | RP11-67F24 | 3 | 161144699 | 161320806 | 0.2097 | 0.2708 | 0.4250 |
| 2172 | RP11-498P15 | 3 | 163532324 | 163648094 | 0.3034 | 0.3100 | 0.4524 |
| 1593 | RP11-81E19 | 3 | 166072602 | 166237067 | 0.3034 | 0.3100 | 0.4524 |
| 2800 | RP11-81E19 | 3 | 166072602 | 166237067 | 0.3034 | 0.3100 | 0.4524 |
| 1166 | RP11-163H6 | 3 | 173183497 | 173348027 | 0.3548 | 0.3673 | 0.4623 |
| 2593 | RP11-44A1 | 3 | 173688016 | 173855765 | 0.3548 | 0.3673 | 0.4623 |
| 2930 | RP11-569P10 | 3 | 174372436 | 174549467 | 0.3548 | 0.3673 | 0.4623 |
| 2 | RP11-477P16 | 3 | 175307349 | 175497951 | 0.3548 | 0.3673 | 0.4623 |
| 4246 | RP11-477P16 | 3 | 175307349 | 175497951 | 0.3548 | 0.3673 | 0.4623 |
| 115 | RP11-71G7 | 3 | 177686929 | 177849652 | 0.3551 | 0.3776 | 0.4694 |
| 2722 | RP11-484D18 | 3 | 178148582 | 178318214 | 0.3551 | 0.3776 | 0.4694 |
| 3672 | RP11-91K9 | 3 | 178971537 | 179129175 | 0.3551 | 0.3776 | 0.4694 |
| 1456 | RP11-682A21 | 3 | 180293412 | 180449253 | 0.3551 | 0.3776 | 0.4694 |
| 3137 | RP11-45I24 | 3 | 180867439 | 181028448 | 0.3549 | 0.3655 | 0.4658 |
| 1447 | RP11-534H15 | 3 | 181435699 | 181436285 | 0.3551 | 0.3565 | 0.4701 |
| 1893 | RP11-534H15 | 3 | 181435699 | 181436285 | 0.3551 | 0.3565 | 0.4701 |
| 638 | RP11-65J14 | 3 | 187299591 | 187370245 | 0.3122 | 0.3086 | 0.4639 |
| 272 | RP11-119E13 | 3 | 188090387 | 188091023 | 0.3111 | 0.2925 | 0.4868 |
| 1896 | RP11-525C11 | 3 | 192500451 | 192584204 | 0.2681 | 0.2401 | 0.5826 |
| 2590 | RP11-47G2 | 3 | 193748157 | 193916452 | 0.2505 | 0.2586 | 0.5198 |
| 105 | RP11-56C4 | 3 | 193972699 | 194023813 | 0.2504 | 0.2540 | 0.5194 |
| 2549 | RP11-279P10 | 3 | 195537798 | 195629784 | 0.2505 | 0.2592 | 0.5197 |
| 1355 | RP11-506F8 | 3 | 196614673 | 196768568 | 0.2505 | 0.2786 | 0.5199 |
| 3636 | RP11-252K11 | 3 | 197391807 | 197555990 | 0.2502 | 0.2838 | 0.5148 |
| 279 | RP11-114F20 | 3 | 198339450 | 198528158 | 0.2516 | 0.2833 | 0.5449 |
| 1379 | GS-56-H22 | 3 | 198976827 | 199126827 | 0.2519 | 0.2751 | 0.5505 |
| 1385 | GS-196-F4 | 3 | 198976877 | 199126877 | 0.2519 | 0.2751 | 0.5505 |
| 3416 | RP11-23M2 | 3 | 198977374 | 199160103 | 0.2519 | 0.2751 | 0.5505 |
| 3764 | RP11-23M2 | 3 | 198977374 | 199160103 | 0.2519 | 0.2751 | 0.5505 |
| 3549 | GS-36-P21 | 4 | 15000 | 165000 | −0.0394 | −0.0302 | 0.3882 |
| 3543 | GS-118-B13 | 4 | 26000 | 176000 | −0.0394 | −0.0302 | 0.3882 |
| 4193 | RP11-270I3 | 4 | 10640517 | 10803372 | −0.0001 | 0.0028 | 0.3846 |
| 1159 | RP11-168E17 | 4 | 11490644 | 11562120 | −0.0001 | 0.0028 | 0.3846 |
| 2149 | RP11-168E17 | 4 | 11490644 | 11562120 | −0.0001 | 0.0028 | 0.3846 |
| 2540 | RP11-22A3 | 4 | 12410325 | 12584750 | −0.0002 | 0.0098 | 0.3811 |
| 1582 | RP11-446J8 | 4 | 16019234 | 16183206 | −0.0002 | 0.0098 | 0.3811 |
| 2125 | RP11-441O12 | 4 | 18028014 | 18213766 | −0.0002 | 0.0098 | 0.3811 |
| 2414 | RP11-3J1 | 4 | 18737376 | 18858211 | −0.0005 | 0.0178 | 0.3752 |
| 3755 | RP11-3J1 | 4 | 18737376 | 18858211 | −0.0005 | 0.0178 | 0.3752 |
| 1776 | RP11-362J17 | 4 | 20347899 | 20529941 | 0.0131 | 0.0178 | 0.3708 |
| 656 | RP11-84G22 | 4 | 21482302 | 21587657 | 0.0131 | 0.0178 | 0.3708 |
| 3757 | RP11-84G22 | 4 | 21482302 | 21587657 | 0.0131 | 0.0178 | 0.3708 |
| 803 | RP11-10G12 | 4 | 22163781 | 22288928 | 0.0131 | 0.0279 | 0.3704 |
| 2478 | RP11-406E22 | 4 | 22956884 | 22976809 | 0.0131 | 0.0279 | 0.3704 |
| 1356 | RP11-519A11 | 4 | 28748334 | 28909642 | 0.0133 | 0.0158 | 0.3750 |
| 856 | RP11-390C19 | 4 | 29381036 | 29556502 | 0.0133 | 0.0158 | 0.3750 |
| 10 | RP11-478O20 | 4 | 31129762 | 31286231 | 0.0133 | 0.0187 | 0.3761 |
| 389 | RP11-280K20 | 4 | 31500912 | 31664000 | 0.0133 | 0.0187 | 0.3761 |
| 3707 | RP11-280K20 | 4 | 31500912 | 31664000 | 0.0133 | 0.0187 | 0.3761 |
| 1806 | RP11-20M7 | 4 | 32138110 | 32290383 | 0.0134 | 0.0359 | 0.3780 |
| 112 | RP11-79E3 | 4 | 33550507 | 33712878 | 0.0134 | 0.0359 | 0.3780 |
| 1268 | RP11-24H13 | 4 | 34561876 | 34578239 | 0.0134 | 0.0359 | 0.3780 |
| 2981 | RP11-135M12 | 4 | 35206808 | 35398317 | 0.0134 | 0.0359 | 0.3780 |
| 4209 | RP11-103J17 | 4 | 36674852 | 36836152 | 0.0134 | 0.0247 | 0.3775 |
| 3088 | RP11-36B15 | 4 | 37192086 | 37351155 | 0.0134 | 0.0247 | 0.3775 |
| 1560 | RP11-343C9 | 4 | 39430681 | 39629614 | 0.0193 | 0.0292 | 0.3753 |
| 1778 | RP11-343C9 | 4 | 39430681 | 39629614 | 0.0193 | 0.0292 | 0.3753 |
| 3686 | RP11-227F19 | 4 | 41340065 | 41525131 | 0.0193 | 0.0379 | 0.3754 |
| 4206 | RP11-109E24 | 4 | 42330834 | 42508587 | 0.0192 | 0.0466 | 0.3728 |
| 1534 | RP11-229G4 | 4 | 43182054 | 43332054 | 0.0190 | 0.0358 | 0.3691 |
| 1451 | RP11-542I3 | 4 | 43837854 | 44039573 | 0.0197 | 0.0251 | 0.3830 |
| 3530 | RP11-542I3 | 4 | 43837854 | 44039573 | 0.0197 | 0.0251 | 0.3830 |
| 101 | RP11-55C6 | 4 | 44529011 | 44675684 | 0.0197 | 0.0251 | 0.3830 |
| 1575 | RP11-416A5 | 4 | 45608185 | 45778656 | 0.0186 | 0.0469 | 0.3608 |
| 2037 | RP11-100N21 | 4 | 47264009 | 47432186 | 0.0186 | 0.0469 | 0.3608 |
| 3735 | RP11-415L23 | 4 | 47677783 | 47787969 | 0.0185 | 0.0545 | 0.3576 |
| 1794 | RP11-19F13 | 4 | 48565289 | 48589289 | 0.0181 | 0.0662 | 0.3495 |
| 2459 | RP11-365H22 | 4 | 52354875 | 52530859 | 0.0611 | 0.0822 | 0.3528 |
| 4245 | RP11-365H22 | 4 | 52354875 | 52530859 | 0.0611 | 0.0822 | 0.3528 |
| 3873 | RP11-157C8 | 4 | 53060046 | 53208038 | 0.0616 | 0.0644 | 0.3647 |
| 2956 | RP11-12J3 | 4 | 54523234 | 54684548 | 0.0616 | 0.0644 | 0.3647 |
| 4222 | RP11-231C18 | 4 | 54822092 | 54952255 | 0.0616 | 0.0644 | 0.3647 |
| 2994 | RP11-148K14 | 4 | 55856180 | 55928233 | 0.0616 | 0.0644 | 0.3647 |
| 4239 | RP11-204H9 | 4 | 64867896 | 65032899 | 0.0376 | 0.0384 | 0.3657 |
| 1576 | RP11-423P14 | 4 | 65766214 | 65968121 | 0.0376 | 0.0384 | 0.3657 |
| 549 | RP11-468N14 | 4 | 69696766 | 69912765 | −0.1156 | 0.0172 | 0.4193 |
| 4196 | RP11-92H22 | 4 | 71879338 | 72031750 | 0.0823 | 0.0273 | 0.4239 |
| 249 | RP11-1J11 | 4 | 72265535 | 72403640 | 0.0823 | 0.0273 | 0.4239 |
| 847 | RP11-373J21 | 4 | 73321374 | 73496366 | 0.0823 | 0.0273 | 0.4239 |
| 1586 | RP11-447E20 | 4 | 74788409 | 74864817 | 0.0823 | 0.0234 | 0.4249 |
| 3426 | RP11-17P8 | 4 | 89621629 | 89807633 | −0.0452 | −0.0607 | 0.3650 |
| 24 | RP11-502A23 | 4 | 90029276 | 90198548 | −0.0452 | −0.0607 | 0.3650 |
| 2971 | RP11-115D19 | 4 | 90755173 | 90922656 | −0.0452 | −0.0607 | 0.3650 |
| 264 | RP11-11N6 | 4 | 91753755 | 91900370 | −0.0452 | −0.0607 | 0.3650 |
| 787 | RP11-9B6 | 4 | 93421437 | 93529296 | −0.0451 | −0.0672 | 0.3615 |
| 3125 | RP11-44P19 | 4 | 93630873 | 93775737 | −0.0482 | −0.0867 | 0.4288 |
| 1801 | RP11-16I17 | 4 | 94662870 | 94836378 | −0.0481 | −0.0947 | 0.4270 |
| 244 | RP11-10L11 | 4 | 96358705 | 96515910 | −0.0452 | −0.0873 | 0.3652 |
| 2092 | RP11-354C17 | 4 | 97073538 | 97124201 | −0.0456 | −0.0815 | 0.3725 |
| 3935 | RP11-354C17 | 4 | 97073538 | 97124201 | −0.0456 | −0.0815 | 0.3725 |
| 3643 | RP11-240J11 | 4 | 98832380 | 98895589 | −0.0456 | −0.0815 | 0.3725 |
| 2112 | RP11-428B4 | 4 | 99946600 | 100058902 | −0.0456 | −0.0815 | 0.3725 |
| 1990 | RP11-56D20 | 4 | 101150764 | 101325336 | −0.0456 | −0.0815 | 0.3725 |
| 911 | RP11-13F20 | 4 | 101510577 | 101670097 | −0.0456 | −0.0815 | 0.3725 |
| 3315 | RP11-167N19 | 4 | 101813198 | 101961364 | −0.0456 | −0.0815 | 0.3725 |
| 2050 | RP11-110C16 | 4 | 103067212 | 103220659 | −0.0456 | −0.0815 | 0.3725 |
| 4273 | RP11-438P8 | 4 | 104406898 | 104431241 | −0.0456 | −0.0815 | 0.3725 |
| 1707 | RP11-174B22 | 4 | 104431242 | 104513122 | −0.0456 | −0.0815 | 0.3725 |
| 292 | RP11-122B24 | 4 | 105288367 | 105355835 | −0.0454 | −0.0867 | 0.3694 |
| 1797 | RP11-16G16 | 4 | 106336237 | 106507303 | −0.0453 | −0.0967 | 0.3663 |
| 4220 | RP11-208O6 | 4 | 107027991 | 107064932 | −0.0457 | −0.0919 | 0.3750 |
| 3667 | RP11-88D10 | 4 | 107736653 | 107943820 | −0.0457 | −0.1002 | 0.3760 |
| 2046 | RP11-109F18 | 4 | 108646932 | 108795103 | −0.0457 | −0.1002 | 0.3760 |
| 651 | RP11-75N20 | 4 | 109877028 | 110025600 | −0.0457 | −0.1069 | 0.3747 |
| 2273 | RP11-57A22 | 4 | 110057781 | 110225193 | −0.0457 | −0.1069 | 0.3747 |
| 2468 | RP11-380D23 | 4 | 111660447 | 111860073 | −0.0458 | −0.1017 | 0.3777 |
| 1237 | RP11-326N15 | 4 | 112359892 | 112502402 | −0.0458 | −0.1017 | 0.3777 |
| 2264 | RP11-77N9 | 4 | 112853134 | 112892477 | −0.0467 | −0.1121 | 0.3971 |
| 3753 | RP11-77N9 | 4 | 112853134 | 112892477 | −0.0467 | −0.1121 | 0.3971 |
| 298 | RP11-402J6 | 4 | 113599819 | 113777515 | −0.0459 | −0.0928 | 0.3800 |
| 1587 | RP11-419E3 | 4 | 116321032 | 116470420 | −0.0476 | −0.0672 | 0.4177 |
| 3964 | RP11-25B14 | 4 | 116748169 | 116935082 | −0.0476 | −0.0672 | 0.4177 |
| 2265 | RP11-55L3 | 4 | 117579708 | 117743758 | −0.0477 | −0.0607 | 0.4184 |
| 1371 | RP11-510D4 | 4 | 118641765 | 118817790 | −0.0477 | −0.0607 | 0.4184 |
| 1513 | RP11-100C9 | 4 | 119888512 | 120033602 | −0.0467 | −0.0733 | 0.3979 |
| 3018 | RP11-414D7 | 4 | 121243300 | 121302803 | −0.0467 | −0.0733 | 0.3979 |
| 2180 | RP11-501E14 | 4 | 121715814 | 121904034 | −0.0467 | −0.0733 | 0.3979 |
| 1565 | RP11-364P2 | 4 | 122489952 | 122658178 | −0.0467 | −0.0733 | 0.3979 |
| 2455 | RP11-364P2 | 4 | 122489952 | 122658178 | −0.0467 | −0.0733 | 0.3979 |
| 2472 | RP11-381N20 | 4 | 124552339 | 124675489 | −0.0467 | −0.0733 | 0.3979 |
| 4200 | RP11-93I21 | 4 | 125513755 | 125668337 | −0.0467 | −0.0733 | 0.3979 |
| 1793 | RP11-15O17 | 4 | 126354712 | 126517758 | −0.0467 | −0.0826 | 0.3968 |
| 1547 | RP11-282B13 | 4 | 127250441 | 127434004 | −0.0467 | −0.0771 | 0.3966 |
| 4178 | RP11-282B13 | 4 | 127250441 | 127434004 | −0.0467 | −0.0771 | 0.3966 |
| 237 | RP11-555L17 | 4 | 129322994 | 129526144 | −0.0466 | −0.0732 | 0.3948 |
| 2060 | RP11-209D20 | 4 | 130001104 | 130120812 | −0.0466 | −0.0732 | 0.3948 |
| 1604 | RP11-24P18 | 4 | 131614434 | 131767271 | −0.0472 | −0.0613 | 0.4090 |
| 1800 | RP11-24P18 | 4 | 131614434 | 131767271 | −0.0472 | −0.0613 | 0.4090 |
| 3488 | RP11-314N14 | 4 | 132653555 | 132677577 | −0.0472 | −0.0613 | 0.4090 |
| 3313 | RP11-149A7 | 4 | 133664206 | 133834624 | −0.0473 | −0.0629 | 0.4098 |
| 3394 | RP11-335K21 | 4 | 134372074 | 134522870 | −0.0473 | −0.0629 | 0.4098 |
| 3017 | RP11-400D2 | 4 | 135464064 | 135638813 | −0.0473 | −0.0629 | 0.4098 |
| 2274 | RP11-63M2 | 4 | 138014415 | 138174314 | −0.0473 | −0.0605 | 0.4102 |
| 4268 | RP11-425J20 | 4 | 138445740 | 138620766 | −0.0473 | −0.0605 | 0.4102 |
| 3665 | RP11-98O2 | 4 | 139829360 | 139995928 | −0.0468 | −0.0769 | 0.3984 |
| 2259 | RP11-69O1 | 4 | 141603563 | 141774290 | −0.0467 | −0.0691 | 0.3964 |
| 3936 | RP11-362F19 | 4 | 142401187 | 142585401 | −0.0467 | −0.0691 | 0.3964 |
| 3996 | RP11-141I1 | 4 | 143754328 | 143872121 | −0.0463 | −0.0574 | 0.3893 |
| 1788 | RP11-364L4 | 4 | 144367994 | 144531240 | −0.0463 | −0.0574 | 0.3893 |
| 3725 | RP11-364L4 | 4 | 144367994 | 144531240 | −0.0463 | −0.0574 | 0.3893 |
| 1870 | RP11-301H24 | 4 | 146558547 | 146722475 | −0.0467 | −0.0506 | 0.3973 |
| 1696 | RP11-181K12 | 4 | 146929833 | 147087801 | −0.0467 | −0.0506 | 0.3973 |
| 1584 | RP11-425A23 | 4 | 148754633 | 148949857 | −0.0467 | −0.0553 | 0.3980 |
| 552 | RP11-503L19 | 4 | 149764895 | 149896425 | −0.0467 | −0.0553 | 0.3980 |
| 550 | RP11-481K13 | 4 | 150581329 | 150736953 | −0.0467 | −0.0553 | 0.3980 |
| 118 | RP11-64M20 | 4 | 151573175 | 151716754 | −0.0467 | −0.0553 | 0.3980 |
| 963 | RP11-28E24 | 4 | 151847129 | 151991147 | −0.0467 | −0.0553 | 0.3980 |
| 4292 | RP11-28E24 | 4 | 151847129 | 151991147 | −0.0467 | −0.0553 | 0.3980 |
| 2016 | RP11-259G7 | 4 | 154858444 | 155057548 | −0.0688 | −0.0946 | 0.3826 |
| 3115 | RP11-27G13 | 4 | 156373845 | 156550048 | −0.0688 | −0.0906 | 0.3826 |
| 3040 | RP11-264E11 | 4 | 156624370 | 156778344 | −0.0688 | −0.0906 | 0.3826 |
| 1699 | RP11-171N4 | 4 | 157665354 | 157836342 | −0.0689 | −0.0940 | 0.3844 |
| 1250 | RP11-17C4 | 4 | 158576037 | 158701473 | −0.0694 | −0.1005 | 0.3940 |
| 2403 | RP11-6C14 | 4 | 160913163 | 161053242 | −0.0693 | −0.0973 | 0.3925 |
| 2409 | RP11-1G8 | 4 | 163467846 | 163611458 | −0.0693 | −0.0973 | 0.3925 |
| 3701 | RP11-218F10 | 4 | 164527241 | 164686093 | −0.0693 | −0.0973 | 0.3925 |
| 3398 | RP11-336N6 | 4 | 166240478 | 166417001 | −0.0693 | −0.0973 | 0.3925 |
| 2579 | RP11-25N12 | 4 | 166740143 | 166917702 | −0.0693 | −0.0973 | 0.3925 |
| 4281 | RP11-440L13 | 4 | 167847352 | 168019175 | −0.0695 | −0.1031 | 0.3974 |
| 325 | RP11-275K4 | 4 | 170624275 | 170779794 | −0.0727 | −0.0744 | 0.4662 |
| 3703 | RP11-275K4 | 4 | 170624275 | 170779794 | −0.0727 | −0.0744 | 0.4662 |
| 3681 | RP11-213L8 | 4 | 171956844 | 172113114 | −0.0707 | −0.0937 | 0.4238 |
| 919 | RP11-13N11 | 4 | 171958495 | 172124798 | −0.0707 | −0.0937 | 0.4238 |
| 840 | RP11-384D10 | 4 | 172313670 | 172377198 | −0.0693 | −0.1113 | 0.3915 |
| 2436 | RP11-119J20 | 4 | 173240623 | 173408179 | −0.0693 | −0.1113 | 0.3915 |
| 282 | RP11-140M23 | 4 | 173960100 | 174135022 | −0.0693 | −0.1113 | 0.3915 |
| 834 | RP11-148L24 | 4 | 175343072 | 175486570 | −0.0693 | −0.1113 | 0.3915 |
| 1559 | RP11-287F9 | 4 | 176347359 | 176566236 | −0.0693 | −0.1113 | 0.3915 |
| 3493 | RP11-287F9 | 4 | 176347359 | 176566236 | −0.0693 | −0.1113 | 0.3915 |
| 3659 | RP11-87F15 | 4 | 177405896 | 177623816 | −0.0693 | −0.1113 | 0.3915 |
| 4157 | CTC-1499I20 | 4 | 179303625 | 179403709 | −0.0460 | −0.1112 | 0.3942 |
| 2820 | RP11-84H6 | 4 | 179406094 | 179461528 | −0.0460 | −0.1112 | 0.3942 |
| 563 | RP11-495H13 | 4 | 179834806 | 180006551 | −0.0460 | −0.1112 | 0.3942 |
| 3001 | RP11-396I22 | 4 | 181215945 | 181420733 | −0.0058 | −0.1112 | 0.4019 |
| 2262 | RP11-62B7 | 4 | 182513493 | 182549904 | −0.0058 | −0.1112 | 0.4019 |
| 2805 | RP11-59I16 | 4 | 184232930 | 184404421 | −0.0060 | −0.1051 | 0.4052 |
| 4274 | RP11-451F20 | 4 | 184365840 | 184561883 | −0.0060 | −0.1051 | 0.4052 |
| 3690 | RP11-228F3 | 4 | 185872338 | 186040225 | −0.0468 | −0.1051 | 0.4069 |
| 1874 | RP11-301L8 | 4 | 186652578 | 186820398 | −0.0489 | −0.1368 | 0.4515 |
| 1543 | RP11-279K24 | 4 | 187170026 | 187291316 | −0.0489 | −0.1368 | 0.4515 |
| 2545 | RP11-279K24 | 4 | 187170026 | 187291316 | −0.0489 | −0.1368 | 0.4515 |
| 3668 | RP11-91J3 | 4 | 188522244 | 188674927 | −0.0980 | −0.1369 | 0.4445 |
| 3939 | RP11-354H17 | 4 | 189958830 | 190149921 | −0.0986 | −0.1330 | 0.4577 |
| 4256 | RP11-354H17 | 4 | 189958830 | 190149921 | −0.0986 | −0.1330 | 0.4577 |
| 973 | RP11-45F23 | 4 | 190520006 | 190668424 | −0.0986 | −0.1330 | 0.4577 |
| 1383 | GS-31-J3 | 4 | 190698063 | 190848063 | −0.0970 | −0.1171 | 0.4238 |
| 1389 | GS-963-K6 | 4 | 190810563 | 190960563 | −0.0965 | −0.1271 | 0.4119 |
| 1934 | GS-24-H17 | 5 | 1 | 125000 | 0.0086 | −0.0130 | 0.4405 |
| 3540 | 1129-C9 | 5 | 1 | 125050 | 0.0086 | −0.0130 | 0.4405 |
| 4090 | 189-N21 | 5 | 1 | 125100 | 0.0086 | −0.0130 | 0.4405 |
| 2843 | CTD-2265D9 | 5 | 2570683 | 2671745 | 0.0733 | 0.1924 | 0.4535 |
| 3450 | RP11-20K9 | 5 | 3172060 | 3340431 | 0.0733 | 0.1924 | 0.4535 |
| 1609 | RP11-121L11 | 5 | 3426806 | 3597254 | 0.0734 | 0.2032 | 0.4553 |
| 2452 | RP11-121L11 | 5 | 3426806 | 3597254 | 0.0734 | 0.2032 | 0.4553 |
| 1611 | RP11-13H10 | 5 | 4351733 | 4533614 | 0.0442 | 0.2081 | 0.4476 |
| 3981 | RP11-13H10 | 5 | 4351733 | 4533614 | 0.0442 | 0.2081 | 0.4476 |
| 1828 | RP11-137P5 | 5 | 5500098 | 5687008 | 0.0442 | 0.2081 | 0.4476 |
| 3024 | RP11-245L6 | 5 | 5879129 | 5995464 | 0.0442 | 0.2056 | 0.4479 |
| 2292 | CTD-2324F15 | 5 | 6350940 | 6430016 | 0.0442 | 0.2056 | 0.4479 |
| 287 | RP11-114M17 | 5 | 6515876 | 6708632 | 0.0442 | 0.2056 | 0.4479 |
| 2300 | CTD-2346M20 | 5 | 7449057 | 7530186 | 0.0443 | 0.2172 | 0.4495 |
| 2973 | RP11-132J20 | 5 | 8413255 | 8600646 | 0.0446 | 0.2294 | 0.4564 |
| 1834 | RP11-32D12 | 5 | 9436026 | 9606181 | 0.0437 | 0.2214 | 0.4374 |
| 2284 | CTD-2274H20 | 5 | 10359620 | 10495937 | 0.0433 | 0.2144 | 0.4293 |
| 2488 | RP11-215G15 | 5 | 10644705 | 10722894 | 0.0433 | 0.2144 | 0.4293 |
| 1944 | RP3-502L6 | 5 | 10925533 | 11148940 | 0.0433 | 0.2089 | 0.4288 |
| 2205 | RP11-44C2 | 5 | 12130015 | 12216435 | 0.0438 | 0.1970 | 0.4389 |
| 1411 | RP1-64M18 | 5 | 12216436 | 12262940 | 0.0431 | 0.1842 | 0.4240 |
| 1407 | RP1-61C2 | 5 | 13138380 | 13288380 | 0.0430 | 0.1761 | 0.4225 |
| 44 | RP11-415K6 | 5 | 14596376 | 14781538 | 0.0433 | 0.1845 | 0.4280 |
| 4099 | RP1-137K24 | 5 | 15191411 | 15315356 | 0.0433 | 0.1845 | 0.4280 |
| 1947 | RP1-167G20 | 5 | 16354087 | 16529263 | 0.0573 | 0.1845 | 0.4321 |
| 1263 | RP11-21H5 | 5 | 16365822 | 16546966 | 0.0573 | 0.1845 | 0.4321 |
| 31 | RP11-260E18 | 5 | 16623433 | 16726662 | 0.0711 | 0.1844 | 0.4330 |
| 2140 | RP11-28P24 | 5 | 19642833 | 19781435 | 0.0710 | 0.1882 | 0.4303 |
| 3990 | RP11-28P24 | 5 | 19642833 | 19781435 | 0.0710 | 0.1882 | 0.4303 |
| 1141 | RP11-414I19 | 5 | 20429524 | 20595579 | 0.0710 | 0.1882 | 0.4303 |
| 866 | RP11-374E21 | 5 | 21573338 | 21757179 | 0.0710 | 0.1882 | 0.4303 |
| 2089 | RP11-374E21 | 5 | 21573338 | 21757179 | 0.0710 | 0.1882 | 0.4303 |
| 1608 | RP11-26L18 | 5 | 22309858 | 22468875 | 0.0710 | 0.1882 | 0.4303 |
| 2591 | RP11-26L18 | 5 | 22309858 | 22468875 | 0.0710 | 0.1882 | 0.4303 |
| 1287 | RP11-57I6 | 5 | 23644140 | 23795207 | 0.0702 | 0.1574 | 0.4138 |
| 4312 | RP11-57I6 | 5 | 23644140 | 23795207 | 0.0702 | 0.1574 | 0.4138 |
| 3679 | RP11-192H6 | 5 | 25187395 | 25333181 | 0.0869 | 0.2154 | 0.4458 |
| 2498 | RP11-351N6 | 5 | 26353438 | 26539899 | 0.1042 | 0.2154 | 0.4455 |
| 2839 | CTD-2219P12 | 5 | 27359951 | 27463814 | 0.1668 | 0.2468 | 0.4779 |
| 2578 | RP11-46C20 | 5 | 27524331 | 27641965 | 0.1668 | 0.2468 | 0.4779 |
| 3435 | RP11-37M16 | 5 | 28851494 | 28918008 | 0.1634 | 0.1829 | 0.4041 |
| 1829 | RP11-16A6 | 5 | 31326937 | 31504934 | 0.1626 | 0.1679 | 0.3866 |
| 3447 | RP11-53L13 | 5 | 32742919 | 32916357 | 0.1626 | 0.1679 | 0.3866 |
| 3989 | RP11-15G6 | 5 | 33280772 | 33449384 | 0.1626 | 0.1679 | 0.3866 |
| 4291 | RP11-15G6 | 5 | 33280772 | 33449384 | 0.1626 | 0.1679 | 0.3866 |
| 2001 | CTD-2052F19 | 5 | 34971009 | 35043293 | 0.1626 | 0.1679 | 0.3866 |
| 2288 | CTD-2291F22 | 5 | 35388192 | 35513594 | 0.1624 | 0.1634 | 0.3807 |
| 683 | CTD-2267H19 | 5 | 36275837 | 36380871 | 0.1618 | 0.1740 | 0.3689 |
| 251 | RP11-7M4 | 5 | 36988555 | 37177051 | 0.1615 | 0.1674 | 0.3616 |
| 2379 | RP11-575G10 | 5 | 42897645 | 42898147 | 0.1249 | 0.1266 | 0.3595 |
| 2507 | RP4-592P18 | 5 | 51083837 | 51232444 | 0.1058 | 0.1300 | 0.4406 |
| 3850 | RP11-506H20 | 5 | 54562681 | 54725678 | −0.1204 | −0.0332 | 0.4188 |
| 862 | RP11-364C6 | 5 | 55408298 | 55588124 | −0.1206 | −0.0803 | 0.4151 |
| 1561 | RP11-364C6 | 5 | 55408298 | 55588124 | −0.1206 | −0.0803 | 0.4151 |
| 3297 | RP11-412L4 | 5 | 55546666 | 55547446 | −0.1207 | −0.0954 | 0.4116 |
| 1048 | RP4-572A3 | 5 | 56921507 | 57065064 | −0.1377 | −0.1291 | 0.4086 |
| 3846 | RP11-495K20 | 5 | 58128952 | 58280997 | −0.1377 | −0.1291 | 0.4086 |
| 3029 | RP11-313P15 | 5 | 60735461 | 60791039 | −0.1218 | −0.1621 | 0.3925 |
| 2596 | RP11-34J15 | 5 | 61659573 | 61769004 | −0.1219 | −0.1745 | 0.3945 |
| 3841 | RP11-432A10 | 5 | 62041315 | 62191844 | −0.1219 | −0.1745 | 0.3945 |
| 1282 | RP11-19B7 | 5 | 64142336 | 64300795 | −0.1219 | −0.1745 | 0.3945 |
| 22 | RP11-480H11 | 5 | 65059150 | 65239020 | −0.1466 | −0.1746 | 0.3906 |
| 2295 | CTC-536A23 | 5 | 65352862 | 65454024 | −0.1466 | −0.1746 | 0.3906 |
| 1133 | RP11-402F5 | 5 | 66695993 | 66888015 | −0.1087 | −0.1746 | 0.3842 |
| 1682 | RP11-494P23 | 5 | 67322915 | 67501695 | −0.1091 | −0.1964 | 0.3937 |
| 217 | RP11-551B22 | 5 | 70361715 | 70517014 | −0.1114 | −0.2206 | 0.4429 |
| 3976 | RP11-115I6 | 5 | 71659788 | 71823446 | −0.0608 | −0.1972 | 0.3747 |
| 2291 | CTC-533D18 | 5 | 72794427 | 72920172 | −0.0610 | −0.1872 | 0.3792 |
| 853 | RP11-399M5 | 5 | 73126031 | 73314954 | −0.0610 | −0.1872 | 0.3792 |
| 1989 | CTC-1347N9 | 5 | 73872906 | 73873453 | −0.0613 | −0.1802 | 0.3854 |
| 1835 | RP11-97L2 | 5 | 73817310 | 73968588 | −0.0613 | −0.1802 | 0.3854 |
| 675 | CTD-2200O3 | 5 | 75600638 | 75734191 | −0.0612 | −0.1858 | 0.3832 |
| 3616 | RP11-667P13 | 5 | 78198045 | 78198547 | −0.0612 | −0.1782 | 0.3845 |
| 1294 | RP11-21K15 | 5 | 78203689 | 78284615 | −0.0612 | −0.1782 | 0.3845 |
| 666 | CTC-431G16 | 5 | 79093741 | 79219539 | −0.0612 | −0.1658 | 0.3839 |
| 910 | RP11-30D15 | 5 | 79172773 | 79351477 | −0.0612 | −0.1658 | 0.3839 |
| 4069 | RP11-538B23 | 5 | 79936331 | 80082792 | −0.0612 | −0.1658 | 0.3839 |
| 1722 | RP11-241J12 | 5 | 80075105 | 80216216 | −0.0617 | −0.1589 | 0.3954 |
| 2774 | RP11-241j12 | 5 | 80075105 | 80216216 | −0.0617 | −0.1589 | 0.3954 |
| 2104 | RP11-356D23 | 5 | 81265606 | 81410507 | −0.0618 | −0.1546 | 0.3974 |
| 3022 | RP11-356D23 | 5 | 81265606 | 81410507 | −0.0618 | −0.1546 | 0.3974 |
| 2173 | RP11-463C5 | 5 | 82570102 | 82742889 | −0.0618 | −0.1546 | 0.3974 |
| 2283 | CTC-480C2 | 5 | 84860884 | 85040367 | −0.0624 | −0.1473 | 0.4106 |
| 3979 | RP11-72L22 | 5 | 86341428 | 86472400 | −0.0626 | −0.1512 | 0.4139 |
| 1285 | RP11-3H15 | 5 | 87297507 | 87443969 | −0.0626 | −0.1512 | 0.4139 |
| 4059 | RP11-512I16 | 5 | 87676680 | 87711396 | −0.0626 | −0.1512 | 0.4139 |
| 792 | RP11-12D3 | 5 | 90806344 | 90964362 | −0.0321 | −0.1449 | 0.4263 |
| 143 | CTD-2011L22 | 5 | 92074742 | 92092165 | −0.0316 | −0.1638 | 0.4143 |
| 1290 | RP11-20O13 | 5 | 93129507 | 93173459 | −0.0317 | −0.1582 | 0.4160 |
| 2585 | RP11-39B14 | 5 | 93540085 | 93709247 | −0.0317 | −0.1540 | 0.4168 |
| 142 | CTC-366N20 | 5 | 94527791 | 94663165 | −0.0318 | −0.1439 | 0.4186 |
| 1904 | RP11-526D16 | 5 | 95842138 | 96007440 | −0.0323 | −0.1125 | 0.4291 |
| 2299 | CTC-564E20 | 5 | 98073104 | 98073634 | −0.0321 | −0.0833 | 0.4247 |
| 3687 | RP11-195A20 | 5 | 98954115 | 99111442 | −0.0321 | −0.0833 | 0.4247 |
| 2827 | CTD-2068C11 | 5 | 99850207 | 100001668 | −0.0321 | −0.0833 | 0.4247 |
| 3747 | RP11-418P19 | 5 | 100715359 | 100906849 | −0.0318 | −0.0738 | 0.4196 |
| 2933 | RP11-560F8 | 5 | 100991080 | 101181927 | −0.0318 | −0.0738 | 0.4196 |
| 3847 | RP11-565F4 | 5 | 119956077 | 120134621 | −0.0436 | −0.0351 | 0.4618 |
| 2293 | CTB-54G2 | 5 | 126657706 | 126865341 | −0.0462 | −0.0201 | 0.4053 |
| 2298 | CTC-352M6 | 5 | 127597190 | 127759109 | −0.0462 | −0.0201 | 0.4053 |
| 3196 | RP1-117N3 | 5 | 128354467 | 128454909 | −0.0463 | −0.0145 | 0.4040 |
| 1040 | RP1-179P12 | 5 | 129269732 | 129450042 | −0.0464 | −0.0063 | 0.4001 |
| 1955 | RP1-236L2 | 5 | 129569617 | 129599992 | −0.0464 | −0.0063 | 0.4001 |
| 2198 | RP11-114H7 | 5 | 130306745 | 130412531 | −0.0464 | −0.0063 | 0.4001 |
| 4119 | RP1-247F3 | 5 | 130616593 | 130630517 | −0.0465 | −0.0023 | 0.3993 |
| 3575 | RP1-66P19 | 5 | 131009149 | 131090780 | −0.0465 | −0.0023 | 0.3993 |
| 2506 | RP1-241C15 | 5 | 131365774 | 131577988 | −0.0463 | −0.0161 | 0.4038 |
| 141 | CTB-104P14 | 5 | 131686843 | 131731311 | −0.0585 | −0.0161 | 0.4013 |
| 3628 | RP11-729C24 | 5 | 131817042 | 131977001 | −0.0590 | −0.0031 | 0.3907 |
| 129 | CTB-49A3 | 5 | 132506140 | 132704549 | −0.0590 | −0.0058 | 0.3898 |
| 2285 | CTB-28J9 | 5 | 135549762 | 135738309 | −0.0372 | 0.0110 | 0.3700 |
| 38 | RP11-114H21 | 5 | 135739990 | 135899842 | −0.0368 | 0.0160 | 0.3797 |
| 3204 | RP1-244J5 | 5 | 136365521 | 136366234 | −0.0368 | 0.0160 | 0.3797 |
| 4111 | RP1-186K10 | 5 | 136892127 | 137064639 | −0.0367 | 0.0277 | 0.3822 |
| 2191 | RP11-256P1 | 5 | 137635258 | 137734281 | −0.0367 | 0.0277 | 0.3822 |
| 3192 | RP1-98O22 | 5 | 137758103 | 137758844 | −0.0370 | 0.0214 | 0.3761 |
| 2289 | CTB-46B19 | 5 | 138230413 | 138414953 | −0.0370 | 0.0214 | 0.3761 |
| 2825 | CTB-133C10 | 5 | 139529321 | 139663275 | −0.0371 | 0.0301 | 0.3736 |
| 1131 | RP11-515C16 | 5 | 139904059 | 140124593 | −0.0371 | 0.0301 | 0.3736 |
| 128 | CTD-2323H12 | 5 | 141041469 | 141198799 | −0.0367 | 0.0363 | 0.3807 |
| 2296 | CTD-2332G20 | 5 | 141852268 | 141998278 | −0.0371 | 0.0543 | 0.3735 |
| 2835 | CTD-2185H17 | 5 | 142505106 | 142742827 | −0.0371 | 0.0543 | 0.3735 |
| 3682 | RP11-226O9 | 5 | 143298228 | 143480824 | −0.0371 | 0.0543 | 0.3735 |
| 2822 | CTC-370H24 | 5 | 145164596 | 145347857 | −0.0371 | 0.0543 | 0.3735 |
| 3310 | RP11-481E16 | 5 | 146313344 | 146482537 | −0.0595 | 0.0603 | 0.3696 |
| 2821 | CTB-107A22 | 5 | 146972544 | 147089688 | −0.0592 | 0.0505 | 0.3751 |
| 2829 | CTB-176I15 | 5 | 147869999 | 147926171 | −0.0592 | 0.0505 | 0.3751 |
| 882 | RP11-394O4 | 5 | 148643204 | 148821858 | −0.0589 | 0.0575 | 0.3823 |
| 2290 | CTC-307M15 | 5 | 149174490 | 149349990 | −0.0589 | 0.0575 | 0.3823 |
| 1453 | CTB-1013H5 | 5 | 149407439 | 149494253 | −0.0589 | 0.0575 | 0.3823 |
| 665 | CTB-137O22 | 5 | 149978532 | 150055332 | −0.0589 | 0.0575 | 0.3823 |
| 677 | CTC-209K13 | 5 | 151319679 | 151506795 | −0.0595 | 0.0455 | 0.3692 |
| 3986 | RP11-26B2 | 5 | 152539684 | 152728888 | −0.0431 | 0.0377 | 0.3670 |
| 2715 | RP11-494C5 | 5 | 152817260 | 152985165 | −0.0431 | 0.0377 | 0.3670 |
| 2286 | CTC-263A14 | 5 | 154417750 | 154418252 | −0.0431 | 0.0377 | 0.3670 |
| 2281 | CTB-3C20 | 5 | 155606865 | 155624831 | −0.0437 | 0.0308 | 0.3553 |
| 661 | CTB-120L21 | 5 | 156441856 | 156481323 | −0.0437 | 0.0308 | 0.3553 |
| 1280 | RP11-102A1 | 5 | 157180096 | 157349365 | −0.0436 | 0.0343 | 0.3572 |
| 878 | RP11-391B7 | 5 | 157384965 | 157573351 | −0.0436 | 0.0343 | 0.3572 |
| 126 | CTC-279E3 | 5 | 159195207 | 159278867 | −0.0436 | 0.0504 | 0.3569 |
| 3511 | RP11-505G12 | 5 | 162295646 | 162296436 | −0.0436 | 0.0504 | 0.3569 |
| 4262 | RP11-505G12 | 5 | 162295646 | 162296436 | −0.0436 | 0.0504 | 0.3569 |
| 130 | CTC-320C6 | 5 | 163176794 | 163287813 | −0.0436 | 0.0504 | 0.3569 |
| 3306 | RP11-469G19 | 5 | 164752357 | 164922098 | −0.0436 | 0.0504 | 0.3569 |
| 3019 | RP11-170P5 | 5 | 165677723 | 165841050 | −0.0727 | 0.0400 | 0.3608 |
| 588 | RP11-511M9 | 5 | 165789662 | 165925473 | −0.0727 | 0.0400 | 0.3608 |
| 669 | CTB-180C19 | 5 | 166550287 | 166647640 | −0.0730 | 0.0346 | 0.3550 |
| 2297 | CTB-55A14 | 5 | 167592450 | 167807599 | −0.0730 | 0.0346 | 0.3550 |
| 1149 | RP11-420L4 | 5 | 169138790 | 169139510 | −0.0731 | 0.0577 | 0.3519 |
| 1869 | RP11-292M11 | 5 | 169915012 | 170086458 | −0.0726 | 0.0489 | 0.3638 |
| 1672 | RP11-20O22 | 5 | 171046969 | 171113733 | −0.0318 | 0.0374 | 0.3728 |
| 2143 | RP11-20O22 | 5 | 171046969 | 171113733 | −0.0318 | 0.0374 | 0.3728 |
| 133 | CTB-54I1 | 5 | 171714383 | 171955339 | −0.0317 | 0.0427 | 0.3757 |
| 3838 | RP11-489P1 | 5 | 173800904 | 173985872 | −0.0317 | 0.0427 | 0.3757 |
| 137 | CTB-73D21 | 5 | 174509436 | 174643725 | −0.0317 | 0.0427 | 0.3757 |
| 138 | CTC-355H1 | 5 | 175139848 | 175176805 | −0.0317 | 0.0427 | 0.3757 |
| 2301 | CTB-87L24 | 5 | 175835967 | 175892322 | −0.0312 | 0.0506 | 0.3856 |
| 2386 | RP11-564G9 | 5 | 176797689 | 176798343 | 0.0353 | 0.0505 | 0.3955 |
| 2014 | RP11-281O15 | 5 | 178243599 | 178455573 | −0.0332 | 0.0506 | 0.3862 |
| 3553 | GS-240-G13 | 5 | 180537866 | 180687866 | −0.1077 | −0.0768 | 0.4313 |
| 1930 | 84-C11 | 5 | 180689187 | 180839187 | −0.3190 | −0.3097 | 0.6022 |
| 1393 | GS-62-L11 | 6 | 110996 | 260996 | 0.0298 | 0.1440 | 0.4207 |
| 1927 | GS-196-I5 | 6 | 225000 | 375000 | 0.0295 | 0.1845 | 0.4135 |
| 3809 | RP11-13J16 | 6 | 1240996 | 1301756 | 0.1256 | 0.1486 | 0.3986 |
| 3817 | RP11-15N12 | 6 | 3292049 | 3425043 | 0.1256 | 0.1486 | 0.3986 |
| 1646 | RP11-174B19 | 6 | 5151674 | 5170849 | 0.1256 | 0.1486 | 0.3986 |
| 3818 | RP11-232H4 | 6 | 6151264 | 6169479 | 0.1256 | 0.1486 | 0.3986 |
| 3330 | RP11-163I22 | 6 | 6217514 | 6382300 | 0.1256 | 0.1486 | 0.3986 |
| 3271 | RP11-320C15 | 6 | 6705338 | 6770147 | 0.1256 | 0.1486 | 0.3986 |
| 3252 | RP3-336K20 | 6 | 7544150 | 7575683 | 0.1256 | 0.1681 | 0.3972 |
| 1527 | RP11-196B15 | 6 | 7898162 | 8078796 | 0.1257 | 0.1759 | 0.4006 |
| 3807 | RP11-385L2 | 6 | 8598346 | 8599694 | 0.1257 | 0.1759 | 0.4006 |
| 1119 | RP11-360O19 | 6 | 10597237 | 10684872 | 0.1254 | 0.1663 | 0.3932 |
| 1623 | RP3-510L9 | 6 | 11352273 | 11482926 | 0.1260 | 0.1625 | 0.4060 |
| 1114 | RP11-97A19 | 6 | 12430744 | 12449045 | 0.1046 | 0.1625 | 0.4014 |
| 3242 | RP1-257A7 | 6 | 13285332 | 13413248 | 0.1045 | 0.1559 | 0.4000 |
| 3262 | RP3-365E2 | 6 | 14536103 | 14673832 | 0.1044 | 0.1513 | 0.3972 |
| 1637 | RP1-232K4 | 6 | 15434571 | 15630732 | 0.1041 | 0.1434 | 0.3903 |
| 3241 | RP1-13D10 | 6 | 16165681 | 16318728 | 0.1041 | 0.1434 | 0.3903 |
| 1106 | RP11-68J15 | 6 | 17735571 | 17805613 | 0.1041 | 0.1386 | 0.3909 |
| 3811 | RP11-408C8 | 6 | 18937804 | 19079559 | 0.1043 | 0.1318 | 0.3961 |
| 1087 | RP4-625H18 | 6 | 19863670 | 20018592 | 0.1046 | 0.1361 | 0.4023 |
| 1634 | RP3-444C7 | 6 | 20630894 | 20759064 | 0.1046 | 0.1361 | 0.4023 |
| 1650 | RP11-204E9 | 6 | 21543541 | 21694512 | 0.1051 | 0.1456 | 0.4128 |
| 3825 | RP11-33I5 | 6 | 22580401 | 22666188 | 0.1051 | 0.1456 | 0.4128 |
| 1666 | RP11-289M23 | 6 | 23499374 | 23534047 | 0.1051 | 0.1456 | 0.4128 |
| 1116 | RP11-176J5 | 6 | 24254612 | 24267701 | 0.1054 | 0.1384 | 0.4196 |
| 3826 | RP11-289G11 | 6 | 25494592 | 25630305 | 0.1342 | 0.1383 | 0.4307 |
| 1093 | RP1-86C11 | 6 | 27152454 | 27241469 | 0.1330 | 0.1332 | 0.4307 |
| 3789 | RP1-153G14 | 6 | 27444496 | 27526951 | 0.1330 | 0.1332 | 0.4307 |
| 2505 | RP3-431A14 | 6 | 36751257 | 36944619 | 0.0957 | 0.1404 | 0.4767 |
| 1635 | RP1-90K10 | 6 | 36944620 | 37077773 | 0.0957 | 0.1404 | 0.4767 |
| 3260 | RP3-441A12 | 6 | 37889292 | 38009626 | 0.0953 | 0.1367 | 0.4697 |
| 1092 | RP3-350J21 | 6 | 39022180 | 39078688 | 0.0953 | 0.1367 | 0.4697 |
| 3246 | RP1-278E11 | 6 | 39977164 | 40076642 | 0.1233 | 0.1366 | 0.4735 |
| 1126 | RP11-162O6 | 6 | 40860313 | 41030209 | 0.1212 | 0.1249 | 0.4270 |
| 3272 | RP11-533O20 | 6 | 42026550 | 42131628 | 0.1049 | 0.1441 | 0.4115 |
| 3823 | RP11-501I18 | 6 | 42815771 | 42914110 | 0.1049 | 0.1441 | 0.4115 |
| 1654 | RP11-227E22 | 6 | 43965908 | 44050879 | 0.1039 | 0.1212 | 0.3906 |
| 1112 | RP11-554O14 | 6 | 44987769 | 45164736 | 0.0597 | 0.1009 | 0.3808 |
| 3808 | RP11-546O15 | 6 | 45777368 | 45960349 | 0.0601 | 0.0842 | 0.3888 |
| 1102 | RP3-365O12 | 6 | 46918495 | 47036031 | 0.0388 | 0.1027 | 0.3835 |
| 1626 | RP3-402H5 | 6 | 47870439 | 48002405 | 0.0379 | 0.0954 | 0.3649 |
| 1111 | RP11-334H12 | 6 | 48824592 | 48956050 | 0.0381 | 0.0916 | 0.3676 |
| 1100 | RP3-442L6 | 6 | 49686791 | 49805314 | 0.0397 | 0.1202 | 0.4028 |
| 1094 | RP3-336H9 | 6 | 50775801 | 50907374 | 0.0375 | 0.0961 | 0.3550 |
| 3268 | RP11-529A21 | 6 | 51408727 | 51472574 | 0.0374 | 0.0899 | 0.3532 |
| 3284 | RP11-362K18 | 6 | 54103953 | 54130898 | 0.0006 | 0.0632 | 0.3637 |
| 1667 | RP11-524H19 | 6 | 54743356 | 54856430 | 0.0415 | 0.0633 | 0.3559 |
| 1659 | RP11-472M19 | 6 | 56747375 | 56905292 | 0.0414 | 0.0586 | 0.3543 |
| 1630 | RP3-422B11 | 6 | 57292500 | 57422028 | 0.0407 | 0.0765 | 0.3398 |
| 3810 | RP11-199A24 | 6 | 58610220 | 58788604 | 0.0407 | 0.0765 | 0.3398 |
| 1124 | RP11-506N21 | 6 | 62639102 | 62725300 | 0.0419 | 0.0672 | 0.3642 |
| 3281 | RP11-767J14 | 6 | 63764581 | 63875481 | 0.0420 | 0.0562 | 0.3681 |
| 1120 | RP11-349P19 | 6 | 65158448 | 65208779 | 0.0426 | 0.0652 | 0.3814 |
| 1627 | RP1-40C9 | 6 | 65839972 | 65862416 | 0.0426 | 0.0652 | 0.3814 |
| 1090 | RP3-324B8 | 6 | 66901062 | 67021596 | 0.0435 | 0.0733 | 0.3992 |
| 1651 | RP11-409K15 | 6 | 68213162 | 68318818 | −0.0054 | 0.0654 | 0.3838 |
| 1089 | RP1-46B1 | 6 | 69438689 | 69596892 | −0.0054 | 0.0654 | 0.3838 |
| 1633 | RP1-160B9 | 6 | 69937981 | 70007502 | −0.0469 | 0.0571 | 0.3952 |
| 3819 | RP11-462G2 | 6 | 71121392 | 71183718 | −0.0220 | 0.0576 | 0.3906 |
| 3282 | RP11-111D8 | 6 | 72159825 | 72169003 | −0.0193 | 0.0725 | 0.4478 |
| 3822 | RP11-256L9 | 6 | 73180824 | 73217550 | −0.0210 | 0.0406 | 0.4113 |
| 3801 | RP1-234P15 | 6 | 75947840 | 76073480 | −0.0219 | 0.0134 | 0.3918 |
| 1642 | RP3-472A9 | 6 | 76600011 | 76685205 | −0.4201 | −0.2326 | 0.7162 |
| 3275 | RP11-343P23 | 6 | 77623996 | 77788369 | −0.0777 | 0.0213 | 0.3952 |
| 3806 | RP11-173D14 | 6 | 79534513 | 79708816 | −0.0777 | 0.0213 | 0.3952 |
| 3793 | RP1-159G19 | 6 | 80482703 | 80571211 | −0.0777 | 0.0213 | 0.3952 |
| 1881 | RP11-296E7 | 6 | 80815400 | 80852964 | −0.0777 | 0.0213 | 0.3952 |
| 3821 | RP11-25O6 | 6 | 83405494 | 83562457 | −0.0771 | 0.0094 | 0.4071 |
| 3781 | RP1-120N9 | 6 | 84309049 | 84476066 | −0.0774 | 0.0187 | 0.4027 |
| 3814 | RP11-223L24 | 6 | 85028178 | 85089451 | −0.0777 | 0.0103 | 0.3944 |
| 1661 | RP11-30P6 | 6 | 86051994 | 86222225 | −0.0777 | 0.0103 | 0.3944 |
| 1118 | RP11-106G15 | 6 | 88309107 | 88415806 | −0.0768 | 0.0008 | 0.4149 |
| 1081 | RP1-23D17 | 6 | 88811014 | 88923225 | −0.0768 | 0.0008 | 0.4149 |
| 3791 | RP1-61F10 | 6 | 89632167 | 89689630 | −0.0768 | 0.0008 | 0.4149 |
| 1621 | RP1-122O8 | 6 | 90373901 | 90514111 | −0.0767 | −0.0158 | 0.4175 |
| 1629 | RP1-154G14 | 6 | 91251386 | 91352280 | −0.1041 | −0.0282 | 0.4204 |
| 3288 | RP11-538A16 | 6 | 93629576 | 93823839 | −0.1041 | −0.0486 | 0.4208 |
| 3269 | RP11-572N15 | 6 | 96322017 | 96469450 | −0.1041 | −0.0486 | 0.4208 |
| 782 | RP11-4D24 | 6 | 96958620 | 97139258 | −0.1041 | −0.0486 | 0.4208 |
| 1622 | RP3-393D12 | 6 | 97092087 | 97184903 | −0.1041 | −0.0486 | 0.4208 |
| 1082 | RP1-273N12 | 6 | 99385962 | 99512744 | −0.1035 | −0.0509 | 0.4323 |
| 3278 | RP11-98I9 | 6 | 99909276 | 100037180 | −0.1033 | −0.0785 | 0.4364 |
| 1122 | RP11-117M4 | 6 | 100808566 | 100935179 | −0.0852 | −0.0725 | 0.4305 |
| 3803 | RP1-167P23 | 6 | 101487105 | 101513554 | −0.0852 | −0.0725 | 0.4305 |
| 3279 | RP11-347H8 | 6 | 102119070 | 102294657 | −0.0852 | −0.0725 | 0.4305 |
| 1104 | RP3-449G2 | 6 | 103440994 | 103493022 | −0.0852 | −0.0725 | 0.4305 |
| 3812 | RP11-721G11 | 6 | 116166720 | 116274627 | −0.0826 | −0.0581 | 0.4867 |
| 3251 | RP4-726A1 | 6 | 124382750 | 124503648 | −0.0864 | −0.0730 | 0.4035 |
| 3795 | RP1-84N20 | 6 | 125389373 | 125541247 | −0.0864 | −0.0730 | 0.4035 |
| 3827 | RP11-524A17 | 6 | 126458967 | 126536599 | −0.0863 | −0.0633 | 0.4054 |
| 3274 | RP11-95M15 | 6 | 137962575 | 138077798 | −0.0527 | −0.0783 | 0.4087 |
| 1653 | RP11-15H7 | 6 | 139976115 | 140131794 | −0.0528 | −0.0817 | 0.4110 |
| 1638 | RP3-460G2 | 6 | 141124534 | 141227085 | −0.0535 | −0.0658 | 0.4272 |
| 3283 | RP11-368P1 | 6 | 142222367 | 142361452 | −0.0910 | −0.1020 | 0.3848 |
| 3270 | RP11-86O4 | 6 | 143501404 | 143622432 | −0.0910 | −0.1020 | 0.3848 |
| 3813 | RP11-3B11 | 6 | 144509482 | 144514258 | −0.0910 | −0.1020 | 0.3848 |
| 1647 | RP11-386H19 | 6 | 145089782 | 145171242 | −0.0909 | −0.1192 | 0.3814 |
| 1128 | RP11-545I5 | 6 | 146157896 | 146266194 | −0.0903 | −0.1236 | 0.3700 |
| 3273 | RP11-631F7 | 6 | 148426158 | 148609993 | −0.0903 | −0.1236 | 0.3700 |
| 1101 | RP1-111D6 | 6 | 149547579 | 149673910 | −0.0902 | −0.1305 | 0.3674 |
| 1099 | RP5-1179L24 | 6 | 150539735 | 150646838 | −0.0902 | −0.1305 | 0.3674 |
| 1084 | RP1-297M16 | 6 | 151496433 | 151562014 | −0.0902 | −0.1305 | 0.3674 |
| 345 | RP3-443C4 | 6 | 152121130 | 152188475 | −0.0917 | −0.1010 | 0.3986 |
| 3344 | RP11-450E24 | 6 | 152186476 | 152326162 | −0.0917 | −0.1010 | 0.3986 |
| 888 | RP1-130E4 | 6 | 152411768 | 152520660 | −0.0910 | −0.1174 | 0.3831 |
| 3787 | RP4-546K19 | 6 | 153104985 | 153131413 | −0.0904 | −0.1377 | 0.3711 |
| 1645 | RP11-495B4 | 6 | 154042907 | 154157510 | −0.0905 | −0.1378 | 0.3729 |
| 3263 | RP1-66H9 | 6 | 155289734 | 155381577 | −0.0907 | −0.1570 | 0.3767 |
| 1125 | RP11-100E6 | 6 | 155961812 | 156120703 | −0.0907 | −0.1570 | 0.3767 |
| 3254 | RP3-336G18 | 6 | 156885767 | 156988048 | −0.0907 | −0.1570 | 0.3767 |
| 3280 | RP11-266C7 | 6 | 158279904 | 158440778 | −0.0910 | −0.1798 | 0.3847 |
| 3243 | RP3-442A17 | 6 | 158818064 | 158854414 | −0.0910 | −0.1798 | 0.3847 |
| 1121 | RP11-13P5 | 6 | 159413709 | 159520135 | −0.0910 | −0.1798 | 0.3847 |
| 1103 | RP1-249F5 | 6 | 160224241 | 160354109 | −0.0908 | −0.1841 | 0.3808 |
| 3894 | RP11-288H12 | 6 | 160354009 | 160500259 | −0.0908 | −0.1841 | 0.3808 |
| 3782 | RP3-366M24 | 6 | 160621320 | 160702239 | −0.0908 | −0.1841 | 0.3808 |
| 3783 | RP3-473J16 | 6 | 161450523 | 161626739 | −0.0910 | −0.1897 | 0.3848 |
| 3250 | RP1-292F10 | 6 | 162710104 | 162803740 | −0.0912 | −0.1793 | 0.3892 |
| 1083 | RP1-257A15 | 6 | 163355878 | 163476865 | −0.0912 | −0.1793 | 0.3892 |
| 3247 | RP3-471D13 | 6 | 164296629 | 164338799 | −0.0912 | −0.1869 | 0.3874 |
| 3805 | RP11-300M24 | 6 | 165061358 | 165244699 | −0.0912 | −0.1869 | 0.3874 |
| 1662 | RP11-289E16 | 6 | 165840859 | 165928214 | −0.0912 | −0.1869 | 0.3874 |
| 3285 | RP11-351J23 | 6 | 167790640 | 167835277 | −0.0912 | −0.1869 | 0.3874 |
| 3802 | RP3-470B24 | 6 | 168036070 | 168121862 | −0.0912 | −0.1869 | 0.3874 |
| 1625 | RP1-137D17 | 6 | 169488216 | 169576118 | −0.0914 | −0.1858 | 0.3931 |
| 3557 | GS-57-H24 | 6 | 170544992 | 170694992 | −0.0914 | −0.1858 | 0.3931 |
| 1397 | GS-164-D18 | 7 | 180000 | 330000 | −0.0858 | −0.1343 | 0.4297 |
| 1891 | RP11-510K8 | 7 | 1611828 | 1805609 | −0.0842 | −0.1944 | 0.3942 |
| 2653 | RP4-607J2 | 7 | 2370558 | 2519955 | −0.0840 | −0.2089 | 0.3898 |
| 1564 | RP11-348A21 | 7 | 3526556 | 3613603 | −0.0840 | −0.2089 | 0.3898 |
| 3395 | RP11-348A21 | 7 | 3526556 | 3613603 | −0.0840 | −0.2089 | 0.3898 |
| 570 | RP11-172O13 | 7 | 5704716 | 5847079 | −0.0835 | −0.1980 | 0.3794 |
| 1403 | RP1-42M2 | 7 | 5969700 | 6049710 | −0.0835 | −0.1980 | 0.3794 |
| 1029 | RP4-810E6 | 7 | 6049510 | 6202437 | −0.0835 | −0.1980 | 0.3794 |
| 505 | RP4-733B9 | 7 | 7176436 | 7292189 | −0.0835 | −0.1902 | 0.3799 |
| 564 | RP11-505D17 | 7 | 7947759 | 8125919 | −0.0836 | −0.1851 | 0.3817 |
| 3487 | RP11-304A10 | 7 | 8950512 | 9040173 | −0.0836 | −0.1851 | 0.3817 |
| 4255 | RP11-304A10 | 7 | 8950512 | 9040173 | −0.0836 | −0.1851 | 0.3817 |
| 4114 | RP5-959C21 | 7 | 9924891 | 10064840 | −0.0836 | −0.1851 | 0.3817 |
| 583 | RP11-352E12 | 7 | 10064640 | 10151963 | −0.0836 | −0.1851 | 0.3817 |
| 2200 | RP11-392P1 | 7 | 10372373 | 10429780 | −0.0834 | −0.1656 | 0.3768 |
| 1402 | RP5-855F16 | 7 | 10904962 | 11000307 | −0.0834 | −0.1566 | 0.3784 |
| 2712 | RP11-502P9 | 7 | 11782368 | 11938707 | −0.0834 | −0.1566 | 0.3784 |
| 2738 | RP11-195L14 | 7 | 12951352 | 13077542 | −0.0835 | −0.1502 | 0.3806 |
| 501 | RP4-685A2 | 7 | 13887063 | 13996422 | −0.0838 | −0.1356 | 0.3865 |
| 1720 | RP11-512E16 | 7 | 14128072 | 14279388 | −0.0838 | −0.1356 | 0.3865 |
| 1455 | RP11-547G15 | 7 | 14863402 | 15056327 | −0.0838 | −0.1356 | 0.3865 |
| 1671 | RP11-547G15 | 7 | 14863402 | 15056327 | −0.0838 | −0.1356 | 0.3865 |
| 875 | RP11-196O16 | 7 | 16023294 | 16205465 | −0.0299 | −0.0836 | 0.3780 |
| 4040 | RP11-323K15 | 7 | 17781327 | 17929240 | −0.0296 | −0.0825 | 0.3713 |
| 111 | RP11-71F18 | 7 | 19406242 | 19580569 | −0.0294 | −0.0729 | 0.3675 |
| 1670 | RP11-486P11 | 7 | 20042179 | 20150597 | −0.0295 | −0.0646 | 0.3682 |
| 1578 | RP11-445O1 | 7 | 21588234 | 21669042 | −0.0291 | −0.0410 | 0.3614 |
| 2490 | RP11-343P21 | 7 | 24511504 | 24521807 | −0.0310 | −0.0310 | 0.4010 |
| 3436 | RP11-99O17 | 7 | 25824267 | 25925677 | −0.0310 | −0.0310 | 0.4010 |
| 3865 | RP11-155G7 | 7 | 36168081 | 36309869 | 0.0047 | 0.0114 | 0.3741 |
| 1882 | RP11-302L6 | 7 | 37482196 | 37597750 | 0.0050 | 0.0187 | 0.3819 |
| 3223 | RP11-243E12 | 7 | 37825117 | 37999358 | 0.0050 | 0.0187 | 0.3819 |
| 3227 | RP11-273L18 | 7 | 38356844 | 38489780 | 0.0049 | 0.0256 | 0.3789 |
| 114 | RP11-64I2 | 7 | 39496099 | 39613863 | 0.0049 | 0.0256 | 0.3789 |
| 1037 | RP5-1032B10 | 7 | 43150163 | 43228683 | 0.0050 | 0.0123 | 0.3811 |
| 2201 | RP11-36H20 | 7 | 43453095 | 43594872 | 0.0050 | 0.0074 | 0.3804 |
| 2657 | RP4-647J21 | 7 | 44926600 | 45048880 | 0.0050 | 0.0074 | 0.3804 |
| 30 | RP11-111G20 | 7 | 45308102 | 45400347 | 0.0050 | 0.0074 | 0.3804 |
| 1737 | RP11-132L11 | 7 | 45887168 | 45956854 | 0.0050 | 0.0074 | 0.3804 |
| 774 | RP11-571N3 | 7 | 45956855 | 46124185 | 0.0050 | 0.0074 | 0.3804 |
| 2056 | RP11-95J15 | 7 | 46567503 | 46734218 | 0.0050 | 0.0074 | 0.3804 |
| 889 | RP5-1091E12 | 7 | 55045081 | 55222878 | 0.0043 | 0.0059 | 0.3671 |
| 1191 | RP11-339F13 | 7 | 55222879 | 55347931 | 0.0043 | 0.0059 | 0.3671 |
| 799 | RP11-10F11 | 7 | 56639424 | 56803089 | −0.0131 | −0.0195 | 0.3525 |
| 47 | RP11-313P13 | 7 | 71970679 | 72156415 | 0.0007 | 0.0035 | 0.3482 |
| 3294 | RP11-450O3 | 7 | 72073705 | 72269192 | 0.0007 | 0.0035 | 0.3482 |
| 1945 | RP4-771P4 | 7 | 73732805 | 73733598 | 0.0314 | 0.0635 | 0.3328 |
| 1949 | RP4-799O8 | 7 | 76190005 | 76361341 | 0.0318 | 0.0499 | 0.3408 |
| 2736 | RP11-467H10 | 7 | 76490656 | 76687315 | 0.0321 | 0.0543 | 0.3472 |
| 4120 | RP4-562A11 | 7 | 76971221 | 77115186 | 0.0324 | 0.0620 | 0.3557 |
| 1909 | RP11-538D15 | 7 | 77710860 | 77899695 | 0.0327 | 0.0681 | 0.3614 |
| 579 | RP11-343J14 | 7 | 78415998 | 78422030 | 0.0327 | 0.0681 | 0.3614 |
| 3206 | RP5-1164F5 | 7 | 78422031 | 78579140 | 0.0327 | 0.0681 | 0.3614 |
| 343 | RP5-1057M1 | 7 | 79539131 | 79644969 | 0.0333 | 0.0574 | 0.3739 |
| 3892 | RP11-575G1 | 7 | 81501227 | 81558313 | 0.0338 | 0.0388 | 0.3861 |
| 876 | RP11-263N12 | 7 | 89329383 | 89449178 | 0.0514 | 0.0537 | 0.3963 |
| 2650 | RP5-1099C19 | 7 | 92000501 | 92074199 | 0.0509 | 0.0534 | 0.3850 |
| 3298 | RP11-455I9 | 7 | 93400348 | 93502745 | 0.0509 | 0.0534 | 0.3850 |
| 3440 | RP11-101N13 | 7 | 94315003 | 94427370 | 0.0509 | 0.0534 | 0.3850 |
| 2192 | RP11-380G21 | 7 | 97314556 | 97410111 | 0.0360 | 0.0339 | 0.3826 |
| 2483 | RP11-148A10 | 7 | 103964266 | 104082406 | 0.0705 | 0.0758 | 0.3680 |
| 388 | RP11-22N19 | 7 | 105622027 | 105748629 | 0.0703 | 0.0829 | 0.3640 |
| 1274 | RP11-17I10 | 7 | 105828598 | 105875439 | 0.0936 | 0.0916 | 0.3656 |
| 4295 | RP11-17I10 | 7 | 105828598 | 105875439 | 0.0936 | 0.0916 | 0.3656 |
| 3443 | RP11-45K3 | 7 | 114973552 | 115099387 | 0.0669 | 0.0895 | 0.3935 |
| 342 | RP11-354H2 | 7 | 115743960 | 115770524 | 0.0668 | 0.0934 | 0.3912 |
| 3617 | CTB-1013N12 | 7 | 116079831 | 116304165 | 0.0666 | 0.1072 | 0.3869 |
| 587 | RP11-374M7 | 7 | 116365845 | 116463440 | 0.0666 | 0.1072 | 0.3869 |
| 1033 | RP5-866N18 | 7 | 116651982 | 116688781 | 0.0666 | 0.1072 | 0.3869 |
| 1824 | RP11-126C19 | 7 | 117988169 | 118140112 | 0.0670 | 0.0988 | 0.3970 |
| 3773 | RP11-126C19 | 7 | 117988169 | 118140112 | 0.0670 | 0.0988 | 0.3970 |
| 4217 | RP11-105B19 | 7 | 118629304 | 118806607 | 0.0670 | 0.0988 | 0.3970 |
| 3015 | RP11-376O14 | 7 | 119703959 | 119857235 | 0.0670 | 0.0988 | 0.3970 |
| 2669 | RP4-745K6 | 7 | 120979600 | 121110006 | 0.0670 | 0.0988 | 0.3970 |
| 32 | RP11-384A20 | 7 | 121371260 | 121438335 | 0.0687 | 0.0816 | 0.4325 |
| 773 | RP11-560I19 | 7 | 121590947 | 121768222 | 0.0673 | 0.1086 | 0.4025 |
| 1467 | RP11-560I19 | 7 | 121590947 | 121768222 | 0.0673 | 0.1086 | 0.4025 |
| 1502 | RP11-106F1 | 7 | 122549026 | 122703244 | 0.0673 | 0.1086 | 0.4025 |
| 1605 | RP11-106F1 | 7 | 122549026 | 122703244 | 0.0673 | 0.1086 | 0.4025 |
| 3582 | RP5-902E20 | 7 | 123592768 | 123711877 | 0.0674 | 0.1141 | 0.4039 |
| 2195 | RP11-264K23 | 7 | 123711878 | 123721155 | 0.0672 | 0.1098 | 0.4016 |
| 2119 | RP11-420H19 | 7 | 124677369 | 124832004 | 0.0672 | 0.1098 | 0.4016 |
| 1264 | RP11-24C13 | 7 | 126157554 | 126225780 | 0.0668 | 0.1223 | 0.3922 |
| 4237 | RP11-224A1 | 7 | 127107823 | 127230031 | 0.0666 | 0.1328 | 0.3877 |
| 2461 | RP11-391H5 | 7 | 127389011 | 127586356 | 0.0661 | 0.1397 | 0.3768 |
| 281 | RP11-128A6 | 7 | 128410251 | 128501561 | 0.0661 | 0.1509 | 0.3764 |
| 1027 | RP5-999D10 | 7 | 129238727 | 129329133 | 0.0661 | 0.1509 | 0.3764 |
| 924 | RP11-36B6 | 7 | 130078069 | 130270836 | 0.0673 | 0.1827 | 0.4025 |
| 1556 | RP11-329I5 | 7 | 130598383 | 130792906 | 0.0676 | 0.1729 | 0.4098 |
| 3933 | RP11-329I5 | 7 | 130598383 | 130792906 | 0.0676 | 0.1729 | 0.4098 |
| 3683 | RP11-193I17 | 7 | 131217474 | 131308648 | 0.0676 | 0.1729 | 0.4098 |
| 1783 | RP11-355K3 | 7 | 131972230 | 132193724 | 0.0676 | 0.1729 | 0.4098 |
| 4260 | RP11-355K3 | 7 | 131972230 | 132193724 | 0.0676 | 0.1729 | 0.4098 |
| 1475 | RP11-239G19 | 7 | 133858770 | 134022400 | 0.0673 | 0.1622 | 0.4017 |
| 4233 | RP11-221G19 | 7 | 134079168 | 134234824 | 0.0673 | 0.1622 | 0.4017 |
| 4294 | RP11-221G19 | 7 | 134079168 | 134234824 | 0.0673 | 0.1622 | 0.4017 |
| 2999 | RP11-371N6 | 7 | 134684519 | 134842817 | 0.0673 | 0.1622 | 0.4017 |
| 3675 | RP11-88K4 | 7 | 136263936 | 136416924 | 0.0673 | 0.1622 | 0.4017 |
| 2045 | RP11-102G17 | 7 | 136606302 | 136766865 | 0.1143 | 0.1621 | 0.4112 |
| 263 | RP11-8P6 | 7 | 136684139 | 136846035 | 0.1143 | 0.1621 | 0.4112 |
| 2029 | RP11-269N18 | 7 | 137919496 | 138093873 | 0.1354 | 0.1621 | 0.4139 |
| 836 | RP11-383F6 | 7 | 138384237 | 138486575 | 0.1350 | 0.1585 | 0.4052 |
| 1046 | RP5-1173P7 | 7 | 140392201 | 140521423 | 0.1350 | 0.1585 | 0.4052 |
| 27 | RP11-237G17 | 7 | 140911726 | 140979677 | 0.1350 | 0.1581 | 0.4059 |
| 3578 | RP5-894A10 | 7 | 140977682 | 141092852 | 0.1350 | 0.1581 | 0.4059 |
| 520 | RP11-426J23 | 7 | 142042929 | 142222513 | 0.1351 | 0.1385 | 0.4077 |
| 1953 | RP4-811H12 | 7 | 146787501 | 146949716 | 0.1150 | 0.1224 | 0.4158 |
| 2733 | RP11-69O3 | 7 | 155193647 | 155346385 | 0.1155 | 0.1169 | 0.4253 |
| 1958 | RP5-982E9 | 7 | 156044134 | 156202353 | 0.1155 | 0.1169 | 0.4253 |
| 1917 | GS-580-L5 | 8 | 175000 | 325000 | −0.1945 | −0.2122 | 0.4143 |
| 3547 | GS-77-L23 | 8 | 275000 | 425000 | −0.1945 | −0.2228 | 0.4138 |
| 2486 | RP11-338B22 | 8 | 477644 | 658409 | −0.1945 | −0.2228 | 0.4138 |
| 3561 | RP4-593A12 | 8 | 1528552 | 1680296 | −0.1945 | −0.2228 | 0.4138 |
| 489 | RP4-605G11 | 8 | 1610535 | 1706678 | −0.1945 | −0.2228 | 0.4138 |
| 1238 | RP11-336N16 | 8 | 2898584 | 3068882 | −0.1977 | −0.2279 | 0.4836 |
| 1283 | RP11-45M12 | 8 | 3899636 | 4066807 | −0.1985 | −0.2435 | 0.5009 |
| 3961 | RP11-16H11 | 8 | 4187855 | 4386741 | −0.1977 | −0.2289 | 0.4837 |
| 2413 | RP11-1K11 | 8 | 4596112 | 4755808 | −0.1977 | −0.2289 | 0.4837 |
| 33 | RP11-29A2 | 8 | 5176646 | 5256106 | −0.1977 | −0.2289 | 0.4837 |
| 1034 | RP5-991O23 | 8 | 5326020 | 5459277 | −0.1977 | −0.2289 | 0.4837 |
| 2832 | CTD-2629I16 | 8 | 6684740 | 6685317 | −0.1957 | −0.2074 | 0.4394 |
| 3514 | RP11-540E4 | 8 | 8029024 | 8179923 | −0.1961 | −0.2107 | 0.4479 |
| 2068 | RP11-211C9 | 8 | 8504285 | 8677721 | −0.1961 | −0.2107 | 0.4479 |
| 3647 | RP11-241P12 | 8 | 9788890 | 9958420 | −0.1217 | −0.1978 | 0.4468 |
| 1156 | RP11-177H2 | 8 | 10696470 | 10796498 | −0.1217 | −0.1978 | 0.4468 |
| 1330 | RP11-297N6 | 8 | 11571002 | 11740080 | −0.0592 | −0.1972 | 0.5011 |
| 1448 | RP11-589N15 | 8 | 11627380 | 11803128 | −0.0592 | −0.1972 | 0.5011 |
| 2124 | RP11-433L7 | 8 | 14278096 | 14461154 | −0.1428 | −0.2008 | 0.4096 |
| 653 | RP11-60C8 | 8 | 15279356 | 15445712 | −0.1428 | −0.2008 | 0.4096 |
| 957 | RP11-44L18 | 8 | 15557561 | 15699688 | −0.1428 | −0.2008 | 0.4096 |
| 3644 | RP11-255E13 | 8 | 16333693 | 16472754 | −0.1427 | −0.2092 | 0.4072 |
| 1139 | RP11-525O22 | 8 | 17784184 | 17950943 | −0.1425 | −0.2167 | 0.4039 |
| 2000 | RP11-809L8 | 8 | 18302963 | 18450086 | −0.1425 | −0.2167 | 0.4039 |
| 1162 | RP11-161I2 | 8 | 18447534 | 18644382 | −0.1425 | −0.2167 | 0.4039 |
| 1361 | RP11-529P14 | 8 | 21756478 | 21872595 | −0.1423 | −0.2379 | 0.3974 |
| 863 | RP11-177H13 | 8 | 23107310 | 23286631 | −0.1423 | −0.2379 | 0.3974 |
| 777 | RP11-561E1 | 8 | 24385106 | 24469397 | −0.1425 | −0.2298 | 0.4031 |
| 2997 | RP11-395I14 | 8 | 25159703 | 25340100 | −0.1425 | −0.2298 | 0.4031 |
| 833 | RP11-138J2 | 8 | 27277246 | 27304557 | −0.1168 | −0.1979 | 0.3969 |
| 3388 | RP11-356F24 | 8 | 28432319 | 28631588 | −0.1169 | −0.1899 | 0.4006 |
| 3705 | RP11-356F24 | 8 | 28432319 | 28631588 | −0.1169 | −0.1899 | 0.4006 |
| 1473 | RP11-263C6 | 8 | 28927769 | 29094558 | −0.1169 | −0.1899 | 0.4006 |
| 299 | RP11-366G13 | 8 | 29818492 | 30006914 | −0.1173 | −0.1728 | 0.4083 |
| 4033 | RP11-293D9 | 8 | 30923281 | 30990492 | −0.1174 | −0.1636 | 0.4108 |
| 1184 | RP11-473A17 | 8 | 30945705 | 31125802 | −0.0853 | −0.1636 | 0.4094 |
| 3879 | RP11-363L24 | 8 | 31125803 | 31243057 | −0.0853 | −0.1636 | 0.4094 |
| 1738 | RP11-301H15 | 8 | 32530006 | 32715284 | −0.0851 | −0.1117 | 0.4067 |
| 3973 | RP11-11N9 | 8 | 32855440 | 33028630 | −0.0851 | −0.1117 | 0.4067 |
| 573 | RP11-75P13 | 8 | 34287764 | 34455012 | −0.0316 | −0.1025 | 0.4322 |
| 4103 | RP1-144M5 | 8 | 34483916 | 34660804 | −0.0316 | −0.1025 | 0.4322 |
| 1943 | RP1-155L11 | 8 | 35234984 | 35343974 | −0.0316 | −0.1025 | 0.4322 |
| 3889 | RP11-98I12 | 8 | 36363042 | 36516657 | −0.0135 | −0.0841 | 0.4308 |
| 1535 | RP11-197P20 | 8 | 37204481 | 37371241 | −0.0136 | −0.0487 | 0.4338 |
| 3762 | RP11-197P20 | 8 | 37204481 | 37371241 | −0.0136 | −0.0487 | 0.4338 |
| 3927 | RP11-350N15 | 8 | 38358847 | 38488999 | 0.2443 | 0.0056 | 0.5921 |
| 3439 | RP11-44K6 | 8 | 39836600 | 39985607 | 0.2983 | 0.0785 | 0.6093 |
| 974 | RP11-51K12 | 8 | 40501066 | 40649254 | 0.2425 | 0.0520 | 0.4954 |
| 25 | RP11-15G16 | 8 | 41287409 | 41424589 | 0.2425 | 0.0564 | 0.4937 |
| 1951 | RP1-198M21 | 8 | 41516869 | 41617043 | 0.3371 | 0.0773 | 0.4982 |
| 3882 | RP11-231D20 | 8 | 42155380 | 42333717 | 0.2687 | 0.0774 | 0.4896 |
| 1686 | RP11-503E24 | 8 | 42503724 | 42674303 | 0.2689 | 0.0825 | 0.4861 |
| 2831 | CTD-2115H11 | 8 | 43315811 | 43438715 | 0.2907 | 0.0907 | 0.4868 |
| 3403 | RP11-350F16 | 8 | 47816603 | 47982207 | 0.0546 | 0.0800 | 0.4071 |
| 1293 | RP11-11C20 | 8 | 52786052 | 52920538 | 0.1624 | 0.1099 | 0.4082 |
| 1531 | RP11-197I11 | 8 | 53135835 | 53175020 | 0.1613 | 0.1300 | 0.4319 |
| 3860 | RP11-182E14 | 8 | 53938355 | 54105603 | 0.1615 | 0.1292 | 0.4269 |
| 97 | RP11-53M11 | 8 | 55513791 | 55667180 | 0.1615 | 0.1240 | 0.4267 |
| 3938 | RP11-342K10 | 8 | 57944550 | 58097264 | 0.1373 | 0.1373 | 0.4038 |
| 3452 | RP11-114M5 | 8 | 59452734 | 59619718 | 0.1373 | 0.1373 | 0.4038 |
| 1567 | RP11-414L17 | 8 | 61675923 | 61851185 | 0.1376 | 0.1511 | 0.4104 |
| 3072 | RP11-35A5 | 8 | 61829586 | 62011027 | 0.1383 | 0.1629 | 0.4255 |
| 4104 | RP3-491L6 | 8 | 61918628 | 61993775 | 0.1383 | 0.1678 | 0.4243 |
| 2492 | RP11-227F6 | 8 | 62411158 | 62572801 | 0.1377 | 0.1598 | 0.4113 |
| 431 | RP11-26L21 | 8 | 63219972 | 63361585 | 0.1377 | 0.1598 | 0.4113 |
| 977 | RP11-45K10 | 8 | 64465942 | 64651178 | 0.1378 | 0.1561 | 0.4152 |
| 3415 | RP11-21C5 | 8 | 69451201 | 69616969 | 0.1383 | 0.1401 | 0.4248 |
| 3454 | RP11-21C17 | 8 | 70222710 | 70378941 | 0.1383 | 0.1401 | 0.4248 |
| 261 | RP11-2B15 | 8 | 72635961 | 72767666 | 0.1388 | 0.1450 | 0.4351 |
| 1767 | RP11-351E7 | 8 | 73498816 | 73677520 | 0.1388 | 0.1450 | 0.4351 |
| 1879 | RP11-313C15 | 8 | 73722091 | 73910845 | 0.1388 | 0.1393 | 0.4367 |
| 1515 | RP11-88N8 | 8 | 75573453 | 75722946 | 0.1056 | 0.1251 | 0.4168 |
| 2418 | RP11-3N13 | 8 | 75850330 | 76026072 | 0.1318 | 0.1374 | 0.4342 |
| 2067 | RP11-202L1 | 8 | 77234275 | 77394656 | 0.1321 | 0.1463 | 0.4407 |
| 2598 | RP11-48D4 | 8 | 77936444 | 78029382 | 0.1325 | 0.1559 | 0.4487 |
| 800 | RP11-12L20 | 8 | 78929594 | 79062039 | 0.1325 | 0.1559 | 0.4487 |
| 4052 | RP11-523D2 | 8 | 79633094 | 79802906 | 0.1325 | 0.1559 | 0.4487 |
| 959 | RP11-27N21 | 8 | 80736766 | 80893803 | 0.1321 | 0.1543 | 0.4391 |
| 2594 | RP11-48B3 | 8 | 81538449 | 81648851 | 0.1323 | 0.1623 | 0.4437 |
| 102 | RP11-62E9 | 8 | 81904332 | 82095689 | 0.1327 | 0.1523 | 0.4533 |
| 3068 | RP11-34M16 | 8 | 82640038 | 82768430 | 0.1323 | 0.1420 | 0.4452 |
| 3138 | RP11-51M18 | 8 | 84421672 | 84550079 | 0.1353 | 0.1725 | 0.5103 |
| 2656 | RP5-1098O20 | 8 | 90947388 | 91066955 | 0.1322 | 0.1967 | 0.4433 |
| 338 | RP11-353D5 | 8 | 92425104 | 92553416 | 0.1321 | 0.2042 | 0.4410 |
| 254 | RP11-3J21 | 8 | 93160371 | 93308372 | 0.1321 | 0.2042 | 0.4410 |
| 874 | RP11-388K12 | 8 | 94214289 | 94424296 | 0.1456 | 0.2150 | 0.4450 |
| 1489 | RP11-266D22 | 8 | 95335790 | 95508534 | 0.1654 | 0.2150 | 0.4440 |
| 4028 | RP11-320N21 | 8 | 96081539 | 96178047 | 0.1819 | 0.2062 | 0.4635 |
| 246 | RP11-3D19 | 8 | 97773101 | 97944401 | 0.1197 | 0.2267 | 0.4470 |
| 3428 | RP11-24H3 | 8 | 99215537 | 99366374 | 0.1199 | 0.2331 | 0.4512 |
| 2963 | RP11-10G10 | 8 | 101279027 | 101431748 | 0.1212 | 0.2647 | 0.4794 |
| 1259 | RP11-21E8 | 8 | 102295847 | 102476213 | 0.1222 | 0.2921 | 0.5007 |
| 3011 | RP11-373L22 | 8 | 102544799 | 102710032 | 0.1221 | 0.2998 | 0.4988 |
| 1161 | RP11-150P21 | 8 | 103802267 | 103959662 | 0.1221 | 0.3151 | 0.5001 |
| 4027 | RP11-310H18 | 8 | 104441333 | 104600167 | 0.1214 | 0.3180 | 0.4836 |
| 3872 | RP11-188I6 | 8 | 104875738 | 105015142 | 0.1226 | 0.2991 | 0.5103 |
| 2453 | RP11-132E3 | 8 | 105218041 | 105402499 | 0.1213 | 0.3247 | 0.4817 |
| 1539 | RP11-200A13 | 8 | 106039025 | 106200563 | 0.1211 | 0.3370 | 0.4777 |
| 2276 | RP11-79F7 | 8 | 107820491 | 107973246 | 0.1215 | 0.3321 | 0.4875 |
| 1810 | RP11-20P9 | 8 | 108006920 | 108167162 | 0.1217 | 0.3378 | 0.4916 |
| 819 | RP11-115M13 | 8 | 108980084 | 109137354 | 0.1599 | 0.3379 | 0.4844 |
| 2115 | RP11-419L20 | 8 | 110461319 | 110612552 | 0.1604 | 0.3957 | 0.4963 |
| 4037 | RP11-294M6 | 8 | 111740331 | 111910034 | 0.1809 | 0.4074 | 0.4927 |
| 2451 | RP11-114O8 | 8 | 112187912 | 112266168 | 0.1809 | 0.4074 | 0.4927 |
| 1374 | RP11-544H16 | 8 | 112566055 | 112683962 | 0.1809 | 0.4074 | 0.4927 |
| 248 | RP11-11A18 | 8 | 113492780 | 113659918 | 0.1809 | 0.3994 | 0.4919 |
| 1472 | RP11-252H14 | 8 | 114428898 | 114574464 | 0.1809 | 0.3994 | 0.4919 |
| 801 | RP11-2K18 | 8 | 115760314 | 115915271 | 0.2136 | 0.4416 | 0.5040 |
| 3307 | RP11-536K17 | 8 | 117379793 | 117484202 | 0.2134 | 0.4466 | 0.4992 |
| 654 | RP11-67N21 | 8 | 118297084 | 118467725 | 0.2141 | 0.4352 | 0.5150 |
| 555 | RP11-494N20 | 8 | 118573210 | 118737745 | 0.2141 | 0.4352 | 0.5150 |
| 2803 | RP11-72M5 | 8 | 119798302 | 119961178 | 0.2142 | 0.4288 | 0.5171 |
| 2979 | RP11-115M9 | 8 | 122743719 | 122893632 | 0.2526 | 0.4608 | 0.5385 |
| 3957 | RP11-16G11 | 8 | 122793257 | 122875995 | 0.2526 | 0.4608 | 0.5385 |
| 2677 | CTB49g22 | 8 | 122849778 | 122999778 | 0.2526 | 0.4608 | 0.5385 |
| 3657 | RP11-96B2 | 8 | 123538667 | 123697997 | 0.2526 | 0.4608 | 0.5385 |
| 3871 | RP11-174I12 | 8 | 124550978 | 124718434 | 0.2528 | 0.4576 | 0.5428 |
| 1369 | RP11-532M24 | 8 | 125633453 | 125840521 | 0.2910 | 0.4661 | 0.5465 |
| 2723 | RP11-495D4 | 8 | 126599186 | 126798853 | 0.2910 | 0.4799 | 0.5469 |
| 3127 | RP11-28I2 | 8 | 127563614 | 127764311 | 0.2909 | 0.4870 | 0.5437 |
| 312 | RP11-382A18 | 8 | 128313668 | 128490427 | 0.2909 | 0.4870 | 0.5437 |
| 3044 | RP1-80K22 | 8 | 128735637 | 128884770 | 0.2913 | 0.4824 | 0.5520 |
| 258 | RP11-3O20 | 8 | 130380459 | 130458325 | 0.2915 | 0.4762 | 0.5562 |
| 1595 | RP11-3O20 | 8 | 130380459 | 130458325 | 0.2915 | 0.4762 | 0.5562 |
| 3414 | RP11-17E16 | 8 | 130562446 | 130578066 | 0.2917 | 0.4806 | 0.5606 |
| 2942 | RP11-4C17 | 8 | 132474524 | 132638132 | 0.2917 | 0.4806 | 0.5606 |
| 2799 | RP11-71N3 | 8 | 133156771 | 133318034 | 0.2908 | 0.4621 | 0.5426 |
| 886 | RP6-98A24 | 8 | 134125496 | 134255043 | 0.2910 | 0.4568 | 0.5464 |
| 2441 | RP11-127J8 | 8 | 134871608 | 135047479 | 0.2910 | 0.4568 | 0.5464 |
| 330 | RP11-343P9 | 8 | 136470355 | 136653421 | 0.2120 | 0.4616 | 0.5205 |
| 1703 | RP11-172M18 | 8 | 139307361 | 139453265 | 0.2120 | 0.4616 | 0.5205 |
| 1912 | RP11-526P7 | 8 | 140285419 | 140443685 | 0.2113 | 0.4316 | 0.5052 |
| 1295 | RP11-65A5 | 8 | 141431413 | 141551818 | 0.2113 | 0.4316 | 0.5052 |
| 315 | RP11-370K2 | 8 | 142620100 | 142790558 | 0.2113 | 0.4316 | 0.5052 |
| 576 | RP11-472K18 | 8 | 144183954 | 144333954 | 0.2320 | 0.4407 | 0.5218 |
| 500 | RP5-1118A7 | 8 | 144404204 | 144527057 | 0.2603 | 0.4633 | 0.5534 |
| 2743 | RP11-349C2 | 8 | 145582815 | 145779789 | 0.2606 | 0.4334 | 0.5612 |
| 1387 | GS-261-I1 | 8 | 146029826 | 146179826 | 0.2201 | 0.4334 | 0.5648 |
| 4081 | GS-489-D14 | 8 | 146029876 | 146179876 | 0.2201 | 0.4334 | 0.5648 |
| 3551 | RG-41-L13 | 9 | 115000 | 265000 | −0.1362 | −0.0273 | 0.4484 |
| 1921 | GS-43-N6 | 9 | 525000 | 675000 | −0.1362 | −0.0273 | 0.4484 |
| 992 | RP11-147I11 | 9 | 991053 | 1101150 | −0.1362 | −0.0273 | 0.4484 |
| 991 | RP11-48M17 | 9 | 2136330 | 2296367 | −0.1362 | −0.0273 | 0.4484 |
| 997 | RP11-320E16 | 9 | 2583689 | 2658649 | −0.1356 | −0.0121 | 0.4619 |
| 3595 | RP11-509J21 | 9 | 3533196 | 3696642 | −0.1356 | −0.0121 | 0.4619 |
| 3144 | RP11-125K10 | 9 | 4819656 | 4991841 | −0.1356 | −0.0121 | 0.4619 |
| 1435 | RP11-509D8 | 9 | 4991742 | 5008809 | −0.1347 | −0.0019 | 0.4817 |
| 2621 | RP11-218I7 | 9 | 5993678 | 6062142 | −0.0510 | 0.0272 | 0.5936 |
| 456 | RP11-106A1 | 9 | 6599727 | 6659690 | −0.1258 | −0.0142 | 0.4584 |
| 979 | RP11-29B9 | 9 | 7904421 | 8056890 | −0.1258 | −0.0142 | 0.4584 |
| 445 | RP11-175E13 | 9 | 8398602 | 8490985 | −0.1258 | −0.0142 | 0.4584 |
| 1439 | RP11-527D15 | 9 | 9657591 | 9823759 | −0.1261 | −0.0212 | 0.4511 |
| 447 | RP11-19G1 | 9 | 9999831 | 10094473 | −0.1261 | −0.0212 | 0.4511 |
| 2615 | RP11-23D5 | 9 | 11227455 | 11303482 | −0.1261 | −0.0262 | 0.4514 |
| 442 | RP11-352F21 | 9 | 11389654 | 11588151 | −0.1261 | −0.0212 | 0.4511 |
| 3162 | RP11-446F13 | 9 | 12281538 | 12346848 | −0.1262 | −0.0130 | 0.4502 |
| 449 | RP11-187K14 | 9 | 12872920 | 12952270 | −0.1262 | −0.0130 | 0.4502 |
| 3142 | RP11-413D24 | 9 | 13729563 | 13912972 | −0.1268 | −0.0214 | 0.4375 |
| 462 | RP11-408A13 | 9 | 14500602 | 14586756 | −0.1268 | −0.0214 | 0.4375 |
| 3587 | RP11-490C5 | 9 | 15298239 | 15399992 | −0.1268 | −0.0214 | 0.4375 |
| 460 | RP11-109M15 | 9 | 16171750 | 16325494 | −0.1268 | −0.0419 | 0.4371 |
| 984 | RP11-132E11 | 9 | 16995146 | 17138368 | −0.1268 | −0.0419 | 0.4371 |
| 980 | RP11-123J20 | 9 | 17933998 | 18013852 | −0.1269 | −0.0366 | 0.4347 |
| 1431 | RP11-503K16 | 9 | 18579960 | 18743122 | −0.1269 | −0.0366 | 0.4347 |
| 3599 | RP11-513M16 | 9 | 19310506 | 19423218 | −0.1269 | −0.0366 | 0.4347 |
| 2607 | RP11-15P13 | 9 | 20172502 | 20351132 | −0.1270 | −0.0446 | 0.4320 |
| 3140 | RP11-113D19 | 9 | 20996401 | 21158465 | −0.1271 | −0.0386 | 0.4303 |
| 3156 | RP11-149I2 | 9 | 21899259 | 22000413 | −0.1271 | −0.0386 | 0.4303 |
| 443 | RP11-11J1 | 9 | 22479496 | 22579721 | −0.1269 | −0.0502 | 0.4353 |
| 3591 | RP11-495L19 | 9 | 23461740 | 23557472 | −0.1269 | −0.0502 | 0.4353 |
| 3147 | RP11-33K8 | 9 | 24090706 | 24243453 | −0.1029 | −0.0468 | 0.4324 |
| 3583 | RP11-468C2 | 9 | 24937707 | 25069411 | −0.1029 | −0.0468 | 0.4324 |
| 987 | RP11-33G16 | 9 | 25690183 | 25853240 | −0.1029 | −0.0468 | 0.4324 |
| 2599 | RP11-5P15 | 9 | 26516496 | 26681794 | −0.0752 | −0.0468 | 0.4303 |
| 459 | RP11-27J8 | 9 | 27449441 | 27590267 | −0.0752 | −0.0468 | 0.4303 |
| 451 | RP11-20P5 | 9 | 28040167 | 28204457 | −0.0752 | −0.0468 | 0.4303 |
| 3141 | RP11-264J11 | 9 | 28670842 | 28755649 | −0.0745 | −0.0640 | 0.4461 |
| 3151 | RP11-48L13 | 9 | 29567030 | 29639069 | −0.0755 | −0.0377 | 0.4237 |
| 532 | RP11-630H9 | 9 | 30404961 | 30535678 | −0.0712 | 0.0095 | 0.5178 |
| 516 | RP11-402B2 | 9 | 31599160 | 31764898 | −0.0761 | −0.0171 | 0.4118 |
| 1055 | RP11-205M20 | 9 | 32452045 | 32607629 | −0.0761 | −0.0171 | 0.4118 |
| 1071 | RP11-326F20 | 9 | 33086353 | 33247752 | −0.0765 | −0.0069 | 0.4028 |
| 3215 | RP11-195F19 | 9 | 34634468 | 34820900 | −0.0411 | −0.0169 | 0.3855 |
| 2687 | RP11-112J3 | 9 | 35670117 | 35865495 | −0.0412 | −0.0110 | 0.3840 |
| 511 | RP11-8N6 | 9 | 36632473 | 36774305 | −0.0410 | −0.0174 | 0.3875 |
| 2676 | RP11-397D12 | 9 | 37263640 | 37455975 | −0.0410 | −0.0174 | 0.3875 |
| 2671 | RP11-3J10 | 9 | 37747915 | 37935186 | −0.0410 | −0.0174 | 0.3875 |
| 527 | RP11-113O24 | 9 | 38263089 | 38427296 | −0.0408 | −0.0274 | 0.3910 |
| 3231 | RP11-290L7 | 9 | 38983970 | 39177643 | −0.2143 | −0.1120 | 0.4990 |
| 512 | RP11-395E19 | 9 | 40645668 | 40774565 | −0.2134 | −0.1327 | 0.5192 |
| 2675 | RP11-15E1 | 9 | 42890129 | 43009824 | −0.3049 | −0.2053 | 0.5779 |
| 2688 | RP11-475I24 | 9 | 44245620 | 44448510 | −0.3049 | −0.2053 | 0.5779 |
| 1067 | RP11-268E1 | 9 | 45843103 | 46009277 | −0.3037 | −0.2316 | 0.6038 |
| 3219 | RP11-211N8 | 9 | 46630312 | 46771375 | −0.3035 | −0.1792 | 0.6076 |
| 515 | RP11-38P6 | 9 | 67106117 | 67192942 | −0.1310 | −0.0154 | 0.4316 |
| 985 | RP11-274B18 | 9 | 70318668 | 70488660 | −0.1241 | −0.0184 | 0.3984 |
| 981 | RP11-265B8 | 9 | 70756001 | 70927381 | −0.1241 | −0.0184 | 0.3984 |
| 2620 | RP11-109D9 | 9 | 71447567 | 71636843 | −0.0941 | −0.0184 | 0.3993 |
| 988 | RP11-141J10 | 9 | 72488784 | 72637616 | −0.0939 | −0.0184 | 0.4032 |
| 4131 | RP11-563H8 | 9 | 73274802 | 73425572 | −0.0939 | −0.0184 | 0.4032 |
| 990 | RP11-429L21 | 9 | 74281669 | 74401046 | −0.0939 | −0.0262 | 0.4038 |
| 440 | RP11-71A24 | 9 | 74808540 | 74977641 | −0.0939 | −0.0338 | 0.4042 |
| 2622 | RP11-401G5 | 9 | 75584335 | 75757100 | −0.0937 | −0.0441 | 0.4089 |
| 441 | RP11-174B4 | 9 | 76502362 | 76697651 | −0.0937 | −0.0441 | 0.4089 |
| 3159 | RP11-66O21 | 9 | 77399675 | 77508705 | −0.0937 | −0.0441 | 0.4089 |
| 986 | RP11-422N19 | 9 | 78111661 | 78213797 | −0.0937 | −0.0441 | 0.4089 |
| 1427 | RP11-490H9 | 9 | 78861728 | 78928696 | −0.0937 | −0.0441 | 0.4089 |
| 3161 | RP11-336N8 | 9 | 79930194 | 80096565 | −0.0939 | −0.0551 | 0.4042 |
| 2605 | RP11-174K23 | 9 | 80495074 | 80676552 | −0.0941 | −0.0422 | 0.3990 |
| 446 | RP11-362L2 | 9 | 81425152 | 81446829 | −0.0941 | −0.0422 | 0.3990 |
| 450 | RP11-384P5 | 9 | 82178554 | 82315521 | −0.0943 | −0.0463 | 0.3952 |
| 999 | RP11-66D1 | 9 | 82951739 | 83064769 | −0.0943 | −0.0463 | 0.3952 |
| 3154 | RP11-432M2 | 9 | 84030884 | 84146321 | −0.0944 | −0.0386 | 0.3924 |
| 4123 | RP11-541F16 | 9 | 84667634 | 84783005 | −0.0944 | −0.0451 | 0.3932 |
| 3158 | RP11-439A18 | 9 | 85290903 | 85375534 | −0.0944 | −0.0451 | 0.3932 |
| 995 | RP11-59M22 | 9 | 86180525 | 86337480 | −0.0718 | −0.0451 | 0.3966 |
| 2601 | RP11-172F7 | 9 | 87074588 | 87248611 | −0.0718 | −0.0381 | 0.3961 |
| 3153 | RP11-280P22 | 9 | 87941616 | 87990836 | −0.0703 | −0.0377 | 0.4292 |
| 3149 | RP11-276H19 | 9 | 88787386 | 88940677 | −0.0703 | −0.0377 | 0.4292 |
| 3736 | RP11-423O13 | 9 | 88883454 | 89058511 | −0.0703 | −0.0377 | 0.4292 |
| 1189 | RP11-40C6 | 9 | 89255601 | 89375411 | −0.0703 | −0.0377 | 0.4292 |
| 1726 | RP11-249H20 | 9 | 89373412 | 89446315 | −0.0703 | −0.0377 | 0.4292 |
| 1717 | RP11-65B23 | 9 | 89444561 | 89614800 | −0.0703 | −0.0377 | 0.4292 |
| 2602 | RP11-345K9 | 9 | 89912028 | 89983781 | −0.0702 | −0.0581 | 0.4306 |
| 2609 | RP11-176L21 | 9 | 90659199 | 90759709 | −0.0702 | −0.0581 | 0.4306 |
| 2603 | RP11-8B23 | 9 | 91718617 | 91756894 | −0.0673 | −0.0330 | 0.4947 |
| 1967 | RP11-555F9 | 9 | 92185900 | 92362321 | −0.0703 | −0.0598 | 0.4299 |
| 998 | RP11-440G5 | 9 | 93170710 | 93341607 | −0.0697 | −0.0666 | 0.4418 |
| 2611 | RP11-19J3 | 9 | 94281893 | 94449626 | −0.0838 | −0.0814 | 0.4397 |
| 3143 | RP11-30L4 | 9 | 95248566 | 95419410 | −0.0838 | −0.0814 | 0.4397 |
| 3945 | RP11-333I7 | 9 | 96375866 | 96551155 | −0.0835 | −0.0729 | 0.4470 |
| 989 | RP11-279121 | 9 | 96493468 | 96616027 | −0.0835 | −0.0729 | 0.4470 |
| 3899 | RP11-435O5 | 9 | 97175312 | 97362955 | −0.0841 | −0.0640 | 0.4334 |
| 3160 | RP11-160D19 | 9 | 97447876 | 97448518 | −0.0841 | −0.0640 | 0.4334 |
| 461 | RP11-240L7 | 9 | 98020511 | 98190157 | −0.0843 | −0.0586 | 0.4305 |
| 455 | RP11-23J9 | 9 | 99080845 | 99246276 | −0.0843 | −0.0524 | 0.4299 |
| 2619 | RP11-23B15 | 9 | 99583820 | 99744615 | −0.0853 | −0.0653 | 0.4073 |
| 452 | RP11-92C4 | 9 | 100604517 | 100754446 | −0.0852 | −0.0717 | 0.4097 |
| 1194 | RP11-192E23 | 9 | 100754347 | 100889311 | −0.0852 | −0.0717 | 0.4097 |
| 1725 | RP11-96L7 | 9 | 100889312 | 100995443 | −0.0852 | −0.0717 | 0.4097 |
| 4127 | RP11-547C13 | 9 | 101231132 | 101374570 | −0.0852 | −0.0717 | 0.4097 |
| 457 | RP11-208F1 | 9 | 102096861 | 102138010 | −0.0853 | −0.0671 | 0.4077 |
| 2608 | RP11-80H12 | 9 | 102748901 | 102915948 | −0.0853 | −0.0671 | 0.4077 |
| 1001 | RP11-342F21 | 9 | 104230495 | 104390758 | −0.0853 | −0.0671 | 0.4077 |
| 994 | RP11-438P9 | 9 | 109317528 | 109517128 | −0.0620 | −0.0587 | 0.4033 |
| 458 | RP11-400A24 | 9 | 110244896 | 110428437 | −0.0620 | −0.0587 | 0.4033 |
| 454 | RP11-388N6 | 9 | 111218628 | 111227230 | −0.0620 | −0.0587 | 0.4033 |
| 1423 | RP11-470J20 | 9 | 111963321 | 112039788 | −0.0620 | −0.0587 | 0.4033 |
| 2613 | RP11-202G18 | 9 | 112915770 | 113092458 | −0.0620 | −0.0587 | 0.4033 |
| 1971 | RP11-570D4 | 9 | 113691272 | 113781581 | −0.0620 | −0.0587 | 0.4033 |
| 2612 | RP11-88M9 | 9 | 114552190 | 114649546 | −0.0620 | −0.0587 | 0.4033 |
| 3603 | RP11-534I8 | 9 | 115621750 | 115806051 | −0.0620 | −0.0587 | 0.4033 |
| 1000 | RP11-165P4 | 9 | 122882717 | 123029745 | −0.0791 | −0.0778 | 0.3884 |
| 3348 | RP11-477J21 | 9 | 123029646 | 123138575 | −0.0791 | −0.0778 | 0.3884 |
| 3150 | RP11-429D3 | 9 | 123651637 | 123824698 | −0.0656 | −0.0683 | 0.4064 |
| 2610 | RP11-373J8 | 9 | 129369677 | 129390787 | 0.1265 | −0.0148 | 0.5034 |
| 1963 | RP11-545E17 | 9 | 130501515 | 130668178 | −0.1621 | −0.0970 | 0.4226 |
| 453 | RP11-202H3 | 9 | 131896794 | 131932670 | −0.1629 | −0.1172 | 0.4053 |
| 4154 | RP11-57C19 | 9 | 132470366 | 132643828 | −0.1629 | −0.1172 | 0.4053 |
| 1721 | RP11-83J21 | 9 | 132641829 | 132818294 | −0.1626 | −0.1337 | 0.4119 |
| 3152 | RP11-143H20 | 9 | 132906902 | 132957071 | −0.1626 | −0.1337 | 0.4119 |
| 439 | RP11-5N16 | 9 | 133982008 | 134077119 | −0.1626 | −0.1337 | 0.4119 |
| 1734 | RP11-295G24 | 9 | 134621360 | 134820472 | −0.1626 | −0.1337 | 0.4119 |
| 996 | RP11-153P4 | 9 | 135531562 | 135650232 | −0.1626 | −0.1337 | 0.4119 |
| 2618 | RP11-399H11 | 9 | 137112170 | 137333442 | −0.1626 | −0.1337 | 0.4119 |
| 448 | RP11-83N9 | 9 | 138121793 | 138276710 | −0.1626 | −0.1337 | 0.4119 |
| 982 | RP11-417A4 | 9 | 139523184 | 139715973 | −0.1619 | −0.1102 | 0.4264 |
| 1391 | GS-135-I17 | 9 | 140133252 | 140283252 | −0.1603 | −0.0742 | 0.4630 |
| 4085 | GS-112-N13 | 9 | 140133302 | 140283302 | −0.1603 | −0.0742 | 0.4630 |
| 1925 | GS-306-F7 | 10 | 245000 | 395000 | 0.0003 | −0.0321 | 0.4794 |
| 3555 | GS-23-B11 | 10 | 245050 | 395050 | 0.0003 | −0.0321 | 0.4794 |
| 2691 | RP11-118K6 | 10 | 3035538 | 3133935 | 0.0376 | 0.0314 | 0.4184 |
| 2890 | RP11-189M8 | 10 | 10064792 | 10122105 | 0.0762 | 0.0816 | 0.4207 |
| 1728 | RP11-566K1 | 10 | 10830787 | 11020034 | 0.0759 | 0.1017 | 0.4139 |
| 1856 | RP1-251M9 | 10 | 10983504 | 11094455 | 0.0759 | 0.1017 | 0.4139 |
| 732 | RP11-401F24 | 10 | 11805219 | 12011805 | 0.0758 | 0.1170 | 0.4111 |
| 2638 | RP11-730A19 | 10 | 13058479 | 13254681 | 0.0758 | 0.1120 | 0.4111 |
| 187 | RP11-275E20 | 10 | 14321741 | 14514319 | 0.0758 | 0.1120 | 0.4111 |
| 2874 | RP11-165O3 | 10 | 21382229 | 21495264 | 0.0499 | 0.0712 | 0.4266 |
| 170 | RP11-108B14 | 10 | 22399352 | 22575928 | 0.0499 | 0.0712 | 0.4266 |
| 2880 | RP11-379L21 | 10 | 23123769 | 23317773 | 0.0499 | 0.0712 | 0.4266 |
| 190 | RP11-162E8 | 10 | 24196692 | 24341441 | 0.0494 | 0.0538 | 0.4152 |
| 2342 | RP11-129O7 | 10 | 25140960 | 25271606 | 0.0497 | 0.0653 | 0.4219 |
| 715 | RP11-307B23 | 10 | 25971696 | 26132567 | 0.0494 | 0.0799 | 0.4150 |
| 178 | RP11-128B16 | 10 | 26797312 | 26941977 | 0.0479 | 0.0652 | 0.3838 |
| 2646 | RP13-355A21 | 10 | 27935285 | 28141734 | 0.0479 | 0.0652 | 0.3838 |
| 1005 | RP11-478H13 | 10 | 29030096 | 29145351 | 0.0263 | 0.0652 | 0.3864 |
| 179 | RP11-254A5 | 10 | 36972908 | 37144255 | 0.0518 | 0.0608 | 0.3547 |
| 3166 | RP13-445N5 | 10 | 38601694 | 38812575 | −0.0678 | −0.0540 | 0.3740 |
| 2338 | RP11-124O11 | 10 | 42660862 | 42819196 | −0.0172 | −0.0157 | 0.3367 |
| 2869 | RP11-38B21 | 10 | 43864788 | 44024289 | −0.0172 | −0.0157 | 0.3367 |
| 481 | RP11-432I13 | 10 | 44988687 | 45157159 | −0.0172 | −0.0157 | 0.3367 |
| 713 | RP11-47O13 | 10 | 52979753 | 53069434 | 0.0249 | −0.0010 | 0.3599 |
| 1025 | RP11-556E13 | 10 | 53912334 | 54101584 | 0.0249 | −0.0010 | 0.3599 |
| 3185 | RP11-548F5 | 10 | 54798024 | 54938956 | 0.0251 | 0.0166 | 0.3560 |
| 2879 | RP11-313B15 | 10 | 55595990 | 55799248 | 0.0251 | 0.0071 | 0.3553 |
| 2641 | RP11-430K23 | 10 | 56709824 | 56882190 | 0.0251 | 0.0071 | 0.3553 |
| 2886 | RP11-183D3 | 10 | 57554198 | 57668808 | 0.0251 | 0.0071 | 0.3553 |
| 2876 | RP11-371O16 | 10 | 59005947 | 59241663 | 0.0711 | 0.0122 | 0.3565 |
| 721 | RP11-79I23 | 10 | 60095350 | 60270673 | 0.0711 | 0.0122 | 0.3565 |
| 2352 | RP11-357A18 | 10 | 60813485 | 60976636 | 0.0711 | 0.0122 | 0.3565 |
| 718 | RP11-166B18 | 10 | 61902197 | 62069022 | 0.0712 | 0.0173 | 0.3536 |
| 482 | RP11-809M12 | 10 | 62866153 | 62996102 | 0.0712 | 0.0173 | 0.3536 |
| 173 | RP11-13A2 | 10 | 63820049 | 63955343 | 0.0709 | 0.0156 | 0.3602 |
| 722 | RP11-176H12 | 10 | 65709954 | 65899759 | 0.0370 | 0.0157 | 0.3674 |
| 186 | RP11-161L14 | 10 | 66811668 | 66948580 | 0.0367 | 0.0239 | 0.3743 |
| 3174 | RP11-210G22 | 10 | 67400045 | 67579279 | 0.0367 | 0.0239 | 0.3743 |
| 2642 | RP11-778O10 | 10 | 67577279 | 67645677 | 0.0367 | 0.0239 | 0.3743 |
| 2637 | RP11-428N21 | 10 | 68138624 | 68290770 | 0.0367 | 0.0239 | 0.3743 |
| 2871 | RP11-297N15 | 10 | 69441097 | 69601754 | 0.0769 | 0.0239 | 0.3681 |
| 2883 | RP11-314J18 | 10 | 70288201 | 70457568 | 0.0769 | 0.0239 | 0.3681 |
| 712 | RP11-367H5 | 10 | 71487221 | 71672097 | 0.0298 | 0.0240 | 0.3792 |
| 2877 | RP11-58O1 | 10 | 72645813 | 72768100 | 0.0298 | 0.0240 | 0.3792 |
| 2340 | RP11-345K20 | 10 | 74818637 | 74895321 | 0.2188 | 0.1553 | 0.5798 |
| 728 | RP11-399K21 | 10 | 76694924 | 76882564 | 0.1151 | 0.0643 | 0.4252 |
| 727 | RP11-325D15 | 10 | 77881141 | 78010383 | 0.1149 | 0.0720 | 0.4290 |
| 1010 | RP11-328K22 | 10 | 78687185 | 78783396 | 0.1147 | 0.0646 | 0.4329 |
| 2630 | RP11-619F23 | 10 | 78781397 | 78833935 | 0.1147 | 0.0646 | 0.4329 |
| 725 | RP11-90J7 | 10 | 79687950 | 79792544 | 0.1148 | 0.0506 | 0.4315 |
| 2626 | RP11-574P20 | 10 | 80580496 | 80624367 | 0.1148 | 0.0506 | 0.4315 |
| 709 | RP11-40F6 | 10 | 81915872 | 81996355 | 0.1148 | 0.0506 | 0.4315 |
| 185 | RP11-20E23 | 10 | 82479159 | 82648710 | 0.0749 | 0.0452 | 0.4312 |
| 729 | RP11-95M17 | 10 | 84424513 | 84577760 | 0.0047 | −0.0004 | 0.4609 |
| 2335 | RP11-219F10 | 10 | 85603794 | 85708505 | 0.0047 | −0.0004 | 0.4609 |
| 465 | RP11-470J18 | 10 | 86321573 | 86390139 | 0.0052 | −0.0168 | 0.4503 |
| 2334 | RP11-113E21 | 10 | 87212538 | 87312980 | 0.0052 | −0.0253 | 0.4506 |
| 2888 | RP11-396M20 | 10 | 88054883 | 88222662 | 0.0053 | −0.0463 | 0.4482 |
| 714 | RP11-165M8 | 10 | 89531916 | 89596957 | 0.0001 | −0.1301 | 0.5603 |
| 1723 | RP11-380G5 | 10 | 89596958 | 89798494 | 0.0044 | −0.1029 | 0.4679 |
| 478 | RP11-765C10 | 10 | 89796494 | 89839511 | 0.0044 | −0.1029 | 0.4679 |
| 2252 | RP11-765c10 | 10 | 89796494 | 89839511 | 0.0044 | −0.1029 | 0.4679 |
| 2889 | RP11-92H8 | 10 | 92926791 | 93134225 | −0.0353 | −0.0837 | 0.3727 |
| 2344 | RP11-348J12 | 10 | 94800116 | 94832262 | 0.0351 | −0.0262 | 0.5178 |
| 2350 | RP11-162K11 | 10 | 95842632 | 95886385 | −0.0382 | −0.1174 | 0.4083 |
| 710 | RP11-164I17 | 10 | 97222399 | 97318079 | −0.0382 | −0.1401 | 0.4096 |
| 2339 | RP11-248J23 | 10 | 97667188 | 97780399 | −0.0382 | −0.1565 | 0.4080 |
| 2331 | RP11-196N24 | 10 | 98419168 | 98525673 | −0.0382 | −0.1587 | 0.4092 |
| 2345 | RP11-19C6 | 10 | 99620296 | 99781530 | −0.0079 | −0.1701 | 0.4045 |
| 2634 | RP11-704L16 | 10 | 100941233 | 101064714 | −0.0078 | −0.1708 | 0.4017 |
| 2351 | RP11-287G8 | 10 | 101701714 | 101837645 | −0.0078 | −0.1708 | 0.4017 |
| 2887 | RP11-324L3 | 10 | 102895109 | 102985100 | −0.0078 | −0.1708 | 0.4017 |
| 1730 | RP11-264H19 | 10 | 103139013 | 103282090 | −0.0078 | −0.1708 | 0.4017 |
| 3356 | RP11-529I10 | 10 | 103280091 | 103386596 | −0.0078 | −0.1708 | 0.4017 |
| 716 | RP11-373N18 | 10 | 105866280 | 105921006 | −0.0071 | −0.1548 | 0.3858 |
| 2878 | RP11-165P9 | 10 | 106789661 | 106827519 | −0.0069 | −0.1628 | 0.3814 |
| 466 | RP11-596L14 | 10 | 107741530 | 107812754 | −0.0067 | −0.1359 | 0.3772 |
| 470 | RP11-699H2 | 10 | 108487790 | 108574828 | −0.0065 | −0.1264 | 0.3741 |
| 3169 | RP11-478K18 | 10 | 109290757 | 109428292 | −0.0075 | −0.1128 | 0.3945 |
| 2870 | RP11-163F15 | 10 | 110243435 | 110340531 | −0.0074 | −0.1090 | 0.3932 |
| 2347 | RP11-271I13 | 10 | 111497654 | 111539088 | −0.0072 | −0.1037 | 0.3891 |
| 2872 | RP11-364E8 | 10 | 112423849 | 112498372 | −0.0072 | −0.1037 | 0.3891 |
| 720 | RP11-381K7 | 10 | 112987340 | 113136937 | −0.0065 | −0.0893 | 0.3734 |
| 2633 | RP11-426E5 | 10 | 113860100 | 114056756 | −0.0199 | −0.0893 | 0.3714 |
| 188 | RP11-357H24 | 10 | 114874395 | 115058209 | −0.0199 | −0.0893 | 0.3714 |
| 2629 | RP11-411P18 | 10 | 115833956 | 115994376 | −0.0193 | −0.0966 | 0.3583 |
| 2336 | RP11-338L11 | 10 | 116774286 | 116971219 | −0.0193 | −0.0966 | 0.3583 |
| 2330 | RP11-96N16 | 10 | 117796864 | 117962983 | −0.0208 | −0.0900 | 0.3918 |
| 169 | RP11-5G18 | 10 | 118760709 | 118853817 | −0.0208 | −0.0900 | 0.3918 |
| 2348 | RP11-355F22 | 10 | 119495410 | 119624604 | −0.0208 | −0.0900 | 0.3918 |
| 473 | RP11-427L15 | 10 | 120419970 | 120485859 | −0.0209 | −0.0990 | 0.3930 |
| 192 | RP11-359E7 | 10 | 121545719 | 121577827 | −0.0210 | −0.1096 | 0.3960 |
| 723 | RP11-323P17 | 10 | 122467430 | 122626379 | −0.0210 | −0.1096 | 0.3960 |
| 717 | RP11-62L18 | 10 | 123214091 | 123388488 | −0.0212 | −0.0925 | 0.4006 |
| 3353 | RP11-45D20 | 10 | 123333380 | 123408484 | −0.0212 | −0.0925 | 0.4006 |
| 2645 | RP11-436O19 | 10 | 124157652 | 124268654 | −0.0212 | −0.0925 | 0.4006 |
| 176 | RP11-338O1 | 10 | 125057753 | 125259149 | −0.0416 | −0.0917 | 0.4009 |
| 175 | RP13-238F13 | 10 | 126007440 | 126190394 | −0.0416 | −0.0856 | 0.4011 |
| 2983 | RP11-115N19 | 10 | 126605954 | 126760588 | −0.0413 | −0.0983 | 0.3943 |
| 182 | RP11-151A10 | 10 | 126760588 | 126955018 | −0.0413 | −0.0983 | 0.3943 |
| 2341 | RP11-16P8 | 10 | 128180750 | 128268594 | −0.0417 | −0.1061 | 0.4034 |
| 724 | RP11-384P18 | 10 | 128798515 | 128966887 | −0.0772 | −0.1060 | 0.4091 |
| 183 | RP11-264E18 | 10 | 129933086 | 130095129 | −0.0772 | −0.1060 | 0.4091 |
| 2882 | RP11-168C9 | 10 | 130845700 | 130997880 | −0.0778 | −0.0977 | 0.4216 |
| 2892 | RP11-400F15 | 10 | 131923441 | 131970517 | −0.0774 | −0.0860 | 0.4131 |
| 719 | RP11-314D6 | 10 | 132451032 | 132635359 | −0.0774 | −0.0860 | 0.4131 |
| 2873 | RP11-45A17 | 10 | 133629568 | 133778458 | −0.0774 | −0.0860 | 0.4131 |
| 1931 | GS-261-B16 | 10 | 135029737 | 135179737 | −0.2825 | −0.2058 | 0.5492 |
| 4089 | GS-137-E24 | 10 | 135029787 | 135179787 | −0.2825 | −0.2058 | 0.5492 |
| 1929 | GS-908-H22 | 11 | 50000 | 200000 | −0.0777 | −0.1197 | 0.4574 |
| 4098 | GS-44-H18 | 11 | 50050 | 200050 | −0.0777 | −0.1197 | 0.4574 |
| 3352 | RP11-496I9 | 11 | 531284 | 635941 | −0.0738 | −0.2314 | 0.3727 |
| 2941 | RP11-2B17 | 11 | 1687193 | 1687713 | −0.0738 | −0.2314 | 0.3727 |
| 333 | RP11-295K3 | 11 | 1672662 | 1785148 | −0.0738 | −0.2314 | 0.3727 |
| 904 | RP3-416J11 | 11 | 2061361 | 2140325 | −0.0734 | −0.2333 | 0.3637 |
| 1733 | RP11-113A6 | 11 | 2261568 | 2430797 | −0.0734 | −0.2333 | 0.3637 |
| 1798 | RP11-19N21 | 11 | 2745620 | 2920188 | −0.0734 | −0.2333 | 0.3637 |
| 3341 | RP11-19N21 | 11 | 2745620 | 2920188 | −0.0734 | −0.2333 | 0.3637 |
| 2440 | RP11-120E20 | 11 | 3573895 | 3758001 | −0.0734 | −0.2240 | 0.3628 |
| 4269 | RP11-438N5 | 11 | 3854180 | 4005785 | −0.0734 | −0.2240 | 0.3628 |
| 1343 | RP11-309J20 | 11 | 4826420 | 4895678 | −0.0734 | −0.2336 | 0.3624 |
| 3948 | RP11-364G22 | 11 | 5753680 | 5927293 | −0.0734 | −0.2336 | 0.3624 |
| 4199 | RP11-89D4 | 11 | 7249883 | 7402046 | −0.0342 | −0.1967 | 0.3918 |
| 300 | RP11-379P15 | 11 | 8144634 | 8368375 | −0.0342 | −0.1967 | 0.3918 |
| 1169 | RP11-152H18 | 11 | 8634957 | 8795697 | −0.0342 | −0.1967 | 0.3918 |
| 4171 | RP11-243M7 | 11 | 9666315 | 9667022 | −0.0340 | −0.1725 | 0.3882 |
| 3405 | RP11-327O2 | 11 | 10289439 | 10454001 | −0.0337 | −0.1777 | 0.3812 |
| 3692 | RP11-206L19 | 11 | 11681593 | 11828351 | −0.0339 | −0.1641 | 0.3861 |
| 3134 | RP11-51B23 | 11 | 12368547 | 12543770 | −0.0339 | −0.1641 | 0.3861 |
| 544 | RP11-502G22 | 11 | 13920040 | 14078651 | −0.0345 | −0.1388 | 0.3985 |
| 255 | RP11-7O20 | 11 | 14830547 | 14997984 | −0.0345 | −0.1388 | 0.3985 |
| 915 | RP11-13J19 | 11 | 15778995 | 15940672 | −0.0345 | −0.1388 | 0.3985 |
| 262 | RP11-4B7 | 11 | 16626917 | 16774089 | −0.0356 | −0.1092 | 0.4216 |
| 4149 | CTC-1277H1 | 11 | 17512270 | 17608985 | −0.0354 | −0.1051 | 0.4183 |
| 3688 | RP11-206C1 | 11 | 18095206 | 18291271 | −0.0354 | −0.0956 | 0.4180 |
| 556 | RP11-504G3 | 11 | 18545502 | 18615506 | −0.0355 | −0.0847 | 0.4198 |
| 2407 | RP11-6K5 | 11 | 20206577 | 20366384 | −0.0346 | −0.0964 | 0.3998 |
| 288 | RP11-121C18 | 11 | 21566335 | 21747083 | −0.0379 | −0.0659 | 0.4734 |
| 908 | RP11-34N19 | 11 | 22392002 | 22530139 | −0.0006 | −0.0950 | 0.3949 |
| 639 | RP11-72C9 | 11 | 24362907 | 24551978 | −0.0015 | −0.0623 | 0.4131 |
| 4198 | RP11-108G3 | 11 | 25458832 | 25606731 | −0.0015 | −0.0517 | 0.4137 |
| 2116 | RP11-430L3 | 11 | 26201313 | 26402986 | −0.0017 | −0.0334 | 0.4173 |
| 3522 | RP11-541L7 | 11 | 27223810 | 27371257 | −0.0014 | −0.0253 | 0.4123 |
| 572 | RP11-466I1 | 11 | 29080323 | 29253188 | −0.0057 | −0.0545 | 0.5064 |
| 3566 | RP5-859D17 | 11 | 29439625 | 29480700 | −0.0013 | −0.0213 | 0.4102 |
| 2740 | RP11-472K20 | 11 | 35698571 | 35767187 | 0.0039 | 0.0103 | 0.4017 |
| 2206 | RP11-115P8 | 11 | 35657471 | 35810959 | 0.0039 | 0.0103 | 0.4017 |
| 1225 | RP11-324K6 | 11 | 37673711 | 37779927 | 0.0040 | 0.0160 | 0.4045 |
| 2541 | RP11-277K23 | 11 | 38558445 | 38673899 | 0.0038 | 0.0099 | 0.3995 |
| 3854 | RP11-164L18 | 11 | 46216229 | 46383009 | 0.0020 | 0.0000 | 0.3597 |
| 289 | RP11-131J4 | 11 | 54839304 | 55007605 | 0.0352 | 0.0296 | 0.3450 |
| 3190 | RP3-466A11 | 11 | 61394968 | 61395556 | 0.0116 | 0.0118 | 0.3462 |
| 832 | RP11-126P21 | 11 | 66841191 | 66872614 | −0.0237 | −0.0300 | 0.4225 |
| 2726 | RP11-138N3 | 11 | 67395614 | 67616066 | −0.0261 | −0.1007 | 0.4740 |
| 766 | RP11-569N5 | 11 | 68072319 | 68278585 | 0.0134 | 0.0436 | 0.3863 |
| 3839 | RP11-554A11 | 11 | 68509551 | 68701448 | 0.0134 | 0.0436 | 0.3863 |
| 4160 | RP11-804L21 | 11 | 69297663 | 69494896 | 0.0139 | 0.0585 | 0.3971 |
| 3419 | RP11-21D20 | 11 | 70236623 | 70391405 | 0.0139 | 0.0585 | 0.3971 |
| 3612 | RP11-598K3 | 11 | 70306687 | 70472869 | 0.0139 | 0.0585 | 0.3971 |
| 1899 | RP11-512I24 | 11 | 70596594 | 70766596 | 0.0139 | 0.0585 | 0.3971 |
| 2584 | RP11-31L22 | 11 | 71992654 | 72170769 | 0.0139 | 0.0585 | 0.3971 |
| 2040 | RP11-93M11 | 11 | 72509790 | 72552991 | 0.0146 | 0.1337 | 0.4124 |
| 2136 | RP11-28L18 | 11 | 73676966 | 73848622 | 0.0143 | 0.1629 | 0.4038 |
| 3131 | RP11-28L18 | 11 | 73676966 | 73848622 | 0.0143 | 0.1629 | 0.4038 |
| 3874 | RP11-167F22 | 11 | 73706065 | 73867546 | 0.0143 | 0.1629 | 0.4038 |
| 4061 | RP11-535A19 | 11 | 75177922 | 75265229 | 0.0141 | 0.1573 | 0.4010 |
| 1748 | RP11-25F7 | 11 | 75965024 | 76130350 | 0.1511 | 0.1565 | 0.4666 |
| 1822 | RP11-25F7 | 11 | 75965024 | 76130350 | 0.1511 | 0.1565 | 0.4666 |
| 2580 | RP11-30J7 | 11 | 76074447 | 76232373 | 0.1511 | 0.1710 | 0.4668 |
| 1548 | RP11-321B9 | 11 | 77210154 | 77360154 | 0.1508 | 0.1654 | 0.4606 |
| 4032 | RP11-321B9 | 11 | 77210204 | 77360204 | 0.1508 | 0.1654 | 0.4606 |
| 247 | RP11-7H7 | 11 | 78034362 | 78206791 | 0.0548 | 0.1591 | 0.3924 |
| 1693 | RP11-153F6 | 11 | 78641698 | 78791854 | 0.0549 | 0.1649 | 0.3938 |
| 422 | RP11-46D24 | 11 | 79636818 | 79817932 | 0.0087 | 0.1700 | 0.3970 |
| 1708 | RP11-187P2 | 11 | 80418018 | 80501017 | 0.0087 | 0.1700 | 0.3970 |
| 3027 | RP11-179A16 | 11 | 81301982 | 81405303 | 0.0087 | 0.1700 | 0.3970 |
| 2439 | RP11-113K21 | 11 | 82426834 | 82518598 | 0.0087 | 0.1700 | 0.3970 |
| 831 | RP11-118L16 | 11 | 83021047 | 83205297 | −0.0073 | 0.1523 | 0.3774 |
| 554 | RP11-482L11 | 11 | 84737802 | 84904751 | −0.0071 | 0.1450 | 0.3815 |
| 3656 | RP11-90K17 | 11 | 85263545 | 85320757 | −0.0065 | 0.1577 | 0.3938 |
| 1864 | RP11-320L11 | 11 | 85613336 | 85682335 | −0.0065 | 0.1577 | 0.3938 |
| 829 | RP11-137O10 | 11 | 86920457 | 87028461 | −0.0180 | 0.1255 | 0.4010 |
| 3389 | RP11-325I16 | 11 | 90903371 | 91076086 | −0.0159 | 0.1009 | 0.4465 |
| 4025 | RP11-291N1 | 11 | 90957432 | 91163512 | −0.0159 | 0.1009 | 0.4465 |
| 1373 | RP11-533H15 | 11 | 91893716 | 92071858 | −0.0175 | 0.1121 | 0.4115 |
| 644 | RP11-83E23 | 11 | 92720473 | 92905772 | −0.0175 | 0.1121 | 0.4115 |
| 4238 | RP11-236L3 | 11 | 93040962 | 93189086 | −0.0175 | 0.1121 | 0.4115 |
| 2004 | RP11-816P15 | 11 | 93724208 | 93796118 | −0.0180 | 0.1345 | 0.3997 |
| 3620 | RP11-685N10 | 11 | 93796119 | 93869551 | −0.0180 | 0.1345 | 0.3997 |
| 334 | RP11-349I16 | 11 | 94000378 | 94161783 | −0.0180 | 0.1345 | 0.3997 |
| 3402 | RP11-338H14 | 11 | 94849629 | 94936449 | −0.0180 | 0.1413 | 0.4003 |
| 1805 | RP11-16K5 | 11 | 95569628 | 95712661 | −0.0180 | 0.1413 | 0.4003 |
| 419 | RP11-25P2 | 11 | 96155646 | 96278738 | −0.0182 | 0.1373 | 0.3970 |
| 2460 | RP11-379J13 | 11 | 97102773 | 97328943 | −0.0182 | 0.1373 | 0.3970 |
| 1601 | RP11-99C10 | 11 | 98312645 | 98420558 | −0.0174 | 0.1476 | 0.4129 |
| 3669 | RP11-99C10 | 11 | 98312645 | 98420558 | −0.0174 | 0.1476 | 0.4129 |
| 962 | RP11-49M9 | 11 | 99613167 | 99747975 | −0.0183 | 0.1322 | 0.3941 |
| 969 | RP11-45C5 | 11 | 100145392 | 100299550 | −0.0568 | 0.1322 | 0.3996 |
| 3423 | RP11-21G19 | 11 | 101247704 | 101397612 | −0.0568 | 0.1322 | 0.3996 |
| 3343 | RP11-315O6 | 11 | 101724655 | 101922841 | −0.0568 | 0.1322 | 0.3996 |
| 1992 | RP11-750P5 | 11 | 102010617 | 102011178 | −0.0564 | 0.1222 | 0.4083 |
| 2957 | RP11-2I22 | 11 | 102613695 | 102771597 | −0.0563 | 0.1073 | 0.4111 |
| 1464 | RP11-725J15 | 11 | 102741000 | 102891000 | −0.0563 | 0.1073 | 0.4111 |
| 222 | RP11-563P16 | 11 | 102966300 | 103066089 | −0.0563 | 0.1041 | 0.4120 |
| 1168 | RP11-179B7 | 11 | 103803543 | 103965030 | −0.0292 | 0.1042 | 0.4065 |
| 3966 | RP11-20H14 | 11 | 104950107 | 105093266 | −0.0292 | 0.1042 | 0.4065 |
| 762 | RP11-569A20 | 11 | 106083757 | 106252007 | −0.0294 | 0.0985 | 0.4035 |
| 1193 | RP11-56J3 | 11 | 107487025 | 107646360 | −0.0296 | 0.0692 | 0.3983 |
| 2242 | RP11-56j3 | 11 | 107487025 | 107646360 | −0.0296 | 0.0692 | 0.3983 |
| 1483 | RP11-241D13 | 11 | 107541895 | 107699828 | −0.0296 | 0.0692 | 0.3983 |
| 1812 | RP11-25I9 | 11 | 108226252 | 108391319 | −0.0294 | 0.0508 | 0.4022 |
| 2944 | RP11-11N15 | 11 | 110352104 | 110510107 | −0.0292 | 0.0548 | 0.4066 |
| 2042 | RP11-108O10 | 11 | 111095144 | 111274317 | −0.0282 | 0.0658 | 0.4295 |
| 3678 | RP11-107P10 | 11 | 111797481 | 111856589 | −0.0282 | 0.0658 | 0.4295 |
| 3663 | RP11-87N22 | 11 | 112570898 | 112764095 | −0.0285 | 0.0577 | 0.4231 |
| 4240 | RP11-212D19 | 11 | 113740432 | 113877494 | −0.0285 | 0.0577 | 0.4231 |
| 2985 | RP11-136I14 | 11 | 114823728 | 114977828 | −0.0285 | 0.0524 | 0.4221 |
| 1288 | RP11-114K7 | 11 | 115593822 | 115721329 | −0.0285 | 0.0524 | 0.4221 |
| 790 | RP11-4N9 | 11 | 116204676 | 116296560 | −0.0285 | 0.0524 | 0.4221 |
| 920 | RP11-35P15 | 11 | 117022825 | 117131495 | −0.0285 | 0.0524 | 0.4221 |
| 2840 | CTD-3245B9 | 11 | 118148996 | 118395819 | −0.0285 | 0.0524 | 0.4221 |
| 2582 | RP11-46D5 | 11 | 118726259 | 118898358 | −0.0285 | 0.0524 | 0.4221 |
| 324 | RP11-215D10 | 11 | 119196989 | 119349896 | −0.0285 | 0.0524 | 0.4221 |
| 2454 | RP11-142I2 | 11 | 120790886 | 120821699 | −0.0281 | 0.0516 | 0.4306 |
| 1706 | RP11-166D19 | 11 | 121326321 | 121516680 | −0.0281 | 0.0516 | 0.4306 |
| 2036 | RP11-93E4 | 11 | 122124382 | 122299430 | −0.0284 | 0.0603 | 0.4250 |
| 2167 | RP11-485A5 | 11 | 123265105 | 123449312 | −0.0284 | 0.0539 | 0.4257 |
| 2408 | RP11-10N17 | 11 | 124487806 | 124646248 | −0.0284 | 0.0539 | 0.4257 |
| 4201 | RP11-100P11 | 11 | 124585409 | 124761537 | −0.0284 | 0.0539 | 0.4257 |
| 2120 | RP11-432I22 | 11 | 125827474 | 126006340 | −0.0282 | 0.0456 | 0.4301 |
| 3670 | RP11-106O22 | 11 | 125962083 | 126160028 | −0.0282 | 0.0456 | 0.4301 |
| 3327 | RP11-168K9 | 11 | 126420601 | 126506792 | −0.0282 | 0.0456 | 0.4301 |
| 1485 | RP11-264E20 | 11 | 127930569 | 128023639 | −0.0274 | 0.0285 | 0.4470 |
| 238 | RP11-567M21 | 11 | 129326430 | 129514601 | −0.0254 | 0.0585 | 0.4892 |
| 1987 | RP11-567M21 | 11 | 129326430 | 129514601 | −0.0254 | 0.0585 | 0.4892 |
| 3930 | RP11-340L13 | 11 | 130972996 | 131149195 | −0.0255 | 0.0261 | 0.4887 |
| 2107 | RP11-419F8 | 11 | 132221425 | 132391430 | −0.0255 | 0.0261 | 0.4887 |
| 4054 | RP11-545G16 | 11 | 132451658 | 132626345 | −0.0255 | 0.0261 | 0.4887 |
| 1153 | RP11-149G17 | 11 | 133514734 | 133706144 | −0.0255 | 0.0261 | 0.4887 |
| 2713 | RP11-469N6 | 11 | 133983990 | 134148703 | −0.0255 | 0.0261 | 0.4887 |
| 1933 | GS-26-N8 | 11 | 134312384 | 134462384 | −0.2840 | −0.1199 | 0.6160 |
| 4093 | GS-770-G7 | 11 | 134312434 | 134462434 | −0.2840 | −0.1199 | 0.6160 |
| 1395 | GS-8-M16 | 12 | 25000 | 175000 | 0.1956 | 0.3179 | 0.5357 |
| 4075 | GS-124-K20 | 12 | 25050 | 175050 | 0.1956 | 0.3179 | 0.5357 |
| 329 | RP11-283I3 | 12 | 152505 | 329906 | 0.1393 | 0.2882 | 0.4929 |
| 1555 | RP11-283I3 | 12 | 152505 | 329906 | 0.1393 | 0.2882 | 0.4929 |
| 4097 | GS-496-A11 | 12 | 640787 | 790787 | 0.1398 | 0.2893 | 0.5030 |
| 513 | RP11-359b12 | 12 | 858588 | 1012956 | 0.1397 | 0.2953 | 0.5018 |
| 1236 | RP11-359B12 | 12 | 858588 | 1012956 | 0.1397 | 0.2953 | 0.5018 |
| 29 | RP11-21K20 | 12 | 1719983 | 1855190 | 0.1382 | 0.2356 | 0.4690 |
| 3202 | RP5-1096D14 | 12 | 1855112 | 2075367 | 0.1382 | 0.2356 | 0.4690 |
| 3534 | RP11-543P15 | 12 | 3101423 | 3265129 | 0.1378 | 0.2277 | 0.4601 |
| 2731 | RP11-320N7 | 12 | 3997826 | 4189702 | 0.1378 | 0.2277 | 0.4601 |
| 3890 | RP11-264F23 | 12 | 4189703 | 4315660 | 0.1378 | 0.2277 | 0.4601 |
| 3504 | RP11-319E16 | 12 | 5163922 | 5344301 | 0.1814 | 0.2228 | 0.4420 |
| 568 | RP11-451H11 | 12 | 5802494 | 5927041 | 0.1814 | 0.2228 | 0.4420 |
| 4100 | RP3-467F14 | 12 | 5990176 | 6100072 | 0.1812 | 0.2143 | 0.4375 |
| 2954 | RP11-4N23 | 12 | 13505605 | 13592296 | 0.2607 | 0.1961 | 0.4749 |
| 2708 | RP11-502N13 | 12 | 14521905 | 14648407 | 0.2608 | 0.2069 | 0.4737 |
| 4024 | RP11-320J20 | 12 | 15496292 | 15658372 | 0.2608 | 0.2069 | 0.4737 |
| 1613 | RP11-127B1 | 12 | 18497855 | 18558864 | 0.2401 | 0.1881 | 0.4210 |
| 2437 | RP11-127B1 | 12 | 18497855 | 18558864 | 0.2401 | 0.1881 | 0.4210 |
| 1765 | RP11-328P13 | 12 | 19403674 | 19416674 | 0.2395 | 0.1667 | 0.4344 |
| 2146 | RP11-248B13 | 12 | 21303649 | 21412934 | 0.2390 | 0.2298 | 0.4469 |
| 4183 | RP11-248B13 | 12 | 21303649 | 21412934 | 0.2390 | 0.2298 | 0.4469 |
| 1244 | RP11-359J14 | 12 | 22472217 | 22628174 | 0.2390 | 0.2058 | 0.4469 |
| 3745 | RP11-437F6 | 12 | 23668225 | 23784534 | 0.2393 | 0.1929 | 0.4386 |
| 3624 | RP11-707G18 | 12 | 25271849 | 25447854 | 0.2398 | 0.1743 | 0.4278 |
| 3033 | RP11-318G8 | 12 | 25445849 | 25520153 | 0.2398 | 0.1743 | 0.4278 |
| 1368 | RP11-522D14 | 12 | 25691330 | 25738010 | 0.2398 | 0.1743 | 0.4278 |
| 1243 | RP11-350G2 | 12 | 26494858 | 26676686 | 0.2183 | 0.1707 | 0.4275 |
| 1669 | RP11-425D17 | 12 | 28228331 | 28409325 | 0.1968 | 0.1780 | 0.4219 |
| 3702 | RP11-230B21 | 12 | 28623872 | 28774457 | 0.1679 | 0.1627 | 0.4122 |
| 1867 | RP11-310I24 | 12 | 29551976 | 29607136 | 0.1679 | 0.1627 | 0.4122 |
| 4259 | RP11-310I24 | 12 | 29551976 | 29607136 | 0.1679 | 0.1627 | 0.4122 |
| 2809 | RP11-59L15 | 12 | 30396155 | 30548869 | 0.1677 | 0.1496 | 0.4151 |
| 2260 | RP11-77I22 | 12 | 30742591 | 30939660 | 0.1674 | 0.1409 | 0.4216 |
| 3130 | RP11-50I19 | 12 | 32002852 | 32053594 | 0.1693 | 0.1143 | 0.3801 |
| 3008 | RP11-388G12 | 12 | 32552936 | 32687563 | 0.1695 | 0.1169 | 0.3760 |
| 5 | RP11-460N10 | 12 | 33170516 | 33333493 | 0.2033 | 0.1183 | 0.4112 |
| 1781 | RP11-333D23 | 12 | 37199957 | 37369589 | 0.1926 | 0.0600 | 0.3668 |
| 1487 | RP11-242B24 | 12 | 37329345 | 37489083 | 0.1926 | 0.0600 | 0.3668 |
| 811 | RP11-115F18 | 12 | 38991138 | 39141490 | 0.1926 | 0.0600 | 0.3668 |
| 1910 | RP11-549E9 | 12 | 39661383 | 39833150 | 0.1924 | 0.0658 | 0.3706 |
| 2117 | RP11-440G2 | 12 | 40718187 | 40834128 | 0.1337 | 0.0653 | 0.3318 |
| 1895 | RP11-510P12 | 12 | 41495094 | 41624443 | 0.1337 | 0.0653 | 0.3318 |
| 4228 | RP11-210N13 | 12 | 42413120 | 42531691 | 0.1337 | 0.0653 | 0.3318 |
| 3719 | RP11-283I17 | 12 | 43152685 | 43346494 | 0.1339 | 0.0611 | 0.3281 |
| 4190 | RP11-283I17 | 12 | 43152685 | 43346494 | 0.1339 | 0.0611 | 0.3281 |
| 3875 | RP11-176J16 | 12 | 43511484 | 43548358 | 0.1339 | 0.0611 | 0.3281 |
| 309 | RP11-394C19 | 12 | 44741501 | 44903830 | 0.1339 | 0.0611 | 0.3281 |
| 580 | RP11-493L12 | 12 | 45864297 | 46054344 | 0.1338 | 0.0505 | 0.3307 |
| 1044 | RP3-432E18 | 12 | 46156911 | 46299979 | 0.1336 | 0.0556 | 0.3353 |
| 2503 | RP5-1057I20 | 12 | 46451113 | 46571184 | 0.1336 | 0.0556 | 0.3353 |
| 2745 | RP11-89H19 | 12 | 46569188 | 46627459 | 0.1336 | 0.0556 | 0.3353 |
| 1878 | RP11-302B13 | 12 | 47561979 | 47666571 | 0.1336 | 0.0556 | 0.3353 |
| 1165 | RP11-152A18 | 12 | 48258682 | 48259463 | 0.1336 | 0.0556 | 0.3353 |
| 3572 | RP3-405J10 | 12 | 48854951 | 48970294 | 0.1336 | 0.0556 | 0.3353 |
| 34 | RP11-112N23 | 12 | 49017640 | 49198741 | 0.1339 | 0.0614 | 0.3295 |
| 4041 | RP11-295B8 | 12 | 49924002 | 50099834 | 0.1341 | 0.0573 | 0.3247 |
| 1497 | RP11-96P3 | 12 | 51027854 | 51174171 | 0.1341 | 0.0573 | 0.3247 |
| 2129 | RP11-443I3 | 12 | 51649634 | 51653639 | 0.3219 | 0.0822 | 0.4801 |
| 3718 | RP11-443I3 | 12 | 51649634 | 51653639 | 0.3219 | 0.0822 | 0.4801 |
| 1449 | RP11-1136G11 | 12 | 51745455 | 51903261 | 0.0911 | 0.0638 | 0.3465 |
| 578 | RP11-202G24 | 12 | 76592229 | 76660224 | 0.0262 | 0.0226 | 0.3803 |
| 427 | RP11-26L7 | 12 | 76975277 | 77145211 | 0.0259 | 0.0126 | 0.3857 |
| 437 | RP11-44H8 | 12 | 78580511 | 78745903 | 0.0259 | 0.0126 | 0.3857 |
| 4175 | RP11-244J10 | 12 | 78967744 | 79140545 | 0.0386 | 0.0126 | 0.3878 |
| 3932 | RP11-362A1 | 12 | 80826960 | 80924954 | 0.0382 | −0.0220 | 0.3962 |
| 1503 | RP11-87P13 | 12 | 81990475 | 82172372 | 0.0381 | −0.0355 | 0.3977 |
| 2960 | RP11-12K13 | 12 | 82690966 | 82845137 | 0.0381 | −0.0355 | 0.3977 |
| 2595 | RP11-26O20 | 12 | 83409425 | 83566636 | 0.0381 | −0.0355 | 0.3977 |
| 2719 | RP11-494O6 | 12 | 83887442 | 83970441 | −0.0073 | −0.0400 | 0.4081 |
| 2063 | RP11-202H2 | 12 | 85524877 | 85686118 | −0.0079 | −0.0596 | 0.4226 |
| 1573 | RP11-435O22 | 12 | 86171716 | 86310791 | −0.0611 | −0.0739 | 0.4422 |
| 1877 | RP11-295A20 | 12 | 87680771 | 87694735 | 0.0342 | −0.0571 | 0.4171 |
| 2424 | RP11-11M4 | 12 | 88092437 | 88268617 | 0.0342 | −0.0571 | 0.4171 |
| 3635 | RP11-239F20 | 12 | 90443259 | 90550237 | 0.0236 | −0.0613 | 0.4156 |
| 2961 | RP11-2K12 | 12 | 91204898 | 91367961 | 0.0236 | −0.0613 | 0.4156 |
| 1792 | RP11-24I19 | 12 | 92994984 | 93158945 | 0.0237 | −0.0759 | 0.4130 |
| 2811 | RP11-74K11 | 12 | 93046134 | 93160933 | 0.0237 | −0.0759 | 0.4130 |
| 3733 | RP11-435E3 | 12 | 94119164 | 94318610 | 0.0236 | −0.0848 | 0.4166 |
| 374 | RP11-410A13 | 12 | 94483197 | 94607734 | 0.0241 | −0.1022 | 0.4043 |
| 4051 | RP11-510I5 | 12 | 95366386 | 95513503 | 0.0241 | −0.1022 | 0.4043 |
| 1358 | RP11-541G9 | 12 | 96065354 | 96245207 | 0.0241 | −0.1022 | 0.4043 |
| 2710 | RP11-481K9 | 12 | 97471300 | 97567505 | 0.0230 | −0.0897 | 0.4283 |
| 318 | RP11-406H4 | 12 | 97941617 | 98048716 | 0.0242 | −0.1074 | 0.4024 |
| 219 | RP11-575G13 | 12 | 99371399 | 99527544 | 0.0242 | −0.1043 | 0.4035 |
| 2108 | RP11-426H24 | 12 | 99942639 | 100140740 | 0.0242 | −0.1043 | 0.4035 |
| 2034 | RP11-285E23 | 12 | 100590405 | 100640299 | 0.0242 | −0.1043 | 0.4035 |
| 3878 | RP11-210L7 | 12 | 101312683 | 101482488 | 0.0242 | −0.0939 | 0.4015 |
| 907 | RP11-13C3 | 12 | 101566355 | 101678102 | 0.0242 | −0.0939 | 0.4015 |
| 1524 | RP11-205I24 | 12 | 102490337 | 102647688 | −0.0171 | −0.0939 | 0.3991 |
| 1571 | RP11-415D21 | 12 | 103552321 | 103714150 | −0.0171 | −0.0939 | 0.3991 |
| 2086 | RP11-478H3 | 12 | 104827123 | 104902202 | −0.0177 | −0.0885 | 0.4107 |
| 2166 | RP11-478H3 | 12 | 104827123 | 104902202 | −0.0177 | −0.0885 | 0.4107 |
| 2041 | RP11-101A9 | 12 | 105069953 | 105070704 | −0.0177 | −0.0885 | 0.4107 |
| 3660 | RP11-90N16 | 12 | 106096622 | 106265244 | −0.0177 | −0.0945 | 0.4122 |
| 241 | RP11-1C11 | 12 | 106901904 | 107044260 | −0.0177 | −0.0945 | 0.4122 |
| 3652 | RP11-256L11 | 12 | 108191164 | 108360951 | −0.0177 | −0.0945 | 0.4122 |
| 3564 | RP1-305I20 | 12 | 109161542 | 109284146 | 0.0052 | −0.0945 | 0.4094 |
| 3567 | RP1-46F2 | 12 | 109800084 | 109949322 | 0.0052 | −0.1036 | 0.4094 |
| 566 | RP11-162P23 | 12 | 110600796 | 110713632 | 0.0052 | −0.1036 | 0.4094 |
| 3560 | RP1-261P5 | 12 | 111103270 | 111175773 | 0.0052 | −0.1036 | 0.4094 |
| 1408 | RP3-363I18 | 12 | 111697722 | 111800274 | 0.0052 | −0.1036 | 0.4094 |
| 2109 | RP11-438N16 | 12 | 112653966 | 112826540 | 0.0060 | −0.1069 | 0.3935 |
| 1808 | RP11-25E2 | 12 | 113938111 | 114127189 | 0.0064 | −0.0960 | 0.3837 |
| 2419 | RP11-8A1 | 12 | 114694711 | 114865871 | 0.0064 | −0.0960 | 0.3837 |
| 8 | RP11-497G19 | 12 | 115487313 | 115638423 | 0.0061 | −0.0805 | 0.3909 |
| 2054 | RP11-110J12 | 12 | 116586716 | 116748345 | 0.0061 | −0.0805 | 0.3909 |
| 2818 | RP11-68H18 | 12 | 117108405 | 117263887 | 0.0060 | −0.0763 | 0.3919 |
| 1872 | RP11-322N7 | 12 | 118059529 | 118238008 | 0.0060 | −0.0763 | 0.3919 |
| 1400 | RP1-267D11 | 12 | 118976136 | 119114667 | 0.0060 | −0.0763 | 0.3919 |
| 2974 | RP11-144B2 | 12 | 119062348 | 119282179 | 0.0060 | −0.0763 | 0.3919 |
| 2189 | RP11-18C24 | 12 | 119397641 | 119534882 | 0.0237 | −0.0763 | 0.3929 |
| 2597 | RP11-44F24 | 12 | 120351695 | 120440866 | 0.0237 | −0.0763 | 0.3929 |
| 1495 | RP11-87C12 | 12 | 120795462 | 120980024 | 0.0237 | −0.0763 | 0.3929 |
| 428 | RP11-32O4 | 12 | 121815155 | 121982522 | 0.0237 | −0.0763 | 0.3929 |
| 326 | RP11-338K17 | 12 | 122658537 | 122877009 | 0.0237 | −0.0763 | 0.3929 |
| 1521 | RP11-214K3 | 12 | 122994584 | 123193120 | 0.0237 | −0.0763 | 0.3929 |
| 1946 | RP5-916L7 | 12 | 124975368 | 125074019 | 0.0238 | −0.0690 | 0.3902 |
| 2412 | RP11-11C16 | 12 | 126779004 | 126932042 | 0.0238 | −0.0690 | 0.3902 |
| 3320 | RP11-178A2 | 12 | 127840870 | 127841450 | 0.0238 | −0.0690 | 0.3902 |
| 3431 | RP11-21K12 | 12 | 127784854 | 127902285 | 0.0238 | −0.0690 | 0.3902 |
| 1340 | RP11-317A20 | 12 | 129082274 | 129082806 | 0.0245 | −0.0767 | 0.3761 |
| 1178 | RP11-143E21 | 12 | 129169568 | 129337249 | 0.0245 | −0.0767 | 0.3761 |
| 253 | RP11-1K22 | 12 | 129648137 | 129839417 | 0.0245 | −0.0767 | 0.3761 |
| 2164 | RP11-495K9 | 12 | 130657863 | 130834848 | 0.0238 | −0.0887 | 0.3907 |
| 426 | RP11-46H11 | 12 | 131857464 | 132036477 | 0.0238 | −0.0887 | 0.3907 |
| 1937 | GS-221-K18 | 12 | 132084534 | 132234534 | 0.0238 | −0.0887 | 0.3907 |
| 931 | RP11-76K19 | 13 | 19007559 | 19137337 | 0.0095 | −0.0541 | 0.4080 |
| 3092 | RP11-187L3 | 13 | 19832836 | 19920914 | 0.0097 | −0.0675 | 0.4036 |
| 943 | RP11-110K8 | 13 | 20994607 | 21089699 | 0.0050 | −0.0692 | 0.5056 |
| 399 | RP11-26A3 | 13 | 21944537 | 22068982 | 0.0096 | −0.0752 | 0.4060 |
| 1983 | RP11-760M1 | 13 | 22887525 | 23056187 | 0.0096 | −0.0752 | 0.4060 |
| 950 | RP11-556N21 | 13 | 23994474 | 24103118 | −0.0177 | −0.0752 | 0.4051 |
| 3107 | RP11-111G7 | 13 | 25124500 | 25300819 | −0.0177 | −0.0804 | 0.4036 |
| 1979 | RP11-570F6 | 13 | 25898592 | 26065378 | −0.0177 | −0.0804 | 0.4036 |
| 1275 | RP11-44J9 | 13 | 26088616 | 26258539 | −0.0177 | −0.0804 | 0.4036 |
| 2556 | RP11-125I23 | 13 | 26843991 | 27010032 | −0.0182 | −0.0876 | 0.4156 |
| 3342 | RP11-153M24 | 13 | 27414162 | 27490882 | −0.0182 | −0.0876 | 0.4156 |
| 3099 | RP11-95G6 | 13 | 27868844 | 27970962 | 0.0283 | −0.0827 | 0.4242 |
| 936 | RP11-218E6 | 13 | 28837843 | 29008462 | 0.0283 | −0.0741 | 0.4236 |
| 946 | RP11-550P23 | 13 | 29914762 | 30048628 | 0.0898 | −0.0312 | 0.4195 |
| 939 | RP11-95N14 | 13 | 31136680 | 31227403 | 0.0903 | −0.0501 | 0.4084 |
| 3349 | RP11-37E23 | 13 | 31703950 | 31870606 | 0.0903 | −0.0501 | 0.4084 |
| 2564 | RP11-141M1 | 13 | 32790709 | 32842539 | 0.0903 | −0.0501 | 0.4084 |
| 3095 | RP11-87G1 | 13 | 32842440 | 33025356 | 0.0903 | −0.0501 | 0.4084 |
| 2553 | RP11-266E6 | 13 | 33995013 | 34159357 | 0.0903 | −0.0544 | 0.4097 |
| 3103 | RP11-98D3 | 13 | 34909905 | 35080902 | 0.0530 | −0.0545 | 0.3985 |
| 2555 | RP11-10M8 | 13 | 37051980 | 37156004 | 0.0149 | −0.0742 | 0.4204 |
| 400 | RP11-131P10 | 13 | 37797278 | 37920029 | 0.0149 | −0.0742 | 0.4204 |
| 2560 | RP11-131F1 | 13 | 38577172 | 38608597 | 0.0150 | −0.0949 | 0.4172 |
| 2554 | RP11-407E23 | 13 | 39306578 | 39498144 | −0.0184 | −0.1008 | 0.4195 |
| 2551 | RP11-2P5 | 13 | 40367434 | 40566000 | −0.0189 | −0.1092 | 0.4305 |
| 392 | RP11-125A7 | 13 | 41105696 | 41263001 | −0.0189 | −0.1092 | 0.4305 |
| 3111 | RP11-117I13 | 13 | 42155411 | 42253968 | −0.0185 | −0.1392 | 0.4211 |
| 2568 | RP11-168P13 | 13 | 43148292 | 43291406 | −0.0184 | −0.1476 | 0.4193 |
| 406 | RP11-442J21 | 13 | 44121277 | 44178227 | −0.0184 | −0.1435 | 0.4195 |
| 413 | RP11-351K3 | 13 | 44939380 | 45114967 | −0.0185 | −0.1385 | 0.4226 |
| 394 | RP11-408L13 | 13 | 46981023 | 47110676 | −0.0186 | −0.1323 | 0.4241 |
| 3339 | RP11-305D15 | 13 | 47760580 | 47897921 | −0.0206 | −0.1513 | 0.4681 |
| 408 | RP11-174I10 | 13 | 47897822 | 47960646 | −0.0206 | −0.1513 | 0.4681 |
| 412 | RP11-185C18 | 13 | 48960782 | 49068912 | −0.0185 | −0.1317 | 0.4217 |
| 2563 | RP11-40A8 | 13 | 50328519 | 50471842 | −0.0188 | −0.0988 | 0.4296 |
| 409 | RP11-327P2 | 13 | 51243102 | 51412205 | −0.0191 | −0.0924 | 0.4346 |
| 2562 | RP11-431O22 | 13 | 52183619 | 52356910 | −0.0189 | −0.1042 | 0.4319 |
| 3105 | RP11-384G23 | 13 | 52856276 | 52955532 | −0.0189 | −0.1042 | 0.4319 |
| 3096 | RP11-200F15 | 13 | 53772767 | 53839190 | −0.0189 | −0.1109 | 0.4317 |
| 410 | RP11-478B20 | 13 | 55372289 | 55430761 | −0.0190 | −0.1061 | 0.4339 |
| 3098 | RP11-516G5 | 13 | 55715878 | 55803708 | −0.0190 | −0.1061 | 0.4339 |
| 3106 | RP11-538C21 | 13 | 57629368 | 57780304 | −0.0190 | −0.1061 | 0.4339 |
| 3102 | RP11-524F1 | 13 | 58618142 | 58784577 | −0.0190 | −0.1061 | 0.4339 |
| 3093 | RP11-359P14 | 13 | 59662064 | 59888923 | −0.0190 | −0.1061 | 0.4339 |
| 2565 | RP11-310K10 | 13 | 60719182 | 60882412 | −0.0190 | −0.1061 | 0.4339 |
| 3104 | RP11-234O23 | 13 | 61999010 | 62167077 | −0.0190 | −0.1061 | 0.4339 |
| 411 | RP11-67L17 | 13 | 62944452 | 63070144 | 0.0082 | −0.1242 | 0.4515 |
| 396 | RP11-129M14 | 13 | 64739934 | 64788659 | 0.0411 | −0.0991 | 0.4396 |
| 398 | RP11-424E21 | 13 | 65400727 | 65415270 | 0.0411 | −0.0991 | 0.4396 |
| 391 | RP11-10M21 | 13 | 66264238 | 66378527 | 0.0411 | −0.0991 | 0.4396 |
| 938 | RP11-521L15 | 13 | 67416969 | 67493555 | 0.0411 | −0.0991 | 0.4396 |
| 937 | RP11-370A2 | 13 | 68205057 | 68256830 | 0.0411 | −0.0991 | 0.4396 |
| 402 | RP11-436I5 | 13 | 69221540 | 69315439 | 0.0411 | −0.0991 | 0.4396 |
| 2572 | RP11-184L18 | 13 | 70296335 | 70417830 | 0.0167 | −0.0897 | 0.4425 |
| 949 | RP11-393H6 | 13 | 71124055 | 71226425 | 0.0167 | −0.0810 | 0.4417 |
| 2561 | RP11-309H15 | 13 | 72606894 | 72687491 | 0.0167 | −0.0810 | 0.4417 |
| 3110 | RP11-552M6 | 13 | 73180719 | 73302614 | 0.0413 | −0.0810 | 0.4478 |
| 3113 | RP11-394J19 | 13 | 74458403 | 74519114 | 0.0192 | −0.0843 | 0.4441 |
| 2573 | RP11-332E3 | 13 | 75289517 | 75440004 | 0.0192 | −0.0843 | 0.4441 |
| 3100 | RP11-226E21 | 13 | 76523033 | 76620509 | 0.0192 | −0.0883 | 0.4446 |
| 3109 | RP11-388E20 | 13 | 77789419 | 77956914 | 0.0196 | −0.1040 | 0.4361 |
| 2567 | RP11-52L5 | 13 | 77954915 | 78112216 | 0.0194 | −0.1093 | 0.4404 |
| 2558 | RP11-421K11 | 13 | 80711170 | 80743754 | −0.0298 | −0.1093 | 0.4334 |
| 942 | RP11-533P8 | 13 | 80910204 | 80958002 | −0.0298 | −0.1093 | 0.4334 |
| 947 | RP11-115N13 | 13 | 82035430 | 82201311 | −0.0298 | −0.1093 | 0.4334 |
| 2570 | RP11-464I4 | 13 | 82592802 | 82779067 | −0.0298 | −0.1093 | 0.4334 |
| 2552 | RP11-120L14 | 13 | 83766477 | 83924559 | −0.0298 | −0.1130 | 0.4354 |
| 3101 | RP11-376H15 | 13 | 84781264 | 84909801 | −0.0298 | −0.1130 | 0.4354 |
| 3097 | RP11-366K1 | 13 | 86241567 | 86431667 | −0.0295 | −0.1227 | 0.4269 |
| 933 | RP11-365J7 | 13 | 86903182 | 86956601 | −0.0295 | −0.1227 | 0.4269 |
| 2574 | RP11-478H12 | 13 | 87930105 | 87985085 | −0.0295 | −0.1227 | 0.4269 |
| 393 | RP11-275J18 | 13 | 88476233 | 88642741 | −0.0295 | −0.1227 | 0.4269 |
| 945 | RP11-388D4 | 13 | 89314344 | 89451286 | −0.0801 | −0.0865 | 0.5394 |
| 2569 | RP11-319L6 | 13 | 90396132 | 90476548 | −0.0757 | −0.1242 | 0.4427 |
| 935 | RP11-95C14 | 13 | 91284428 | 91449527 | −0.0757 | −0.1153 | 0.4428 |
| 4143 | RP11-632L2 | 13 | 92499761 | 92681327 | −0.0755 | −0.1035 | 0.4400 |
| 2571 | RP11-62D23 | 13 | 93229515 | 93403250 | −0.0512 | −0.1034 | 0.4415 |
| 3091 | RP11-74A12 | 13 | 94378172 | 94494192 | −0.0513 | −0.0967 | 0.4436 |
| 405 | RP11-318K19 | 13 | 95333171 | 95461724 | −0.0513 | −0.0967 | 0.4436 |
| 944 | RP11-235O20 | 13 | 96143662 | 96286081 | −0.0278 | −0.0969 | 0.4292 |
| 941 | RP11-383H17 | 13 | 97306085 | 97467156 | −0.0278 | −0.0969 | 0.4292 |
| 2566 | RP11-442I9 | 13 | 98901053 | 98920998 | −0.0280 | −0.0856 | 0.4325 |
| 2557 | RP11-279D17 | 13 | 99118125 | 99217923 | −0.0279 | −0.0913 | 0.4319 |
| 951 | RP11-118F16 | 13 | 100348990 | 100402525 | −0.0284 | −0.0948 | 0.4426 |
| 954 | RP11-564N10 | 13 | 101318689 | 101507384 | −0.0284 | −0.0890 | 0.4420 |
| 3112 | RP11-255P5 | 13 | 102337585 | 102377477 | −0.0286 | −0.0841 | 0.4468 |
| 3114 | RP11-562E17 | 13 | 104304190 | 104442099 | −0.0286 | −0.0841 | 0.4468 |
| 953 | RP11-406G20 | 13 | 105286188 | 105459683 | −0.0286 | −0.0841 | 0.4468 |
| 940 | RP11-232K22 | 13 | 106527417 | 106657339 | −0.0277 | −0.0720 | 0.4259 |
| 934 | RP11-513N16 | 13 | 107333660 | 107395169 | −0.0277 | −0.0720 | 0.4259 |
| 404 | RP11-141M24 | 13 | 108167626 | 108341364 | −0.0468 | −0.1081 | 0.4339 |
| 403 | RP11-40E6 | 13 | 109253824 | 109357572 | −0.0671 | −0.1267 | 0.4371 |
| 395 | RP11-17E4 | 13 | 110602913 | 110681747 | −0.0671 | −0.1334 | 0.4367 |
| 401 | RP11-310D8 | 13 | 111638256 | 111785821 | −0.0671 | −0.1334 | 0.4367 |
| 414 | RP11-480K16 | 13 | 112025897 | 112208258 | −0.0671 | −0.1288 | 0.4369 |
| 952 | RP11-265C7 | 13 | 112776669 | 112779673 | −0.0675 | −0.1432 | 0.4453 |
| 3108 | RP11-245B11 | 13 | 113770511 | 113930806 | −0.0675 | −0.1432 | 0.4453 |
| 1378 | GS-1-L16 | 13 | 113897980 | 114047980 | −0.0675 | −0.1432 | 0.4453 |
| 3538 | GS-163-C9 | 13 | 114047980 | 114197980 | −0.0675 | −0.1432 | 0.4453 |
| 1501 | RP11-98N22 | 14 | 19570792 | 19728992 | 0.0893 | 0.0746 | 0.4290 |
| 1059 | RP11-242H9 | 14 | 20949376 | 21092316 | 0.0574 | 0.0725 | 0.4130 |
| 2047 | RP11-89K22 | 14 | 24548289 | 24713726 | 0.0282 | 0.0478 | 0.3802 |
| 3937 | RP11-330O19 | 14 | 25538833 | 25757239 | 0.0282 | 0.0478 | 0.3802 |
| 814 | RP11-144C18 | 14 | 26574257 | 26745001 | 0.0285 | 0.0625 | 0.3860 |
| 280 | RP11-120I18 | 14 | 27534082 | 27695894 | 0.0288 | 0.0477 | 0.3923 |
| 2576 | RP11-30H9 | 14 | 28381631 | 28530540 | 0.0288 | 0.0477 | 0.3923 |
| 3309 | RP11-419C10 | 14 | 29355162 | 29403141 | 0.2903 | 0.0751 | 0.7324 |
| 1158 | RP11-159L20 | 14 | 30160191 | 30325327 | 0.0025 | 0.0636 | 0.4397 |
| 3868 | RP11-187E13 | 14 | 31443208 | 31600340 | 0.0025 | 0.0585 | 0.4391 |
| 20 | RP11-501E21 | 14 | 32427439 | 32540812 | 0.0067 | 0.0659 | 0.5293 |
| 2447 | RP11-114L8 | 14 | 32835734 | 32999496 | 0.0038 | 0.0484 | 0.4682 |
| 2937 | RP11-561B11 | 14 | 34839169 | 35020664 | 0.0037 | 0.0412 | 0.4644 |
| 3844 | RP11-259K15 | 14 | 35613605 | 35777003 | 0.0035 | 0.0243 | 0.4614 |
| 3392 | RP11-356O9 | 14 | 37010388 | 37212627 | 0.0013 | 0.0042 | 0.4135 |
| 2993 | RP11-138H18 | 14 | 37351226 | 37487778 | 0.0013 | 0.0042 | 0.4135 |
| 3941 | RP11-332O9 | 14 | 40363695 | 40563204 | −0.0224 | −0.0070 | 0.4147 |
| 3319 | RP11-168D12 | 14 | 41353854 | 41500293 | −0.0508 | −0.0069 | 0.4092 |
| 3386 | RP11-333K19 | 14 | 41996958 | 42171562 | −0.0508 | −0.0069 | 0.4092 |
| 817 | RP11-134J10 | 14 | 43288612 | 43453124 | −0.0510 | 0.0005 | 0.4058 |
| 3673 | RP11-99L13 | 14 | 43907856 | 44043642 | −0.0510 | 0.0005 | 0.4058 |
| 912 | RP11-35B20 | 14 | 44799277 | 44957674 | −0.0510 | 0.0005 | 0.4058 |
| 2390 | RP11-565J15 | 14 | 45964782 | 46138593 | −0.0876 | −0.0719 | 0.5117 |
| 2494 | RP11-346L24 | 14 | 48879617 | 49050961 | −0.0163 | 0.0217 | 0.4021 |
| 117 | RP11-58E21 | 14 | 49439912 | 49597311 | −0.0163 | 0.0217 | 0.4021 |
| 1484 | RP11-255G12 | 14 | 50856205 | 51015468 | −0.0170 | 0.0119 | 0.3854 |
| 1562 | RP11-463J10 | 14 | 51390055 | 51556954 | −0.0170 | 0.0119 | 0.3854 |
| 2177 | RP11-463J10 | 14 | 51390055 | 51556954 | −0.0170 | 0.0119 | 0.3854 |
| 2032 | RP11-262M8 | 14 | 51907834 | 51964775 | −0.0170 | 0.0119 | 0.3854 |
| 4053 | RP11-533L7 | 14 | 53101869 | 53262854 | −0.0662 | 0.0222 | 0.3894 |
| 3451 | RP11-60K23 | 14 | 53577087 | 53721826 | −0.0660 | 0.0020 | 0.3940 |
| 1331 | RP11-304L20 | 14 | 54378845 | 54570403 | −0.0660 | 0.0020 | 0.3940 |
| 3830 | RP11-484F16 | 14 | 55649062 | 55827391 | −0.0659 | −0.0197 | 0.3958 |
| 4202 | RP11-108M12 | 14 | 56456898 | 56622286 | −0.0659 | −0.0271 | 0.3952 |
| 1459 | RP11-550M19 | 14 | 57270635 | 57420134 | −0.0659 | −0.0347 | 0.3965 |
| 2377 | RP11-550M19 | 14 | 57270635 | 57420134 | −0.0659 | −0.0347 | 0.3965 |
| 271 | RP11-112J1 | 14 | 58322732 | 58470270 | −0.0664 | −0.0525 | 0.3840 |
| 3769 | RP11-112J1 | 14 | 58322732 | 58470270 | −0.0664 | −0.0525 | 0.3840 |
| 1339 | RP11-307P22 | 14 | 60238356 | 60397779 | −0.0662 | −0.0626 | 0.3881 |
| 430 | RP11-47I22 | 14 | 61037391 | 61208390 | −0.0662 | −0.0597 | 0.3887 |
| 1183 | RP11-316E14 | 14 | 74386055 | 74503657 | −0.0665 | −0.0620 | 0.3834 |
| 307 | RP11-368K8 | 14 | 75210598 | 75354768 | −0.0665 | −0.0620 | 0.3834 |
| 2085 | RP11-368K8 | 14 | 75210598 | 75354768 | −0.0665 | −0.0620 | 0.3834 |
| 1248 | RP11-361H10 | 14 | 75653309 | 75832542 | −0.0667 | −0.0561 | 0.3790 |
| 3713 | RP11-361H10 | 14 | 75653309 | 75832542 | −0.0667 | −0.0561 | 0.3790 |
| 3302 | RP11-463C8 | 14 | 76653300 | 76831630 | −0.0662 | −0.0448 | 0.3886 |
| 657 | RP11-61F4 | 14 | 77244986 | 77511223 | −0.0973 | −0.0457 | 0.3939 |
| 1325 | RP11-285P21 | 14 | 77458135 | 77664678 | −0.0973 | −0.0457 | 0.3939 |
| 4072 | RP11-526N18 | 14 | 79340663 | 79518766 | −0.0965 | −0.0233 | 0.4098 |
| 1292 | RP11-114N19 | 14 | 80497720 | 80657456 | −0.0965 | −0.0233 | 0.4098 |
| 1522 | RP11-226P1 | 14 | 81191225 | 81376452 | −0.0965 | −0.0233 | 0.4098 |
| 3069 | RP11-406A9 | 14 | 82169980 | 82316561 | −0.0959 | −0.0448 | 0.4240 |
| 1172 | RP11-179O11 | 14 | 83056583 | 83215764 | −0.0959 | −0.0448 | 0.4240 |
| 3754 | RP11-179O11 | 14 | 83056583 | 83215764 | −0.0959 | −0.0448 | 0.4240 |
| 2071 | RP11-203D9 | 14 | 83853577 | 84001147 | −0.0959 | −0.0448 | 0.4240 |
| 2724 | RP11-504I1 | 14 | 84168823 | 84169632 | −0.0959 | −0.0448 | 0.4240 |
| 3995 | RP11-96H16 | 14 | 86077244 | 86231535 | −0.0969 | −0.0532 | 0.4011 |
| 3496 | RP11-315O17 | 14 | 86413307 | 86581958 | −0.0969 | −0.0532 | 0.4011 |
| 3708 | RP11-315O17 | 14 | 86413307 | 86581958 | −0.0969 | −0.0532 | 0.4011 |
| 3437 | RP11-2E15 | 14 | 86833840 | 86986583 | −0.0973 | −0.0436 | 0.3937 |
| 341 | RP11-300J18 | 14 | 87489467 | 87634856 | −0.0969 | −0.0530 | 0.4024 |
| 2087 | RP11-300J18 | 14 | 87489467 | 87634856 | −0.0969 | −0.0530 | 0.4024 |
| 1170 | RP11-164H13 | 14 | 95199486 | 95364094 | −0.0638 | −0.0619 | 0.4206 |
| 1051 | RP11-185P18 | 14 | 95364095 | 95527190 | −0.0638 | −0.0619 | 0.4206 |
| 3083 | RP11-13O24 | 15 | 8632 | 331718 | −0.2556 | −0.0820 | 0.5068 |
| 3501 | RP11-289D12 | 15 | 20428073 | 20542381 | −0.2237 | −0.0629 | 0.3779 |
| 2449 | RP11-131I21 | 15 | 22736020 | 22894878 | −0.1874 | −0.0688 | 0.3806 |
| 3746 | RP11-446P9 | 15 | 23661828 | 23823376 | −0.1874 | −0.0688 | 0.3806 |
| 770 | RP11-570N16 | 15 | 24960279 | 25130030 | −0.1875 | −0.0740 | 0.3781 |
| 4036 | RP11-322N14 | 15 | 25800215 | 25980080 | −0.1875 | −0.0740 | 0.3781 |
| 370 | RP11-408F10 | 15 | 27500519 | 27613844 | −0.1875 | −0.0740 | 0.3781 |
| 2581 | RP11-38E12 | 15 | 28255532 | 28543404 | −0.1658 | −0.0740 | 0.3745 |
| 1174 | RP11-164K24 | 15 | 29207958 | 29366727 | −0.1424 | −0.0587 | 0.3690 |
| 2027 | RP11-250G15 | 15 | 31026423 | 31138717 | −0.1399 | −0.0285 | 0.4216 |
| 2406 | RP11-3D4 | 15 | 31826214 | 31975488 | −0.1422 | −0.0502 | 0.3719 |
| 2808 | RP11-83J16 | 15 | 32893104 | 33055200 | −0.1418 | −0.0718 | 0.3818 |
| 2263 | RP11-70A1 | 15 | 34700382 | 34860406 | −0.1418 | −0.0751 | 0.3823 |
| 2028 | RP11-261P5 | 15 | 35740031 | 35906900 | −0.1418 | −0.0751 | 0.3823 |
| 4165 | CTD-2033D15 | 15 | 37614149 | 37799843 | −0.1236 | −0.0751 | 0.3855 |
| 3455 | RP11-64K12 | 15 | 38393770 | 38570669 | −0.1236 | −0.0751 | 0.3855 |
| 3529 | RP11-532F12 | 15 | 38766143 | 38944390 | −0.1236 | −0.0751 | 0.3855 |
| 304 | RP11-380D11 | 15 | 39583713 | 39754407 | −0.1235 | −0.0826 | 0.3867 |
| 3334 | RP11-164J13 | 15 | 40399163 | 40544395 | −0.0989 | −0.0825 | 0.3806 |
| 1779 | RP11-355D13 | 15 | 41409158 | 41591921 | −0.0986 | −0.0883 | 0.3871 |
| 3641 | RP11-263I19 | 15 | 41680206 | 41851733 | −0.0986 | −0.0883 | 0.3871 |
| 3325 | RP11-151N17 | 15 | 42593665 | 42756159 | −0.0986 | −0.0883 | 0.3871 |
| 3520 | RP11-519G16 | 15 | 43413001 | 43613803 | −0.0986 | −0.0883 | 0.3871 |
| 1332 | RP11-315O8 | 15 | 44046010 | 44246699 | −0.0986 | −0.0883 | 0.3871 |
| 4226 | RP11-232J12 | 15 | 51579153 | 51736194 | −0.0921 | −0.0951 | 0.3849 |
| 2137 | RP11-141F2 | 15 | 52559590 | 52719684 | −0.0921 | −0.0951 | 0.3849 |
| 2936 | RP11-141F2 | 15 | 52559590 | 52719684 | −0.0921 | −0.0951 | 0.3849 |
| 3693 | RP11-215J7 | 15 | 52652476 | 52815253 | −0.0921 | −0.0951 | 0.3849 |
| 1542 | RP11-231A23 | 15 | 58182960 | 58247408 | −0.0972 | −0.0888 | 0.3650 |
| 1175 | RP11-169M2 | 15 | 60717917 | 60895399 | −0.0972 | −0.0888 | 0.3650 |
| 3956 | RP11-24N10 | 15 | 60932059 | 61119643 | −0.0978 | −0.0960 | 0.3517 |
| 2078 | RP11-236P11 | 15 | 62379521 | 62510410 | −0.0977 | −0.0881 | 0.3544 |
| 3393 | RP11-325L12 | 15 | 63070257 | 63255120 | −0.0975 | −0.0841 | 0.3586 |
| 4042 | RP11-302G2 | 15 | 63873345 | 64073088 | −0.0975 | −0.0867 | 0.3589 |
| 1235 | RP11-348J6 | 15 | 63987575 | 64164144 | −0.0975 | −0.0867 | 0.3589 |
| 4244 | RP11-348J6 | 15 | 63987575 | 64164144 | −0.0975 | −0.0867 | 0.3589 |
| 3347 | RP11-321F6 | 15 | 64661286 | 64838835 | −0.0975 | −0.0867 | 0.3589 |
| 3893 | RP11-100A11 | 15 | 64818125 | 64818855 | −0.0975 | −0.0867 | 0.3589 |
| 3518 | RP11-540N15 | 15 | 64985110 | 65179655 | −0.0975 | −0.0867 | 0.3589 |
| 1195 | RP11-342M21 | 15 | 65164138 | 65330922 | −0.0975 | −0.0867 | 0.3589 |
| 793 | RP11-2I17 | 15 | 70142505 | 70313627 | −0.0542 | −0.0723 | 0.3942 |
| 1553 | RP11-361M10 | 15 | 70693862 | 70907606 | −0.0542 | −0.0723 | 0.3945 |
| 1768 | RP11-361M10 | 15 | 70693862 | 70907606 | −0.0542 | −0.0723 | 0.3945 |
| 2033 | RP11-272D12 | 15 | 71270952 | 71440843 | −0.0542 | −0.0723 | 0.3945 |
| 2943 | RP11-8P11 | 15 | 71764663 | 71904838 | −0.0542 | −0.0801 | 0.3937 |
| 3727 | RP11-414J4 | 15 | 72768830 | 72953361 | −0.0549 | −0.0614 | 0.4097 |
| 4212 | RP11-94P14 | 15 | 75185320 | 75348639 | 0.0065 | 0.0048 | 0.5147 |
| 12 | RP11-499F3 | 15 | 79676203 | 79756580 | −0.0130 | 0.0248 | 0.4952 |
| 3329 | RP11-152F13 | 15 | 80887175 | 81024207 | −0.0130 | 0.0319 | 0.4961 |
| 2051 | RP11-90B9 | 15 | 81348690 | 81488675 | −0.0130 | 0.0319 | 0.4961 |
| 295 | RP11-365F16 | 15 | 82005246 | 82186756 | −0.0116 | 0.0644 | 0.5251 |
| 230 | RP11-565O12 | 15 | 83040659 | 83070787 | −0.0122 | 0.0789 | 0.5131 |
| 1905 | RP11-535P8 | 15 | 83685716 | 83883618 | −0.0134 | 0.0625 | 0.4867 |
| 813 | RP11-133L19 | 15 | 85186168 | 85362806 | −0.0317 | 0.0503 | 0.4923 |
| 1787 | RP11-356B18 | 15 | 86472170 | 86639789 | −0.0317 | 0.0503 | 0.4923 |
| 4264 | RP11-356B18 | 15 | 86472170 | 86639789 | −0.0317 | 0.0503 | 0.4923 |
| 3397 | RP11-326A19 | 15 | 87338936 | 87527181 | −0.0226 | 0.0686 | 0.4939 |
| 2066 | RP11-233C13 | 15 | 88091170 | 88207896 | −0.0219 | 0.0787 | 0.5089 |
| 4298 | RP11-233C13 | 15 | 88091170 | 88207896 | −0.0219 | 0.0787 | 0.5089 |
| 3576 | RP3-443N8 | 15 | 88522448 | 88672448 | −0.0219 | 0.0781 | 0.5110 |
| 3056 | RP1-138O23 | 15 | 88798992 | 88948992 | −0.0220 | 0.0949 | 0.5071 |
| 1727 | RP11-405A15 | 15 | 89241954 | 89260209 | 0.0087 | 0.0991 | 0.5082 |
| 1493 | RP11-266O8 | 15 | 91550182 | 91714423 | 0.0074 | 0.1041 | 0.4800 |
| 429 | RP11-41P8 | 15 | 92324496 | 92491964 | 0.0075 | 0.0949 | 0.4835 |
| 436 | RP11-34L4 | 15 | 93038001 | 93169755 | 0.0075 | 0.0949 | 0.4835 |
| 2144 | RP11-34L4 | 15 | 93038001 | 93169755 | 0.0075 | 0.0949 | 0.4835 |
| 4308 | RP11-44B21 | 15 | 93987934 | 94138221 | −0.0099 | 0.0949 | 0.4830 |
| 1328 | RP11-315L6 | 15 | 95508704 | 95699734 | −0.0099 | 0.0949 | 0.4830 |
| 1700 | RP11-183E24 | 15 | 96460021 | 96572341 | −0.0092 | 0.1174 | 0.4982 |
| 3355 | RP11-342L10 | 15 | 96860057 | 97061845 | −0.0092 | 0.1174 | 0.4982 |
| 3633 | RP11-262P8 | 15 | 97047742 | 97225942 | −0.0092 | 0.1174 | 0.4982 |
| 4215 | RP11-90E5 | 15 | 98388046 | 98574562 | −0.0092 | 0.1174 | 0.4982 |
| 3323 | RP11-168G16 | 15 | 98677983 | 98814208 | −0.0092 | 0.1174 | 0.4982 |
| 4022 | RP11-299G20 | 15 | 99550339 | 99734009 | −0.0092 | 0.1174 | 0.4982 |
| 1382 | GS-124-5 | 15 | 99963915 | 100113915 | −0.0079 | 0.0925 | 0.5276 |
| 3546 | GS-154-P1 | 15 | 99963965 | 100113965 | −0.0079 | 0.0925 | 0.5276 |
| 3886 | RP11-243K18 | 16 | 266993 | 431793 | −0.0447 | −0.0548 | 0.3994 |
| 1182 | RP11-161M6 | 16 | 919651 | 1081992 | −0.0435 | −0.0717 | 0.3735 |
| 4158 | RP11-304L19 | 16 | 2081103 | 2267414 | −0.0435 | −0.0717 | 0.3735 |
| 4288 | RP11-433P17 | 16 | 3292433 | 3471943 | −0.0433 | −0.0823 | 0.3693 |
| 2169 | RP11-462G12 | 16 | 3918289 | 4131849 | −0.0433 | −0.0625 | 0.3690 |
| 3084 | RP11-35P16 | 16 | 4441438 | 4520536 | −0.0432 | −0.0577 | 0.3687 |
| 1710 | RP11-167B4 | 16 | 6171414 | 6339845 | −0.0629 | −0.0617 | 0.3523 |
| 2990 | RP11-148F10 | 16 | 7933143 | 8120601 | −0.0629 | −0.0617 | 0.3523 |
| 3853 | RP11-152P23 | 16 | 8777495 | 8852419 | −0.0625 | −0.0380 | 0.3604 |
| 4309 | RP11-152P23 | 16 | 8777495 | 8852419 | −0.0625 | −0.0380 | 0.3604 |
| 3490 | RP11-297M9 | 16 | 9634532 | 9827751 | −0.0626 | −0.0319 | 0.3583 |
| 3119 | RP11-27M24 | 16 | 10507665 | 10596534 | −0.0626 | −0.0319 | 0.3583 |
| 543 | RP11-490O6 | 16 | 11635175 | 11796320 | −0.0608 | −0.0251 | 0.3976 |
| 424 | RP11-31O11 | 16 | 11848758 | 12014145 | −0.0608 | −0.0251 | 0.3976 |
| 4304 | RP11-31O11 | 16 | 11848758 | 12014145 | −0.0608 | −0.0251 | 0.3976 |
| 373 | RP11-276H1 | 16 | 12014799 | 12176022 | −0.0623 | −0.0391 | 0.3647 |
| 2804 | RP11-82O18 | 16 | 14602445 | 14723189 | −0.0625 | −0.0375 | 0.3617 |
| 1674 | RP11-489O1 | 16 | 15411955 | 15573494 | −0.0625 | −0.0375 | 0.3617 |
| 2009 | CTD-2504F3 | 16 | 15986518 | 16192266 | −0.0625 | −0.0375 | 0.3617 |
| 296 | RP11-378B23 | 16 | 16783566 | 16933566 | −0.0626 | −0.0409 | 0.3598 |
| 3497 | RP11-288I13 | 16 | 17137869 | 17316758 | −0.0626 | −0.0409 | 0.3598 |
| 2692 | RP11-626G11 | 16 | 18850659 | 19066922 | −0.0625 | −0.0379 | 0.3605 |
| 2684 | RP11-429K17 | 16 | 20151302 | 20315827 | −0.0625 | −0.0379 | 0.3605 |
| 3834 | RP11-489A11 | 16 | 21304276 | 21473433 | −0.0628 | −0.0301 | 0.3544 |
| 4205 | RP11-101E7 | 16 | 21936632 | 22116416 | −0.0628 | −0.0301 | 0.3544 |
| 1676 | RP11-105C19 | 16 | 22508689 | 22679811 | −0.0628 | −0.0301 | 0.3544 |
| 3765 | RP11-105C19 | 16 | 22508689 | 22679811 | −0.0628 | −0.0301 | 0.3544 |
| 650 | RP11-67I10 | 16 | 26107482 | 26264124 | −0.0236 | −0.0249 | 0.3537 |
| 1836 | RP11-142A12 | 16 | 26595064 | 26727359 | −0.0236 | −0.0249 | 0.3537 |
| 4301 | RP11-142A12 | 16 | 26595064 | 26727359 | −0.0236 | −0.0249 | 0.3537 |
| 4094 | F21283 | 16 | 28579078 | 28729078 | −0.0236 | −0.0371 | 0.3547 |
| 2962 | RP11-5L1 | 16 | 45067244 | 45231075 | −0.0267 | −0.0829 | 0.3739 |
| 2022 | RP11-283C7 | 16 | 45627546 | 45688113 | −0.0568 | −0.0923 | 0.3856 |
| 3711 | RP11-283C7 | 16 | 45627546 | 45688113 | −0.0568 | −0.0923 | 0.3856 |
| 822 | RP11-147B17 | 16 | 49090412 | 49252652 | −0.1116 | −0.1251 | 0.3714 |
| 3740 | RP11-424K7 | 16 | 49618230 | 49794206 | −0.1116 | −0.1294 | 0.3725 |
| 812 | RP11-122K22 | 16 | 50431358 | 50611758 | −0.1119 | −0.1369 | 0.3783 |
| 2701 | RP11-467J12 | 16 | 51466013 | 51629836 | −0.1120 | −0.1450 | 0.3812 |
| 1232 | RP11-357N13 | 16 | 52448777 | 52626499 | −0.1120 | −0.1450 | 0.3812 |
| 3709 | RP11-357N13 | 16 | 52448777 | 52626499 | −0.1120 | −0.1450 | 0.3812 |
| 2415 | RP11-7O2 | 16 | 53823781 | 53975887 | −0.1120 | −0.1585 | 0.3818 |
| 1702 | RP11-165M2 | 16 | 54486638 | 54559962 | −0.1118 | −0.1703 | 0.3771 |
| 1229 | RP11-325K4 | 16 | 55378542 | 55559305 | −0.1120 | −0.1793 | 0.3809 |
| 2470 | RP11-405F3 | 16 | 56181076 | 56331523 | −0.1122 | −0.1848 | 0.3850 |
| 2090 | RP11-481J2 | 16 | 56965501 | 57139976 | −0.1120 | −0.1928 | 0.3807 |
| 2706 | RP11-481J2 | 16 | 56965501 | 57139976 | −0.1120 | −0.1928 | 0.3807 |
| 2057 | RP11-105C20 | 16 | 58235437 | 58349316 | −0.1122 | −0.1984 | 0.3859 |
| 4282 | RP11-457D20 | 16 | 58776524 | 58993500 | −0.1122 | −0.1984 | 0.3859 |
| 3314 | RP11-157H19 | 16 | 60426260 | 60527522 | −0.1131 | −0.2146 | 0.4044 |
| 2705 | RP11-467O15 | 16 | 61098231 | 61286993 | −0.1131 | −0.2146 | 0.4044 |
| 830 | RP11-148F12 | 16 | 61943888 | 62080690 | −0.1130 | −0.2200 | 0.4018 |
| 2995 | RP11-370P15 | 16 | 62221465 | 62396983 | −0.1124 | −0.2102 | 0.3898 |
| 541 | RP11-467L24 | 16 | 62921648 | 63071733 | −0.1124 | −0.2102 | 0.3898 |
| 1538 | RP11-229O3 | 16 | 63462867 | 63616454 | −0.1124 | −0.2102 | 0.3898 |
| 3498 | RP11-298C15 | 16 | 63934753 | 64142097 | −0.0844 | −0.2185 | 0.3897 |
| 4243 | RP11-298C15 | 16 | 63934753 | 64142097 | −0.0844 | −0.2185 | 0.3897 |
| 1291 | RP11-63M22 | 16 | 65152748 | 65333456 | −0.1265 | −0.2227 | 0.3957 |
| 1155 | RP11-167P11 | 16 | 66238838 | 66393297 | −0.1265 | −0.2227 | 0.3957 |
| 2819 | RP11-76H6 | 16 | 67078490 | 67134347 | −0.1262 | −0.2189 | 0.3896 |
| 3359 | RP11-354N7 | 16 | 67318526 | 67478802 | −0.0749 | −0.2061 | 0.3893 |
| 816 | RP11-123C5 | 16 | 67554082 | 67731555 | −0.0749 | −0.2061 | 0.3893 |
| 4031 | RP11-311C24 | 16 | 68224900 | 68296605 | −0.0750 | −0.1990 | 0.3912 |
| 1563 | RP11-296I10 | 16 | 68690128 | 68870723 | −0.0753 | −0.2044 | 0.3984 |
| 3486 | RP11-296I10 | 16 | 68690128 | 68870723 | −0.0753 | −0.2044 | 0.3984 |
| 2469 | RP11-394B2 | 16 | 69203922 | 69365103 | −0.0752 | −0.2120 | 0.3967 |
| 3305 | RP11-417N10 | 16 | 70330095 | 70453950 | −0.0978 | −0.2121 | 0.3931 |
| 2267 | RP11-70E3 | 16 | 70619026 | 70788093 | −0.0978 | −0.2121 | 0.3931 |
| 2735 | RP11-328J14 | 16 | 70783654 | 70973636 | −0.0978 | −0.2121 | 0.3931 |
| 3194 | RP5-991G20 | 16 | 71316768 | 71457881 | −0.0986 | −0.2008 | 0.4107 |
| 3485 | RP11-285K4 | 16 | 72300075 | 72453658 | −0.0986 | −0.2008 | 0.4107 |
| 100 | RP11-77K12 | 16 | 73977115 | 74134472 | −0.0986 | −0.2008 | 0.4107 |
| 4219 | RP11-200H5 | 16 | 74775676 | 74819173 | −0.0987 | −0.2104 | 0.4136 |
| 3516 | RP11-518I8 | 16 | 75914617 | 76024261 | −0.0987 | −0.2104 | 0.4136 |
| 3432 | RP11-24I3 | 16 | 76343711 | 76539056 | −0.1210 | −0.2104 | 0.4102 |
| 4174 | RP11-281J9 | 16 | 76447923 | 76606529 | −0.1210 | −0.2104 | 0.4102 |
| 2917 | RP11-556H2 | 16 | 77599698 | 77781811 | −0.1214 | −0.2258 | 0.4182 |
| 3483 | RP11-303E16 | 16 | 79571775 | 79773939 | −0.1214 | −0.2258 | 0.4182 |
| 4251 | RP11-303E16 | 16 | 79571775 | 79773939 | −0.1214 | −0.2258 | 0.4182 |
| 3290 | RP11-446C5 | 16 | 79834964 | 80033098 | −0.1216 | −0.2167 | 0.4229 |
| 3849 | RP11-437L22 | 16 | 80148499 | 80307562 | −0.1216 | −0.2167 | 0.4229 |
| 2402 | RP11-2L4 | 16 | 81162708 | 81279605 | −0.1217 | −0.2113 | 0.4242 |
| 558 | RP11-483P21 | 16 | 82361608 | 82534818 | −0.1219 | −0.1943 | 0.4288 |
| 4254 | RP11-483P21 | 16 | 82361608 | 82534818 | −0.1219 | −0.1943 | 0.4288 |
| 1480 | RP11-254F19 | 16 | 83357615 | 83531735 | −0.1215 | −0.1926 | 0.4197 |
| 2170 | RP11-478M13 | 16 | 84379908 | 84540021 | −0.1213 | −0.1876 | 0.4158 |
| 1907 | RP11-514D23 | 16 | 84812502 | 84922043 | −0.1422 | −0.2044 | 0.3923 |
| 1251 | RP11-21B21 | 16 | 87086505 | 87260489 | −0.1421 | −0.1974 | 0.3909 |
| 1401 | RP4-597G12 | 16 | 88403055 | 88495451 | −0.1429 | −0.1873 | 0.4071 |
| 3533 | RP11-533D19 | 16 | 88421159 | 88643456 | −0.1429 | −0.1873 | 0.4071 |
| 3550 | GS-191-P24 | 16 | 88552254 | 88702254 | −0.1429 | −0.1873 | 0.4071 |
| 3697 | RP11-216P6 | 17 | 800495 | 1008155 | −0.1480 | −0.1417 | 0.4192 |
| 786 | RP11-4F24 | 17 | 1564767 | 1565500 | −0.1480 | −0.1417 | 0.4192 |
| 821 | RP11-135N5 | 17 | 2316192 | 2492179 | −0.1480 | −0.1417 | 0.4192 |
| 1198 | RP11-199F11 | 17 | 7516212 | 7599153 | −0.1691 | −0.1925 | 0.4146 |
| 2466 | RP11-404G1 | 17 | 7563684 | 7682367 | −0.1691 | −0.1925 | 0.4146 |
| 3977 | RP11-12H18 | 17 | 8446409 | 8574345 | −0.1691 | −0.1925 | 0.4146 |
| 3700 | RP11-208F13 | 17 | 9466335 | 9611536 | −0.1683 | −0.2213 | 0.3969 |
| 3293 | RP11-401O9 | 17 | 10055057 | 10253638 | −0.1683 | −0.2213 | 0.3969 |
| 2717 | RP11-471L13 | 17 | 11940105 | 12065840 | −0.1681 | −0.2304 | 0.3928 |
| 15 | RP11-488L1 | 17 | 12940551 | 13028533 | −0.1677 | −0.2429 | 0.3828 |
| 3034 | RP11-388F14 | 17 | 13226766 | 13375795 | −0.1677 | −0.2429 | 0.3828 |
| 3559 | RP1-27J12 | 17 | 14285924 | 14360892 | −0.1677 | −0.2429 | 0.3828 |
| 569 | RP11-64B12 | 17 | 14531958 | 14598528 | −0.1677 | −0.2429 | 0.3828 |
| 3004 | RP11-385D13 | 17 | 15374763 | 15435531 | −0.1676 | −0.2461 | 0.3805 |
| 2061 | RP11-219A15 | 17 | 16646746 | 16718827 | −0.2025 | −0.2553 | 0.3820 |
| 3299 | RP11-524F11 | 17 | 17338138 | 17521471 | −0.2022 | −0.2478 | 0.3767 |
| 1712 | RP11-189D22 | 17 | 17940393 | 18114679 | −0.2022 | −0.2478 | 0.3767 |
| 3200 | RP1-162E17 | 17 | 19126535 | 19251679 | −0.2022 | −0.2478 | 0.3767 |
| 3629 | CTB-1187M2 | 17 | 19211694 | 19360796 | −0.2022 | −0.2478 | 0.3767 |
| 2737 | RP11-78O7 | 17 | 19614001 | 19748680 | −0.2016 | −0.2400 | 0.3625 |
| 1961 | RP5-836L9 | 17 | 20014357 | 20134155 | −0.2016 | −0.2400 | 0.3625 |
| 46 | RP11-121A13 | 17 | 20134151 | 20230382 | −0.2016 | −0.2400 | 0.3625 |
| 519 | RP11-64J19 | 17 | 21055083 | 21191541 | −0.2016 | −0.2400 | 0.3625 |
| 3216 | RP11-744K17 | 17 | 21822900 | 21916486 | −0.2014 | −0.2465 | 0.3581 |
| 4180 | RP11-260A9 | 17 | 22287134 | 22465691 | −0.1545 | −0.2397 | 0.3515 |
| 2434 | RP11-138P22 | 17 | 23133770 | 23308272 | −0.1545 | −0.2397 | 0.3515 |
| 3035 | RP11-192H23 | 17 | 23864147 | 24008957 | −0.0864 | −0.2291 | 0.3995 |
| 4213 | RP11-104I20 | 17 | 25164209 | 25184918 | −0.0864 | −0.2291 | 0.3995 |
| 302 | RP11-403E9 | 17 | 25591017 | 25693302 | −0.0237 | −0.2201 | 0.4841 |
| 3698 | RP11-229K15 | 17 | 26294586 | 26453072 | −0.1541 | −0.2123 | 0.3622 |
| 3338 | RP11-142O6 | 17 | 26429957 | 26575104 | −0.1541 | −0.2123 | 0.3622 |
| 557 | RP11-474K4 | 17 | 27416322 | 27596064 | −0.1541 | −0.2123 | 0.3622 |
| 1399 | RP1-29G21 | 17 | 28308287 | 28423825 | −0.1566 | −0.2124 | 0.4185 |
| 3840 | RP11-139K6 | 17 | 28664934 | 28816067 | −0.1566 | −0.2124 | 0.4185 |
| 1529 | RP11-215E13 | 17 | 29522483 | 29667899 | −0.1567 | −0.2012 | 0.4193 |
| 3562 | RP5-837J1 | 17 | 30346236 | 30441459 | −0.1315 | −0.1844 | 0.3738 |
| 45 | RP11-47L3 | 17 | 30523516 | 30685989 | −0.1313 | −0.1933 | 0.3712 |
| 3738 | RP11-445F12 | 17 | 32246319 | 32371528 | −0.0897 | −0.1894 | 0.3865 |
| 4122 | RP5-986F12 | 17 | 33099924 | 33206361 | −0.0903 | −0.1804 | 0.4011 |
| 4280 | RP11-432I21 | 17 | 33794727 | 33977073 | −0.0903 | −0.1804 | 0.4011 |
| 3621 | CTB-1058E17 | 17 | 33964845 | 34251503 | −0.0906 | −0.1688 | 0.4064 |
| 4106 | RP5-906A24 | 17 | 34510775 | 34624022 | −0.0906 | −0.1688 | 0.4064 |
| 2196 | RP11-390P24 | 17 | 34803854 | 34979173 | −0.0904 | −0.1802 | 0.4026 |
| 3885 | RP11-94L15 | 17 | 35065321 | 35227135 | −0.0906 | −0.1632 | 0.4062 |
| 3053 | RP5-1112G21 | 17 | 35682018 | 35682629 | −0.1429 | −0.1634 | 0.3939 |
| 637 | RP11-58O9 | 17 | 35754712 | 35920945 | −0.1429 | −0.1634 | 0.3939 |
| 2660 | RP5-1110E20 | 17 | 36260511 | 36373721 | −0.1430 | −0.1529 | 0.3960 |
| 3869 | RP11-156E6 | 17 | 37192545 | 37370889 | −0.1430 | −0.1494 | 0.3956 |
| 2744 | RP11-506G7 | 17 | 38139932 | 38327654 | −0.1077 | −0.1495 | 0.3889 |
| 3883 | RP11-376M2 | 17 | 38221253 | 38425998 | −0.1077 | −0.1495 | 0.3889 |
| 2008 | RP11-948G15 | 17 | 38425359 | 38425924 | −0.1079 | −0.1382 | 0.3925 |
| 36 | RP11-392O1 | 17 | 38932046 | 39091576 | −0.1078 | −0.1325 | 0.3907 |
| 1942 | RP5-905N1 | 17 | 39182384 | 39277131 | −0.1078 | −0.1325 | 0.3907 |
| 1045 | RP5-1067M6 | 17 | 39683991 | 39821481 | −0.0361 | −0.1261 | 0.4186 |
| 2654 | RP5-1169K15 | 17 | 40698240 | 40742647 | −0.0363 | −0.1084 | 0.4235 |
| 3193 | RP5-843B9 | 17 | 41441703 | 41565982 | −0.0363 | −0.1084 | 0.4235 |
| 4118 | RP5-971F3 | 17 | 41818827 | 41939592 | −0.0420 | −0.2518 | 0.5467 |
| 2065 | RP11-220N20 | 17 | 42226771 | 42268630 | −0.0375 | −0.1378 | 0.4492 |
| 3739 | RP11-416K7 | 17 | 42875373 | 43043115 | −0.0373 | −0.1060 | 0.4454 |
| 3928 | RP11-361K8 | 17 | 43957672 | 44125738 | −0.0030 | −0.0579 | 0.4155 |
| 3571 | RP1-62O9 | 17 | 44647599 | 44753692 | −0.0030 | −0.0579 | 0.4155 |
| 577 | RP11-81K2 | 17 | 44908966 | 44942436 | −0.0030 | −0.0579 | 0.4155 |
| 1410 | RP5-875H18 | 17 | 45492779 | 45587911 | −0.0030 | −0.0579 | 0.4155 |
| 585 | RP11-94C24 | 17 | 45874046 | 46041875 | −0.0035 | −0.0497 | 0.4258 |
| 3515 | RP11-506D12 | 17 | 46263624 | 46333071 | −0.0035 | −0.0449 | 0.4255 |
| 2702 | RP11-481C4 | 17 | 46505878 | 46634205 | −0.0048 | −0.0305 | 0.4542 |
| 4250 | RP11-481C4 | 17 | 46505878 | 46634205 | −0.0048 | −0.0305 | 0.4542 |
| 2023 | RP11-248L3 | 17 | 48434263 | 48595726 | −0.0297 | −0.0533 | 0.4083 |
| 3201 | RP5-1043H15 | 17 | 48606238 | 48664580 | −0.0296 | −0.0451 | 0.4056 |
| 3025 | RP11-312B18 | 17 | 49013794 | 49212728 | −0.0296 | −0.0451 | 0.4056 |
| 1537 | RP11-217N19 | 17 | 49753469 | 49922957 | −0.0291 | −0.0547 | 0.3936 |
| 3003 | RP11-372K20 | 17 | 50112652 | 50268101 | −0.0291 | −0.0547 | 0.3936 |
| 1911 | RP11-515O17 | 17 | 50675072 | 50836282 | −0.0291 | −0.0547 | 0.3936 |
| 2102 | RP11-515O17 | 17 | 50675072 | 50836282 | −0.0291 | −0.0547 | 0.3936 |
| 2435 | RP11-112J9 | 17 | 52088163 | 52172786 | −0.0291 | −0.0547 | 0.3936 |
| 3958 | RP11-19F16 | 17 | 52616475 | 52760769 | −0.0291 | −0.0547 | 0.3936 |
| 3608 | RP11-579A4 | 17 | 54149949 | 54280513 | −0.0291 | −0.0547 | 0.3936 |
| 3070 | RP11-567L7 | 17 | 54696369 | 54872935 | −0.0291 | −0.0402 | 0.3935 |
| 1160 | RP11-178C3 | 17 | 55387410 | 55498115 | −0.0291 | −0.0402 | 0.3935 |
| 3351 | RP11-332H18 | 17 | 56659719 | 56847075 | −0.0290 | −0.0329 | 0.3916 |
| 1774 | RP11-342K2 | 17 | 57418528 | 57473362 | −0.0290 | −0.0329 | 0.3916 |
| 1701 | RP11-155C2 | 17 | 69446939 | 69639765 | −0.0206 | 0.0071 | 0.3923 |
| 2174 | RP11-478P5 | 17 | 69778939 | 69931286 | −0.0206 | 0.0071 | 0.3923 |
| 3691 | RP11-196E5 | 17 | 71063165 | 71247066 | −0.0209 | −0.0116 | 0.3863 |
| 1499 | RP11-87G24 | 17 | 72342642 | 72523921 | −0.0207 | −0.0108 | 0.3908 |
| 286 | RP11-141D15 | 17 | 73636230 | 73813841 | −0.0206 | −0.0044 | 0.3943 |
| 1880 | RP11-323N12 | 17 | 74200806 | 74384944 | −0.0206 | −0.0044 | 0.3943 |
| 849 | RP11-398J5 | 17 | 74610889 | 74792977 | −0.0206 | −0.0044 | 0.3943 |
| 4043 | RP11-313F15 | 17 | 76183534 | 76354885 | −0.0203 | 0.0026 | 0.3996 |
| 1900 | RP11-525L23 | 17 | 77988926 | 78221351 | −0.0199 | 0.0090 | 0.4074 |
| 2106 | RP11-525L23 | 17 | 77988926 | 78221351 | −0.0199 | 0.0090 | 0.4074 |
| 758 | RP11-567O16 | 17 | 78311474 | 78374827 | −0.0199 | 0.0090 | 0.4074 |
| 1919 | GS-50-C4 | 17 | 78499742 | 78649742 | −0.0199 | 0.0090 | 0.4074 |
| 3554 | GS-362-K4 | 17 | 78609742 | 78759742 | −0.0202 | −0.0146 | 0.4030 |
| 1394 | GS-52-M11 | 18 | 1 | 125000 | −0.1875 | −0.1621 | 0.5212 |
| 1380 | GS-74-G18 | 18 | 137949 | 287949 | 0.0361 | −0.0161 | 0.4287 |
| 4044 | RP11-324G2 | 18 | 168428 | 340445 | 0.0361 | −0.0161 | 0.4287 |
| 4173 | RP11-267C19 | 18 | 957554 | 1114347 | 0.0361 | −0.0161 | 0.4287 |
| 1861 | RP11-291G24 | 18 | 1551101 | 1734182 | 0.0361 | −0.0161 | 0.4287 |
| 4279 | RP11-419P8 | 18 | 2479734 | 2665807 | 0.0365 | −0.0050 | 0.4203 |
| 1506 | RP11-106J7 | 18 | 3268738 | 3442992 | 0.0367 | 0.0019 | 0.4169 |
| 548 | RP11-502P1 | 18 | 3863227 | 4046692 | 0.0369 | 0.0115 | 0.4131 |
| 1516 | RP11-92G19 | 18 | 4926254 | 5083821 | 0.0370 | 0.0071 | 0.4109 |
| 3946 | RP11-344B7 | 18 | 5260008 | 5444854 | 0.0370 | 0.0071 | 0.4109 |
| 1998 | RP11-39D15 | 18 | 6041920 | 6213636 | 0.0378 | 0.0159 | 0.3923 |
| 2982 | RP11-146G7 | 18 | 6752909 | 6933645 | 0.0378 | 0.0159 | 0.3923 |
| 970 | RP11-51B9 | 18 | 7827406 | 7985296 | 0.0381 | 0.0232 | 0.3869 |
| 3418 | RP11-17J14 | 18 | 19024430 | 19181612 | −0.0408 | 0.0076 | 0.4007 |
| 1164 | RP11-178F10 | 18 | 20259694 | 20371250 | 0.0369 | 0.0183 | 0.4893 |
| 1514 | RP11-107K17 | 18 | 22172290 | 22273859 | 0.1597 | 0.0275 | 0.4851 |
| 1908 | RP11-526I8 | 18 | 22781318 | 22931013 | 0.1418 | −0.0033 | 0.4911 |
| 1673 | RP11-430E17 | 18 | 23970883 | 24165115 | 0.1418 | −0.0033 | 0.4911 |
| 3962 | RP11-20A14 | 18 | 25599565 | 25763888 | 0.0105 | −0.0177 | 0.4313 |
| 1337 | RP11-289A1 | 18 | 26696058 | 26878315 | 0.0386 | −0.0092 | 0.4378 |
| 1906 | RP11-549B18 | 18 | 27447010 | 27569489 | 0.0383 | −0.0218 | 0.4445 |
| 850 | RP11-413I9 | 18 | 28294303 | 28415096 | 0.0387 | −0.0363 | 0.4347 |
| 2048 | RP11-94E2 | 18 | 28808364 | 28955525 | 0.0387 | −0.0363 | 0.4347 |
| 218 | RP11-562H1 | 18 | 29409484 | 29472060 | 0.0365 | −0.0534 | 0.4824 |
| 542 | RP11-481E24 | 18 | 30773825 | 30859605 | 0.0011 | −0.0373 | 0.4073 |
| 316 | RP11-383C19 | 18 | 32335236 | 32502050 | 0.0011 | −0.0373 | 0.4073 |
| 2797 | RP11-58G13 | 18 | 32721707 | 32885642 | −0.0096 | −0.0373 | 0.4092 |
| 106 | RP11-62M21 | 18 | 34297490 | 34473929 | −0.0465 | −0.0539 | 0.4156 |
| 1694 | RP11-164M8 | 18 | 37363566 | 37482575 | −0.0467 | −0.0637 | 0.4202 |
| 1510 | RP11-107G7 | 18 | 38035420 | 38205929 | −0.0470 | −0.0582 | 0.4247 |
| 11 | RP11-486C18 | 18 | 40044271 | 40244003 | −0.0584 | −0.0631 | 0.4272 |
| 13 | RP11-463D17 | 18 | 41216566 | 41408713 | −0.0585 | −0.0839 | 0.4287 |
| 2423 | RP11-8H2 | 18 | 41851567 | 41984947 | −0.0585 | −0.0839 | 0.4287 |
| 865 | RP11-313C14 | 18 | 42350383 | 42350954 | −0.0585 | −0.0839 | 0.4287 |
| 2275 | RP11-71F23 | 18 | 43069061 | 43274200 | −0.0585 | −0.0839 | 0.4287 |
| 4152 | RP11-748M14 | 18 | 43589519 | 43745379 | −0.0584 | −0.0873 | 0.4274 |
| 418 | RP11-46D1 | 18 | 44393176 | 44552924 | −0.0585 | −0.1103 | 0.4276 |
| 655 | RP11-76E22 | 18 | 46462730 | 46633371 | −0.0585 | −0.1311 | 0.4293 |
| 1468 | RP11-729G3 | 18 | 46732284 | 46888494 | −0.0585 | −0.1311 | 0.4293 |
| 245 | RP11-1E21 | 18 | 47274828 | 47443303 | −0.0585 | −0.1311 | 0.4293 |
| 1181 | RP11-25O3 | 18 | 47958320 | 48119508 | −0.0585 | −0.1311 | 0.4293 |
| 643 | RP11-73I6 | 18 | 48386193 | 48496258 | −0.0588 | −0.1368 | 0.4361 |
| 823 | RP11-116K4 | 18 | 49795841 | 49971830 | −0.1009 | −0.1492 | 0.4232 |
| 1505 | RP11-99A1 | 18 | 50563151 | 50702093 | −0.1009 | −0.1492 | 0.4232 |
| 845 | RP11-397A16 | 18 | 51445553 | 51648118 | −0.1013 | −0.1653 | 0.4311 |
| 2996 | RP11-383D22 | 18 | 52656265 | 52867730 | −0.1017 | −0.1442 | 0.4408 |
| 3080 | RP11-35G9 | 18 | 53447744 | 53561700 | −0.1017 | −0.1442 | 0.4408 |
| 98 | RP11-61J14 | 18 | 54567051 | 54747660 | −0.1013 | −0.1505 | 0.4308 |
| 1247 | RP11-350K6 | 18 | 54867252 | 55027999 | −0.1013 | −0.1642 | 0.4328 |
| 4248 | RP11-350K6 | 18 | 54867252 | 55027999 | −0.1013 | −0.1642 | 0.4328 |
| 3005 | RP11-396N11 | 18 | 56063594 | 56151592 | −0.1013 | −0.1642 | 0.4328 |
| 3524 | RP11-520K18 | 18 | 56874822 | 57034619 | −0.1018 | −0.1729 | 0.4418 |
| 1525 | RP11-215A20 | 18 | 58572412 | 58756503 | −0.1018 | −0.1729 | 0.4418 |
| 3345 | RP11-28F1 | 18 | 59074167 | 59175738 | −0.1018 | −0.1729 | 0.4418 |
| 4230 | RP11-233O10 | 18 | 59886252 | 59971318 | −0.1158 | −0.1958 | 0.4352 |
| 852 | RP11-389J22 | 18 | 61594898 | 61752947 | −0.1158 | −0.1958 | 0.4352 |
| 3420 | RP11-23N5 | 18 | 62491384 | 62645196 | −0.1478 | −0.2044 | 0.4402 |
| 2528 | RP11-21L20 | 18 | 64827903 | 64947832 | −0.1481 | −0.2354 | 0.4476 |
| 3934 | RP11-342G3 | 18 | 64985278 | 65144962 | −0.1481 | −0.2354 | 0.4476 |
| 2163 | RP11-484N16 | 18 | 66000833 | 66160916 | −0.1481 | −0.2354 | 0.4476 |
| 4276 | RP11-430H7 | 18 | 66753077 | 66929602 | −0.1481 | −0.2354 | 0.4476 |
| 965 | RP11-45A1 | 18 | 67916649 | 68041627 | −0.1481 | −0.2354 | 0.4476 |
| 3331 | RP11-169F17 | 18 | 68809458 | 69000813 | −0.1481 | −0.2354 | 0.4476 |
| 415 | RP11-25L3 | 18 | 69588036 | 69755177 | −0.1481 | −0.2354 | 0.4476 |
| 3631 | RP11-238P13 | 18 | 70484649 | 70635265 | −0.1479 | −0.2467 | 0.4422 |
| 837 | RP11-396D4 | 18 | 71168342 | 71337306 | −0.1423 | −0.2615 | 0.4347 |
| 2070 | RP11-234N1 | 18 | 72266630 | 72448118 | −0.1423 | −0.2615 | 0.4347 |
| 2991 | RP11-118I2 | 18 | 73613846 | 73764173 | −0.1418 | −0.2813 | 0.4254 |
| 3969 | RP11-16L7 | 18 | 73908671 | 74017409 | −0.1418 | −0.2813 | 0.4254 |
| 2382 | RP11-563B11 | 18 | 74707626 | 74870951 | −0.1418 | −0.2813 | 0.4254 |
| 4147 | RP11-563B11 | 18 | 74707626 | 74870951 | −0.1418 | −0.2813 | 0.4254 |
| 1544 | RP11-315M18 | 18 | 75519491 | 75520038 | −0.1418 | −0.2813 | 0.4254 |
| 3492 | RP11-315M18 | 18 | 75519491 | 75520038 | −0.1418 | −0.2813 | 0.4254 |
| 1697 | RP11-154H12 | 18 | 75586355 | 75701258 | −0.1418 | −0.2813 | 0.4254 |
| 3558 | GS-964-M9 | 18 | 75752153 | 75902153 | −0.1422 | −0.2723 | 0.4337 |
| 4083 | GS-75-F20 | 18 | 75752203 | 75902203 | −0.1422 | −0.2723 | 0.4337 |
| 672 | CTD-3113P16 | 19 | 183069 | 327646 | −0.2224 | −0.2798 | 0.4038 |
| 1398 | GS-546-C11 | 19 | 300000 | 450000 | −0.2224 | −0.2798 | 0.4038 |
| 3544 | RG-129-F16 | 19 | 300050 | 450050 | −0.2224 | −0.2798 | 0.4038 |
| 125 | CTB-31C16 | 19 | 2595842 | 2773344 | −0.2216 | −0.2897 | 0.3868 |
| 2021 | RP11-268O21 | 19 | 2928774 | 3109041 | −0.2216 | −0.2897 | 0.3868 |
| 3848 | RP11-500M22 | 19 | 4806367 | 5009897 | −0.2224 | −0.2475 | 0.4033 |
| 123 | CTC-482H14 | 19 | 4998784 | 5154792 | −0.2225 | −0.2395 | 0.4054 |
| 1406 | RP5-859H16 | 19 | 5549421 | 5722324 | −0.2225 | −0.2338 | 0.4059 |
| 1687 | RP11-565J3 | 19 | 5713441 | 5893445 | −0.2225 | −0.2338 | 0.4059 |
| 1187 | RP11-330I7 | 19 | 6523485 | 6701760 | −0.2224 | −0.2277 | 0.4046 |
| 1457 | CTB-1025J19 | 19 | 7075600 | 7076170 | −0.2233 | −0.2310 | 0.4225 |
| 1450 | CTD-2623N2 | 19 | 9725333 | 9826741 | −0.0216 | −0.0447 | 0.4551 |
| 3039 | RP11-197O4 | 19 | 10248852 | 10410584 | 0.0053 | −0.0206 | 0.4540 |
| 135 | CTC-539A10 | 19 | 10528447 | 10699602 | 0.0051 | −0.0015 | 0.4589 |
| 2302 | CTC-359D24 | 19 | 11978969 | 12159478 | 0.0750 | 0.0119 | 0.4593 |
| 1454 | CTD-2659N19 | 19 | 12627096 | 12774845 | 0.1671 | 0.0286 | 0.4501 |
| 681 | CTC-250I14 | 19 | 13100858 | 13216789 | 0.3636 | 0.0441 | 0.5152 |
| 1684 | RP11-285H8 | 19 | 13815739 | 13816285 | 0.4097 | 0.0565 | 0.5062 |
| 2679 | RP11-56K21 | 19 | 14326904 | 14476904 | 0.3575 | 0.0559 | 0.4556 |
| 2837 | CTB-187L3 | 19 | 15415833 | 15568896 | 0.1500 | 0.0559 | 0.4149 |
| 679 | CTD-2231E14 | 19 | 16012544 | 16124610 | 0.1500 | 0.0559 | 0.4149 |
| 1137 | RP11-413M18 | 19 | 16712801 | 16899931 | 0.1509 | 0.0694 | 0.3944 |
| 676 | CTD-3149D2 | 19 | 17561413 | 17787582 | 0.1514 | 0.0226 | 0.3826 |
| 3220 | RP11-943H6 | 19 | 17677637 | 17827637 | 0.1514 | 0.0226 | 0.3826 |
| 2282 | CTC-251H24 | 19 | 18202507 | 18412025 | 0.1108 | 0.0226 | 0.3782 |
| 528 | RP11-520G3 | 19 | 19024191 | 19194051 | 0.1111 | −0.0181 | 0.3719 |
| 122 | CTC-260F20 | 19 | 19409711 | 19574832 | 0.1114 | −0.0283 | 0.3635 |
| 132 | CTD-2332E11 | 19 | 20532129 | 20637787 | 0.0167 | −0.0280 | 0.3848 |
| 2672 | RP11-352D15 | 19 | 20860912 | 21010912 | 0.0167 | −0.0280 | 0.3848 |
| 2583 | RP11-26D24 | 19 | 21345149 | 21504328 | 0.0167 | −0.0280 | 0.3848 |
| 2396 | RP11-165F1 | 19 | 22133724 | 22288871 | −0.0295 | −0.0428 | 0.3717 |
| 3404 | RP11-359H18 | 19 | 23555009 | 23732307 | 0.0072 | −0.0181 | 0.3823 |
| 678 | CTC-459F4 | 19 | 32889410 | 33099546 | 0.2281 | 0.0647 | 0.5117 |
| 663 | CTD-2043I16 | 19 | 33293108 | 33399253 | 0.3636 | 0.0523 | 0.5555 |
| 667 | CTD-2081K17 | 19 | 34149277 | 34316693 | 0.3636 | 0.0523 | 0.5555 |
| 4169 | CTD-2057D4 | 19 | 34956732 | 35092062 | 0.4170 | 0.0428 | 0.6081 |
| 139 | CTD-2001J20 | 19 | 35629515 | 35766589 | 0.1208 | 0.0409 | 0.4471 |
| 668 | CTD-2583G20 | 19 | 36159014 | 36322238 | 0.0606 | 0.0465 | 0.4469 |
| 2294 | CTC-325L16 | 19 | 37623634 | 37664852 | 0.0708 | 0.0354 | 0.4340 |
| 337 | RP11-298M15 | 19 | 38091322 | 38282890 | 0.0713 | 0.0261 | 0.4241 |
| 2083 | RP11-298M15 | 19 | 38091322 | 38282890 | 0.0713 | 0.0261 | 0.4241 |
| 2101 | RP11-413M10 | 19 | 39226664 | 39427144 | 0.0716 | 0.0362 | 0.4172 |
| 3014 | RP11-413M10 | 19 | 39226664 | 39427144 | 0.0716 | 0.0362 | 0.4172 |
| 144 | CTD-2527I21 | 19 | 40169242 | 40339132 | 0.0716 | 0.0362 | 0.4172 |
| 2577 | RP11-38C1 | 19 | 40876527 | 41059171 | 0.0716 | 0.0362 | 0.4172 |
| 2304 | CTD-2525J15 | 19 | 42010271 | 42199756 | 0.2261 | 0.0371 | 0.4944 |
| 2951 | RP11-9B17 | 19 | 42852412 | 43004473 | 0.1780 | 0.0262 | 0.4950 |
| 673 | CTB-186G2 | 19 | 43766631 | 43893312 | 0.2084 | 0.0184 | 0.4966 |
| 2841 | CTC-246B18 | 19 | 44401379 | 44539916 | 0.2084 | 0.0184 | 0.4966 |
| 682 | CTC-471F3 | 19 | 45079506 | 45253514 | 0.0329 | 0.0176 | 0.4259 |
| 2830 | CTC-435M10 | 19 | 46498597 | 46628707 | 0.0359 | −0.0331 | 0.3616 |
| 3292 | RP11-569M1 | 19 | 47423994 | 47633625 | 0.0357 | −0.0410 | 0.3655 |
| 127 | CTC-490G23 | 19 | 48347697 | 48590893 | 0.1787 | −0.0397 | 0.4705 |
| 3949 | RP11-15A1 | 19 | 49084683 | 49206647 | 0.1787 | −0.0397 | 0.4705 |
| 652 | RP11-84C16 | 19 | 49912622 | 50122984 | 0.0330 | −0.0634 | 0.3809 |
| 121 | CTB-14D10 | 19 | 51027521 | 51151415 | −0.0006 | −0.0809 | 0.3863 |
| 797 | RP11-2J15 | 19 | 51665795 | 51835146 | −0.0003 | −0.0818 | 0.3788 |
| 551 | RP11-492P7 | 19 | 51988000 | 52169534 | −0.0003 | −0.0818 | 0.3788 |
| 2287 | CTC-483I11 | 19 | 52341328 | 52496454 | −0.0003 | −0.0818 | 0.3788 |
| 3449 | RP11-3N16 | 19 | 53269624 | 53431702 | 0.0000 | −0.0812 | 0.3733 |
| 3614 | CTD-2639E6 | 19 | 53957736 | 54182006 | −0.0004 | −0.0716 | 0.3813 |
| 1135 | RP11-521I20 | 19 | 54525652 | 54718270 | −0.0004 | −0.0716 | 0.3813 |
| 3610 | CTD-2545M3 | 19 | 55596764 | 55697589 | −0.0011 | −0.0572 | 0.3982 |
| 868 | RP11-256B9 | 19 | 57561867 | 57749886 | −0.0007 | −0.0397 | 0.3891 |
| 1279 | RP11-44L20 | 19 | 58164197 | 58336597 | −0.0007 | −0.0397 | 0.3891 |
| 2495 | RP11-158G19 | 19 | 59041063 | 59235473 | −0.0007 | −0.0397 | 0.3891 |
| 136 | CTD-2337J16 | 19 | 59383667 | 59464093 | −0.0007 | −0.0397 | 0.3891 |
| 3042 | RP11-394L10 | 19 | 60914385 | 61088444 | −0.0007 | −0.0397 | 0.3891 |
| 2834 | CTC-444N24 | 19 | 62375405 | 62566112 | 0.0252 | −0.0396 | 0.3943 |
| 2836 | CTD-3138B18 | 19 | 63355845 | 63516696 | 0.0253 | −0.0323 | 0.3923 |
| 1384 | GS-325-I23 | 19 | 63361651 | 63511651 | 0.0253 | −0.0323 | 0.3923 |
| 1938 | GS-82-O2 | 20 | 105000 | 255000 | −0.0962 | −0.1245 | 0.6558 |
| 4078 | GS-1061-L1 | 20 | 105050 | 255050 | −0.0962 | −0.1245 | 0.6558 |
| 2910 | RP5-852M4 | 20 | 327037 | 385563 | 0.2181 | 0.0964 | 0.5006 |
| 1432 | RP11-314N13 | 20 | 1186672 | 1305743 | 0.2203 | 0.1149 | 0.4537 |
| 733 | RP4-686C3 | 20 | 2441424 | 2600595 | 0.2203 | 0.1149 | 0.4537 |
| 741 | RP4-741H3 | 20 | 3475976 | 3580882 | 0.2201 | 0.1102 | 0.4571 |
| 2373 | RP4-599I11 | 20 | 4706647 | 4766372 | 0.2204 | 0.1218 | 0.4498 |
| 745 | RP4-764O22 | 20 | 7012215 | 7095026 | 0.2204 | 0.1218 | 0.4498 |
| 2902 | RP5-1140M3 | 20 | 8198435 | 8214296 | 0.2205 | 0.1272 | 0.4484 |
| 2354 | RP4-811H13 | 20 | 9197701 | 9335479 | 0.2208 | 0.1385 | 0.4416 |
| 1968 | RP11-204H22 | 20 | 10398740 | 10537181 | 0.2199 | 0.1454 | 0.4615 |
| 2905 | RP4-742J24 | 20 | 11812345 | 11979419 | 0.2202 | 0.1337 | 0.4546 |
| 3588 | RP11-104O6 | 20 | 12592830 | 12650613 | 0.2202 | 0.1337 | 0.4546 |
| 4124 | RP11-526K24 | 20 | 13618307 | 13720095 | 0.2202 | 0.1337 | 0.4546 |
| 194 | RP5-822J19 | 20 | 15512828 | 15602699 | 0.2202 | 0.1337 | 0.4546 |
| 205 | RP3-348M17 | 20 | 16588690 | 16635277 | 0.2205 | 0.1018 | 0.4495 |
| 1444 | RP11-504H3 | 20 | 17822016 | 17961860 | 0.2223 | 0.0780 | 0.4085 |
| 746 | RP1-167O22 | 20 | 21492993 | 21567966 | 0.2228 | 0.0805 | 0.3991 |
| 749 | RP4-788L20 | 20 | 22476757 | 22597625 | 0.2240 | 0.0654 | 0.3731 |
| 197 | RP1-234M6 | 20 | 23600046 | 23638470 | 0.2240 | 0.0654 | 0.3731 |
| 210 | RP5-1025A1 | 20 | 24956308 | 25050971 | 0.2241 | 0.0805 | 0.3699 |
| 1436 | RP11-348I14 | 20 | 28197652 | 28267569 | 0.2217 | 0.1152 | 0.4222 |
| 3064 | RP3-324O17 | 20 | 29577867 | 29657285 | 0.2247 | 0.0942 | 0.3584 |
| 357 | RP5-857M17 | 20 | 29657186 | 29756778 | 0.2247 | 0.0942 | 0.3584 |
| 201 | RP1-310O13 | 20 | 29939119 | 30102094 | 0.2247 | 0.0942 | 0.3584 |
| 3604 | RP11-410N8 | 20 | 30605464 | 30669072 | 0.2247 | 0.0942 | 0.3584 |
| 346 | RP5-1085F17 | 20 | 30791562 | 30910460 | 0.2455 | 0.0997 | 0.3625 |
| 2901 | RP4-733O23 | 20 | 31169190 | 31184191 | 0.2455 | 0.0997 | 0.3625 |
| 738 | RP5-1137F22 | 20 | 31582324 | 31675130 | 0.2455 | 0.0997 | 0.3625 |
| 3600 | RP11-353C18 | 20 | 33744285 | 33824849 | 0.2448 | 0.0726 | 0.3788 |
| 3592 | RP11-234K24 | 20 | 34193483 | 34346674 | 0.2448 | 0.0680 | 0.3788 |
| 2369 | RP3-469A13 | 20 | 34762708 | 34886536 | 0.2448 | 0.0680 | 0.3788 |
| 2893 | RP4-633O20 | 20 | 35834704 | 35931163 | 0.2448 | 0.0680 | 0.3788 |
| 1428 | RP11-122O1 | 20 | 36744259 | 36840557 | 0.2449 | 0.0619 | 0.3768 |
| 213 | RP4-600E6 | 20 | 37565916 | 37666119 | 0.2449 | 0.0619 | 0.3768 |
| 750 | RP5-892M9 | 20 | 38231698 | 38293618 | 0.2449 | 0.0619 | 0.3768 |
| 753 | RP4-796I11 | 20 | 39313326 | 39390727 | 0.2444 | 0.0675 | 0.3856 |
| 2353 | RP1-128O17 | 20 | 39934889 | 40041261 | 0.2444 | 0.0675 | 0.3856 |
| 2357 | RP1-232N11 | 20 | 40680769 | 40792409 | 0.2441 | 0.0899 | 0.3932 |
| 193 | RP1-138B7 | 20 | 41584658 | 41668980 | 0.2669 | 0.0958 | 0.4005 |
| 3045 | RP5-1028D15 | 20 | 41668881 | 41804999 | 0.2668 | 0.1208 | 0.4023 |
| 2365 | RP3-337O18 | 20 | 43889651 | 43975730 | 0.2658 | 0.1557 | 0.4242 |
| 206 | RP5-1005L2 | 20 | 44369220 | 44380337 | 0.2658 | 0.1557 | 0.4242 |
| 3596 | RP11-347D21 | 20 | 46001209 | 46064435 | 0.2651 | 0.1621 | 0.4380 |
| 3060 | RP1-155G6 | 20 | 47049373 | 47129972 | 0.2651 | 0.1621 | 0.4380 |
| 2509 | RP3-470L14 | 20 | 47129873 | 47243708 | 0.2651 | 0.1621 | 0.4380 |
| 2913 | RP4-791K14 | 20 | 47382984 | 47538301 | 0.2648 | 0.1631 | 0.4453 |
| 2906 | RP5-1185N5 | 20 | 48091851 | 48205439 | 0.2648 | 0.1631 | 0.4453 |
| 349 | RP4-530I15 | 20 | 48590356 | 48701394 | 0.2648 | 0.1631 | 0.4453 |
| 2366 | RP5-994O24 | 20 | 49519272 | 49575686 | 0.2635 | 0.1822 | 0.4737 |
| 2897 | RP4-715N11 | 20 | 50717369 | 50828741 | 0.2637 | 0.1779 | 0.4702 |
| 353 | RP4-724E16 | 20 | 51561511 | 51648112 | 0.2638 | 0.1907 | 0.4680 |
| 2894 | RP5-1075G21 | 20 | 52147783 | 52227376 | 0.2638 | 0.1830 | 0.4663 |
| 350 | RP5-1162C3 | 20 | 52227277 | 52329237 | 0.2638 | 0.1830 | 0.4663 |
| 3584 | RP11-6L15 | 20 | 52483567 | 52583948 | 0.2638 | 0.1830 | 0.4663 |
| 893 | RP5-1167H4 | 20 | 54336458 | 54472150 | 0.2652 | 0.1681 | 0.4360 |
| 742 | RP5-1153D9 | 20 | 54472051 | 54566171 | 0.2652 | 0.1681 | 0.4360 |
| 1424 | RP11-46O3 | 20 | 54804991 | 54897122 | 0.2014 | 0.1685 | 0.4660 |
| 4128 | RP13-379L11 | 20 | 55913984 | 56121677 | 0.2822 | 0.1854 | 0.4137 |
| 2361 | RP1-309F20 | 20 | 56868317 | 56911123 | 0.2822 | 0.1854 | 0.4137 |
| 2358 | RP5-827E24 | 20 | 59427000 | 59538724 | 0.2822 | 0.1854 | 0.4137 |
| 2898 | RP5-1107C24 | 20 | 59894657 | 60016638 | 0.2800 | 0.2158 | 0.4623 |
| 209 | RP4-563E14 | 20 | 61012762 | 61041280 | 0.2816 | 0.2046 | 0.4263 |
| 4132 | RP13-152O15 | 20 | 62108666 | 62163452 | 0.2815 | 0.1949 | 0.4297 |
| 1918 | GS-81-F12 | 20 | 62310964 | 62460964 | 0.2815 | 0.1949 | 0.4297 |
| 3051 | RP1-126N20 | 21 | 14634832 | 14731794 | −0.0823 | −0.3210 | 0.5819 |
| 760 | RP11-304D2 | 21 | 18224008 | 18383964 | −0.0366 | −0.0329 | 0.4108 |
| 2392 | RP11-49B5 | 21 | 20224452 | 20292462 | −0.0366 | −0.0329 | 0.4108 |
| 2748 | RP11-509A1 | 21 | 21128294 | 21149667 | −0.0366 | −0.0329 | 0.4108 |
| 3047 | RP1-50A12 | 21 | 21237180 | 21411332 | −0.0051 | −0.0043 | 0.4208 |
| 2940 | RP11-410P24 | 21 | 32849565 | 33019511 | −0.0197 | −0.0239 | 0.3592 |
| 3059 | RP1-245P17 | 21 | 34483960 | 34602938 | −0.0197 | −0.0239 | 0.3592 |
| 899 | RP1-255P7 | 21 | 35282309 | 35532845 | −0.0202 | −0.0524 | 0.3711 |
| 2932 | RP11-102E10 | 21 | 36765627 | 36936688 | −0.0198 | −0.0819 | 0.3609 |
| 768 | RP11-98O13 | 21 | 37915905 | 38078949 | −0.0209 | −0.0659 | 0.3865 |
| 776 | RP11-164E1 | 21 | 38601021 | 38740380 | −0.0209 | −0.0659 | 0.3865 |
| 891 | RP1-128M19 | 21 | 39567315 | 39698959 | −0.0209 | −0.0659 | 0.3865 |
| 344 | RP5-1031P17 | 21 | 39699017 | 39788341 | −0.0209 | −0.0659 | 0.3865 |
| 895 | RP1-171F15 | 21 | 40630421 | 40630982 | −0.0209 | −0.0733 | 0.3863 |
| 3063 | RP1-265B9 | 21 | 41559315 | 41716976 | −0.0207 | −0.0656 | 0.3824 |
| 232 | RP11-113F1 | 21 | 42507240 | 42689355 | −0.0209 | −0.0611 | 0.3854 |
| 2924 | RP11-351D2 | 21 | 43550744 | 43727862 | −0.0209 | −0.0528 | 0.3868 |
| 764 | RP11-397E9 | 21 | 44672693 | 44846917 | −0.0210 | −0.0434 | 0.3871 |
| 2097 | RP11-397E9 | 21 | 44672693 | 44846917 | −0.0210 | −0.0434 | 0.3871 |
| 2400 | RP11-178H12 | 21 | 46424050 | 46607897 | −0.0210 | −0.0434 | 0.3871 |
| 1922 | GS-2-H14 | 21 | 46694323 | 46844323 | −0.0210 | −0.0434 | 0.3871 |
| 4082 | GS-63-H24 | 21 | 46694373 | 46844373 | −0.0210 | −0.0434 | 0.3871 |
| 4008 | XX-p8708 | 22 | 15621248 | 15745966 | −0.0073 | −0.0574 | 0.3928 |
| 2911 | XX-bac32 | 22 | 16899186 | 17026897 | −0.0073 | −0.0574 | 0.3928 |
| 2903 | XX-461k10 | 22 | 17368953 | 17412487 | −0.0073 | −0.0574 | 0.3928 |
| 3211 | RP11-179H3 | 22 | 20578709 | 20719542 | −0.1023 | −0.0666 | 0.3808 |
| 1056 | RP11-757F24 | 22 | 21112875 | 21296725 | −0.1023 | −0.0666 | 0.3808 |
| 2899 | RP11-50L23 | 22 | 21400636 | 21552718 | −0.1029 | −0.0640 | 0.3672 |
| 739 | RP11-80O7 | 22 | 22510132 | 22789754 | −0.1017 | −0.0637 | 0.3933 |
| 3468 | LL22NC03-95F10 | 22 | 23387722 | 23427691 | −0.1017 | −0.0637 | 0.3933 |
| 2356 | CTA-221G9 | 22 | 23879817 | 23982359 | −0.1004 | −0.0893 | 0.4224 |
| 2915 | CTA-125H2 | 22 | 24560701 | 24734213 | −0.1004 | −0.0893 | 0.4224 |
| 2896 | CTA-445C9 | 22 | 25228499 | 25359896 | −0.1005 | −0.0947 | 0.4195 |
| 2907 | XXbac-677f7 | 22 | 31498418 | 31627100 | −0.1154 | −0.1380 | 0.4127 |
| 2376 | CTA-415G2 | 22 | 31695938 | 31825894 | −0.1154 | −0.1380 | 0.4127 |
| 1304 | LL22NC01-95B1 | 22 | 32340168 | 32367572 | −0.1150 | −0.1641 | 0.4026 |
| 200 | CTA-221H1 | 22 | 32901471 | 32904493 | −0.1146 | −0.1464 | 0.3952 |
| 1308 | RP1-215F16 | 22 | 33798722 | 33828353 | −0.1145 | −0.1538 | 0.3920 |
| 196 | CTA-212A2 | 22 | 34741486 | 34954238 | −0.1144 | −0.1585 | 0.3899 |
| 3460 | LL22NC01-132D12 | 22 | 35478908 | 35505203 | −0.1144 | −0.1585 | 0.3899 |
| 208 | CTA-390B3 | 22 | 36120349 | 36212755 | −0.1146 | −0.1707 | 0.3937 |
| 204 | CTA-228A9 | 22 | 36811298 | 36906672 | −0.1465 | −0.1770 | 0.3966 |
| 755 | CTA-150C2 | 22 | 37610145 | 37811287 | −0.1465 | −0.1722 | 0.3973 |
| 2514 | LL22NC03-10C3 | 22 | 37938484 | 37976499 | −0.1465 | −0.1678 | 0.3966 |
| 1852 | RP1-172B20 | 22 | 38345395 | 38559115 | −0.1465 | −0.1722 | 0.3969 |
| 2368 | CTA-229A8 | 22 | 39305277 | 39427833 | −0.1462 | −0.1810 | 0.3915 |
| 2006 | RP11-12M9 | 22 | 39701569 | 39877855 | −0.1462 | −0.1810 | 0.3915 |
| 1731 | RP11-422A16 | 22 | 39877756 | 39890808 | −0.1462 | −0.1888 | 0.3906 |
| 884 | RP1-85F18 | 22 | 39890708 | 39932624 | −0.1462 | −0.1888 | 0.3906 |
| 1844 | RP5-979N1 | 22 | 40022373 | 40124523 | −0.1462 | −0.1888 | 0.3906 |
| 2360 | CTA-250D10 | 22 | 40582711 | 40803605 | −0.1698 | −0.1883 | 0.4036 |
| 3476 | RP3-437M21 | 22 | 41529049 | 41616450 | −0.1697 | −0.1954 | 0.4017 |
| 1316 | RP3-388M5 | 22 | 42473525 | 42651092 | −0.1697 | −0.1954 | 0.4017 |
| 2372 | CTA-397C4 | 22 | 43212696 | 43260941 | −0.1697 | −0.1954 | 0.4017 |
| 2364 | CTA-268H5 | 22 | 43952888 | 44141720 | −0.1697 | −0.1954 | 0.4017 |
| 3480 | RP3-398C22 | 22 | 44454571 | 44543898 | −0.1697 | −0.1954 | 0.4017 |
| 736 | CTA-29F11 | 22 | 45493480 | 45582798 | −0.1703 | −0.2084 | 0.4155 |
| 1300 | LL22NC03-75H12 | 22 | 46190401 | 46236544 | −0.1696 | −0.2242 | 0.4003 |
| 1848 | RP5-925J7 | 22 | 47740349 | 47838795 | −0.2419 | −0.2580 | 0.4401 |
| 1312 | RP3-355C18 | 22 | 48767848 | 48869117 | −0.1833 | −0.1980 | 0.4019 |
| 743 | CTA-799F10 | 22 | 49425750 | 49449200 | −0.1831 | −0.1896 | 0.3974 |
| 4091 | GS-3018-K1 | 22 | 49461432 | 49611432 | −0.1450 | −0.1652 | 0.3899 |
| 4086 | GS-99-K24 | 22 | 49496432 | 49646432 | −0.1021 | −0.2078 | 0.5619 |
| 1923 | GS-839-D20 | 23 | 85000 | 235000 | −0.1468 | −0.1884 | 0.5611 |
| 1583 | RP11-418N20 | 23 | 2939509 | 3122882 | −0.3227 | −0.2103 | 0.4587 |
| 1260 | RP11-23N11 | 23 | 3707732 | 3754886 | −0.3227 | −0.2103 | 0.4587 |
| 2270 | RP11-62N12 | 23 | 4616007 | 4758929 | −0.3227 | −0.2103 | 0.4587 |
| 2817 | RP11-60N3 | 23 | 5403637 | 5566017 | −0.3227 | −0.2103 | 0.4587 |
| 1150 | RP11-483M24 | 23 | 6957719 | 7136905 | −0.3227 | −0.2103 | 0.4587 |
| 571 | RP11-323F16 | 23 | 7455550 | 7633882 | −0.3227 | −0.2103 | 0.4587 |
| 3041 | RP11-323F16 | 23 | 7455550 | 7633882 | −0.3227 | −0.2103 | 0.4587 |
| 1032 | RP1-108M6 | 23 | 9561147 | 9684179 | −0.3777 | −0.2286 | 0.4472 |
| 3046 | RP6-1O2 | 23 | 10270814 | 10437697 | −0.3495 | −0.2285 | 0.4408 |
| 3188 | RP1-27C22 | 23 | 11152958 | 11412159 | −0.3495 | −0.2285 | 0.4408 |
| 1591 | RP11-1J4 | 23 | 12448478 | 12542635 | −0.3514 | −0.2311 | 0.3994 |
| 3433 | RP11-1J4 | 23 | 12448478 | 12542635 | −0.3514 | −0.2311 | 0.3994 |
| 4107 | RP1-164K3 | 23 | 12952343 | 13058902 | −0.3514 | −0.2311 | 0.3994 |
| 1036 | RP1-122K4 | 23 | 13838730 | 14015083 | −0.3514 | −0.2311 | 0.3994 |
| 1028 | RP1-93D11 | 23 | 14768520 | 14808635 | −0.3516 | −0.2443 | 0.3934 |
| 1677 | RP11-431J24 | 23 | 16028650 | 16242650 | −0.3516 | −0.2443 | 0.3934 |
| 1042 | RP5-1129A6 | 23 | 18089214 | 18198231 | −0.3516 | −0.2443 | 0.3934 |
| 3888 | RP11-558P14 | 23 | 18352566 | 18447658 | −0.3516 | −0.2443 | 0.3934 |
| 3728 | RP11-421K1 | 23 | 18964945 | 19156262 | −0.3516 | −0.2443 | 0.3934 |
| 2474 | RP11-406A18 | 23 | 21448265 | 21601094 | −0.3516 | −0.2443 | 0.3934 |
| 872 | RP11-261M11 | 23 | 24176952 | 24341732 | −0.3349 | −0.2488 | 0.3758 |
| 369 | RP11-272N24 | 23 | 25247627 | 25248382 | −0.3349 | −0.2488 | 0.3758 |
| 2587 | RP11-26L4 | 23 | 26803849 | 26971177 | −0.3349 | −0.2488 | 0.3758 |
| 3768 | RP11-26L4 | 23 | 26803849 | 26971177 | −0.3349 | −0.2488 | 0.3758 |
| 2017 | RP11-268G12 | 23 | 27060718 | 27210570 | −0.3349 | −0.2488 | 0.3758 |
| 19 | RP11-489K4 | 23 | 28029871 | 28187101 | −0.3349 | −0.2488 | 0.3758 |
| 1171 | RP11-169J21 | 23 | 28257706 | 28427554 | −0.3344 | −0.2608 | 0.3868 |
| 41 | RP11-37E19 | 23 | 28928507 | 28929130 | −0.3344 | −0.2608 | 0.3868 |
| 1301 | RP6-27C10 | 23 | 29091437 | 29143794 | −0.3590 | −0.2609 | 0.3938 |
| 497 | RP4-662N3 | 23 | 29347326 | 29491215 | −0.3590 | −0.2609 | 0.3938 |
| 2976 | RP11-122N14 | 23 | 31429070 | 31590268 | −0.3588 | −0.2432 | 0.3966 |
| 3467 | RP4-639D23 | 23 | 32142380 | 32238245 | −0.3588 | −0.2432 | 0.3966 |
| 1843 | RP5-1147O16 | 23 | 32347144 | 32479433 | −0.3588 | −0.2432 | 0.3966 |
| 3602 | RP13-46M24 | 23 | 36337423 | 36531334 | −0.3588 | −0.2432 | 0.3966 |
| 1817 | RP11-12J5 | 23 | 37553875 | 37716467 | −0.3589 | −0.2381 | 0.3955 |
| 3771 | RP11-12J5 | 23 | 37553875 | 37716467 | −0.3589 | −0.2381 | 0.3955 |
| 1985 | RP11-469E19 | 23 | 40755108 | 40918627 | −0.3582 | −0.2445 | 0.4096 |
| 1849 | RP1-169I5 | 23 | 41107931 | 41241498 | −0.3582 | −0.2445 | 0.4096 |
| 3462 | RP1-308O1 | 23 | 42106089 | 42272704 | −0.3582 | −0.2445 | 0.4096 |
| 3479 | RP5-879N19 | 23 | 43352635 | 43432926 | −0.2571 | −0.2450 | 0.4504 |
| 1973 | RP11-386N14 | 23 | 44547837 | 44688553 | −0.3379 | −0.2205 | 0.4097 |
| 3477 | RP1-54B20 | 23 | 47651854 | 47861472 | −0.1876 | −0.2047 | 0.4851 |
| 1434 | RP11-576P23 | 23 | 50147678 | 50302524 | −0.2160 | −0.1973 | 0.4934 |
| 1425 | RP11-56H2 | 23 | 51259019 | 51394297 | −0.3483 | −0.1963 | 0.4130 |
| 1446 | RP13-188A5 | 23 | 56037801 | 56140557 | −0.2911 | −0.1821 | 0.3904 |
| 1981 | RP11-445O16 | 23 | 56976937 | 57113775 | −0.2911 | −0.1780 | 0.3904 |
| 1839 | RP5-966K21 | 23 | 57905135 | 57932055 | −0.2911 | −0.1832 | 0.3899 |
| 3598 | RP13-34C21 | 23 | 62281936 | 62462786 | −0.3099 | −0.1867 | 0.3915 |
| 1969 | RP11-284B18 | 23 | 63351090 | 63455623 | −0.3094 | −0.1985 | 0.4019 |
| 1306 | RP3-323B6 | 23 | 64514210 | 64626872 | −0.3094 | −0.1985 | 0.4019 |
| 1442 | RP13-130F17 | 23 | 65362816 | 65515922 | −0.2844 | −0.1816 | 0.4041 |
| 3461 | RP6-22P16 | 23 | 66474359 | 66577725 | −0.3334 | −0.1818 | 0.4237 |
| 2517 | RP4-808O4 | 23 | 66577626 | 66716658 | −0.3334 | −0.1818 | 0.4237 |
| 1314 | RP3-360E18 | 23 | 67342258 | 67474160 | −0.3334 | −0.1818 | 0.4237 |
| 4145 | RP11-446J19 | 23 | 68510619 | 68634148 | −0.2948 | −0.1894 | 0.4066 |
| 3463 | RP4-583H20 | 23 | 70619662 | 70620234 | −0.2949 | −0.1949 | 0.4056 |
| 4126 | RP13-260P4 | 23 | 72691735 | 72766161 | −0.2949 | −0.1808 | 0.4058 |
| 1965 | RP11-253L3 | 23 | 73522518 | 73533894 | −0.2949 | −0.1808 | 0.4058 |
| 4125 | RP11-236O12 | 23 | 74730746 | 74851000 | −0.2949 | −0.1808 | 0.4058 |
| 1315 | RP5-875J14 | 23 | 76640561 | 76788983 | −0.2949 | −0.1808 | 0.4058 |
| 4014 | RP3-465G10 | 23 | 77111566 | 77195728 | −0.2949 | −0.1808 | 0.4058 |
| 1437 | RP11-159L8 | 23 | 78252504 | 78419313 | −0.2949 | −0.1808 | 0.4058 |
| 3605 | RP11-217H19 | 23 | 79352997 | 79447858 | −0.2949 | −0.1808 | 0.4058 |
| 3593 | RP11-102P23 | 23 | 80307276 | 80471287 | −0.2949 | −0.1808 | 0.4058 |
| 4007 | RP5-1156N12 | 23 | 81388801 | 81535160 | −0.2948 | −0.1899 | 0.4070 |
| 4005 | RP1-105K17 | 23 | 82482372 | 82563001 | −0.2948 | −0.1899 | 0.4070 |
| 3585 | RP11-54F22 | 23 | 83231140 | 83374359 | −0.2948 | −0.1899 | 0.4070 |
| 4003 | RP5-1109K10 | 23 | 83845334 | 83846061 | −0.2948 | −0.1899 | 0.4070 |
| 1841 | RP1-93L7 | 23 | 84848591 | 85055374 | −0.2948 | −0.1899 | 0.4070 |
| 3458 | RP1-225D2 | 23 | 85407563 | 85482124 | −0.2948 | −0.1899 | 0.4070 |
| 1441 | RP11-192B18 | 23 | 86196880 | 86368726 | −0.2948 | −0.1899 | 0.4070 |
| 1297 | RP6-22D12 | 23 | 86741677 | 86811503 | −0.2948 | −0.1899 | 0.4070 |
| 4018 | RP4-542O23 | 23 | 87708917 | 87785193 | −0.2944 | −0.1837 | 0.4169 |
| 3606 | RP13-166C10 | 23 | 90182084 | 90304132 | −0.2944 | −0.1837 | 0.4169 |
| 4133 | RP11-361B11 | 23 | 93061893 | 93118369 | −0.2943 | −0.1939 | 0.4180 |
| 1318 | RP3-380C13 | 23 | 94580184 | 94717628 | −0.2943 | −0.1939 | 0.4180 |
| 1857 | RP1-199L16 | 23 | 95213815 | 95348777 | −0.2943 | −0.1939 | 0.4180 |
| 4129 | RP11-274M8 | 23 | 96356903 | 96509340 | −0.2943 | −0.1939 | 0.4180 |
| 4141 | RP11-400P10 | 23 | 97508539 | 97716150 | −0.2943 | −0.1939 | 0.4180 |
| 1433 | RP11-151N12 | 23 | 98412392 | 98497852 | −0.2943 | −0.1939 | 0.4180 |
| 1310 | RP3-341D10 | 23 | 100082182 | 100164698 | −0.2946 | −0.1932 | 0.4118 |
| 1858 | RP4-545K15 | 23 | 100732251 | 100789070 | −0.2644 | −0.1933 | 0.4218 |
| 1837 | RP1-77O19 | 23 | 101631035 | 101660222 | −0.2644 | −0.1933 | 0.4218 |
| 4006 | RP3-421I20 | 23 | 102569986 | 102622582 | −0.2641 | −0.1872 | 0.4302 |
| 1445 | RP11-230E14 | 23 | 103557065 | 103623441 | −0.2641 | −0.1858 | 0.4285 |
| 1430 | RP11-539A6 | 23 | 104447295 | 104458809 | −0.2641 | −0.1858 | 0.4285 |
| 1302 | RP1-312P4 | 23 | 104929751 | 104940096 | −0.2641 | −0.1858 | 0.4285 |
| 3997 | RP1-75H8 | 23 | 106053097 | 106162561 | −0.2642 | −0.1976 | 0.4270 |
| 1311 | RP5-820B18 | 23 | 106921582 | 107015662 | −0.2642 | −0.1976 | 0.4270 |
| 1313 | RP1-31B8 | 23 | 108431925 | 108500258 | −0.2642 | −0.1976 | 0.4270 |
| 3052 | RP1-136J15 | 23 | 108678922 | 108804386 | −0.2642 | −0.1976 | 0.4270 |
| 1299 | RP4-557A17 | 23 | 109509387 | 109525239 | −0.2643 | −0.2066 | 0.4244 |
| 1319 | RP5-914P14 | 23 | 110343476 | 110498850 | −0.2643 | −0.2066 | 0.4244 |
| 1845 | RP1-115K14 | 23 | 111801639 | 111820528 | −0.2644 | −0.1981 | 0.4233 |
| 4001 | RP1-93I3 | 23 | 114632105 | 114737667 | −0.2642 | −0.1783 | 0.4273 |
| 1838 | RP3-394H4 | 23 | 116516765 | 116589055 | −0.2646 | −0.1698 | 0.4175 |
| 1305 | RP6-155F9 | 23 | 117482470 | 117605044 | −0.2651 | −0.1732 | 0.4082 |
| 4002 | RP3-404F18 | 23 | 118438874 | 118566990 | −0.2651 | −0.1695 | 0.4076 |
| 3466 | RP1-318C15 | 23 | 119444046 | 119591147 | −0.2651 | −0.1695 | 0.4076 |
| 3478 | RP3-368G6 | 23 | 120476625 | 120534500 | −0.2651 | −0.1695 | 0.4076 |
| 1853 | RP1-181N1 | 23 | 122218383 | 122344757 | −0.2651 | −0.1695 | 0.4076 |
| 3998 | RP3-394F12 | 23 | 123854482 | 123963507 | −0.2651 | −0.1695 | 0.4076 |
| 1307 | RP4-657J8 | 23 | 124726405 | 124825259 | −0.2651 | −0.1695 | 0.4076 |
| 1846 | RP3-428A13 | 23 | 126290610 | 126444844 | −0.2651 | −0.1695 | 0.4076 |
| 3465 | RP6-109P7 | 23 | 127817947 | 127921935 | −0.2651 | −0.1695 | 0.4076 |
| 3474 | RP3-345B16 | 23 | 128151281 | 128272312 | −0.2651 | −0.1695 | 0.4076 |
| 4013 | RP1-179D3 | 23 | 129036262 | 129085478 | −0.2650 | −0.1668 | 0.4093 |
| 1850 | RP3-463A9 | 23 | 129672525 | 129773621 | −0.2650 | −0.1668 | 0.4093 |
| 3999 | RP5-965E19 | 23 | 131271371 | 131294729 | −0.2650 | −0.1668 | 0.4093 |
| 3469 | RP6-198C21 | 23 | 132223249 | 132365010 | −0.2652 | −0.1584 | 0.4051 |
| 3586 | RP11-481F23 | 23 | 134479916 | 134510981 | −0.2652 | −0.1584 | 0.4051 |
| 4017 | RP1-196E23 | 23 | 135361435 | 135484339 | −0.2652 | −0.1432 | 0.4045 |
| 1309 | RP1-20J23 | 23 | 136272111 | 136409221 | −0.2652 | −0.1432 | 0.4045 |
| 1429 | RP11-65G20 | 23 | 137322239 | 137377012 | −0.2652 | −0.1432 | 0.4045 |
| 3475 | RP5-833B2 | 23 | 137835938 | 137909069 | −0.2652 | −0.1354 | 0.4060 |
| 1438 | RP13-34G21 | 23 | 138587336 | 138777284 | −0.2652 | −0.1354 | 0.4060 |
| 1303 | RP4-595A18 | 23 | 139456147 | 139571994 | −0.2652 | −0.1354 | 0.4060 |
| 1426 | RP11-518F7 | 23 | 140211892 | 140377252 | −0.2652 | −0.1354 | 0.4060 |
| 3459 | RP4-552K20 | 23 | 140718870 | 140770787 | −0.2652 | −0.1354 | 0.4060 |
| 3470 | RP3-324C6 | 23 | 141360527 | 141476821 | −0.2646 | −0.1300 | 0.4182 |
| 1966 | GS1-91O18 | 23 | 142126632 | 142173638 | −0.2637 | −0.1363 | 0.4375 |
| 3473 | RP1-29A6 | 23 | 143420952 | 143534939 | −0.2637 | −0.1363 | 0.4375 |
| 1977 | RP11-390O24 | 23 | 143983780 | 144162387 | −0.2637 | −0.1363 | 0.4375 |
| 4137 | RP11-387H19 | 23 | 144906312 | 145003054 | −0.2637 | −0.1363 | 0.4375 |
| 1317 | RP1-73A14 | 23 | 145778695 | 145884842 | −0.2637 | −0.1363 | 0.4375 |
| 4010 | RP3-433G13 | 23 | 146471558 | 146503967 | −0.2631 | −0.1467 | 0.4499 |
| 487 | RP6-224C24 | 23 | 146503968 | 146682511 | −0.2631 | −0.1467 | 0.4499 |
| 4110 | RP5-937E21 | 23 | 148437100 | 148548659 | −0.2634 | −0.1354 | 0.4448 |
| 1414 | RP5-892C22 | 23 | 150185779 | 150298000 | −0.2631 | −0.1297 | 0.4515 |
| 3133 | RP11-45D17 | 23 | 150493592 | 150625362 | −0.2631 | −0.1297 | 0.4515 |
| 2261 | RP11-54I20 | 23 | 152359276 | 152538530 | −0.2626 | −0.1458 | 0.4611 |
| 2729 | RP11-54I20 | 23 | 152359276 | 152538530 | −0.2626 | −0.1458 | 0.4611 |
| 488 | RP5-1087L19 | 23 | 153404680 | 153547872 | −0.2626 | −0.1458 | 0.4611 |
| 40 | RP11-402H20 | 23 | 153861611 | 153862276 | −0.2626 | −0.1458 | 0.4611 |
| 1326 | RP11-296N8 | 23 | 153783896 | 153962641 | −0.2626 | −0.1458 | 0.4611 |
| 1541 | RP11-218L14 | 23 | 154334463 | 154482340 | −0.2626 | −0.1458 | 0.4611 |
| 4087 | GS-225-F6 | 23 | 154738754 | 154888754 | −0.2626 | −0.1458 | 0.4611 |

TABLE 2
BRCA-2 like classifiers. The indicated locations are on genome version ‘hg18’. Of
course, in case a genome version changes, the person skilled in the art is able to
assign the below mentioned positions to any new and/or different genome version. Thus, variations
in the Start and End positions are also encompassed by the present disclosure. Furthermore, the
sc_control values, the sc_BRCA2 values and/or the sd values can vary dependent
on the number of probes used and/or if the classifiers are retrained.

| Order | Clone | chrom | Start | End | sc_control | sc_BRCA2 | sd |
|---|---|---|---|---|---|---|---|
| 2112 | RP11-428B4 | 4 | 99946600 | 100058902 | −0.0456 | −0.0815 | 0.3725 |
| 1990 | RP11-56D20 | 4 | 101150764 | 101325336 | −0.0456 | −0.0815 | 0.3725 |
| 911 | RP11-13F20 | 4 | 101510577 | 101670097 | −0.0456 | −0.0815 | 0.3725 |
| 3315 | RP11-167N19 | 4 | 101813198 | 101961364 | −0.0456 | −0.0815 | 0.3725 |
| 2050 | RP11-110C16 | 4 | 103067212 | 103220659 | −0.0456 | −0.0815 | 0.3725 |
| 4273 | RP11-438P8 | 4 | 104406898 | 104431241 | −0.0456 | −0.0815 | 0.3725 |
| 1707 | RP11-174B22 | 4 | 104431242 | 104513122 | −0.0456 | −0.0815 | 0.3725 |
| 292 | RP11-122B24 | 4 | 105288367 | 105355835 | −0.0454 | −0.0867 | 0.3694 |
| 1797 | RP11-16G16 | 4 | 106336237 | 106507303 | −0.0453 | −0.0967 | 0.3663 |
| 4220 | RP11-208O6 | 4 | 107027991 | 107064932 | −0.0457 | −0.0919 | 0.3750 |
| 3667 | RP11-88D10 | 4 | 107736653 | 107943820 | −0.0457 | −0.1002 | 0.3760 |
| 2046 | RP11-109F18 | 4 | 108646932 | 108795103 | −0.0457 | −0.1002 | 0.3760 |
| 651 | RP11-75N20 | 4 | 109877028 | 110025600 | −0.0457 | −0.1069 | 0.3747 |
| 2273 | RP11-57A22 | 4 | 110057781 | 110225193 | −0.0457 | −0.1069 | 0.3747 |
| 2468 | RP11-380D23 | 4 | 111660447 | 111860073 | −0.0458 | −0.1017 | 0.3777 |
| 1237 | RP11-326N15 | 4 | 112359892 | 112502402 | −0.0458 | −0.1017 | 0.3777 |
| 2264 | RP11-77N9 | 4 | 112853134 | 112892477 | −0.0467 | −0.1121 | 0.3971 |
| 3753 | RP11-77N9 | 4 | 112853134 | 112892477 | −0.0467 | −0.1121 | 0.3971 |
| 298 | RP11-402J6 | 4 | 113599819 | 113777515 | −0.0459 | −0.0928 | 0.3800 |
| 1604 | RP11-24P18 | 4 | 131614434 | 131767271 | −0.0472 | −0.0613 | 0.4090 |
| 1800 | RP11-24P18 | 4 | 131614434 | 131767271 | −0.0472 | −0.0613 | 0.4090 |
| 3488 | RP11-314N14 | 4 | 132653555 | 132677577 | −0.0472 | −0.0613 | 0.4090 |
| 1611 | RP11-13H10 | 5 | 4351733 | 4533614 | 0.0442 | 0.2081 | 0.4476 |
| 3981 | RP11-13H10 | 5 | 4351733 | 4533614 | 0.0442 | 0.2081 | 0.4476 |
| 1828 | RP11-137P5 | 5 | 5500098 | 5687008 | 0.0442 | 0.2081 | 0.4476 |
| 3024 | RP11-245L6 | 5 | 5879129 | 5995464 | 0.0442 | 0.2056 | 0.4479 |
| 2292 | CTD-2324F15 | 5 | 6350940 | 6430016 | 0.0442 | 0.2056 | 0.4479 |
| 287 | RP11-114M17 | 5 | 6515876 | 6708632 | 0.0442 | 0.2056 | 0.4479 |
| 2300 | CTD-2346M20 | 5 | 7449057 | 7530186 | 0.0443 | 0.2172 | 0.4495 |
| 2973 | RP11-132J20 | 5 | 8413255 | 8600646 | 0.0446 | 0.2294 | 0.4564 |
| 1834 | RP11-32D12 | 5 | 9436026 | 9606181 | 0.0437 | 0.2214 | 0.4374 |
| 2284 | CTD-2274H20 | 5 | 10359620 | 10495937 | 0.0433 | 0.2144 | 0.4293 |
| 2488 | RP11-215G15 | 5 | 10644705 | 10722894 | 0.0433 | 0.2144 | 0.4293 |
| 1393 | GS-62-L11 | 6 | 110996 | 260996 | 0.0298 | 0.1440 | 0.4207 |
| 1927 | GS-196-15 | 6 | 225000 | 375000 | 0.0295 | 0.1845 | 0.4135 |
| 3809 | RP11-13J16 | 6 | 1240996 | 1301756 | 0.1256 | 0.1486 | 0.3986 |
| 3817 | RP11-15N12 | 6 | 3292049 | 3425043 | 0.1256 | 0.1486 | 0.3986 |
| 1646 | RP11-174B19 | 6 | 5151674 | 5170849 | 0.1256 | 0.1486 | 0.3986 |
| 3818 | RP11-232H4 | 6 | 6151264 | 6169479 | 0.1256 | 0.1486 | 0.3986 |
| 3330 | RP11-163I22 | 6 | 6217514 | 6382300 | 0.1256 | 0.1486 | 0.3986 |
| 3271 | RP11-320C15 | 6 | 6705338 | 6770147 | 0.1256 | 0.1486 | 0.3986 |
| 3252 | RP3-336K20 | 6 | 7544150 | 7575683 | 0.1256 | 0.1681 | 0.3972 |
| 1527 | RP11-196B15 | 6 | 7898162 | 8078796 | 0.1257 | 0.1759 | 0.4006 |
| 3807 | RP11-385L2 | 6 | 8598346 | 8599694 | 0.1257 | 0.1759 | 0.4006 |
| 1119 | RP11-360O19 | 6 | 10597237 | 10684872 | 0.1254 | 0.1663 | 0.3932 |
| 1623 | RP3-510L9 | 6 | 11352273 | 11482926 | 0.1260 | 0.1625 | 0.4060 |
| 1114 | RP11-97A19 | 6 | 12430744 | 12449045 | 0.1046 | 0.1625 | 0.4014 |
| 3242 | RP1-257A7 | 6 | 13285332 | 13413248 | 0.1045 | 0.1559 | 0.4000 |
| 3262 | RP3-365E2 | 6 | 14536103 | 14673832 | 0.1044 | 0.1513 | 0.3972 |
| 1637 | RP1-232K4 | 6 | 15434571 | 15630732 | 0.1041 | 0.1434 | 0.3903 |
| 3241 | RP1-13D10 | 6 | 16165681 | 16318728 | 0.1041 | 0.1434 | 0.3903 |
| 1106 | RP11-68J15 | 6 | 17735571 | 17805613 | 0.1041 | 0.1386 | 0.3909 |
| 3811 | RP11-408C8 | 6 | 18937804 | 19079559 | 0.1043 | 0.1318 | 0.3961 |
| 1087 | RP4-625H18 | 6 | 19863670 | 20018592 | 0.1046 | 0.1361 | 0.4023 |
| 1634 | RP3-444C7 | 6 | 20630894 | 20759064 | 0.1046 | 0.1361 | 0.4023 |
| 1650 | RP11-204E9 | 6 | 21543541 | 21694512 | 0.1051 | 0.1456 | 0.4128 |
| 3825 | RP11-33I5 | 6 | 22580401 | 22666188 | 0.1051 | 0.1456 | 0.4128 |
| 1666 | RP11-289M23 | 6 | 23499374 | 23534047 | 0.1051 | 0.1456 | 0.4128 |
| 1116 | RP11-176J5 | 6 | 24254612 | 24267701 | 0.1054 | 0.1384 | 0.4196 |
| 3826 | RP11-289G11 | 6 | 25494592 | 25630305 | 0.1342 | 0.1383 | 0.4307 |
| 3286 | RP11-150A6 | 6 | 29321010 | 29495872 | 0.0895 | 0.0895 | 0.4020 |
| 3249 | RP1-34F7 | 6 | 32031923 | 32200821 | 0.0823 | 0.0823 | 0.3915 |
| 3255 | RP5-1077I5 | 6 | 32404070 | 32498505 | 0.0606 | 0.0606 | 0.4543 |
| 991 | RP11-48M17 | 9 | 2136330 | 2296367 | −0.1362 | −0.0273 | 0.4484 |
| 997 | RP11-320E16 | 9 | 2583689 | 2658649 | −0.1356 | −0.0121 | 0.4619 |
| 3595 | RP11-509J21 | 9 | 3533196 | 3696642 | −0.1356 | −0.0121 | 0.4619 |
| 3144 | RP11-125K10 | 9 | 4819656 | 4991841 | −0.1356 | −0.0121 | 0.4619 |
| 1435 | RP11-509D8 | 9 | 4991742 | 5008809 | −0.1347 | −0.0019 | 0.4817 |
| 456 | RP11-106A1 | 9 | 6599727 | 6659690 | −0.1258 | −0.0142 | 0.4584 |
| 979 | RP11-29B9 | 9 | 7904421 | 8056890 | −0.1258 | −0.0142 | 0.4584 |
| 445 | RP11-175E13 | 9 | 8398602 | 8490985 | −0.1258 | −0.0142 | 0.4584 |
| 1439 | RP11-527D15 | 9 | 9657591 | 9823759 | −0.1261 | −0.0212 | 0.4511 |
| 447 | RP11-19G1 | 9 | 9999831 | 10094473 | −0.1261 | −0.0212 | 0.4511 |
| 2615 | RP11-23D5 | 9 | 11227455 | 11303482 | −0.1261 | −0.0262 | 0.4514 |
| 442 | RP11-352F21 | 9 | 11389654 | 11588151 | −0.1261 | −0.0212 | 0.4511 |
| 3162 | RP11-446F13 | 9 | 12281538 | 12346848 | −0.1262 | −0.0130 | 0.4502 |
| 449 | RP11-187K14 | 9 | 12872920 | 12952270 | −0.1262 | −0.0130 | 0.4502 |
| 3142 | RP11-413D24 | 9 | 13729563 | 13912972 | −0.1268 | −0.0214 | 0.4375 |
| 462 | RP11-408A13 | 9 | 14500602 | 14586756 | −0.1268 | −0.0214 | 0.4375 |
| 3587 | RP11-490C5 | 9 | 15298239 | 15399992 | −0.1268 | −0.0214 | 0.4375 |
| 460 | RP11-109M15 | 9 | 16171750 | 16325494 | −0.1268 | −0.0419 | 0.4371 |
| 984 | RP11-132E11 | 9 | 16995146 | 17138368 | −0.1268 | −0.0419 | 0.4371 |
| 980 | RP11-123J20 | 9 | 17933998 | 18013852 | −0.1269 | −0.0366 | 0.4347 |
| 1431 | RP11-503K16 | 9 | 18579960 | 18743122 | −0.1269 | −0.0366 | 0.4347 |
| 3599 | RP11-513M16 | 9 | 19310506 | 19423218 | −0.1269 | −0.0366 | 0.4347 |
| 2607 | RP11-15P13 | 9 | 20172502 | 20351132 | −0.1270 | −0.0446 | 0.4320 |
| 3140 | RP11-113D19 | 9 | 20996401 | 21158465 | −0.1271 | −0.0386 | 0.4303 |
| 3156 | RP11-149I2 | 9 | 21899259 | 22000413 | −0.1271 | −0.0386 | 0.4303 |
| 443 | RP11-11J1 | 9 | 22479496 | 22579721 | −0.1269 | −0.0502 | 0.4353 |
| 3591 | RP11-495L19 | 9 | 23461740 | 23557472 | −0.1269 | −0.0502 | 0.4353 |
| 3147 | RP11-33K8 | 9 | 24090706 | 24243453 | −0.1029 | −0.0468 | 0.4324 |
| 3583 | RP11-468C2 | 9 | 24937707 | 25069411 | −0.1029 | −0.0468 | 0.4324 |
| 3219 | RP11-211N8 | 9 | 46630312 | 46771375 | −0.3035 | −0.1792 | 0.6076 |
| 1931 | GS-261-B16 | 10 | 135029737 | 135179737 | −0.2825 | −0.2058 | 0.5492 |
| 4089 | GS-137-E24 | 10 | 135029787 | 135179787 | −0.2825 | −0.2058 | 0.5492 |
| 969 | RP11-45C5 | 11 | 100145392 | 100299550 | −0.0568 | 0.1322 | 0.3996 |
| 3423 | RP11-21G19 | 11 | 101247704 | 101397612 | −0.0568 | 0.1322 | 0.3996 |
| 3343 | RP11-315O6 | 11 | 101724655 | 101922841 | −0.0568 | 0.1322 | 0.3996 |
| 1992 | RP11-750P5 | 11 | 102010617 | 102011178 | −0.0564 | 0.1222 | 0.4083 |
| 2957 | RP11-2I22 | 11 | 102613695 | 102771597 | −0.0563 | 0.1073 | 0.4111 |
| 1464 | RP11-725J15 | 11 | 102741000 | 102891000 | −0.0563 | 0.1073 | 0.4111 |
| 222 | RP11-563P16 | 11 | 102966300 | 103066089 | −0.0563 | 0.1041 | 0.4120 |
| 931 | RP11-76K19 | 13 | 19007559 | 19137337 | 0.0095 | −0.0541 | 0.4080 |
| 3092 | RP11-187L3 | 13 | 19832836 | 19920914 | 0.0097 | −0.0675 | 0.4036 |
| 943 | RP11-110K8 | 13 | 20994607 | 21089699 | 0.0050 | −0.0692 | 0.5056 |
| 399 | RP11-26A3 | 13 | 21944537 | 22068982 | 0.0096 | −0.0752 | 0.4060 |
| 1983 | RP11-760M1 | 13 | 22887525 | 23056187 | 0.0096 | −0.0752 | 0.4060 |
| 950 | RP11-556N21 | 13 | 23994474 | 24103118 | −0.0177 | −0.0752 | 0.4051 |
| 3107 | RP11-111G7 | 13 | 25124500 | 25300819 | −0.0177 | −0.0804 | 0.4036 |
| 1979 | RP11-570F6 | 13 | 25898592 | 26065378 | −0.0177 | −0.0804 | 0.4036 |
| 1275 | RP11-44J9 | 13 | 26088616 | 26258539 | −0.0177 | −0.0804 | 0.4036 |
| 2556 | RP11-125I23 | 13 | 26843991 | 27010032 | −0.0182 | −0.0876 | 0.4156 |
| 3342 | RP11-153M24 | 13 | 27414162 | 27490882 | −0.0182 | −0.0876 | 0.4156 |
| 3099 | RP11-95G6 | 13 | 27868844 | 27970962 | 0.0283 | −0.0827 | 0.4242 |
| 936 | RP11-218E6 | 13 | 28837843 | 29008462 | 0.0283 | −0.0741 | 0.4236 |
| 946 | RP11-550P23 | 13 | 29914762 | 30048628 | 0.0898 | −0.0312 | 0.4195 |
| 939 | RP11-95N14 | 13 | 31136680 | 31227403 | 0.0903 | −0.0501 | 0.4084 |
| 3349 | RP11-37E23 | 13 | 31703950 | 31870606 | 0.0903 | −0.0501 | 0.4084 |
| 2564 | RP11-141M1 | 13 | 32790709 | 32842539 | 0.0903 | −0.0501 | 0.4084 |
| 3095 | RP11-87G1 | 13 | 32842440 | 33025356 | 0.0903 | −0.0501 | 0.4084 |
| 2553 | RP11-266E6 | 13 | 33995013 | 34159357 | 0.0903 | −0.0544 | 0.4097 |
| 3103 | RP11-98D3 | 13 | 34909905 | 35080902 | 0.0530 | −0.0545 | 0.3985 |
| 2555 | RP11-10M8 | 13 | 37051980 | 37156004 | 0.0149 | −0.0742 | 0.4204 |
| 400 | RP11-131P10 | 13 | 37797278 | 37920029 | 0.0149 | −0.0742 | 0.4204 |
| 2560 | RP11-131F1 | 13 | 38577172 | 38608597 | 0.0150 | −0.0949 | 0.4172 |
| 2554 | RP11-407E23 | 13 | 39306578 | 39498144 | −0.0184 | −0.1008 | 0.4195 |
| 2551 | RP11-2P5 | 13 | 40367434 | 40566000 | −0.0189 | −0.1092 | 0.4305 |
| 392 | RP11-125A7 | 13 | 41105696 | 41263001 | −0.0189 | −0.1092 | 0.4305 |
| 3111 | RP11-117I13 | 13 | 42155411 | 42253968 | −0.0185 | −0.1392 | 0.4211 |
| 2568 | RP11-168P13 | 13 | 43148292 | 43291406 | −0.0184 | −0.1476 | 0.4193 |
| 406 | RP11-442J21 | 13 | 44121277 | 44178227 | −0.0184 | −0.1435 | 0.4195 |
| 413 | RP11-351K3 | 13 | 44939380 | 45114967 | −0.0185 | −0.1385 | 0.4226 |
| 394 | RP11-408L13 | 13 | 46981023 | 47110676 | −0.0186 | −0.1323 | 0.4241 |
| 3339 | RP11-305D15 | 13 | 47760580 | 47897921 | −0.0206 | −0.1513 | 0.4681 |
| 408 | RP11-174I10 | 13 | 47897822 | 47960646 | −0.0206 | −0.1513 | 0.4681 |
| 412 | RP11-185C18 | 13 | 48960782 | 49068912 | −0.0185 | −0.1317 | 0.4217 |
| 2563 | RP11-40A8 | 13 | 50328519 | 50471842 | −0.0188 | −0.0988 | 0.4296 |
| 3051 | RP1-126N20 | 21 | 14634832 | 14731794 | −0.0823 | −0.3210 | 0.5819 |

### BRIEF DESCRIPTION OF THE DRAWINGS

FIGS. 1A and 1B: Average copy number aberration profiles of BRCA1- and BRCA2-mutated ovarian cancer. FIG. 1A) Average copy number aberration profile of 50 gBRCA1m ovarian cancers and 13 control ovarian cancers. On the x-axis, the cumulative genomic position, in mega bases, and the y-axis, the average 2 log ratio of tumor over normal DNA. FIG. 1B) Average copy number aberration profile of gBRCA2m ovarian cancers and 13 control ovarian cancers. On the x-axis the genomic position and on the y-axis the average 2 log ratio of tumor DNA over normal DNA.

FIG. 2: BRCA-like phenotype according to molecular genetic background. Each bar shows the distribution by percentage (y-axis) of BRCA1- and 2-like, only BRCA1-like, only BRCA2-like and non-BRCA-like profiles within the presented molecular genetic subgroup (x-axis). methyl: promoter hypermethylation, WT: wildtype.

FIG. 3: Molecular genetic background of BRCA-like cases. 223 samples are classified asBRCA1- and/or BRCA2-like. The pie chart shows the portion of samples with a specified (epi-)genetic alteration of a gene, involved in homologous recombination in this subgroup. In 25.10% of the cases no HR-affecting variant was detected by NGS or methylation analyses. methyl: promoter hypermethylation.

FIG. 4: CONSORT-like flow diagram of the AGO-TR1 trial. The sample for BRCA-like classification has been selected with the aim to enrich for germline (g) and somatic (s) mutation status. All available samples with a deleterious mutation in BRCA1/2 (g n=91, s n=27) and other ovarian cancer related genes (g n=34, s n=9) as well as somatic BRCA1/2-variants of unknown significance (VUS) (n=4) have been included, whereas gBRCA1/2-VUS (n=15) have been excluded. The remaining 135 samples were considered random sample of the main cohort, taking primary ovarian cancer (PR)-/platinum sensitive recurrent disease (RE)-status, age at diagnosis and sTP53-mutations into account. EOC: epithelial ovarian cancer; PR: primary disease; RE: relapsed; NGS: next generation sequencing; g: germline; lcWGS: low coverage whole genome sequencing.

FIG. 5: Diagrams showing the results of the evaluating of the predictive value of the method of the present disclosure based on survival analysis on tumor samples from 85 PAOLA-1 BRCA wild type patients (n=84), wherein the Diagram (A) shows that there is additional benefit of olaparib in BRCA1-like patients in phase 2 (HR (hazard rate)=0.609, 0.295-1.303, p=0.201 and wherein the Diagram (B) shows hat there is no additional benefit of olaparib in non-BRCA1-like patients in phase 2 (HR (hazard rate)=0.925, 0.411-2.084, p=0.851.

FIG. 6: Diagrams showing the results of the evaluating of the predictive value of the method of the present disclosure based on survival analysis on tumor samples from 383 additional PAOLA-1 BRCA wild type patients, wherein the Diagram (A) shows that there is additional benefit of olaparib in BRCA1-like patients in phase 3 (HR (hazard rate)=0.438, 0.316-0.607, p<0.001 and wherein the Diagram (B) shows hat there is no additional benefit of olaparib in non-BRCA1-like patients in phase 3 (HR (hazard rate)=0.959, 0.600-1.534.

### DETAILED DESCRIPTION OF THE DISCLOSURE

Tumors with HRD have been shown to be particularly sensitive to DNA crosslinking agents, such as alkylators, platinum drugs or platinating agents as well as to PARP inhibitors. The breast and ovarian cancer germline predisposition genes BRCA1 and BRCA2 play crucial roles in homologous recombination. Thus, ovarian cancers associated with a germline BRCA1 or BRCA2 mutation derive benefit of targeted therapy, for example with PARP inhibitors or specific classes of chemotherapy, such as platinum compounds as mentioned above. Furthermore, up to approximately 50% of tumors in ovarian cancer share characteristics with germline mutated cancers, because of other alterations in the BRCA1/BRCA2 pathway of homologous recombination, and therefore those benefit of such therapies as well. This phenotype is called BRCAness or BRCA-like.

Breast cancer BRCA1-like and BRCA2-like copy number profile classifiers predictive for mutation status and response to therapy, targeting HRD have been developed before, but breast cancer classifiers did not sufficiently predict mutation status in ovarian cancer. Hence, new classifiers had to be developed.

Accordingly, the present disclosure provides predictive biomarkers to identify the germline mutated as well as said BRCAness group for therapy specifically targeting BRCAness in ovarian cancer. In other words, the method of the present disclosure is useful for identifying patients with ovarian cancer, wherein the cancer cells have a defective DNA repair system, and thus, which are responsive to the mentioned targeted anti-cancer therapy.

Furthermore, the method of the present disclosure is also useful for identifying a group of patients that is non-BRCA1/2 and non-BRCA1/2-like, respectively, and that does not benefit of this therapy. Until now, no reliably method has been described that also identifies the non-benefitting population correctly.

Cancers and cell lines with BRCA mutations or mutations in the BRCA pathway exhibit genomic instability, manifesting in abnormal copy number profiles. Thus, in general, the present disclosure is based on the discovery that certain chromosomal copy number aberrations or variations, respectively (CNVs) in tumor cells allow tumors to be classified as either BRCA-associated-/BRCA-like tumors or sporadic tumors. This classification of a tumor allows for the prospective prediction of responsiveness of the patient from which the tumor was removed to anti-cancer therapy.

In the literature, other assays have been described measuring HRD in ovarian cancer (15, 17, 21, 46), some of which are also partly based on derivatives of SNP/copy number profiles. The approach of the present disclosure differs inter alia from other tests because it uses genomic location-specific aberrations. This leads to additional information being retained, such as aberrations that collaborate with the underlying HRD mechanism. Additionally, the presented classifiers are tissue-specific, which is an advantage compared to other HRD assays, as a response to targeted strategies can depend on the tissue of origin.

The terms copy number aberrations, copy number variants/variations and CNVs are used interchangeable herein. Furthermore, when reference is made to BRCA1-like or BRCA2-like, for example BRCA1-like tumors, or BRCA2-like tumors, if not indicated otherwise, this includes tumors having a mutation in the BRCA1 gene or BRCA2 gene itself and tumors which share characteristics with those germline mutated cancers, because of other alterations in the BRCA1/BRCA2 pathway of homologous recombination.

HRD is a defect in DNA repair by hampered homologous recombination repair (HRR), which is a form of DNA recombination often used to repair DNA double strand breaks. In cancers, this is often caused by loss of function mutations in BRCA1, BRCA2, RAD51C, RAD51D or PALB2, promoter hypermethylation of the BRCA1 gene promoter (leading to reduced expression of BRCA1) or a series of as yet to be defined causes. HRD is also characterized by the cellular sensitivity to for example PARP inhibitors, topoisomerase inhibitors or platinum salts.

The method of the present disclosure comprises the determination of a CNV of a genomic locus in a test sample from a subject comprising cancer cells, e.g. a DNA test sample of cancer cells, wherein the locus is selected from a pool of genomic loci comprising the loci set forth in Table 1 or in Table 2, and wherein similarity between the CNVs in the pool of genomic loci in the test sample from the subject and the CNVs in a corresponding pool of genomic loci in the DNA of a reference sample of BRCA-like mutated cancer cells and a control sample of BRCA non-mutated cancer cells, is indicative for the HR deficiency status in the subject.

When reference is made to a genomic locus and a pool of genomic loci, respectively as set forth in Table 1 and/or Table 2, the locus is identifiable by its start and end position on the respective chromosome of genome version ‘hgl8’.

The test sample is a sample of material comprising cancer cells, in particular ovarian cancer cells. Samples include, but are not limited to material obtained from the subject of interest, i.e. from the ovarian cancer patient and may be directly obtained from the source, for example via tumor biopsy, or indirectly after culturing and/or further processing of the tumor cells. In particular, the test sample is a DNA sample or a sample that comprises DNA from the tumor cells of the subject of interest. Accordingly, the test sample is derived from a subject that is to be diagnosed of having HRD status or not and that is to be diagnosed of being responsive to a particular anti-cancer therapy or not. Any appropriate type of sample can be assessed to determine if the genome of the cancer cells contains an HRD signature, or lacks an HRD signature. Examples of samples containing cancer cells, in particular ovarian cancer cells that can be assessed as described herein include, without limitation, tumor biopsy samples (e.g., ovarian tumor biopsy samples), formalin-fixed, paraffin-embedded tissue samples containing cancer cells, core needle biopsies, fine needle aspirates, and samples containing cancer cells shed from a tumor. For formalin-fixed, paraffin-embedded tissue samples, the sample can be prepared by DNA extraction using a genomic DNA extraction kit optimized for FFPE tissue, for example Qiagen DNA mini kit (Qiagen, cat nr. 51306), QuickExtract™ FFPE DNA Extraction Kit (Epicentre™), and QIAamp™ DNA FFPE Tissue Kit (Qiagen™)).

In some cases, laser dissection techniques can be performed on a tissue sample to minimize the number of non-cancer cells within a cancer cell sample to be assessed. In some cases, antibody based purification methods can be used to enrich for cancer cells and/or deplete non-cancer cells. Examples of antibodies that could be used for cancer cell enrichment include, without limitation, anti-EpCAM, anti-TROP-2, anti-c-Met, anti-Folate binding protein, anti-N-Cadherin, anti-CD318, anti-antimesencymal stem cell antigen, anti-Her2, anti-MUC1, anti-EGFR, anti-cytokeratins (e.g., cytokeratin 7, cytokeratin 20, etc.), anti-Caveolin-1, anti-PSA, anti-CA125, and anti-surfactant protein antibodies. Even if dynamic ranges of the profiles are influenced, validation can be performed by the person skilled in the art as described herein.

The control sample used in the method of the present disclosure is a corresponding sample comprising ovarian cancer cells and the corresponding DNA, respectively, which are not associated with BRCA1-like or BRCA2-like mutations and do not have a HRD signature.

The reference sample is a corresponding sample comprising BRCA mutated ovarian cancer cells and the corresponding DNA, respectively. Based on such reference sample and a plurality of such reference samples, respectively, ovarian cancer specific DNA copy number aberration classifiers have been developed that identify a population enriched for BRCA1 and HRD-associated gene mutations (BRCA1-like), i.e. BRCA1-like classifiers, and a population enriched for BRCA2 and HR-associated gene mutations (BRCA2-like), i.e. BRCA2-like classifiers and a population enriched for BRCA1-like and BRCA2-like mutations. Those classifiers are listed in Table 1 (BRCA1-like) and Table 2 (BRCA2-like).

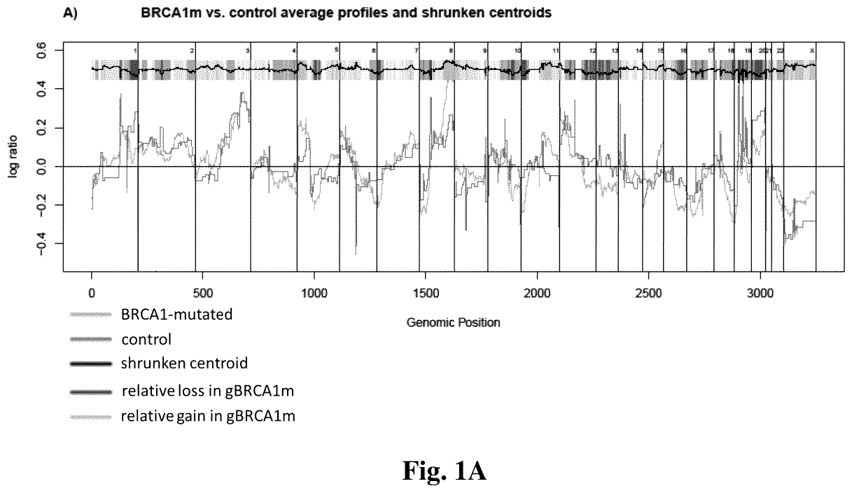

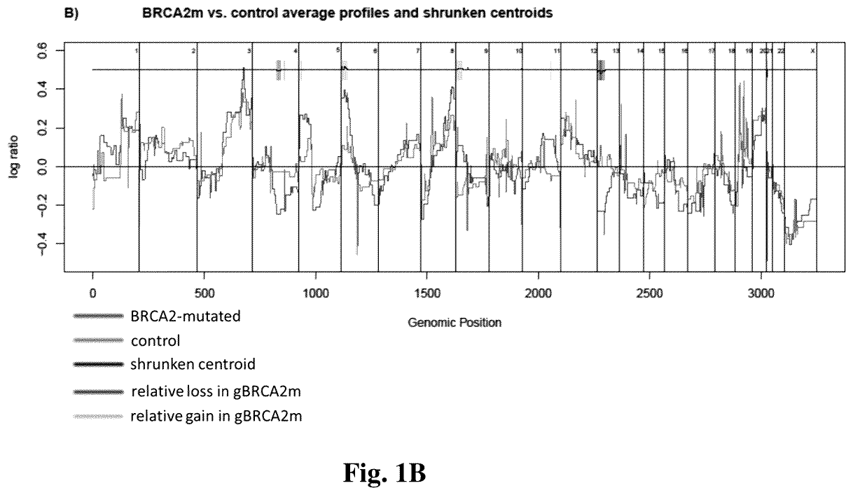

In particular, those classifiers have been developed by generating copy number profiles of control samples, i.e. samples derived from patients without a family history of ovarian or breast cancer, of gBRCA1m samples, i.e. samples derived from patients having ovarian cancer and having mutations in the BRCA1 gene, and of gBRCA2m samples, i.e. samples derived from patients having ovarian cancer and having mutations in the BRCA2 gene. FIG. 1 presents the average profiles of gBRCA1m and gBRCA2m versus control ovarian cancers. The generation of the copy number profiles is described in Example 1 in more detail. Thus, the present disclosure also relates to the development of BRCA1-like and BRCA2-like classifiers by generating copy number profiles of control samples, of gBRCA1m samples and of gBRCA2m samples, respectively.

After generating the copy number profiles, the ovarian cancer specific shrunken-centroid classifiers have been trained on ovarian cancer data. These shrunken centroids select genomic regions and weights that are discriminative for the BRCA-like and non-BRCA-like class. For this, ten-fold cross-validation has been carried out and training of the shrunken centroid classifiers was performed on the class labels gBRCA1m vs. control and gBRCA2m vs. control to classify ovarian cancer as being similar to gBRCA1m (BRCA1-like) or gBRCA2m(BRCA2-like) or controls (C). Subsequently, samples were classified as BRCA-like if the predicted probability was >0.5 and non-BRCA-like if the predicted probability was <=0.5, as was pre-defined in the training set. This is described in more detail in Example 2. Thus, the present disclosure also relates to the training of the above-mentioned classifiers on ovarian cancer data. In principle any data can be used as long as they comprise data of control samples and of gBRCA1m samples and gBRCA2m samples, respectively.

The training was followed by validation of the classifiers as for example described in Examples 3 and 4. As described in Example 3 validation was performed by using the TCGA dataset. In particular, 358 of 583 samples have been classified as having a BRCA1-like profile. Within these 358, 25/26 gBRCA1, 10/11 somatic BRCA1-mutated tumors, and 67/69 BRCA1-methylated tumors were classified as BRCA1-like, resulting in an overall sensitivity of 94.7% (95% CI: 0.82-0.99) and a specificity of 40% (95% Cl: 0.36-0.45). Furthermore, 350 of 583 samples were assigned to be BRCA2-like. Within these 350, 20/25 gBRCA2m 309 and 8/11 sBRCA2m samples were classified as BRCA2-like, resulting in a sensitivity of 77% (95% CI: 0.6-0.9) and a specificity of 41% (95% CI: 0.37-0.49).

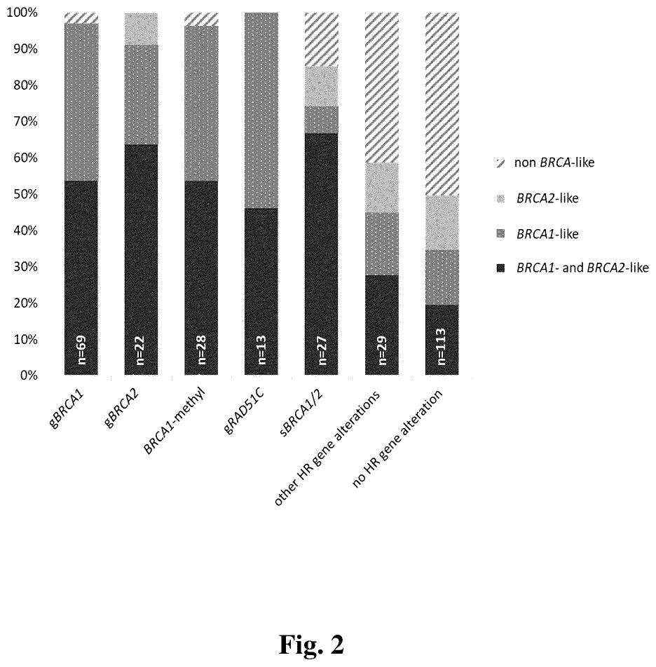

Validation was also performed within AGOTR1 study samples, wherein in total 300 samples with complete genetic and epigenetic information available, were analyzed. The results of the validation are presented in Example 4. In particular, focus was first put on the detection-rate of tumors derived from deleterious germline mutations in BRCA1/2 (see Table 6, FIG. 2). 67 of 69 BRCA1-mutated cases were BRCA1-like (detection rate of 97.1%), whereas 16 of 22 samples with a BRCA2 class4/5 variant in germline were BRCA2-like (detection rate of 72.7%). All remaining BRCA2-associated cases were identified by combining both classifiers, i.e. being BRCA1- or BRCA2-like. Regarding deleterious somatic variants, 15 of 17 (88.2%) samples with a BRCA1 mutation were identified by the BRCA1-like classifier. As the other two tumors displayed a BRCA2-like phenotype, both classifiers' application detected 100% of the mutated cases. The BRCA2-like classifier confirmed 6 of 10 sBRCA2m cases. The BRCA1-like classifier found no additional sample. Of the 28 examined samples with a BRCA1 promoter hypermethylation, 27 displayed a BRCA1-like profile (96.4%), one was classified as non-BRCA-like. Thus, in one embodiment, the present disclosure relates to the validation of the classifiers. In principle, validation can be performed with any samples or dataset which comprise samples and data, respectively comprising a BRCA-like (BRCA1-like and BRCA2-like) profile and a control profile. Furthermore, the validation set also comprises samples and data, respectively for which the class is known, i.e. with pathogenic variants/mutations in for example the BRCA1 and BRCA2 gene, respectively or other HRD related genes as mentioned below. Optionally, the validation data set further comprises samples and data, respectively that are non-BRCA-like i.e. that do not comprise a mutation in any of the HRD pathway related genes. Alternatively or in addition, validation is also possible by determining the therapy response, i.e. whether a patient responds to a treatment with a treatment regimen comprising homologous recombination deficiency-targeting drugs, drugs that directly cause double strand DNA breaks, drugs that indirectly cause double strand DNA breaks; and/or drugs which have been identified in mechanistic studies to target homologous recombination deficiency.

As mentioned above, HRD is not only associated with alterations in the BRCA genes. Thus, alterations in further HR genes (ATM, BARD1, BRIP1, CHEK1, CHEK2, FAM175A, FANCM, MRE11A, NBN, PALB2, RAD50, RAD51C, RAD51D, and XRCC2) are being classified as BRCA-like and as yet unknown genes. Within the study cohort, 44 tumors without BRCA mutation or BRCA1 methylation displayed a genetic alteration in another OC-risk- or HR-gene (except sTP53 mutations), of which 32 were classified as BRCA-like (72.7%; only BRCA1-like n=11, only BRCA2-like n=4, BRCA1- and BRCA2-like n=15). The detailed results are presented in Example 5.

Thus, the present disclosure also relates to the development and/or training and optionally validation of the classifiers which are used in the method of the present disclosure, i.e. the method of determining HRD status in a subject having ovarian cancer and the method of predicting the response of a subject having ovarian cancer to an anti-cancer therapy, respectively.

The method of the present disclosure comprises the determination of a CNV of a genomic locus in a test sample from a subject comprising cancer cells, e.g. a DNA test sample of cancer cells, wherein the locus is selected from a pool of genomic loci comprising the loci set forth in Table 1 or in Table 2, and wherein similarity between the CNVs in the pool of genomic loci in the test sample from the subject and the CNVs in a corresponding pool of genomic loci in the DNA of a reference sample of BRCA-like mutated cancer cells and a control sample of BRCA non-mutated cancer cells, is indicative for the HRD status in the subject, i.e. whether the cancer cells have a HRD signature or not.

In particular, similarity between the CNVs of the test sample and the CNVs of the reference sample identifies the subject as HR deficient, wherein similarity between the CNVs of the test sample and the CNVs of the control sample identifies the subject as not being HR deficient. Furthermore, non-similarity between the CNVs of the test sample and the CNVs of the reference sample can also identify a subject as not being HR deficient.

Determining CNVs is usually performed at one or more genomic loci. In principle, any appropriate technique can be used to determine genotypes at loci of interest within the genome of a cell. For example, array-based comparative genomic hybridization (array-CGH) can be used. CGH refers generally to molecular-cytogenetic techniques for the analysis of copy number changes, gains or losses, in the DNA content of a given subject's DNA. CGH can be used to identify chromosomal alterations, such as unbalanced chromosomal changes, in any number of cells including, for example ovarian cancer cells. CGH can be utilized to detect one or more chromosomal amplifications and/or deletions of regions between a test sample and a reference/control sample. In particular, nucleic acids from a test sample and nucleic acids from a reference/control sample are labelled differentially, hybridized to an array comprising a plurality of probes, preferentially covering the whole genome, and wherein the ratio of the signal intensity of the test sample to that of the reference/control sample is calculated to measure the copy number changes for a particular location in the genome. This is exemplarily described in WO 2012/038837 A2 as well as in the references cited therein. Furthermore, single nucleotide polymorphism (SNP) arrays, molecular inversion probe (MIP) assays, targeted sequencing of loci of interest, and large-scale sequencing (e.g., whole exome, transcriptome, or genome sequencing) can be used to identify CNVs (as long as they cover or sufficiently approximate the loci of interest). Preferably, low coverage whole genome sequencing (LC-WGS) is used, preferably single-read sequencing, wherein the read length can vary and is for example between 30 to 200 bp, preferably between 30 and 100 bp and most preferably about 65 bp. Furthermore, the LC-WGS can be performed with a coverage of for example 0.1-3×, and preferably with a coverage of 0.5×. The sequencing data can then by analyzed regarding the copy numbers of the DNA in the genomic loci.

Due to the development of the classifiers set forth in Tables 1 and 2 and as explained above, it was possible to establish the method of the present disclosure which detects highly reliable HRD status in a subject. However, the person skilled in the art is well aware of the fact that not all genomic loci set forth in Table 1 and Table 2, respectively need to be considered in order to receive a reliable result on the HRD status of the subjects. In particular, as soon as the BRCA1-like classifiers as set forth in Table 1 and the BRCA2-like classifiers set forth in Table 2 have been developed, the person skilled in the art knows how to vary the method so that still reliable results are achieved. For example, it is possible that the CNVs in additional loci are considered, or that the CNVs in less loci are considered, or that some different loci are considered in comparison to those listed in Table 1 and Table 2, respectively. In particular, variations can be made as long as the method still reaches an appropriate sensitivity, preferably a sensitivity of 70% to 100%, more preferably of 72% to 97%, that allows to make a reliable statement about the HRD status of the subject to be tested.

As regards the BRCA1-like classifiers, the pool of genomic loci can vary as long as the method of the present disclosure still shows a sensitivity of at least 90%, i.e. a sensitivity between 90% and 100%, preferably of at least 94% to 97%, i.e. a sensitivity between 94 to 97% and 100%. In one embodiment, the pool of genomic loci comprises at least 200, preferably 400, more preferably 600, more preferably 800, more preferably 1000, more preferably 1200, more preferably 1400, more preferably 1600, more preferably 1800, more preferably 2000, more preferably 2200, more preferably 2400, and most preferably all of those loci listed in Table 1 as long as the above-mentioned sensitivities are reached. Of course any numbers of loci in between are also encompassed and just not mentioned for simplicity.

As regards the BRCA2-like classifiers, the pool of genomic loci can vary as long as the method of the present disclosure still shows a sensitivity of at least 70%, i.e. a sensitivity between 70% and 100%, preferably of at least 72% to 77%, i.e. a sensitivity between 72 to 77% and 100%. In one embodiment, the pool of genomic loci comprises at least 20, preferably 40, more preferably 60, more preferably 80, more preferably 100, more preferably 120, and most preferably all of those loci listed in Table 2 as long as the above-mentioned sensitivities are reached. Of course any numbers of loci in between are also encompassed and just not mentioned for simplicity.

Thus, similarity or non-similarity between the CNVs in a pool of genomic loci in the test sample from the subject and the CNVs in a corresponding pool of genomic loci in the DNA of a reference sample of BRCA-like mutated cancer cells and a control sample of BRCA non-mutated cancer cells, can be determined in the method of the present disclosure by considering at least 200, preferably 400, more preferably 600, more preferably 800, more preferably 1000, more preferably 1200, more preferably 1400, more preferably 1600, more preferably 1800, more preferably 2000, more preferably 2200, more preferably 2400, and most preferably all of those loci listed in Table 1 and/or by considering at least 20, preferably 40, more preferably 60, more preferably 80, more preferably 100, more preferably 120, and most preferably all of those loci listed in Table 2, respectively.

The method of the present disclosure also encompasses the determination of CNV of one genomic locus in a test sample from a subject comprising cancer cells, e.g. a DNA test sample of cancer cells, wherein the locus is selected from a pool of genomic loci comprising the loci set forth in Table 1, wherein determination of the CNV and the CNVs, respectively, can be made in one of the genomic loci, in two, three, four, five [ . . . ], or in all 2597 of the genomic loci. [ . . . ] represents all numbers from six to 2596. The method of the present disclosure also encompasses the determination of a CNV of one genomic locus in a test sample from a subject comprising cancer cells, e.g. a DNA test sample of cancer cells, wherein the locus is selected from a pool of genomic loci comprising the loci set forth in Table 2, wherein determination of the CNV and the CNVs, respectively, can be made in one of the genomic loci, in two, three, four, five [ . . . ], or in all 138 of the genomic loci. [ . . . ] represents all numbers from six to 137.

As mentioned above, CNVs include losses and gains of genomic segments. The similarity is defined as distance measure between the centroids of the two classes, i.e. the control and reference sample, and the centroid of the test sample. A centroid of a class, which is in the present case the control and reference sample, respectively, is constructed by shrinking the class centroid by an optimization routine towards the overall centroid of both classes after standardizing by the within-class standard deviation for each CNVs.

A similarity score, i.e. a posterior probability score, is computed by providing a shrunken centroid value which is derived from the CNV of the genomic locus and wherein the comparison of the shrunken centroids of the test sample from the subject with the shrunken centroids of the reference sample and the shrunken centroids of the control sample is converted into a posterior probability score according to Tibshirani PNAS 2002 (29). This score ranges from 0-1, with 0-0.49 being non-BRCA-like and 0.5-1 being BRCA-like.

Thus, in one embodiment the method of the present disclosure comprises, or essentially consists of the determination of a CNV of a genomic locus in a test sample from a subject, wherein the test sample comprises cancer cells, e.g. a DNA test sample of cancer cells, wherein the locus is selected from a pool of genomic loci comprising the loci set forth in Table 1, the loci set forth in Table 2, or the loci set forth in Tables 1 and 2; and the provision of a shrunken centroid value which is derived from the CNV of the genomic locus, and wherein a posterior probability score of ≥0.5 identifies the subject as HRD; and/or wherein a posterior probability score of <0.5 identifies the subject as not being HRD.

As mentioned above, the determination of the CNV and the CNVs, respectively, can be made in one of the genomic loci, in two, three, four, five [ . . . ], or in all 2597 of the genomic loci listed in Table 1 ([ . . . ] represents all numbers from six to 2596), and/or in one of the genomic loci, in two, three, four, five [ . . . ], or in all 138 of the genomic loci listed in Table 2 ([ . . . ] represents all numbers from six to 137).

The posterior probability score is generally obtainable by comparing the shrunken centroid values derived from the CNVs of a pool of genomic loci in the test sample from the subject with the shrunken centroid values derived from the CNVs of a reference sample of BRCA-like mutated cancer cells and of a control sample of BRCA non-mutated cancer cells. In general, the loci comprised in the pool of genomic loci as regards the BRCA1-like classifiers are those listed in Table 1 and the loci comprised in the pool of genomic loci as regards the BRCA2-like classifiers are those listed in Table 2. The CNV profile of the reference and the control sample can be a profile that has been established before performing the method of the present disclosure or it can in principle also be established in the course of the execution of the method of the present disclosure. Thus, optionally, the method of the present disclosure further comprises the determination of the posterior probability score by comparison of the shrunken centroid values derived from the CNVs of the pool of genomic loci in the DNA of the cancer cells in the test sample from the subject with the shrunken centroid values derived from the CNVs of a reference set of samples of BRCA-like mutated cancer cells and of control samples of BRCA non-mutated cancer cells.

As mentioned above and as regards the BRCA1-like classifiers, the loci in the pool of genomic loci can vary as long as the method still shows a sensitivity of at least 90%, i.e. a sensitivity between 90% and 100%, preferably of at least 94% to 97%, i.e. a sensitivity between 94 to 97% and 100%. In one embodiment, the pool of genomic loci comprises at least 200, preferably 400, more preferably 600, more preferably 800, more preferably 1000, more preferably 1200, more preferably 1400, more preferably 1600, more preferably 1800, more preferably 2000, more preferably 2200, more preferably 2400, and most preferably all of those loci listed in Table 1 as long as the above-mentioned sensitivities are reached. Of course any numbers of loci in between are also encompassed and just not mentioned for simplicity. Furthermore, as regards the BRCA2-like classifiers, the loci in the pool of genomic loci can vary as long as the method still shows a sensitivity of at least 70%, i.e. a sensitivity between 70% and 100%, preferably of at least 72% to 77%, i.e. a sensitivity between 72 to 77% and 100%. In one embodiment, the pool of genomic loci comprises at least 20, preferably 40, more preferably 60, more preferably 80, more preferably 100, more preferably 120, and most preferably all of those loci listed in Table 2 as long as the above-mentioned sensitivities are reached. Of course any numbers of loci in between are also encompassed and just not mentioned for simplicity.

A posterior probability score of ≥0.5 identifies the subject as HRD and thus, it identifies the subject as being a likely responder to an anti-cancer therapy. A posterior probability score of <0.5 identifies the subject as not being HRD and thus, it identifies the subject a not being a likely responder to an anti-cancer therapy. In other words, similarity between the CNVs of the test sample and the CNVs of the reference sample identifies the subject as HR deficient and thus, identifies the subject as being a likely responder to an anti-cancer therapy, wherein similarity between the CNVs of the test sample and the CNVs of the control sample identifies the subject as not being HR deficient and thus, it identifies the subject a not being a likely responder to an anti-cancer therapy.

The present disclosure is thus also directed to a method of predicting the response of a subject having cancer, in particular ovarian cancer, to an anti-cancer therapy, i.e. to a cancer treatment regimen, wherein the method comprises the steps as defined above. In particular, a posterior probability score of ≥0.5 indicates an increased likelihood that the subject will respond to the anti-cancer therapy, and/or a posterior probability score of <0.5 indicates an increased likelihood that the subject will not respond to the anti-cancer therapy. In other words, similarity between the CNVs of the test sample and the CNVs of the reference sample indicates an increased likelihood that the subject will respond to the anti-cancer therapy, wherein similarity between the CNVs of the test sample and the CNVs of the control sample indicates an increased likelihood that the subject will not respond to the anti-cancer therapy.

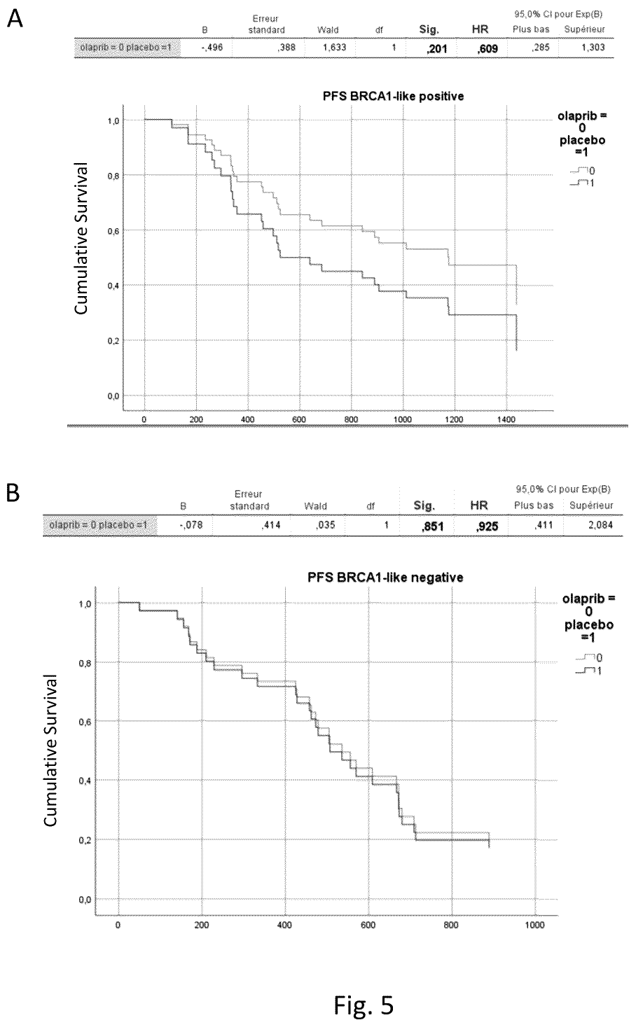

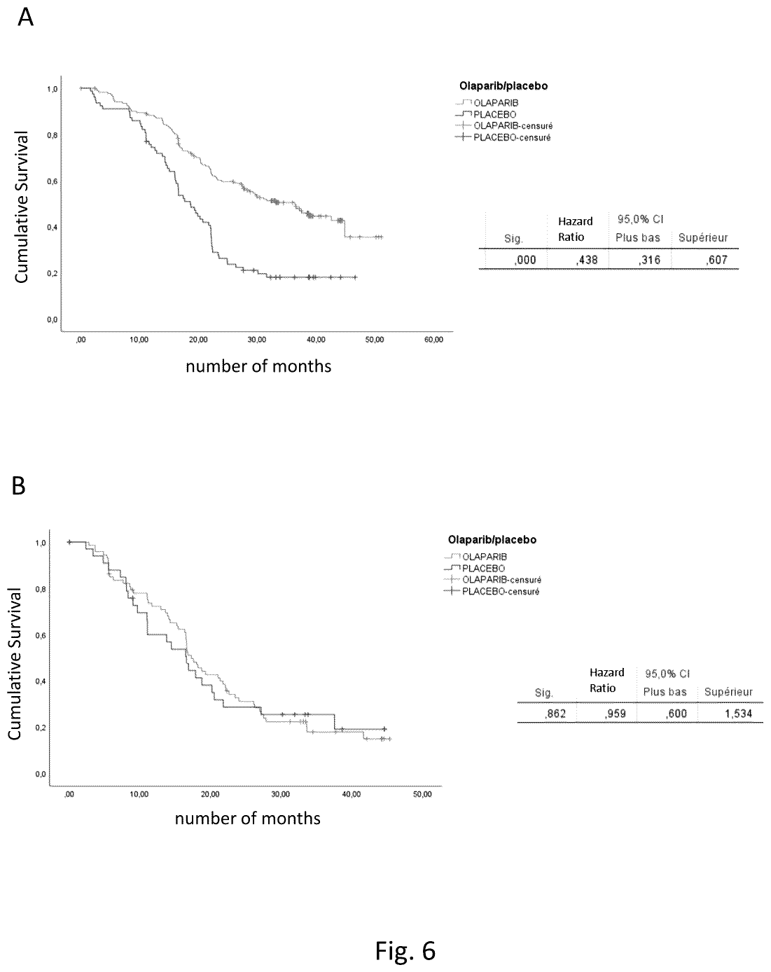

The efficiency of the method of the present disclosure to predict the response of a subject having ovarian cancer to an anti-cancer therapy, in particular to an anti-cancer therapy comprising PARP inhibitors, was evaluated as explained in Example 6. As shown in Example 6 as well as in FIGS. 5 and 6, the method of the present inventive has a high predictive value to distinguish between patients that will benefit from an anti-cancer therapy, in particular from an anti-cancer therapy comprising homologous recombination deficiency-targeting drugs, drugs that directly cause double strand DNA breaks, drugs that indirectly cause double strand DNA breaks and/or drugs that target homologous recombination deficiency, more particularity from an anti-cancer therapy comprising PARP inhibitors, and patients which will not benefit from such an anti-cancer therapy.

This was also confirmed by Koole et al., 2022 (54) which showed that treatment of patients having ovarian cancer with an HRD signature, which were identified in accordance with the method of the present disclosure, with platinum-based chemotherapy, in particular with hyperthermic intraperitoneal chemotherapy (HIPEC), was effective. HIPEC was found to prolong recurrence-free and overall survival in patients with HRD ovarian cancer, in particular with HRD/BRCA1 wild-type ovarian cancers, wherein responses to HR-proficient cancers to HIPEC was less pronounced. Thus, the method of the present disclosure is a promising tool for identifying ovarian cancer patients who benefit from HIPEC.

Thus, patients having cancer cells (or samples derived therefrom), and in particular ovarian cancer cells, identified as having an HRD signature status can be classified, based at least in part on such HRD signature, as being likely to respond to a particular cancer treatment regimen. For example, patients having cancer cells with an HRD signature can be classified as being likely to respond to a cancer treatment regimen that includes the use of drugs that target homologous recombination deficiency, that directly or indirectly cause double strand DNA breaks, and/or where the drug has been identified in mechanistic studies to target homologous recombination deficiency like DNA damaging agents, synthetic lethality agents (e.g., a PARP inhibitor), radiation, or a combination thereof. Examples of DNA damaging agents include, without limitation, platinum-based chemotherapy drugs (e.g., cisplatin, carboplatin, oxaliplatin, picoplatin, including administration as HIPEC), anthracyclines (e.g., epirubicin and doxorubicin), topoisomerase I inhibitors (e.g. campothecin, topotecan, and irinotecan), DNA crosslinkers such as mitomycin C, and triazene compounds (e.g., dacarbazine and temozolomide). Preferably, the cancer treatment regime includes PARP inhibitors or DNA double strand break inducing agents, e.g. platinum compounds, preferably administered as HIPEC, and interstrand crosslinking agents.

Synthetic lethality therapeutic approaches typically involve administering an agent that inhibits at least one critical component of a biological pathway that is especially important to a particular tumor cell's survival. For example, when a tumor cell has a deficient homologous repair pathway (e.g., as determined according to the present disclosure), inhibitors of poly ADP ribose polymerase (or platinum drugs, double strand break repair inhibitors, etc.) can be especially potent against such tumors because two pathways critical to survival become obstructed (one biologically, e.g., by BRCAT/2-like mutation, and the other synthetically, e.g., by administration of a pathway drug). Synthetic lethality approaches to cancer therapy are described in, e.g., O'Brien et al., Converting cancer mutations into therapeutic opportunities, EMBO MOL. MED. (2009) 1:297-299. Examples of synthetic lethality agents include, without limitation, PARP inhibitors or double strand break repair inhibitors in homologous repair-deficient tumor cells, PARP inhibitors in PTEN-deficient tumor cells, methotrexate in MSH2-deficient tumor cells, etc. Examples of PARP inhibitors include, without limitation, olaparib, iniparib, veliparib and niraparib. Examples of double strand break repair inhibitors include, without limitation, KU55933 (ATM inhibitor) and NU7441 (DNA-PKcs inhibitor). Examples of information that can be used in addition to the presence of an HRD signature to base a classification of being likely to respond to a particular cancer treatment regimen include, without limitation, previous treatment results, germline or somatic DNA mutations, gene or protein expression profiling (e.g. BRCA1 and/or BRCA2 status), tumor histology (e.g., epithelial ovarian tumors like serous, mucinous, endometrioid, clear cell, transitional cell tumors (Brenner tumors), carcinosarcoma, mixed epithelial tumor, undifferentiated carcinoma, etc.), disease stage, tumor or cancer grade, number of previous courses of treatment, etc. Preferably, the information that can be used in addition to the presence of an HRD signature to base a classification of being likely to respond to a particular cancer treatment regimen is the tumor histology, and in particular of serous tumors.

Treatment regimens can also comprise the addition of olaparib to bevacizumab maintenance therapy after carboplatin and paclitaxel treatment. In particular, a treatment regime comprises hyperthermic intraperitoneal chemotherapy with cisplatin (100 mg per square meter) at interval debulking after standard neoadjuvant carbotaxol, i.e. paclitaxel and carboplatin, in stage III epithelial ovarian cancer.

Once classified as being likely to respond to a particular cancer treatment regimen as defined above, a treatment regime can be recommended to the patient, and/or the cancer patient can be treated with such a cancer treatment regimen. The disclosure thus relates to a method of the present disclosure which further comprises recommending a subject which has been identified as being HRD and/or having an increased likelihood of responding to the anti-cancer therapy, a treatment regimen comprising the above-mentioned drugs, i.a. homologous recombination deficiency-targeting drugs, drugs that directly cause double strand DNA breaks, drugs that indirectly cause double strand DNA breaks; and/or drugs which have been identified in mechanistic studies to target homologous recombination deficiency. On the other hand, the method may also comprise recommending a subject which has been identified as not being HRD and/or not having an increased likelihood of responding to the anti-cancer therapy, a standard treatment in the specific clinical setting of the patient, which might not include the above-mentioned drugs, i.a. homologous recombination deficiency-targeting drugs, drugs that directly cause double strand DNA breaks, drugs that indirectly cause double strand DNA breaks, and/or drugs which have been identified in mechanistic studies to target homologous recombination deficiency. In turn, such a subject can be classified as likely to respond to a cancer treatment regimen that includes the use of one or more cancer treatment agents not associated with HRD, such as (but not limited to) a taxane agent (e.g., paclitaxel, docetaxel, abraxane), and/or a growth factor receptor inhibitor (e.g., bevacizumab). However, in case no other treatment approach is available, the standard treatment may still comprise homologous recombination deficiency-targeting drugs, drugs that directly cause double strand DNA breaks, drugs that indirectly cause double strand DNA breaks, and/or drugs which have been identified in mechanistic studies to target homologous recombination deficiency.

Accordingly, the present disclosure further relates to a treatment regime comprising homologous recombination deficiency-targeting drugs, drugs that directly cause double strand DNA breaks, and/or drugs that indirectly cause double strand DNA breaks, or that otherwise have been identified as specifically targeting homologous recombination deficient tumors like a DNA damaging agent (e.g., platinum-based chemotherapy, including administration as HIPEC), a synthetically lethal agent (e.g., a PARP inhibitor), or a combination thereof for use in treating ovarian cancer in a subject, wherein said subject has been identified as having an HRD signature and/or identified to have an increased likelihood of responding to the anti-cancer treatment regimen as described before.

Furthermore, the present disclosure relates to a method of treating a patient comprising detecting an HRD signature as described herein and administering (or recommending or prescribing) a treatment regimen comprising the use of homologous recombination deficiency-targeting drugs, drugs that directly cause double strand DNA breaks, and/or drugs that indirectly cause double strand DNA breaks, or that otherwise have been identified as specifically targeting homologous recombination deficient tumors like PARP inhibitors or DNA double strand break inducing agents, e.g. platinum compounds including administration as HIPEC, and interstrand crosslinking agents or any combination thereof. Any appropriate method for treating the cancer at issue can be used to treat a cancer patient identified as having cancer cells having an HRD signature. For example, platinum-based chemotherapy drugs or a combination of platinum-based chemotherapy drugs can be used to treat cancer as described elsewhere (see, e.g., U.S. Pat. Nos. 3,892,790, 3,904,663, 7,759,510, 7,759,488 and 7,754,684). In some cases, anthracyclines or a combination of anthracyclines can be used to treat cancer as described elsewhere (see, e.g., U.S. Pat. Nos. 3,590,028, 4,138,480, 4,950,738, 6,087,340, 7,868,040, and 7,485,707). In some cases, topoisomerase I inhibitors or a combination of topoisomerase I inhibitors can be used to treat cancer as described elsewhere (see, e.g., U.S. Pat. Nos. 5,633,016 and 6,403,563). In some cases, PARP inhibitors or a combination of PARP inhibitors can be used to treat cancer as described elsewhere (see, e.g., U.S. Pat. Nos. 5,177,075, 7,915,280, and 7,351,701). In some cases, radiation can be used to treat cancer as described elsewhere (see, e.g., U.S. Pat. No. 5,295,944). In some cases, a combination comprising different agents (e.g., a combination comprising any of platinum-based chemotherapy drugs, anthracyclines, topoisomerase I inhibitors, and/or PARP inhibitors) with or without radiation treatments can be used to treat cancer. In some cases, a combination treatment may comprise any of the above agents or treatments (e.g., PARP inhibitors or DNA double strand break inducing agents, e.g. platinum compounds and interstrand crosslinking agents) together with another agent or treatment, e.g., a taxane agent (e.g., doxetaxel, paclitaxel, abraxane, preferably paclitaxel), and/or a growth factor receptor inhibitor (e.g., bevacizumab).

Furthermore, patients identified as having cancer cells lacking an HRD signature can be classified, based at least in part on a sample lacking an HRD signature, as being less likely to respond to a treatment regimen that includes a homologous recombination deficiency-targeting drug, drug that directly causes double strand DNA breaks, drug that indirectly causes double strand DNA breaks, and/or drugs which have been identified in mechanistic studies to target homologous recombination deficiency, like PARP inhibitors, DNA double strand break inducing agents, e.g. platinum compounds including administration as HIPEC and interstrand crosslinking agents or a combination thereof. In turn, such a patient can be classified as likely to respond to a cancer treatment regimen that includes the use of one or more cancer treatment agents not associated with HRD, such as a taxane agent (e.g., doxetaxel, paclitaxel, abraxane), and/or a growth factor receptor inhibitor (e.g., bevacizumab). However, in case no other treatment approach is available, the standard treatment may still comprise homologous recombination deficiency-targeting drugs, drugs that directly cause double strand DNA breaks, drugs that indirectly cause double strand DNA breaks, and/or drugs which have been identified in mechanistic studies to target homologous recombination deficiency.

Once classified as being likely to respond to a particular cancer treatment regimen (e.g., a cancer treatment regimen that includes the use of a cancer treatment agent not associated with HRD), the cancer patient can be treated with such a cancer treatment regimen. The disclosure thus provides a method of treating a patient comprising detecting the absence of an HRD signature as described herein and administering (or recommending or prescribing) a treatment regimen not comprising the use of a homologous recombination deficiency-targeting drugs, drugs that directly cause double strand DNA breaks, drugs that indirectly cause double strand DNA breaks, and/or drugs which have been identified in mechanistic studies to target homologous recombination deficiency, like PARP inhibitors, DNA double strand break inducing agents, e.g. platinum compounds and interstrand crosslinking agents, or a combination thereof. In some embodiments the treatment regimen comprises one or more of a taxane agent (e.g., doxetaxel, paclitaxel, abraxane, preferably paclitaxel), and/or a growth factor receptor inhibitor (e.g., bevacizumab). Any appropriate method for the cancer being treated can be used to treat a cancer patient identified as having cancer cells lacking an HRD signature. Examples of information that can be used in addition to the presence of an HRD signature to base a classification of being likely to respond to a particular cancer treatment regimen include, without limitation, previous treatment results, germline or somatic DNA mutations, gene or protein expression profiling (e.g. BRCA1 and/or BRCA2 status), tumor histology (e.g., epithelial ovarian tumors like serous, mucinous, endometrioid, clear cell, transitional cell tumors (Brenner tumors), carcinosarcoma, mixed epithelial tumor, undifferentiated carcinoma, etc.), disease stage, tumor or cancer grade, number of previous courses of treatment, etc. Preferably, the information that can be used in addition to the presence of an HRD signature to base a classification of being likely to respond to a particular cancer treatment regimen is the tumor histology, and in particular of serous tumors.

Once treated for a particular period of time (e.g., between one to six months), the patient can be assessed to determine whether or not the treatment regimen has an effect. If a beneficial effect is detected, the patient can continue with the same or a similar cancer treatment regimen. If a minimal or no beneficial effect is detected, then adjustments to the cancer treatment regimen can be made. For example, the dose, frequency of administration, or duration of treatment can be increased. In some cases, additional anti-cancer agents can be added to the treatment regimen or a particular anti-cancer agent can be replaced with one or more different anti-cancer agents. The patient being treated can continue to be monitored as appropriate, and changes can be made to the cancer treatment regimen as appropriate.

In addition to predicting likely treatment response or selecting desirable treatment regimens, an HRD signature can be used to determine a patient's prognosis. Thus, in one aspect, a method is provided for determining a patient's prognosis based at least in part of detecting the presence or absence of an HRD signature in a sample from the patient. The method comprises, or consists essentially of, (a) determining whether a sample from the patient comprises cancer cells (or whether a sample comprises DNA derived from such cells) having an HRD signature as described herein, and (b)(1) determining, based at least in part on the presence of the HRD signature, that the patient has a relatively good prognosis, or (b)(2) determining, based at least in part on the absence of the HRD signature, that the patient has a relatively poor prognosis. Prognosis may include the patient's likelihood of survival (e.g., progression-free survival, overall survival), wherein a relatively good prognosis would include an increased likelihood of survival as compared to some reference population (e.g., average patient with this patient's cancer type/subtype, average patient not having an HRD signature, etc.). Conversely, a relatively poor prognosis in terms of survival would include a decreased likelihood of survival as compared to some reference population (e.g., average patient with this patient's cancer type/subtype, average patient having an HRD signature, etc.).

As described herein, the present disclosure relates to methods for assessing patients for ovarian cancer cells having an HRD signature. In some embodiments, one or more clinicians or medical professionals can determine whether a sample from the patient comprises cancer cells (or whether a sample comprises DNA derived from such cells) having an HRD signature. In some cases, one or more clinicians or medical professionals can determine if a patient contains cancer cells having an HRD signature by obtaining a cancer cell sample from the patient and assessing the DNA of cancer cells of the cancer cell sample to determine the presence or absence of an HRD signature as described herein.

In some cases, one or more clinicians or medical professionals can obtain a cancer cell sample from a patient and provide that sample to a testing laboratory having the ability to assess DNA of cancer cells of the cancer cell sample to provide an indication about the presence or absence of an HRD signature as described herein. In such cases, the one or more clinicians or medical professionals can determine if a sample from the patient comprises cancer cells (or whether a sample comprises DNA derived from such cells) having an HRD signature by receiving information about the presence or absence of an HRD signature as described herein directly or indirectly from the testing laboratory. For example, a testing laboratory, after assessing DNA of cancer cells for presence or absence of an HRD signature as described herein, can provide a clinician or medical professional with, or access to, a written, electronic, or oral report or medical record that provides an indication about the presence or absence of an HRD signature for a particular patient (or patient sample) being assessed. Such a written, electronic, or oral report or medical record can allow the one or more clinicians or medical professionals to determine if a particular patient being assessed contains cancer cells having an HRD signature. Once a clinician or medical professional or group of clinicians or medical professionals determines that a particular patient being assessed contains cancer cells having an HRD signature, the clinician or medical professional (or group) can classify that patient as having cancer cells whose genome contains the presence of an HRD signature. In some cases, a clinician or medical professional or group of clinicians or medical professionals can diagnose a patient determined to have cancer cells whose genome contains the presence of an HRD signature as having cancer cells deficient in (or likely to be deficient in) HRD. Such a diagnosis can be based solely on a determination that a sample from the patient comprises cancer cells (or whether a sample comprises DNA derived from such cells) having an HRD signature or can be based at least in part on a determination that a sample from the patient comprises cancer cells (or whether a sample comprises DNA derived from such cells) having an HRD signature. For example, a patient determined to have cancer cells having an HRD signature can be diagnosed as likely to be deficient in HRD based on the combination of the presence of an HRD signature and deficient status in one or more tumor suppressor genes (e.g., BRCA1/2, RAD51C), a family history of cancer, or the presence of behavioral risk factors (e.g., smoking). Thus, patients can be detected that have germline mutations in HRD associated genes.

In some cases, a clinician or medical professional or group of clinicians or medical professionals can diagnose a patient determined to have cancer cells whose genome contains the presence of an HRD signature as having cancer cells which can contain genetic mutations in one or more genes in the HRD pathway. Such a diagnosis can be based solely on a determination that a particular patient being assessed contains cancer cells having a genome containing an HRD signature or can be based at least in part on a determination that a particular patient being assessed contains cancer cells having a genome containing an HRD signature. For example, a patient determined to have cancer cells whose genome contains the presence of an HRD signature can be diagnosed as having cancer cells likely to contain genetic mutations in one or more genes in the HRD pathway based on the combination of the presence of an HRD signature and a family history of cancer, or the presence of behavioral risk factors (e.g., smoking).

In some cases, a clinician or medical professional or group of clinicians or medical professionals can diagnose a patient determined to have cancer cells having an HRD signature as having cancer cells likely to respond to a particular cancer treatment regimen. Such a diagnosis can be based solely on a determination that a sample from the patient comprises cancer cells (or whether a sample comprises DNA derived from such cells) having an HRD signature or can be based at least in part on a determination that a sample from the patient comprises cancer cells (or whether a sample comprises DNA derived from such cells) having an HRD signature. For example, a patient determined to have cancer cells having an HRD signature can be diagnosed as being likely to respond to a particular cancer treatment regimen based on the combination of the presence of an HRD signature and deficient status in one or more tumor suppressor genes (e.g., BRCA1/2, RAD51), a family history of cancer, or the presence of behavioral risk factors (e.g., smoking). As described herein, a patient determined to have cancer cells having an HRD signature can be diagnosed as likely to respond to a cancer treatment regimen that includes the use of homologous recombination deficiency-targeting drugs, drugs that directly cause double strand DNA breaks, drugs that indirectly cause double strand DNA breaks; and/or drugs which have been identified in mechanistic studies to target homologous recombination deficiency, for example interstrand crosslinking agents, platinum compounds, PARP inhibitors, anthracyclines, and topoisomerase I and II inhibitors, preferably PARP inhibitors or DNA double strand break inducing agents, e.g. platinum compounds such as cisplatin, carboplatin, oxaliplatin, or picoplatin, including administration as HIPEC, and interstrand crosslinking agents, a combination thereof, or a combination of any of the preceding with another anti-cancer agent.

Once a clinician or medical professional or group of clinicians or medical professionals determines that a sample from the patient comprises cancer cells (or whether a sample comprises DNA derived from such cells) having a genome lacking an HRD signature, the clinician or medical professional (or group) can classify that patient as having cancer cells whose genome lacks an HRD signature. In some cases, a clinician or medical professional or group of clinicians or medical professionals can diagnose a patient determined to have cancer cells containing a genome lacking an HRD signature as having cancer cells likely to have functional HRD. In some cases, a clinician or medical professional or group of clinicians or medical professionals can diagnose a patient determined to have cancer cells containing a genome lacking an HRD signature as having cancer cells that do not likely contain genetic mutations in one or more genes in the HRD pathway. In some cases, a clinician or medical professional or group of clinicians or medical professionals can diagnose a patient determined to have cancer cells containing a genome lacking an HRD signature and that are less likely to respond to a platinum-based chemotherapy drug such as cisplatin, carboplatin, oxalaplatin, or picoplatin, including administration as HIPEC, an anthracycline such as epirubicin or doxorubicin, a topoisomerase I inhibitor such as campothecin, topotecan, or irinotecan, a PARP inhibitor, or radiation and/or more likely to respond to a cancer treatment regimen that includes the use of a cancer treatment agent not associated with HRD such as one or more taxane agents, growth factor or growth factor receptor inhibitors, anti-metabolite agents, etc.

The subjects/patients who are tested, diagnosed, treated, etc. are either treatment naïve subjects or subjects that have already undergone treatment. For example, the subjects display primary or platinum-sensitive relapsed ovarian cancer defined as relapse after a platinum-free interval of at least 6 months.

The present disclosure also provides methods for performing a diagnostic analysis of a nucleic acid sample (e.g., a genomic nucleic acid sample or nucleic acids amplified therefrom) of a cancer patient to determine if a sample from the patient comprises cancer cells (or whether a sample comprises DNA derived from such cells) containing an HRD signature. In some cases, one or more laboratory technicians or laboratory professionals can detect the presence or absence of an HRD signature in the genome of cancer cells of the patient by (a) receiving a cancer cell sample obtained from the patient, receiving a genomic nucleic acid sample obtained from cancer cells obtained from the patient, or receiving a sample containing nucleic acids enriched and/or amplified from such a genomic nucleic acid sample obtained from cancer cells obtained from the patient and (b) performing an analysis (e.g., microarray-based assay or a sequencing-based assay, preferably LC-WGS) using the received material to detect the presence or absence of an HRD signature as described herein. In some cases, one or more laboratory technicians or laboratory professionals can receive a sample to be analyzed (e.g., a cancer cell sample obtained from the patient, a genomic nucleic acid sample obtained from cancer cells obtained from the patient, or a sample containing nucleic acids enriched and/or amplified from such a genomic nucleic acid sample obtained from cancer cells obtained from the patient) directly or indirectly from a clinician or medical professional.

Once a laboratory technician or laboratory professional or group of laboratory technicians or laboratory professionals detects the presence of an HRD signature as described herein, the laboratory technician or laboratory professional (or group) can associate that HRD signature or the result (or results or a summary of results) of the performed diagnostic analysis with the corresponding patient's name, medical record, symbolic/numerical identifier, or a combination thereof. Such identification can be based solely on detecting the presence of an HRD signature or can be based at least in part on detecting the presence of an HRD signature. For example, a laboratory technician or laboratory professional can identify a patient having cancer cells that were detected to have an HRD signature as having cancer cells potentially deficient in HRD (or as having an increased likelihood of responding to a particular treatment as described herein) based on a combination of the presence of an HRD signature and the results of other genetic and biochemical tests performed at the testing laboratory.

The converse of the preceding is also true. Namely, once a laboratory technician or laboratory professional or group of laboratory technicians or laboratory professionals detects the absence of an HRD signature, the laboratory technician or laboratory professional (or group) can associate the absence of an HRD signature or the result (or results or a summary of results) of the performed diagnostic analysis with the corresponding patient's name, medical record, symbolic/numerical identifier, or a combination thereof. In some cases, a laboratory technician or laboratory professional or group of laboratory technicians or laboratory professionals can identify a patient having cancer cells that were detected to lack an HRD signature as having cancer cells with potentially intact HRD (or having a decreased likelihood of responding to a particular treatment as described herein) either based solely on the absence of an HRD signature or based on a combination of the presence of an HRD signature and the results of other genetic and biochemical tests performed at the testing laboratory.

The results of any analyses according to the disclosure will often be communicated to physicians, genetic counselors, or a clinic and/or patients (or other interested parties such as researchers) in a transmittable form that can be communicated or transmitted to any of the above parties. Such a form can vary and can be tangible or intangible. The results can be embodied in descriptive statements, diagrams, photographs, charts, images or any other visual forms. For example, graphs or diagrams showing genotype or HRD status information can be used in explaining the results. The statements and visual forms can be recorded on a tangible medium such as papers, computer readable media such as floppy disks, compact disks, flash memory, etc., or in an intangible medium, e.g., an electronic medium in the form of email or website on internet or intranet. In addition, results can also be recorded in a sound form and transmitted through any suitable medium, e.g., analog or digital cable lines, fiber optic cables, etc., via telephone, facsimile, wireless mobile phone, internet phone and the like.

Thus, the information and data on a test result can be produced anywhere in the world and transmitted to a different location. As an illustrative example, when an assay is conducted outside Germany, the Netherlands, or the EU, the information and data on a test result may be generated, cast in a transmittable form as described above, and then imported into Germany, the Netherlands or the EU. Accordingly, the present disclosure also encompasses a method for producing a transmittable form of information on an HRD signature for at least one patient sample. The method comprises the steps of (1) determining an HRD signature according to methods of the present disclosure; and (2) embodying the result of the determining step in a transmittable form. The transmittable form is a product of such a method.

Several embodiments of the disclosure described herein involve a step of correlating the presence of an HRD signature according to the present disclosure (e.g., the CNVs in comparison to reference and control samples) to a particular clinical feature (e.g., an increased likelihood of HRD deficiency; an increased likelihood of response to a treatment regimen comprising a DNA damaging agent, an anthracycline, a topoisomerase I inhibitor, radiation, and/or a PARP inhibitor; etc.) and optionally correlating the absence of a HRD signature to one or more other clinical features. Throughout this document, wherever such an embodiment is described, another embodiment of the disclosure may involve, in addition to or instead of a correlating step, one or both of the following steps: (a) concluding that the patient has the clinical feature based at least in part on the presence or absence of the HRD signature; or (b) communicating that the patient has the clinical feature based at least in part on the presence or absence of the HRD signature.

As used herein, “communicating” a particular piece of information means to make such information known to another person or transfer such information to a thing (e.g., a computer). In some methods of the disclosure, a patient's prognosis or likelihood of response to a particular treatment is communicated. In some embodiments, the information used to arrive at such a prognosis or response prediction (e.g., HRD signature according to the present disclosure, etc.) is communicated. This communication may be auditory (e.g., verbal), visual (e.g., written), electronic (e.g., data transferred from one computer system to another), etc. In some embodiments, communicating a cancer classification (e.g., prognosis, likelihood of response, appropriate treatment, etc.) comprises generating a report that communicates the cancer classification. In some embodiments the report is a paper report, an auditory report, or an electronic record. In some embodiments the report is displayed and/or stored on a computing device (e.g., handheld device, desktop computer, smart device, website, etc.). In some embodiments the cancer classification is communicated to a physician (e.g., a report communicating the classification is provided to the physician). In some embodiments the cancer classification is communicated to a patient (e.g., a report communicating the classification is provided to the patient). Communicating a cancer classification can also be accomplished by transferring information (e.g., data) embodying the classification to a server computer and allowing an intermediary or end-user to access such information (e.g., by viewing the information as displayed from the server, by downloading the information in the form of one or more files transferred from the server to the intermediary or end-user's device, etc.).

The disclosure further relates to a system for detecting HRD in a sample. The system comprises, or consists essentially of, (a) a sample analyzer configured to produce a plurality of signals about genomic DNA in the test sample, and (b) a computer sub-system programmed to calculate, based on the plurality of signals, the CNVs in the pool of genomic loci. The computer sub-system can be programmed to compare the CNVs of the test sample with the CNVs in a corresponding pool of genomic loci in the DNA of a reference sample of BRCA-like mutated cancer cells and a control sample of BRCA non-mutated cancer cells, respectively, and/or to provide a shrunken centroid value which is derived from the CNVs of the genomic locus, by comparison of the shrunken centroid values derived from the CNVs of the pool of genomic loci in the DNA of the cancer cells in the test sample from the subject with the shrunken centroid values derived from the CNVs of a reference set of samples of BRCA-like mutated cancer cells and of control samples of BRCA non-mutated cancer cells, to detect (a) HRD or likelihood of HRD (e.g., an HRD signature) in the sample, or (b) an increased likelihood that the cancer patient will respond to a cancer treatment regimen as defined above. The system can comprise an output module configured to display (a) or (b). The system can comprise an output module configured to display a recommendation for the use of the cancer treatment regimen.

The disclosure further provides a corresponding computer sub-system, i.e. computer program product embodied in a computer readable medium that, when executing on a computer, performs the calculations as described above.

Several documents are cited throughout the text of this specification. The contents of all cited references (including literature references, issued patents, published patent applications as cited throughout this application including the background section and manufacturer's specifications, instructions, etc.) are hereby expressly incorporated by reference; however, there is no admission that any document cited is indeed prior art as to the present disclosure.

A more complete understanding can be obtained by reference to the following specific Examples which are provided herein for purposes of illustration only and are not intended to limit the scope of the disclosure.

### EXAMPLES

### Methods

### OC-BRCA1/2-Like Classifier Training

### Training Cohort

Fifty confirmed gBRCA1-mutated (m), 10 confirmed gBRCA2m cases and 13 patients without a family history of ovarian or breast cancer (controls) were identified through the Netherlands Cancer Institute (NKI) tumor registration database and the Erasmus Medical Center (see Table 3). This study was conducted in concordance with Dutch law and national guidelines that allow for the analysis of residual tissue specimens obtained for diagnostic purposes and anonymized publication of the results (38).

TABLE 3
Pathological characteristics of samples within the training
set. Histological subtyping and two-tier grading of
the ovarian cancer samples in the training cohort.

| | | Control | gBRCA1 | gBRCA2 |
|---|---|---|---|---|
| Subtype | Serous | 13 | 48 | 10 |
| | Endometrial | 0 | 1 | 0 |
| | Other | 0 | 1 | 0 |
| Grade | Low grade | 1 | 1 | 0 |
| | High grade | 12 | 48 | 10 |
| | Missing | 0 | 1 | 0 |

### DNA Isolation and BRCA1 Methylation Assay

Histological classification and grading, according to the 2-tiered system, were performed by two expert gynaecopathologists. We isolated DNA from formalin-fixed paraffin-embedded (FFPE) tumor slides, using (micro)-dissection to obtain 60% tumor percentage with the Qiagen DNA mini kit (Qiagen, cat nr. 51306) and for BRCA1 methylation, using an MLPA kit (MRC-Holland) as described before (30). In particular, hypermethylation of the BRCA1 promoter was assessed using a custom methylation specific MLPA set according to the manufacturer's protocol (ME005-custom; MRC-Holland, Amsterdam, the Netherlands). Probe sequences of the MLPA set are available on request (info@mlpa.com). DNA fragments were analyzed on a 3730 DNA Analyzer (Applied Biosystems, Foster City, CA, USA). For normalization and analysis, the Coffalyzer program was used (MRC-Holland, Amsterdam, The Netherlands); peak heights below 250 were excluded from further analyses. When the BRCA1 probes showed methylation (threshold of 0.2; MRC-Holland), we classified the result as BRCA1 promoter methylation.

### Quantitative Methylation Assay

A PCR-based methylation assay was performed centrally (Institute of Pathology, University Hospital Bonn, Germany), as previously described (37). Quantitative methylation-specific PCR (qMSP) assays were designed to allow specific amplification of the bisulfite-converted (convertion using the innuCONVERT Bisulfite Basic Kit; Analytik Jena, Jena, Germany) methylated gene promoter sequences of BRCA1, PALB2 and RAD51C. Triplicate measurements were carried out for each sample, and median methylation levels were computed with values ≥5% considered positive.

NimbleGen 135K Oligonucleotide Array Comparative Genomic Hybridization (aCGH)

We obtained NimbleGen 135K aCGH profiles from the training set samples. The 135K array data were mapped to the BAC aCGH platform for dimension and noise reduction by averaging probes covered by the BAC clone, as described before (39,40). The data discussed here have been deposited in NCBI's Gene Expression Omnibus and are accessible through GEO Series accession number GSE111688 (https://www.ncbi.nlm.nih.gov/geo/query/acc.cgi?acc=GSE111688).

### Estimating the Performance of the Classifier in a 10-Fold Double Loop Cross Validation Loop

We carried out nested double loop 10-fold cross validation of shrunken centroid classifiers to classify OC as being similar to gBRCA1m(BRCA1-like) or gBRCA2m(BRCA2-like) or controls (C) (29). We obtained the delta threshold by optimizing the classification error and selecting the sparsest model within one standard error of the optimal solution (41). Subsequently, we used the model at the selected threshold to predict the samples in the outer loop. The area under the curve (AUC) of the receiver/operator curve (ROC) of our predictions of the class labels is computed using samples that are left out of the training procedure. After estimating this unbiased performance, we trained the full dataset's final model using the inner loop. See Supplementary Methods for pseudocode.

### Validation in TCGA Data Set

We downloaded the Infinium HumanMethylation27 BeadChip methylation, segmented genome-wide human SNP6.0 copy number, and gene expression data from the firebrowse.org archives of the ovarian cancer TCGA data (version 2015082100.0.0). BRCA1 and BRCA2 mutation status were obtained from cBioPortal, which stores the somatic and germline mutation status used in the original TCGA manuscripts (42). The BRCA1 promoter methylation status was obtained by correlating the methylation and gene expression data. We used methylation probe cg10893007 because of the strongest Pearson correlation with log BRCA1 gene expression ratio among the BRCA1 promoter probes and close location to the MLPA probe used in the previous study (30) among those covering the BRCA1 promoter in the TCGA dataset.

### Classifying TCGA Data

TCGA data were mapped to the NKI BAC array CGH positions, as described before (39). In short, SNP6.0 probes within the start and end positions of the BAC clone were averaged (mapped TCGA). We adjusted for differences in scaling and centering by using a method similar to quantile normalization. Briefly, we performed linear regression by fitting a generalized linear Gaussian model with identity link function to the sorted location-wise average DNA copy number values of the NimbleGen data and the sorted location-wise DNA copy number averages of the mapped TGCA dataset. Subsequently, we used the obtained alpha coefficient to correct the centering and the obtained beta coefficient to correct the scaling of the mapped TCGA data, followed by classification. We validated this method on samples that had been analyzed before (39) both on NimbleGen 135K aCGH and SNP6.0. Subsequently, samples were classified as BRCA-like if the predicted probability was >0.5 and non-BRCA-like if the predicted probability was <=0.5, as was pre-defined in the training set.

### Performance in TCGA Data

The sensitivity and specificity of detecting the class labels gBRCA1/BRCA2 mutation, sBRCA1/BRCA2 mutation, and BRCA1 promoter hypermethylation were calculated. The Youden index, (sensitivity+specificity)−1, was used as a balanced measure to assess performance. This readout resembles the equal weighting of sensitivity and specificity in the training process.

Independent Validation within AGO-TR1 Cohort Study

Within the consecutive cohort study AGO-TR1 (NCT02222883) 523 OC patients were counseled and enrolled in 20 AGO study group centers in Germany. The ethical committee approved the study protocol of the Landesaerztekammer Nordrhein (Nr. 2014340). Written informed consent was obtained before any study-related procedure. All individuals were 18 years or older and displayed a primary (PR: n=281) or platinum-sensitive relapsed (RE: n=242) OC, defined as relapse after a platinum-free interval of at least 6 months. The AGO study group documented clinical data including demographics, medical and family history as well as disease characteristics.

The Center of Hereditary Breast and Ovarian Cancer in Cologne performed the genetic analyses on blood samples of all participants and FFPE-tumor samples of 496 patients as previously described (36,37). In short, a paired multi-gene panel sequencing of germline and tumor samples analyzing 27 OC related and DNA repair genes was performed with a complementary use of CNV-analysis for the detection of large genomic rearrangements in germline samples. In particular, paired mutation sequencing of germline and tumor samples was performed at the Center of Hereditary Breast and Ovarian Cancer, Cologne, using a customer-tailored SureSelect gene panel (TruRisk® Panel, Agilent, Santa Clara, U.S.) covering the entire coding and exon-flanking regions (+/−15 nt) of the below specified 27 genes. A hybridization capture-based NGS protocol, suitable for DNA derived from blood as well as FFPE-tissue, was applied (Agilent SureSelect XT; optimized for 200 ng genomic DNA input). Sequencing was carried out on Mi-Seq and Hi-Seq 4000 devices (Illumina, San Diego, U.S.). For bioinformatical analyses we applied the VARBANK version 2.10 pipeline of the Cologne Center for Genomics and the SOPHiA DDM® platform (Sophia Genetics, Saint-Sulpice, Switzerland). For the germline samples CNV-detection via MLPA (multiple Ligation-dependent Probe Amplification, MRC Holland, Amsterdam, The Netherlands), CNV-tool of the SOPHIA DDM® platform, and arrayCGH (array comparative genomic hybridization) were applied, if appropriate. The analyses of all germline and 488 of 496 FFPE samples was successfully performed. HR genes: ATM, NM_000051.3; BARD1, NM_000465.3; BRCA1, NM_007294.3; BRCA2, NM_000059.3; BRIP1, NM_032043.2; CHEK1, NM_001330427.1; CHEK2, NM_007194.3; FAM175A, NM_139076.2; FANCM, NM_020937.3; MRE11A, NM_005591.3; NBN, NM_002485.4; PALB2, NM_024675.3; RAD50, NM_005732.3; RAD51C, NM_058216.2; RAD51D, NM_002878.3; XRCC2, NM_005431.1.

HNPCC genes: MLH1, NM_000249.3; MSH2, NM_000251.2; MSH6, NM_000179.2; PMS2, NM_000535.6

other OC associated genes: BUB1B, NM_001211.5; CDH1, NM_004360.4; MAP3K1, NM_005921.1; PIK3CA, NM 006218.3; PTEN, NM 000314.6; STK11, NM 000455.4; TP53, NM_000546.5

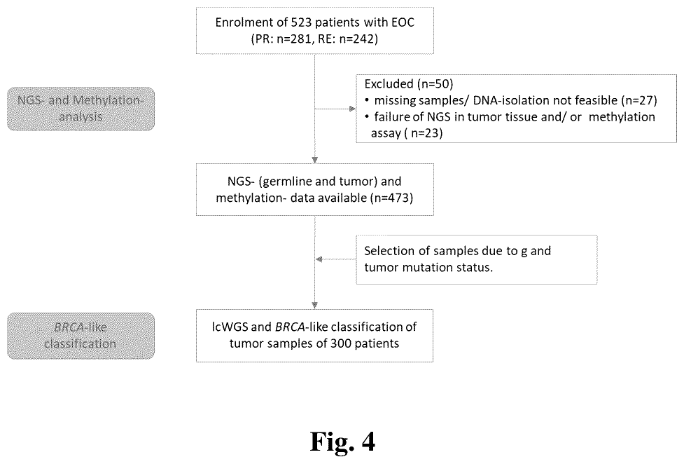

All variants were classified using a five-tier variant classification system as proposed by the International Agency for Research on Cancer Unclassified Genetic Variants Working Group, namely, deleterious=class 5, likely deleterious=class 4, variant of uncertain significance=class 3, likely benign=class 2 and benign=class 1. For somatic variants, the My Cancer Genome database (http://www.mycancergenome.org), the IARC TP53 database (https://p53.iarc.fr) and the ClinVar database (https://www.ncbi.nlm.nih.gov/clinvar/) were also considered for variant classification. Variants reported to occur in large outbred control reference groups at an allele frequency of >1% were generally considered benign. Class 4/5 variants were subsequently defined as ‘deleterious variants’. Variants were considered somatic if they were not identified in a paired germline analysis of the corresponding blood sample. Also, quantitative methylation assays analyzing BRCA1-, PALB2-, and RAD51C-promoter regions were carried out as described before (37). In total, the complete data of 473 individuals were successfully generated (37) (see CONSORT-like flow diagram, FIG. 4).

### Quality Control of AGO-TR1 Samples

The obtained copy number profiles were visually assessed supported by signal to noise ratios and noise variance as quality measures. The unsegmented² log ratios were assumed to represent the noise, whereas the segmented profiles were assumed to represent the signal. Both values were calculated per profile in R using the base function var as follows in pseudocode:

Noise_variance=var(segmented−unsegmented profile)

Signal to noise_ratio=var(segmented profile)/noise_variance

As observed earlier (39), cutoffs are experiment dependent, therefore these measures formed support for the visual assessment. In general, samples with a signal to noise ratio lower than 1 are suspicious for lower quality and with higher level of noise variance. However, determination of too low quality was done visually by identifying those samples for which visually present large aberrations were not called by the segmentation algorithm and thus resulted in an incorrect ‘flat’ copy number profile. Flat copy number profiles in general were not excluded as they might contain biology if not based on high noise variance. In a post-hoc analysis, flat profiles associated with non-HGS histology and non-HR-associated mutations. Samples with higher noise but also visually correctly segmented profiles were retained.

### Selection of the Study Sample

We selected 300 samples with matching germline and somatic mutation status and successful performed methylation analyses for BRCA-like classification (see Table 4), including all available samples with deleterious germline and somatic variants (IARC class 4/5) in BRCA1/2 (n=118), other HR-related and hereditary non-polyposis colorectal cancer (HNPCC) genes (n=43), and somatic (s) BRCA1/2-VUS (variants of unknown significance) (n=4). As controls, we randomly selected a similarly sized group (n=135) from the rest of the main cohort taking PR-/RE-status, age at diagnosis, and sTP53 mutations into account. Samples with only gBRCA1/2 VUS (n=15) were excluded.

TABLE 4
Occurrence of genetic and clinical features within the samples
of the presented study cohort. A) lists all samples, whereas B)
excludes all samples with an BRCA or other HR gene
mutation, as those were enriched within the study cohort.
Genetic and epigenetic features coincided within some samples
and were listed in each subcategory concerned. The mean age
at diagnosis was 57.6 years (range 18-83 years) in the main
cohort and 59.4 years (range 21-83 years) excluding all BRCA1/2-
or HR gene mutated cases. PR: primary ovarian cancer, RE:
recurrent disease

| | | | | B | |
|---|---|---|---|---|---|
| | | A | | study cohort | |
| | | study | | (n) without | |
| | | cohort | | BRCA/HR | |
| | | (n) | [%] | gene mutations | [%] |
| germline and | gBRCA1 | 69 | 23.0 | | |
| somatic | gBRCA2 | 23 | 7.7 | | |
| mutation status | gHR genes | 37 | 12.3 | | |
| | sBRCA1/2 | 28 | 9.3 | | |
| | sHR genes | 12 | 4.0 | | |
| | gHNPCC genes | 3 | 1.0 | 3 | 2.1 |
| | sTP53 | 251 | 83.7 | 106 | 75.2 |
| | sPTEN/PIK3CA | 17 | 5.7 | 10 | 7.1 |
| methylation | BRCA1 methyl | 28 | 9.3 | 26 | 18.4 |
| status | RAD51C methyl | 4 | 1.3 | 2 | 1.4 |
| histological | high grade serous | 246 | 82.0 | 110 | 78.0 |
| subtype | high grade | 16 | 5.3 | 9 | 6.4 |
| | endometrioid | | | | |
| | serous, grade | 6 | 2.0 | 3 | 2.1 |
| | unknown | | | | |
| | low grade serous | 7 | 2.3 | 4 | 2.8 |
| | low grade | 4 | 1.3 | 1 | 0.7 |
| | endometrioid | | | | |
| | clear cell | 2 | 0.7 | 2 | 1.4 |
| | mucinous | 4 | 1.3 | 3 | 2.1 |
| | other/missing | 15 | 5.0 | 9 | 6.4 |
| clinical | PR | 160 | 53.3 | 83 | 58.9 |
| features | RE | 140 | 46.7 | 58 | 41.1 |
| | Total | 300 | | 141 | |

### DNA Isolation

Hematoxylin and eosin-stained 3 μm tissue sections were centrally investigated (Institute of Pathology, University Hospital Bonn, Germany). Tumor areas containing >80% tumor nuclei were chosen and dissected for DNA isolation. DNA isolation from FFPE tumor samples was conducted using standard procedures, as previously described (37).

Low-Coverage Whole Genome Sequencing (lcWGS) and BRCA1/2-Like Classification

LcWGS was centrally performed (NKI), as described earlier (39). Library preparation was performed with an input of 200 ng double-stranded DNA derived from FFPE-tumor samples using the TruSeq® DNA LT Sample Preparation kit (Illumina, San Diego, U.S.). Ten explicit indexed samples were equimolarly pooled and sequenced in one lane on an Illumina HiSeq 2500 device (Illumina, San Diego, U.S). Single-read sequencing (read-length 65 bp) was performed with an aimed coverage of 0.5× and sequences were aligned to reference genome GRCh38. Reads were counted in 20 kb non-overlapping bins, corrected for CG bias and corrected for local alignment-bases estimated mappability, resulting in² log count ratios. The 20 kb resolution² log ratios were mapped to the 1 MB resolution input for the classifier (mapped AGO-TR1). This was done by averaging the² log count ratios within the 1 MB bins (surrounding the BAC clone locations of the BAC platform).

Since the classifier's training was performed on oligonucleotide array CGH data, we performed a correction of centering and scaling of the data with the next-generation sequencing (NGS) platform in this study. This correction is akin to quantile normalization and was performed by fitting a linear regression model with Gaussian distribution and identity link function using the R glm function to the sorted location-wise average of the training set and the mapped AGOTR1 dataset. Subsequently, we used the obtained alpha coefficient to correct the centering and the obtained beta coefficient to correct the scaling of the new data. We validated this method on samples that had been analyzed both on NimbleGen 135K array CGH and NGS in the cross-platform robustness dataset we previously described (39). Subsequently, samples were classified as BRCA-like if the predicted probability was >0.5 and non-BRCA-like if the predicted probability was <=0.5 as was pre-defined in the training set. The cross validation algorithm, pseudo code for platform correction, analysis of misclassified samples and error curves and the validation of platform correction is described in the following:

### Cross Validation Algorithm:

1) Split the data in 10 folds using the pamr:::balanced.folds function from the pamr package.
2) Take 9/10 of the data (outer loop start).
a. Train a classifier on 8/9 of this selection of the data (inner loop start).
b. Estimate optimal shrinkage penalty from 100 shrinkage penalties on left-out 1/9 of this selection. Shrinkage penalties range from exp(−3) to exp(1.5); with step size between −3 and 1.5 at 0.045)
c. Calculate error between class (0 or 1) and predicted probability (range: [0-1])
d. Save error
e. Repeat until all 9 parts have been left out
3) Find threshold with lowest error over 9 inner loops; in case of multiple values, take the mean rounded down. Subsequently we used the one standard error rule to select the highest threshold within 1 standard error of the optimal solution, yielding a sparser model with similar prediction performance as that with the minimal error (1) (inner loop end)
4) Train model on 9/10 of the data
5) Predict with the model and threshold from 3) on 1/10 of the left-out data and save predictions
6) Repeat until all 10 parts have been left out once (outer loop end)
7) Calculate area under the receiver operator curve (AUROC) and associated confidence interval (CI) of the outer-loop predictions

### Building the Final Classifier Models

In the cross validation loops a sample of the data is used to obtain the unbiased performance measures. The final classifier, which uses all the data rather than only a sample, was trained on all the data using the inner loop described above.

### Pseudo Code for Platform Correction

1) calculate the location-wise mean of the 3277 mapped BAC clones over all the samples of the TCGA data. E.g. BAC Clone 1 has an average value of 0.03 in the dataset.
2) sort (order from lowest to highest) the location-wise mean of the 3277 mapped BAC clones over all the patients of the TCGA data
3) calculate the location-wise mean of the 3277 mapped BAC clones of the training set Nimblegen data
4) sort (order from lowest to highest) the location-wise mean of the 3277 mapped BAC clones of the training set Nimblegen data
5) fit a linear regression model using glm with gaussian distribution and identity link function between the sorted mapped TCGA means and the sorted mapped Nimblegen means using the R function: glm(sorted.mean.Nimblegen˜sorted.mean.TCGA)
6) Scale the TCGA data (3277 mapped BAC clones in the rows with x columns equal to the number of samples with the beta coefficient of the linear model and re-center the mean using the alpha coefficient: (TCGAdata*beta coefficient)+alpha coefficient

### Validation of Platform Correction

We used the cross-platform comparison dataset described before (39). In this dataset we analyzed the same sample with multiple techniques. Since we are interested in the direct comparison between NGS (NGS) and NimbleGen 135K array CGH (NG135), we intersected the dataset for overlap between these samples. For 170 samples we obtained a copy number profile with NGS and NG135. Without applying the platform correction we obtained an accuracy of 0.86 (95% CI: 0.80-0.91) and a kappa value of 0.71 (95% CI: 0.56-0.86) for the BRCA1-like classifier and an accuracy of 0.90 (95% CI: 0.84-0.94) and a kappa of 0.80 (95% CI: 0.65-0.94) for the BRCA2-like classifier. With applying the platform correction, we obtained an accuracy of NGS 0.89 (95% CI: 0.835-0.934) and a kappa of 0.79 (95% CI: 0.64-0.93) for the BRCA1-like classifier and an accuracy of 0.86 (95% CI: 0.80-0.91) and a kappa of 0.71 (95% CI 0.56-0.86) for the BRCA2-like classifier. For SNP6 we retained an accuracy of 0.84 (95% CI 0.66-0.94) for BRCA1-like classification and 0.97 (95% CI 0.82-0.99) for BRCA2-like classification.

### Statistical Analyses

Fisher's exact- and χ²-square-test were applied, where appropriate, to calculate the level of significance. All tests were two-sided, and a p-value<0.05 after correction for multiple testing using the Benjamini-Hochberg approach was considered significant.

### Example 1: Generation of Copy Number Profiles

We generated copy number profiles of 13 control, 50 gBRCA1m and 10 gBRCA2m OC, and show the pathological characteristics of these tumors in Table 3. FIG. 1 presents the average profiles of gBRCA1m and gBRCA2m versus control ovarian cancers.

### Example 2: Training OC-Specific Classifiers

First, we classified the ovarian cancer copy number profiles with the breast cancer BRCA1-like and BRCA2-like classifiers (30,31) and obtained an AUC of 0.51 (95% CI: 0.4-0.63) for gBRCA1m and an AUC of 0.63 (95% CI: 0.28-0.98) for gBRCA2m OC in the training set. Given this low performance, we trained shrunken centroid classifiers on ovarian cancer data to investigate whether the performance could be improved. These shrunken centroids select genomic regions and weights that are discriminative for the BRCA-like and non-BRCA-like class. We used ten-fold cross-validation and trained on the class labels gBRCA1m vs. control and gBRCA2m vs. control. We observed a cross-validated AUC of 0.67 (0.55-0.78) and 0.91 (0.79-1) respectively for BRCA1-like and BRCA2-like classification (Table 5). Since the NimbleGen aCGH platform is not available anymore, we validated cross-platform compatibility of our classifier using the same methods and dataset as previously described (39), see the Methods section above.

TABLE 5
Cross validated results of training shrunken centroids classifiers to distinguish
BRCA1- or BRCA2-mutated from control ovarian cancers. AUC: Area under the curve
of the receiver operating characteristic. CI: confidence interval.

| | AUC | Sensitivity | Specificity | | | | |
|---|---|---|---|---|---|---|---|
| | (95% CI) | (95% CI) | (95% CI) | | Contr. | BRCA1 | BRCA2 |
| gBRCA1 | 0.67 | 0.76 | 0.69 | Predicted Control | 9 | 12 | 0 |
| vs.control | (0.55-0.78) | (0.62-0.87) | (0.39-0.91) | Predicted BRCA1 | 4 | 38 | 0 |
| gBRCA2 | 0.91 | 0.90 | 0.92 | Predicted Control | 12 | 0 | 1 |
| vs. control | (0.79-1) | (0.55-1) | (0.64-1) | Predicted BRCA2 | 1 | 0 | 9 |

### Example 3: External Validation of Ovarian Cancer BRCA Classifiers in the TCGA Data

We used the TCGA ovarian cancer data as an external validation set for our newly trained ovarian cancer classifiers. We classified 358 of 583 samples as having a BRCA1-like profile. Within these 358, 25/26 gBRCA1, 10/11 somatic BRCA1-mutated tumors, and 67/69 BRCA1-methylated tumors were classified as BRCA1-like, resulting in an overall sensitivity of 94.7% (95% CI: 0.82-0.99). Specificity, however, is lower, at 40% (95% CI: 0.36-0.45). 350 of 583 samples were assigned to be BRCA2-like. Within these 350, 20/25 gBRCA2m and 8/11 sBRCA2m samples were classified as BRCA2-like, resulting in a sensitivity of 77% (95% CI: 0.6-0.9) and a specificity of 41% (95% CI: 0.37-0.49).

### Example 4: Validation within the AGO-TR1 Cohort

We further analyzed both the germline/somatic mutation status of BRCA1/2 and BRCA1 promoter hypermethylation in the AGO TR1 study. In addition, we aimed to characterize those patients called BRCA-like without a mutation in BRCA1 or BRCA2.

### Study Sample

In total, 300 samples with complete genetic and epigenetic information available, were analyzed. 129 germline and 43 somatic deleterious variants in BRCA1/2 and other HR genes were present in 159 OC samples (see Table 4). The majority displayed a BRCA1 mutation (g: n=69, s: n=17), aBRCA2 mutation (g: n=23, s: n=11) or aRAD51C mutation (g: n=13). One of these samples presented a germline double mutation of gBRCA1 and -2 and was analyzed with the gBRCA1m group; one somatic BRCA2 mutation coincided with a gBRCA2 mutation and was assigned to the gBRCA2 group. Eight other samples with a BRCA mutation (g: n=6, s: n=2) displayed a class 4/5 in another HR gene as well and were analyzed with the respective BRCA-mutated group.

Most OCs presented with high-grade serous histology (82%), and a tumor TP53 mutation was found in 83.7%. Promoter hypermethylation was detected in 9.3% of samples for BRCA1 (n=28) and in 1.3% of samples (n=4) for RAD51C. The mean age at diagnosis was 57.6 years, and 160 primary OC and 140 platinum-sensitive recurrent cases were included. 223 tumors showed a BRCA1-like or BRCA2-like classification (74.3%). 78 OC thereof were BRCA1-like, 26 BRCA2-like, and 119 were both BRCA1- and BRCA2-like.

Detection of BRCA-Associated Ovarian Cancers with BRCA-Like Classification

As there is no established gold standard to define BRCAness in general, we first focused on the detection-rate of tumors derived from deleterious germline mutations in BRCA1/2 (see Table 6, FIG. 2). 67 of 69 BRCA1-mutated cases had a BRCA1-like (detection rate of 97.1%), whereas 16 of 22 samples with a BRCA2 class4/5 variant in germline were BRCA2-like (detection rate of 72.7%). All remaining BRCA2-associated cases were identified by combining both classifiers, i.e. being BRCA1- or BRCA2-like.

TABLE 6
Detection rate of the BRCA1-(A) and BRCA2-like classifier (B) in
BRCA-associated cancers. (C) shows the complementary
application of both classifiers; BRCA1- and/ or BRCA2-like.

| | A) | | |
|---|---|---|---|
| | BRCA1-like | [%] | total |
| gBRCA1 | 67 | 97.1 | 69 |
| sBRCA1 | 15 | 88.2 | 17 |
| BRCA1 methyl | 27 | 96.4 | 28 |
| BRCA1 WT | 88 | 47.3 | 186 |
| Total | 197 | 65.7 | 300 |
| | B) | | |
| | BRCA2-like | [%] | total |
| gBRCA2 | 16 | 72.7 | 22 |
| sBRCA2 | 6 | 60.0 | 10 |
| BRCA2 WT | 124 | 46.3 | 268 |
| Total | 146 | 48.7 | 300 |
| | C) | | |
| | BRCA1/2-like | [%] | total |
| gBRCA1 | 67 | 97.1 | 69 |
| gBRCA2 | 22 | 100.0 | 22 5 |
| sBRCA1 | 17 | 100.0 | 17 |
| sBRCA2 | 6* | 60.0 | 10* |
| BRCA1 methyl | 27* | 96.4 | 28* |
| BRCA WT | 85 | 54.8 | 155 |
| Total | 223 | 74.3 | 300 |

*One sample displayed both a somatic BRCA2 mutation and a BRCA1 methylation and was classified as BRCA1- and BRCA2-like. Therefore, it was presented in both groups but counted only once for the total sum. The sample displaying both a gBRCA and 2 mutation was counted among the gBRCA1 samples, the sample with a BRCA2 mutation in germline and tumor was counted among the gBRCA2 samples.
WT: wild type,
g: germline,
s: somatic,
methyl: promoter hypermethylation.

Regarding deleterious somatic variants, 15 of 17 (88.2%) samples with aBRCA1 mutation were identified by the BRCA1-like classifier. As the other two tumors displayed a BRCA2-like phenotype, both classifiers' application detected 100% of the mutated cases. The BRCA2-like classifier confirmed 6 of 10 sBRCA2m cases. The BRCA1-like classifier found no additional sample. Of the 28 examined samples with a BRCA1 promoter hypermethylation, 27 displayed a BRCA1-like profile (96.4%), one was classified as non-BRCA-like.

### Example 5: Classification of Samples with HR Gene Alterations Beyond BRCA1/2 and Mutations in HNPCC Genes

Alterations in further HR genes (ATM, BARD1, BRIP1, CHEK1, CHEK2, FAM175A, FANCM, MRE11A, NBN, PALB2, RAD50, RAD51C, RAD51D, XRCC2) varied in being classified as BRCA-like (see Table 7). Within the study cohort, 44 tumors without BRCA mutation or BRCA1 methylation displayed a genetic alteration in another OC-risk- or HR-gene (except sTP53 mutations), of which 32 were classified as BRCA-like (72.7%; only BRCA1-like n=11, only BRCA2-like n=4, BRCA1- and BRCA2-like n=15). A distinct statistical association was observed for the largest subgroup, the RAD51C-associated tumors. All germline mutation-carriers developed a BRCA-like OC (only BRCA1-like n=6, BRCA1- and BRCA2-like n=7). No clear statistical association could be observed for most other HR gene mutated samples and samples with RAD51C methylation (see Table 7, FIG. 2) possibly due to small numbers and coinciding aberrations.

TABLE 7
BRCA-like classification of BRCA1/2 wildtype samples with alterations in HR-and HNPCC-genes. Two samples are
described twice, each due to mutual alterations (see * and **). Both samples displayed a non-BRCA-like phenotype.
m: mutation/class4/5 variant; methyl: promotor hypermethylation; b1: BRCA1-like; b2: BRCA2-like

| | | | | | (only b1; | | | |
|---|---|---|---|---|---|---|---|---|
| | | | additional | | only b2; | | | |
| Genetic alteration | g (n) | s (n) | alterations | BRCA-like (n) | b1 + 2) | [%] | non-BRCA-like (n) | [%] |
| RAD51Cm | 13 | 0 | X | 13 | (6; 0; 7) | 100.0 | 0 | 0 |
| RAD51Dm | 3 | 1 | X | 3 | (0; 0; 3) | 75.0 | 1 | 25.0 |
| FANCMm | 2 | 3 | X | 4 | (1; 0; 3) | 80.0 | 1 | 20.0 |
| PALB2m | 3 | 2 | 1x BRCA1-methyl (s) | 2 | (1; 0; 1) | 40.0 | 3 | 60.0 |
| PALB2 + ATMm | 1 | 0 | X | 1 | (1; 0; 0) | 100.0 | 0 | 0.0 |
| ATMm | 1 | 2 | X | 3 | (0; 2; 1) | 100.0 | 0 | 0.0 |
| BRIP1m | 1 | 1 | 1x g + sMSH6 (s) * | 1 | (0; 1; 0) | 50.0 | 1 | 50.0 |
| BRIP1 + CHEK2m | 1 | 0 | X | 1 | (1; 0; 0) | 100.0 | 0 | 0.0 |
| CHEK2m | 1 | 0 | X | 0 | (0; 0; 0) | 0.0 | 1 | 100.0 |
| MRE11Am | 2 | 0 | 1x RAD51C-methyl ** | 1 | (0; 1; 0) | 50.0 | 1 | 50.0 |
| NBNm | 1 | 0 | X | 1 | (1; 0; 0) | 100.0 | 0 | 0.0 |
| CHEK1m | 1 | 0 | X | 0 | (0; 0; 0) | 0.0 | 1 | 100.0 |
| FAM175Am | 1 | 0 | X | 0 | (0; 0; 0) | 0.0 | 1 | 100.0 |
| BARD1m | 0 | 1 | X | 0 | (0; 0; 0) | 0.0 | 1 | 100.0 |
| RAD51C-methyl | 0 | 3 | 1x gMRE11A ** | 1 | (0; 0; 1) | 33.3 | 2 | 66.7 |
| HNPCC-genes | 3 | 0 | 1x gMSH6 + sMSH6 & sBRIP1 * | 0 | (0; 0; 0) | 0.0 | 3 | 100.0 |
| (2x MSH2m, 1x MSH6m) | | | | | | | | |

Samples with a class 4/5 variant in RAD51D (g: n=3, s: n=1) presented a BRCA1- and 2-like profile in 3 of 4 cases and FANCM-associated OC (g: n=2, s: n=3) in 3 of 5 samples, one sample further presented with only aBRCA1-like phenotype. All four ATM-associated samples showed a BRCA-like profile (only BRCA1-like: n=1, only BRCA2-like: n=2, BRCA1- and 2-like: n=1). Three of 6 PALB2-associated cancers presented a BRCA-like profile, including two samples with each an ATM mutation (see above) and a BRCA1 methylation. Therefore, the underlying mechanism leading to a BRCA-like phenotype was unclear. Samples with each a mutation in CHEK1 (g), FAM175A (g), and BARD1 (s) as well as three tumors with a germline mutation in HNPCC genes (MSH2 n=2, MSH6 with additional somatic MSH6- and BRIP1 mutation, n=1) were examined and showed no BRCA-like profile. Epigenetic alterations in RAD51C were rare. Four samples with a RAD51C promoter hypermethylation were analyzed, of which one was derived from a germline BRCA1 mutation carrier, presenting a BRCA1- and BRCA2-like profile, assumingly due to the known mutation. Of the three remaining samples, one was classified as BRCA1- and BRCA2-like; the other two (one of them from a gMRE11A mutation-carrier) displayed a non-BRCA-like phenotype.

### Molecular Genetic Background of BRCA-Like Samples

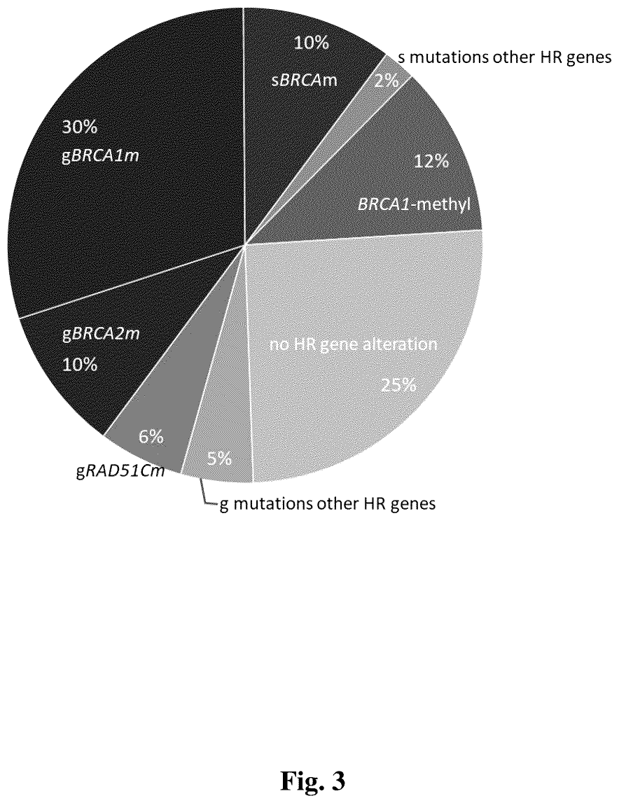

Within the cohort, 223 samples displayed a BRCA1/2-like phenotype. These profiles were associated with a germline mutation in BRCA1/2 in 89 and a somatic mutation in BRCA1/2 respectively a BRCA1 promoter hypermethylation in 49 of these samples. In another 29 different samples, a somatic or germline class 4/5 variant in another HR gene (esp. RAD51C) or aRAD51C promoter hypermethylation can explain the presence of a BRCA-like phenotype. For the remaining 56 BRCA1/2-like samples (25.1%) no aberration affecting the HR could be detected by gene panel or methylation analyses (see FIG. 3).

Regarding all OC without a genetic or epigenetic alteration in BRCA1/2 (n=155), 54.8% (n=85) display a BRCA1/2-like phenotype. Excluding all HR gene mutated cases (n=113), as the study cohort was enriched for those, the BRCA1/2-like rate was 48.7% (n=55). Furthermore, a BRCA-like phenotype was correlated with high-grade serous histology, sTP53 mutations and could be observed more often, when the patient was included with platinum-sensitive recurring disease than at primary diagnosis. A non-BRCA-like profile was seen in association with low-grade serous histology, in combination with PTEN- or PIK3CA mutations and with gHNPCC mutations (see Table 8).

In summary, BRCA1-like and BRCA2-like copy number classifiers have been identified and these signatures identify patients with germline and somatic mutations and promoter hypermethylation in BRCA1/2. In addition, we were able to investigate underlying molecular mechanisms in BRCA-like cases without a deleterious variant in BRCA1 or BRCA2.

Another advantage of this study is that after initial promising results within cross-validation of the training set and in the TCGA test dataset were obtained, the AGO-TR1 study provided a large and well characterized cohort in which we could validate the prediction of mutation status. Overall, the BRCA1-like classifier showed a convincing performance with detecting BRCA1-mutated and -methylated cancers in more than 90% of the cases. When applying both classifiers in the AGO-TR1 trial, more than 95% of all BRCA1/2-associated cancers in the cohort were detected. Extensive germline and tumor genetic analysis provided additional support for the BRCA-like class showing alterations in BRCA1/2 or other HR genes in 74.9% of cases. Thus, a convincing performance of the BRCA1-like classifier and the combination of both classifiers in detecting BRCA mutations/methylation has been shown. Additionally, around half of the non-BRCA-associated OC cases displayed a BRCA-like phenotype as well.

Table 8A-C: Rate of BRCA1-, BRCA2-like status and either according to genetic (germline and somatic), epigenetic, and clinical features. In the first section, alterations in the HR-pathway are in focus. The sample with both a germline mutation in BRCA1 and BRCA2 is analyzed within the gBRCA1 group, the sample with the germline and somatic deleterious variant in BRCA2 with the gBRCA2 group and the samples with an additional HR gene mutation (n=8) or a RAD51C methylation (n=1) in the presence of a g/sBRCA mutation are assigned to the respective BRCA group as the latter is supposed to be the superordinate feature in developing a BRCA-like profile. Three other samples with two mutual alterations (BRCA1-methyl and sBRCA2; BRCA1-methyl and sPALB2; RAD51C-methyl and gMRE11A) were mentioned in both categories, as the predominant mechanism was not obvious. Two of the three gHNPCC samples are included in the “no HR gene alteration” group as well, the other one within the “sHR gene” group.

In the following somatic TP53, PIK3CA and PTEN mutations, the histological subtype and the presence of primary or recurrent disease were analyzed irrespective of alterations in the HR-pathway. As sTP53, sPIK3CA and sPTENmutations could co-occur, the affected samples were mentioned in each subcategory.

p<0.05 is considered significant (Benjamini-Hochberg correction was applied). *marks p-values with a significant correlation with non-BRCA-like status.

G: germline, s: somatic, methyl: promoter hypermethylation, HR: homologous recombination, HNPCC: hereditary non-polyposis colorectal cancer, HGS: high grade serous ovarian cancer (OC), HGE: high grade endometrioid OC, LGS: low grade serous OC, LGE: low grade endometrioid OC, CC: clear cell cancer, muc: mucinous OC, G: grading, PR: primary OC, RE: platinum-sensitive recurrent OC.

TABLE 8A

| | | BRCA1- | | non-BRCA1- | | p- |
|---|---|---|---|---|---|---|
| | n | like | [%] | like | [%] | value |
| all | 300 | 197 | 65.7 | 103 | 34.3 | |
| gBRCA1 | 69 | 67 | 97.1 | 2 | 2.9 | <0.001 |
| gBRCA2 | 22 | 20 | 90.9 | 2 | 9.1 | 0.023 |
| gHRgenes | 31 | 21 | 67.4 | 10 | 32.1 | 1.000 |
| gRAD51C | 13 | 13 | 100.0 | 0 | 0.0 | 0.015 |
| sBRCA1 | 17 | 15 | 88.2 | 2 | 11.8 | 0.114 |
| sBRCA2 | 10 | 5 | 50.0 | 5 | 50.0 | 0.457 |
| sHRgenes | 10 | 5 | 50.0 | 5 | 50.0 | 0.457 |
| BRCA1-meth | 28 | 27 | 96.4 | 1 | 3.6 | <0.001 |
| RAD51C-meth | 3 | 1 | 33.3 | 2 | 66.7 | 0.450 |
| no HR gene | 113 | 38 | 33.6 | 75 | 66.4 | <0.001* |
| alteration | | | | | | |
| gHNPCCgenes | 3 | 0 | 0.0 | 3 | 100.0 | 0.079* |
| sTP53 | 251 | 185 | 73.7 | 66 | 26.3 | <0.001 |
| sPIK3CA | 11 | 3 | 27.3 | 8 | 72.7 | 0.003* |
| sPTEN | 10 | 2 | 20.0 | 8 | 80.0 | 0.079* |
| sPIK3CA/ | 17 | 4 | 23.5 | 14 | 76.5 | 0.001* |
| PTEN | | | | | | |
| HGS | 246 | 172 | 69.9 | 74 | 30.1 | 0.011 |
| HGE | 16 | 6 | 37.5 | 10 | 62.5 | 0.072* |
| LGS | 7 | 1 | 14.3 | 6 | 85.7 | 0.030* |
| LGE | 4 | 1 | 25.0 | 3 | 75.0 | 0.192 |
| CC | 2 | 0 | 0.0 | 2 | 100.0 | 0.192 |
| muc | 4 | 3 | 75.0 | 1 | 25.0 | 1.000 |
| serous, G | 6 | 3 | 50.0 | 3 | 50.0 | 0.560 |
| unknown | | | | | | |
| other/missing | 15 | 11 | 73.3 | 4 | 26.7 | 0.674 |
| PR | 160 | 97 | 60.6 | 63 | 39.4 | 0.052* |
| RE | 140 | 100 | 71.4 | 40 | 28.6 | 0.052 |

TABLE 8B

| | | BRCA2- | | non-BRCA2- | | p- |
|---|---|---|---|---|---|---|
| | n | like | [%] | like | [%] | value |
| all | 300 | 146 | 48.7 | 154 | 51.3 | |
| gBRCA1 | 69 | 37 | 55.2 | 32 | 46.4 | 0.867 |
| gBRCA2 | 22 | 16 | 72.7 | 6 | 27.3 | 0.090 |
| gHRgenes | 31 | 14 | 45.2 | 17 | 54.8 | 0.867 |
| gRAD51C | 13 | 7 | 53.8 | 6 | 46.2 | 0.867 |
| sBRCA1 | 17 | 15 | 88.2 | 2 | 11.8 | 0.005 |
| sBRCA2 | 10 | 6 | 60.0 | 4 | 40.0 | 0.867 |
| sHRgenes | 10 | 4 | 40.0 | 6 | 60.0 | 0.867 |
| BRCA1-meth | 28 | 15 | 53.6 | 13 | 46.4 | 0.867 |
| RAD51C-meth | 3 | 1 | 33.3 | 2 | 66.7 | 1.000 |
| no HR gene | 113 | 39 | 34.5 | 76 | 65.5 | 0.001* |
| alteration | | | | | | |
| gHNPCCgenes | 3 | 0 | 0.0 | 3 | 100.0 | 0.714 |
| sTP53 | 251 | 138 | 55.0 | 113 | 45.0 | <0.001 |
| sPIK3CA | 11 | 1 | 9.1 | 10 | 90.9 | 0.053* |
| sPTEN | 10 | 4 | 40.0 | 6 | 60.0 | 0.867 |
| sPIK3CA/ | 17 | 4 | 23.5 | 14 | 76.5 | 0.090* |
| PTEN | | | | | | |
| HGS | 246 | 128 | 52.0 | 118 | 48.0 | 0.076 |
| HGE | 16 | 8 | 50.0 | 8 | 50.0 | 1.000 |
| LGS | 7 | 0 | 0.0 | 7 | 100.0 | 0.076* |
| LGE | 4 | 3 | 75.0 | 1 | 25.0 | 0.720 |
| CC | 2 | 0 | 0.0 | 2 | 100.0 | 0.789 |
| muc | 4 | 1 | 25.0 | 3 | 75.0 | 0.789 |
| serous, G | 6 | 2 | 33.3 | 4 | 66.7 | 0.789 |
| unknown | | | | | | |
| other/missing | 15 | 4 | 26.7 | 11 | 73.3 | 0.293 |
| PR | 160 | 75 | 46.9 | 85 | 53.1 | 0.563 |
| RE | 140 | 71 | 50.7 | 69 | 49.3 | 0.563 |

TABLE 8C

| | | | | non- | | |
|---|---|---|---|---|---|---|
| | | BRCA1/ | | BRCA1/ | | p- |
| | n | 2-like | [%] | 2-like | [%] | value |
| all | 300 | 223 | 74.3 | 77 | 25.7 | |
| gBRCA1 | 69 | 67 | 97.1 | 2 | 2.9 | <0.001 |
| gBRCA2 | 22 | 22 | 100.0 | 0 | 0.0 | 0.009 |
| gHRgenes | 31 | 24 | 77.4 | 7 | 22.6 | 0.125 |
| gRAD51C | 13 | 13 | 100.0 | 0 | 0.0 | 0.050 |
| sBRCA1 | 17 | 17 | 100.0 | 0 | 0.0 | 0.021 |
| sBRCA2 | 10 | 6 | 60.0 | 4 | 40.0 | 0.341 |
| sHRgenes | 10 | 6 | 60.0 | 4 | 40.0 | 0.341 |
| BRCA1-meth | 28 | 27 | 96.4 | 1 | 3.6 | 0.010 |
| RAD51C-meth | 3 | 1 | 33.3 | 2 | 66.7 | 0.246 |
| no HR gene | 113 | 55 | 48.7 | 58 | 51.3 | <0.001* |
| alteration | | | | | | |
| gHNPCCgenes | 3 | 0 | 0.0 | 3 | 100.0 | 0.036* |
| sTP53 | 251 | 206 | 82.1 | 45 | 17.9 | <0.001 |
| sPIK3CA | 11 | 4 | 36.4 | 7 | 63.6 | 0.021* |
| sPTEN | 10 | 5 | 50.0 | 5 | 50.0 | 0.217 |
| sPIK3CA/ | 17 | 7 | 41.2 | 10 | 58.8 | 0.010* |
| PTEN | | | | | | |
| HGS | 246 | 191 | 77.6 | 55 | 22.4 | 0.036 |
| HGE | 16 | 9 | 56.3 | 7 | 43.7 | 0.272 |
| LGS | 7 | 1 | 14.3 | 6 | 85.7 | 0.011* |
| LGE | 4 | 3 | 75.0 | 1 | 25.0 | 1.000 |
| CC | 2 | 0 | 0.0 | 2 | 100.0 | 0.173 |
| muc | 4 | 3 | 75.0 | 1 | 25.0 | 1.000 |
| serous, G | 6 | 4 | 67.7 | 2 | 33.3 | 1.000 |
| unknown | | | | | | |
| other/missing | 15 | 12 | 80.0 | 3 | 20.0 | 1.000 |
| PR | 160 | 113 | 70.6 | 47 | 29.4 | 0.145 |
| RE | 140 | 110 | 78.6 | 30 | 21.4 | 0.145 |

### Example 6: Validation of the Predictive Value of the Method of the Present Disclosure to Predict the Response of a Subject Having Ovarian Cancer to an Anti-Cancer Therapy Comprising PARP Inhibitors

Recently 3 Phase III first-line studies, PAOLA-1/ENGOT-ov25 (51), PRIMA/ENGOT-ov26/GOG-3012 (52) and VELIA/GOG-3005 (53) have demonstrated that the addition of a PARP inhibitor (PARPi) to platinum-based therapy±bevacizumab improved progression-free survival in advanced ovarian cancer (AOC) patients. The benefit was greater when the tumor was HRD positive with or without a BRCA mutation. The PAOLA-1 olaparib+bevacizumab maintenance regimen was approved in USA/Europe/Japan for HRD positive patients.

Accordingly, the method of the present disclosure was tested for its capability to predict the response of a subject with OC to an anti-cancer therapy comprising PARP inhibitors. The method was tested in three phases: phase 1 (2019/12) was to bring together European RL. Phase 2 evaluated the predictive value of the method based on survival analysis on tumor samples from 85 PAOLA-1 BRCA wild type patients. Phase 3 was the final PFS evaluation on more than 350 additional patients.

### Methods

DNA samples of PAOLA1 study were obtained from Arcagy. The samples were sequenced at Genomics Core Facility of the Netherlands Cancer Institute (Phase 2, n=84) and the Cologne Center for Genomics (Phase 3, n=383). Reads were aligned, counted in bins, normalized within the dataset and to the reference training dataset, and subsequently classified as BRCA1-like (posterior probability >0.5) or non-BRCA1-like (posterior probability=<0.5).

### Results

In both phases of the trial it was observed that there is additional benefit of Olaparib in BRCA1-like patients, respectively in phase 2 hazard rate 0.609, 95% CI 0.295-1.303, p=0.201, and in phase 3 hazard rate 0.438, 95% CI 0.316-0.607, p<0.001. There was no additional benefit of Olaparib in non-BRCA1-like patients, respectively in phase 2 hazard rate 0.925, 95% CI 0.411-2.084, p=0.851, and in phase 3 hazard rate 0.959 (95% CI 0.600-1.534). The survival curves are presented in FIGS. 5 and 6.

In phase 2 four samples were flagged of poorer quality, but were analyzed with these 4 samples because of the low frequency.

In phase 3 flagged 15/375 (4%) samples were flagged as poor quality and were excluded from the presented results. 8 samples were not sequenced. In total 23/383 (6%) samples were thus not available for analysis. Current results show a 44/383 (11.4%) dropout rate.

### CONCLUSION

The results show that the predictive value of the method of the present disclosure is high and thus, OC patients that will benefit from treatment with PARP inhibitors due to their BRCA-1-like status can be distinguished from patients that will not benefit from treatment with PARP inhibitors due to their non-BRAC1-like status. Accordingly, the BRCA1-like classifier is a predictive biomarker for maintenance treatment with the PARP inhibitor olaparib in AOC.

### REFERENCES

1. Fitzmaurice C, Abate D, Abbasi N, Abbastabar H, Abd-Allah F, Abdel-Rahman 0, et al. Global, Regional, and National Cancer Incidence, Mortality, Years of Life Lost, Years Lived With Disability, and Disability-Adjusted Life-Years for 29 Cancer Groups, 1990 to 2017: A Systematic Analysis for the Global Burden of Disease Study. JAMA Oncol 2019; 5(12):1749-68.
2. National Cancer Institue. Cancer Stat Facts: Ovarian Cancer. <https://seer.cancer.gov/statfacts/html/ovary.html>. Accessed 2020 Sep. 30th.
3. du Bois A, Reuss A, Pujade-Lauraine E, Harter P, Ray-Coquard I, Pfisterer J. Role of surgical outcome as prognostic factor in advanced epithelial ovarian cancer: a combined exploratory analysis of 3 prospectively randomized phase 3 multicenter trials: by the Arbeitsgemeinschaft Gynaekologische Onkologie Studiengruppe Ovarialkarzinom (AGO-OVAR) and the Groupe d'Investigateurs Nationaux Pour les Etudes des Cancers de l'Ovaire (GINECO). Cancer 2009; 115(6):1234-44.
4. Piver M S. Treatment of ovarian cancer at the crossroads: 50 years after single-agent melphalan chemotherapy. Oncology (Williston Park) 2006; 20(10):1156, 58.
5. Ledermann J A, Raja F A, Fotopoulou C, Gonzalez-Martin A, Colombo N, Sessa C, et al. Newly diagnosed and relapsed epithelial ovarian carcinoma: ESMO Clinical Practice Guidelines for diagnosis, treatment and follow-up. Ann Oncol 2013; 24 Suppl 6:vi24-32.
6. Ledermann J A, Drew Y, Kristeleit R S. Homologous recombination deficiency and ovarian cancer. European journal of cancer (Oxford, England: 1990) 2016; 60:49-58.
7. Moynahan M E, Chiu J W, Koller B H, Jasin M. Breal controls homology-directed DNA repair. Mol Cell 1999; 4(4):511-8.
8. Moynahan M E, Pierce A J, Jasin M. BRCA2 is required for homology-directed repair of chromosomal breaks. Mol Cell 2001; 7(2):263-72.
9. Moore K, Colombo N, Scambia G, Kim B G, Oaknin A, Friedlander M, et al. Maintenance Olaparib in Patients with Newly Diagnosed Advanced Ovarian Cancer. N Engl J Med 2018; 379(26):2495-505.
10. Robson M, Im S A, Senkus E, Xu B, Domchek S M, Masuda N, et al. Olaparib for Metastatic Breast Cancer in Patients with a Germline BRCA Mutation. N Engl J Med 2017; 377(6):523-33.
11. Turner N, Tutt A, Ashworth A. Hallmarks of ‘BRCAness’ in sporadic cancers. Nat Rev Cancer 2004; 4(10):814-9.
12. Lord C J, Ashworth A. BRCAness revisited. Nat Rev Cancer 2016; 16(2):110-20.
13. Ledermann J, Harter P, Gourley C, Friedlander M, Vergote I, Rustin G, et al. Olaparib maintenance therapy in patients with platinum-sensitive relapsed serous ovarian cancer: a preplanned retrospective analysis of outcomes by BRCA status in a randomised phase 2 trial. Lancet Oncol 2014; 15(8):852-61.
14. Mirza M R, Monk B J, Herrstedt J, Oza A M, Mahner S, Redondo A, et al. Niraparib Maintenance Therapy in Platinum-Sensitive, Recurrent Ovarian Cancer. N Engl J Med 2016; 375(22):2154-64.
15. Swisher E M, Lin K K, Oza A M, Scott C L, Giordano H, Sun J, et al. Rucaparib in relapsed, platinum-sensitive high-grade ovarian carcinoma (ARIEL2 Part 1): an international, multicentre, open-label, phase 2 trial. Lancet Oncol 2017; 18(1):75-87.
16. Coleman R L, Oza A M, Lorusso D, Aghajanian C, Oaknin A, Dean A, et al. Rucaparib maintenance treatment for recurrent ovarian carcinoma after response to platinum therapy (ARIEL3): a randomised, double-blind, placebo-controlled, phase 3 trial. Lancet 2017; 390(10106):1949-61.
17. Abkevich V, Timms K M, Hennessy B T, Potter J, Carey M S, Meyer L A, et al. Patterns of genomic loss of heterozygosity predict homologous recombination repair defects in epithelial ovarian cancer. Br J Cancer 2012; 107(10):1776-82.
18. Davies H, Glodzik D, Morganella S, Yates L R, Staaf J, Zou X, et al. HRDetect is a predictor of BRCA1 and BRCA2 deficiency based on mutational signatures. Nat Med 2017; 23(4):517-25.
19. Birkbak N J, Wang Z C, Kim J Y, Eklund A C, Li Q, Tian R, et al. Telomeric allelic imbalance indicates defective DNA repair and sensitivity to DNA-damaging agents. Cancer Discov 2012; 2(4):366-75.
20. Popova T, Manie E, Rieunier G, Caux-Moncoutier V, Tirapo C, Dubois T, et al. Ploidy and large-scale genomic instability consistently identify basal-like breast carcinomas with BRCA1/2 inactivation. Cancer Res 2012; 72(21):5454-62.
21. Konstantinopoulos P A, Spentzos D, Karlan B Y, Taniguchi T, Fountzilas E, Francoeur N, et al. Gene expression profile of BRCAness that correlates with responsiveness to chemotherapy and with outcome in patients with epithelial ovarian cancer. J Clin Oncol 2010; 28(22):3555-61.
22. Timms K M, Abkevich V, Hughes E, Neff C, Reid J, Morris B, et al. Association of BRCA1/2 defects with genomic scores predictive of DNA damage repair deficiency among breast cancer subtypes. Breast Cancer Res 2014; 16(6):475.
23. Moore K N, Secord A A, Geller M A, Miller D S, Cloven N, Fleming G F, et al. Niraparib monotherapy for late-line treatment of ovarian cancer (QUADRA): a multicentre, open-label, single-arm, phase 2 trial. Lancet Oncol 2019; 20(5):636-48.
24. Coleman R L, Fleming G F, Brady M F, Swisher E M, Steffensen K D, Friedlander M, et al. Veliparib with First-Line Chemotherapy and as Maintenance Therapy in Ovarian Cancer. N Engl J Med 2019; 381(25):2403-15.
25. Gonzalez-Martin A, Pothuri B, Vergote I, DePont Christensen R, Graybill W, Mirza M R, et al. Niraparib in Patients with Newly Diagnosed Advanced Ovarian Cancer. N Engl J Med 2019; 381(25):2391-402.
26. Ray-Coquard I, Pautier P, Pignata S, Perol D, Gonzalez-Martin A, Berger R, et al. Olaparib plus Bevacizumab as First-Line Maintenance in Ovarian Cancer. N Engl J Med 2019; 381(25):2416-28.
27. European Medicines Agency. 2020. Lynparza, Opinion. <https://www.ema.europa.eu/en/medicines/human/summaries-opinion/lynparza-0>. Accessed 2020 Sep. 30.
28. Joosse S A, van Beers E H, Tielen I H, Horlings H, Peterse J L, Hoogerbrugge N, et al. Prediction of BRCA1-association in hereditary non-BRCA1/2 breast carcinomas with array-CGH. Breast Cancer Res Treat 2009; 116(3):479-89.
29. Tibshirani R, Hastie T, Narasimhan B, Chu G. Diagnosis of multiple cancer types by shrunken centroids of gene expression. Proc Natl Acad Sci USA 2002; 99(10):6567-72.
30. Vollebergh M A, Lips E H, Nederlof P M, Wessels L F, Schmidt M K, van Beers E H, et al. An aCGH classifier derived from BRCA1-mutated breast cancer and benefit ofhigh-dose platinum-based chemotherapy in HER2-negative breast cancer patients. Ann Oncol 2011; 22(7):1561-70.
31. Vollebergh M A, Lips E H, Nederlof P M, Wessels L F, Wesseling J, Vd Vijver M J, et al. Genomic patterns resembling BRCA1- and BRCA2-mutated breast cancers predict benefit of intensified carboplatin-based chemotherapy. Breast Cancer Res 2014; 16(3):R47.
32. Schouten P C, Marine F, Aulmann S, Sinn H P, van Essen H F, Ylstra B, et al. Breast cancers with a BRCA1-like DNA copy number profile recur less often than expected after high-dose alkylating chemotherapy. Clin Cancer Res 2015; 21(4):763-70.
33. Schouten P C, Gluz O, Harbeck N, Mohrmann S, Diallo-Danebrock R, Pelz E, et al. BRCA1-like profile predicts benefit of tandem high dose epirubicin-cyclophospamide-thiotepa in high risk breast cancer patients randomized in the WSG-AMO1 trial. Int J Cancer 2016; 139(4):882-9.
34. Vliek S, Jager A, Jonge-Lavrencic M, Lotz J-P, Gongalves A, Graeser M, et al. Abstract OT2-07-08: Substantially improving the cure rate of high-risk BRCA1-like breast cancer patients with personalized therapy (SUBITO)—an international randomized phase III trial. Cancer Research 2018; 78(4 Supplement):OT2-07-08-OT2-07-08.
35. Severson™, Wolf D M, Yau C, Peeters J, Wehkam D, Schouten P C, et al. The BRCAlness signature is associated significantly with response to PARP inhibitor treatment versus control in the I-SPY 2 randomized neoadjuvant setting. Breast Cancer Res 2017; 19(1):99.
36. Harter P, Hauke J, Heitz F, Reuss A, Kommoss S, Marine F, et al. Prevalence of deleterious germline variants in risk genes including BRCA1/2 in consecutive ovarian cancer patients (AGO-TR-1). PLoS One 2017; 12(10):e0186043.
37. Hauke J, Hahnen E, Schneider S, Reuss A, Richters L, Kommoss S, et al. Deleterious somatic variants in 473 consecutive individuals with ovarian cancer: results of the observational AGO-TR1 study (NCT02222883). J Med Genet 2019; 56(9):574-80.
38. Federation of Dutch Medical Scientific Societies. 2011. Human Tissue and Medical Research: Code of Conduct for responsible use (2011). <https://www.federa.org/sites/default/files/images/print_version_code_of_conduct_en glish.pdf>. Accessed 2020 Sep. 30.
39. Schouten P C, Grigoriadis A, Kuilman T, Mirza H, Watkins J A, Cooke S A, et al. Robust BRCA1-like classification of copy number profiles of samples repeated across different datasets and platforms. Mol Oncol 2015; 9(7):1274-86.
40. Schouten P C, van Dyk E, Braaf L M, Mulder L, Lips E H, de Ronde J J, et al. Platform comparisons for identification of breast cancers with a BRCA-like copy number profile. Breast Cancer Res Treat 2013; 139(2):317-27.
41. Hastie T, Tibshirani R, Friedman J. The Elements of Statistical Learning. Springer, New York, NY; 2009.
42. Bell D, Berchuck A, Birrer M, Chien J, Cramer D W, Dao F, et al. Integrated genomic analyses of ovarian carcinoma. Nature 2011; 474(7353):609-15.
43. Domanska K, Malander S, Staaf J, Karlsson A, Borg A, Jonsson G, et al. Genetic profiles distinguish different types of hereditary ovarian cancer. Oncol Rep 2010; 24(4):885-95.
44. Kamieniak M M, Munoz-Repeto I, Rico D, Osorio A, Urioste M, Garcia-Donas J, et al. DNA copy number profiling reveals extensive genomic loss in hereditary BRCA1 and BRCA2 ovarian carcinomas. Br J Cancer 2013; 108(8):1732-42.
45. Maxwell K N, Wubbenhorst B, Wenz B M, De Sloover D, Pluta J, Emery L, et al. BRCA locus-specific loss of heterozygosity in germline BRCA1 and BRCA2 carriers. Nature Communications 2017; 8(1):319.
46. Macintyre G, Goranova T E, De Silva D, Ennis D, Piskorz A M, Eldridge M, et al. Copy number signatures and mutational processes in ovarian carcinoma. Nature genetics 2018; 50(9):1262-70.
47. Prahallad A, Sun C, Huang S, Di Nicolantonio F, Salazar R, Zecchin D, et al. Unresponsiveness of colon cancer to BRAF(V600E) inhibition through feedback activation of EGFR. Nature 2012; 483(7387):100-3.
48. Alexandrov L B, Kim J, Haradhvala N J, Huang M N, Tian Ng A W, Wu Y, et al. The repertoire of mutational signatures in human cancer. Nature 2020; 578(7793):94-101.
49. Watkins J A, Irshad S, Grigoriadis A, Tutt A N. Genomic scars as biomarkers of homologous recombination deficiency and drug response in breast and ovarian cancers. Breast Cancer Res 2014; 16(3):211.
50. Castroviejo-Bermejo M, Cruz C, Llop-Guevara A, Gutierrez-Enriquez S, Ducy M, Ibrahim Y H, et al. A RAD51 assay feasible in routine tumor samples calls PARP inhibitor response beyond BRCA mutation. EMBO molecular medicine 2018; 10(12).
51. Ray-Coquard I, Pautier P, Pignata S, Perol D, González-Martin A, Berger R, et al. Olaparib plus Bevacizumab as First-Line Maintenance in Ovarian Cancer. N Engl J Med. 2019; 381(25):2416-2428.
52. González-Martin A, Pothuri B, Vergote I, DePont Christensen R, Graybill W, Mirza M R, et al. Niraparib in Patients with Newly Diagnosed Advanced Ovarian Cancer. N Engl J Med. 2019; 381(25):2391-2402.
53. Coleman R L, Fleming G F, Brady M F, Swisher E M, Steffensen K D, Friedlander M, et al. Veliparib with First-Line Chemotherapy and as Maintenance Therapy in Ovarian Cancer. N Engl J Med. 2019; 381(25):2403-2415.
54. Koole S N, Schouten P C, Hauke J, Kluin R J C, Nederlof P, Richters L K, et al. Effect of HIPEC according to HRD/BRCAwt genomic profile in stage III ovarian cancer: Results from the phase III OVHIPEC trial. Int J Cancer. 2022; 151(8):1394-1404.

1. An in vitro method of determining homologous recombination (HR) deficiency status in a subject having ovarian cancer, wherein the method includes the following steps:
(a) determining copy number variation (CNV) of a genomic locus in a DNA test sample of cancer cells from the subject, wherein the genomic locus is selected from a pool of genomic loci comprising or consisting of the loci set forth in Table 1 and/or Table 2;
wherein similarity between the CNVs of the pool of genomic loci in the DNA test sample and the CNVs of the pool of genomic loci of a reference sample of BRCA-like mutated cancer cells identifies the subject as HR deficient; and/or similarity between the CNVs in the pool of genomic loci in the DNA of the test sample and the CNVs in the pool of genomic loci of the control sample of BRCA non-mutated cancer cells identifies the subject as not being HR deficient, wherein similarity is the distance measure between centroids of the control sample, the reference sample, and the test sample and wherein a similarity score is calculated by comparison of the shrunken centroids of the test sample with the shrunken centroids of the reference sample and of the control sample, wherein the shrunken centroids are derived from the CNVs of the genomic loci; and optionally
(b) transmitting the result to the subject or a third party.

2. An in vitro method of determining homologous recombination (HR) deficiency status in a subject having ovarian cancer, wherein the method includes the following steps:
(a) determining copy number variation (CNV) of a genomic locus in a DNA test sample of cancer cells from the subject, wherein the locus is selected from a pool of genomic loci comprising or consisting of the loci set forth in Table 1 and/or the loci set forth in Table 2,
(b) providing a shrunken centroid value which is derived from the CNV of the genomic locus; and
wherein a posterior probability score of ≥0.5 identifies the subject as HR deficient; and/or wherein a posterior probability score of <0.5 identifies the subject as not being HR deficient, wherein the posterior probability score is obtainable by comparison of the shrunken centroid values derived from the CNVs of the pool of genomic loci in the DNA of the cancer cells in the test sample from the subject with the shrunken centroid values derived from the CNVs of a reference set of samples of BRCA-like mutated cancer cells and of control samples of BRCA non-mutated cancer cells, and optionally
(c) transmitting the result to the subject or a third party.

3. The method according to claim 1, wherein a posterior probability score of ≥0.5 and a similarity between the CNVs of the test sample and the CNVs of the reference sample, respectively identifies the subject as being a likely responder to an anti-cancer therapy; or wherein a posterior probability score of <0.5 and a similarity between the CNVs of the test sample and the CNVs of the control sample, respectively identifies the subject a not being a likely responder to an anti-cancer therapy.

4. A method of in vitro predicting the response of a subject having ovarian cancer to an anti-cancer therapy, wherein the method includes the steps (a) of claim 1 and the following steps:
(b) determining copy number variation (CNV) of a genomic locus in a DNA test sample of cancer cells from the subject, wherein the locus is selected from a pool of genomic loci comprising or consisting of the loci set forth in Table 1 and/or the loci set forth in Table 2,
(c) providing a shrunken centroid value which is derived from the CNV of the genomic locus; and
wherein a posterior probability score of ≥0.5 identifies the subject as HR deficient; and/or wherein a posterior probability score of <0.5 identifies the subject as not being HR deficient, wherein the posterior probability score is obtainable by comparison of the shrunken centroid values derived from the CNVs of the pool of genomic loci in the DNA of the cancer cells in the test sample from the subject with the shrunken centroid values derived from the CNVs of a reference set of samples of BRCA-like mutated cancer cells and of control samples of BRCA non-mutated cancer cells, wherein
(i) a posterior probability score of ≥0.5 and a similarity between the CNVs of the test sample and the CNVs of the reference sample, respectively indicates an increased likelihood that the subject will respond to the anti-cancer therapy, and/or
(ii) a posterior probability score of <0.5 and a similarity between the CNVs of the test sample and the CNVs of the control sample, respectively indicates an increased likelihood that the subject will not respond to the anti-cancer therapy, and optionally
(iii) transmitting the result to the subject or a third party.

5. The method according to claim 3, wherein the anti-cancer therapy is selected from homologous recombination deficiency-targeting drugs, drugs that directly cause double strand DNA breaks, drugs that indirectly cause double strand DNA breaks and/or where the drug has been identified in mechanistic studies to target homologous recombination deficiency.

6. The method of claim 1, wherein the anti-cancer therapy comprises poly(ADP-ribose) polymerase inhibitors (PARPi) and/or platinum compounds.

7. The method of claim 6, wherein the PARPi is veliparib, niraparib, or olaparib.

8. The method of claim 6, wherein the anti-cancer therapy with platinum compounds includes administration as hyperthermic intraperitoneal chemotherapy (HIPEC).

9. The method of claim 1, wherein the determination of the CNVs is performed by DNA sequencing.

10. The method of claim 1, wherein the determination of the CNVs is performed prior to administration of the anti-cancer therapy.

11. The method of claim 1 further comprising recommending a subject which
(a) has been identified as being HR deficient and/or having an increased likelihood of responding to the anti-cancer therapy, a treatment regimen comprising homologous recombination deficiency-targeting drugs, drugs that directly cause double strand DNA breaks, drugs that indirectly cause double strand DNA breaks and/or where the drug has been identified in mechanistic studies to target homologous recombination deficiency; or
(b) has been identified as not being HR deficient and not having an increased likelihood of responding to the anti-cancer therapy, a treatment regimen not comprising homologous recombination deficiency-targeting drugs, drugs that directly cause double strand DNA breaks, drugs that indirectly cause double strand DNA breaks and/or where the drug has been identified in mechanistic studies to target homologous recombination deficiency or a treatment regime which comprises standard therapy in the specific clinical setting of the patient.

12. A treatment regime comprising homologous recombination deficiency-targeting drugs, drugs that directly cause double strand DNA breaks, and/or drugs that indirectly cause double strand DNA breaks, or that otherwise have been identified as specifically targeting homologous recombination deficient tumors for use in treating ovarian cancer in a subject, wherein said subject has been identified as HR deficient and identified to have an increased likelihood of responding to the anti-cancer treatment regimen according to claim 1.

13. The treatment regime according to claim 12, which comprises poly(ADP-ribose) polymerase inhibitors (PARPi) and/or platinum compounds.

14. The treatment regime according to claim 13, wherein the PARPi is veliparib, niraparib, or olaparib.

15. The treatment regimen according to claim 13, wherein the platinum compounds include administration as hyperthermic intraperitoneal chemotherapy (HIPEC).

16. The method according to claim 2, wherein a posterior probability score of ≥0.5 and a similarity between the CNVs of the test sample and the CNVs of the reference sample, respectively identifies the subject as being a likely responder to an anti-cancer therapy; or wherein a posterior probability score of <0.5 and a similarity between the CNVs of the test sample and the CNVs of the control sample, respectively identifies the subject a not being a likely responder to an anti-cancer therapy.

17. The method according to claim 4, wherein the anti-cancer therapy is selected from homologous recombination deficiency-targeting drugs, drugs that directly cause double strand DNA breaks, drugs that indirectly cause double strand DNA breaks and/or where the drug has been identified in mechanistic studies to target homologous recombination deficiency.
